(19) United States
(12) Patent Application Publication
(10) Pub. No.: US 2004/0180354 A1
(43) Pub. Date: Sep. 16, 2004

(54) Epitope sequences

(76) Inventors: John J.L. Simard, Vancouver (CA); David C. Diamond, West Hills, CA (US); Liping Liu, Northridge, CA (US); Zheng Liu, Woodland Hills, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/657,022

(22) Filed: Sep. 5, 2003

Related U.S. Application Data
(60) Provisional application No. 60/409,123, filed on Sep. 6, 2002.

(51) Int. Cl. C12Q 1/68; C07H 21/04; C07K 14/74

(52) U.S. Cl. 435/6; 435/69.1; 530/350; 435/325; 536/23.5

(57) ABSTRACT

Disclosed herein are polypeptides, including epitopes, clusters, and antigens. Also disclosed are compositions that include said polypeptides and methods for their use.

### CROSS REFERENCE TO RELATED APPLICATIONS

This application claims priority under 35 U.S.C. 119(e) to U.S. Provisional Patent Application Serial No. 60/409,123, filed on Sep. 6, 2002, entitled “EPITOPE SEQUENCES,” and which provisional application is incorporated herein by reference in its entirety, including the compact disks submitted with the provisional application.

### BACKGROUND OF THE INVENTION

1. Field of the Invention

The present invention generally relates to peptides, and nucleic acids encoding peptides, that are useful epitopes of target-associated antigens. More specifically, the invention relates to epitopes that have a high affinity for MHC class I and that are produced by target-specific proteasomes.

2. Description of the Related Art

Neoplasia and the Immune System

The neoplastic disease state commonly known as cancer is thought to result generally from a single cell growing out of control. The uncontrolled growth state typically results from a multi-step process in which a series of cellular systems fail, resulting in the genesis of a neoplastic cell. The resulting neoplastic cell rapidly reproduces itself, forms one or more tumors, and eventually may cause the death of the host.

Because the progenitor of the neoplastic cell shares the host's genetic material, neoplastic cells are largely unassailed by the host's immune system. During immune surveillance, the process in which the host's immune system surveys and localizes foreign materials, a neoplastic cell will appear to the host's immune surveillance machinery as a “self” cell.

Viruses and the Immune System

In contrast to cancer cells, virus infection involves the expression of clearly non-self antigens. As a result, many virus infections are successfully dealt with by the immune system with minimal clinical sequela. Moreover, it has been possible to develop effective vaccines for many of those infections that do cause serious disease. A variety of vaccine approaches have been used successfully to combat various diseases. These approaches include subunit vaccines consisting of individual proteins produced through recombinant DNA technology. Notwithstanding these advances, the selection and effective administration of minimal epitopes for use as viral vaccines has remained problematic.

In addition to the difficulties involved in epitope selection stands the problem of viruses that have evolved the capability of evading a host's immune system. Many viruses, especially viruses that establish persistent infections, such as members of the herpes and pox virus families, produce immunomodulatory molecules that permit the virus to evade the host's immune system. The effects of these immunomodulatory molecules on antigen presentation may be overcome by the targeting of select epitopes for administration as immunogenic compositions. To better understand the interaction of neoplastic cells and virally infected cells with the host's immune system, a discussion of the system's components follows below.

The immune system functions to discriminate molecules endogenous to an organism (“self” molecules) from material exogenous or foreign to the organism (“non-self” molecules). The immune system has two types of adaptive responses to foreign bodies based on the components that mediate the response: a humoral response and a cell-mediated response. The humoral response is mediated by antibodies, while the cell-mediated response involves cells classified as lymphocytes. Recent anticancer and antiviral strategies have focused on mobilizing the host immune system as a means of anticancer or antiviral treatment or therapy.

The immune system functions in three phases to protect the host from foreign bodies: the cognitive phase, the activation phase, and the effector phase. In the cognitive phase, the immune system recognizes and signals the presence of a foreign antigen or invader in the body. The foreign antigen can be, for example, a cell surface marker from a neoplastic cell or a viral protein. Once the system is aware of an invading body, antigen specific cells of the immune system proliferate and differentiate in response to the invader-triggered signals. The last stage is the effector stage in which the effector cells of the immune system respond to and neutralize the detected invader.

An array of effector cells implements an immune response to an invader. One type of effector cell, the B cell, generates antibodies targeted against foreign antigens encountered by the host. In combination with the complement system, antibodies direct the destruction of cells or organisms bearing the targeted antigen. Another type of effector cell is the natural killer cell (NK cell), a type of lymphocyte having the capacity to spontaneously recognize and destroy a variety of virus infected cells as well as malignant cell types. The method used by NK cells to recognize target cells is poorly understood.

Another type of effector cell, the T cell, has members classified into three subcategories, each playing a different role in the immune response. Helper T cells secrete cytokines which stimulate the proliferation of other cells necessary for mounting an effective immune response, while suppressor T cells down-regulate the immune response. A third category of T cell, the cytotoxic T cell (CTL), is capable of directly lysing a targeted cell presenting a foreign antigen on its surface.

The Major Histocompatibility Complex and T Cell Target Recognition

T cells are antigen-specific immune cells that function in response to specific antigen signals. B lymphocytes and the antibodies they produce are also antigen-specific entities. However, unlike B lymphocytes, T cells do not respond to antigens in a free or soluble form. For a T cell to respond to an antigen, it requires the antigen to be processed to peptides which are then bound to a presenting structure encoded in the major histocompatibility complex (MHC). This requirement is called “MHC restriction” and it is the mechanism by which T cells differentiate “self” from “non-self” cells. If an antigen is not displayed by a recognizable MHC molecule, the T cell will not recognize and act on the antigen signal. T cells specific for a peptide bound to a recognizable MHC molecule bind to these MHC-peptide complexes and proceed to the next stages of the immune response.

There are two types of MHC, class I MHC and class II MHC. T Helper cells (CD4⁺) predominately interact with class II MHC proteins while cytolytic T cells (CD8⁺) predominately interact with class I MHC proteins. Both classes of MHC protein are transmembrane proteins with a majority of their structure on the external surface of the cell. Additionally, both classes of MHC proteins have a peptide binding cleft on their external portions. It is in this cleft that small fragments of proteins, endogenous or foreign, are bound and presented to the extracellular environment.

Cells called “professional antigen presenting cells” (pAPCs) display antigens to T cells using the MHC proteins but additionally express various co-stimulatory molecules depending on the particular state of differentiation/activation of the pAPC. When T cells, specific for the peptide bound to a recognizable MHC protein, bind to these MHC-peptide complexes on pAPCs, the specific co-stimulatory molecules that act upon the T cell direct the path of differentiation/activation taken by the T cell. That is, the co-stimulation molecules affect how the T cell will act on antigenic signals in future encounters as it proceeds to the next stages of the immune response.

As discussed above, neoplastic cells are largely ignored by the immune system. A great deal of effort is now being expended in an attempt to harness a host's immune system to aid in combating the presence of neoplastic cells in a host. One such area of research involves the formulation of anticancer vaccines.

Anticancer Vaccines

Among the various weapons available to an oncologist in the battle against cancer is the immune system of the patient. Work has been done in various attempts to cause the immune system to combat cancer or neoplastic diseases. Unfortunately, the results to date have been largely disappointing. One area of particular interest involves the generation and use of anticancer vaccines.

To generate a vaccine or other immunogenic composition, it is necessary to introduce to a subject an antigen or epitope against which an immune response may be mounted. Although neoplastic cells are derived from and therefore are substantially identical to normal cells on a genetic level, many neoplastic cells are known to present tumor-associated antigens (TuAAs). In theory, these antigens could be used by a subject's immune system to recognize these antigens and attack the neoplastic cells. In reality, however, neoplastic cells generally appear to be ignored by the host's immune system.

A number of different strategies have been developed in an attempt to generate vaccines with activity against neoplastic cells. These strategies include the use of tumor-associated antigens as immunogens. For example, U.S. Pat. No. 5,993,828, describes a method for producing an immune response against a particular subunit of the Urinary Tumor Associated Antigen by administering to a subject an effective dose of a composition comprising inactivated tumor cells having the Urinary Tumor Associated Antigen on the cell surface and at least one tumor associated antigen selected from the group consisting of GM-2, GD-2, Fetal Antigen and Melanoma Associated Antigen. Accordingly, this patent describes using whole, inactivated tumor cells as the immunogen in an anticancer vaccine.

Another strategy used with anticancer vaccines involves administering a composition containing isolated tumor antigens. In one approach, MAGE-A1 antigenic peptides were used as an immunogen. (See Chaux, P., et al., “Identification of Five MAGE-A1 Epitopes Recognized by Cytolytic T Lymphocytes Obtained by In Vitro Stimulation with Dendritic Cells Transduced with MAGE-A1,” J. Immunol., 163(5):2928-2936 (1999)). There have been several therapeutic trials using MAGE-A1 peptides for vaccination, although the effectiveness of the vaccination regimes was limited. The results of some of these trials are discussed in Vose, J. M., “Tumor Antigens Recognized by T Lymphocytes,” 10^{th} European Cancer Conference, Day 2, Sep. 14, 1999.

In another example of tumor associated antigens used as vaccines, Scheinberg, et al. treated 12 chronic myelogenous leukemia (CML) patients already receiving interferon (IFN) or hydroxyurea with 5 injections of class I-associated bcr-abl peptides with a helper peptide plus the adjuvant QS-21. Scheinberg, D. A., et al., “BCR-ABL Breakpoint Derived Oncogene Fusion Peptide Vaccines Generate Specific Immune Responses in Patients with Chronic Myelogenous Leukemia (CML) [Abstract 1665], American Society of Clinical Oncology 35^{th} Annual Meeting, Atlanta (1999). Proliferative and delayed type hypersensitivity (DTH) T cell responses indicative of T-helper activity were elicited, but no cytolytic killer T cell activity was observed within the fresh blood samples.

Additional examples of attempts to identify TuAAs for use as vaccines are seen in the recent work of Cebon, et al. and Scheibenbogen, et al. Cebon, et al. immunized patients with metastatic melanoma using intradermallly administered MART-I₂₆₋₃₅ peptide with IL-12 in increasing doses given either subcutaneously or intravenously. Of the first 15 patients, 1 complete remission, 1 partial remission, and 1 mixed response were noted. Immune assays for T cell generation included DTH, which was seen in patients with or without IL-12. Positive CTL assays were seen in patients with evidence of clinical benefit, but not in patients without tumor regression. Cebon, et al., “Phase I Studies of Immunization with Melan-A and IL-12 in HLA A2+ Positive Patients with Stage III and IV Malignant Melanoma,” [Abstract 1671], American Society of Clinical Oncology 35^{th} Annual Meeting, Atlanta (1999).

Scheibenbogen, et al. immunized 18 patients with 4 HLA class I restricted tyrosinase peptides, 16 with metastatic melanoma and 2 adjuvant patients. Scheibenbogen, et al., “Vaccination with Tyrosinase peptides and GM-CSF in Metastatic Melanoma: a Phase 11 Trial,” [Abstract 1680], American Society of Clinical Oncology 35^{th} Annual Meeting, Atlanta (1999). Increased CTL activity was observed in 4/15 patients, 2 adjuvant patients, and 2 patients with evidence of tumor regression. As in the trial by Cebon, et al., patients with progressive disease did not show boosted immunity. In spite of the various efforts expended to date to generate efficacious anticancer vaccines, no such composition has yet been developed.

Antiviral Vaccines

Vaccine strategies to protect against viral diseases have had many successes. Perhaps the most notable of these is the progress that has been made against the disease small pox, which has been driven to extinction. The success of the polio vaccine is of a similar magnitude.

Viral vaccines can be grouped into three classifications: live attenuated virus vaccines, such as vaccinia for small pox, the Sabin poliovirus vaccine, and measles mumps and rubella; whole killed or inactivated virus vaccines, such as the Salk poliovirus vaccine, hepatitis A virus vaccine and the typical influenza virus vaccines; and subunit vaccines, such as hepatitis B. Due to their lack of a complete viral genome, subunit vaccines offer a greater degree of safety than those based on whole viruses.

The paradigm of a successful subunit vaccine is the recombinant hepatitis B vaccine based on the viruses envelope protein. Despite much academic interest in pushing the reductionist subunit concept beyond single proteins to individual epitopes, the efforts have yet to bear much fruit. Viral vaccine research has also concentrated on the induction of an antibody response although cellular responses also occur. However, many of the subunit formulations are particularly poor at generating a CTL response.

### SUMMARY OF THE INVENTION

Previous methods of priming professional antigen presenting cells (pAPCs) to display target cell epitopes have relied simply on causing the pAPCs to express target-associated antigens (TAAs), or epitopes of those antigens which are thought to have a high affinity for MHC I molecules. However, the proteasomal processing of such antigens results in presentation of epitopes on the pAPC that do not correspond to the epitopes present on the target cells.

Using the knowledge that an effective cellular immune response requires that pAPCs present the same epitope that is presented by the target cells, the present invention provides epitopes that have a high affinity for MHC I, and that correspond to the processing specificity of the housekeeping proteasome, which is active in peripheral cells. These epitopes thus correspond to those presented on target cells. The use of such epitopes in compositions, such as vaccines and other immunogenic compositions (including pharmaceutical and immunotherapeutic compositions) can activate the cellular immune response to recognize the correctly processed TAA and can result in removal of target cells that present such epitopes. In some embodiments, the housekeeping epitopes provided herein can be used in combination with immune epitopes, generating a cellular immune response that is competent to attack target cells both before and after interferon induction. In other embodiments the epitopes are useful in the diagnosis and monitoring of the target-associated disease and in the generation of immunological reagents for such purposes.

Embodiments of the invention relate to isolated epitopes, antigens and/or polypeptides. The isolated antigens and/or polypeptides can include the epitopes. Preferred embodiments include an epitope or antigen having the sequence as disclosed in Tables 1A or 1B. Other embodiments can include an epitope cluster comprising a polypeptide from Tables 1A or 1B. Further, embodiments include a polypeptide having substantial similarity to the already mentioned epitopes, polypeptides, antigens, or clusters. Other preferred embodiments include a polypeptide having functional similarity to any of the above. Still further embodiments relate to a nucleic acid encoding the polypeptide of any of the epitopes, clusters, antigens, and polypeptides from Tables 1A or 1B and mentioned herein.

For purposes of the following summary and discussion of other embodiments of the invention, reference to “the epitope,” “the epitopes,” or “epitope from Tables 1A or 1B” may include without limitation to all of the foregoing forms of the epitope including an epitope with the sequence set forth in the Tables or elsewhere herein, a cluster comprising such an epitope or epitopes, a polypeptide having substantial or functional similarity to those epitopes or clusters, and the like.

The polypeptide or epitope can be immunologically active. The polypeptide comprising the epitope can be less than about 30 amino acids in length, more preferably, the polypeptide is 8 to 10 amino acids in length, for example. Substantial or functional similarity can include addition of at least one amino acid, for example, and the at least one additional amino acid can be at an N-terminus of the polypeptide. The substantial or functional similarity can include a substitution of at least one amino acid.

The epitope, cluster, or polypeptide comprising the same can have affinity to an HLA-A2 molecule. The affinity can be determined by an assay of binding, by an assay of restriction of epitope recognition, by a prediction algorithm, and the like. The epitope, cluster, or polypeptide comprising the same can have affinity to an HLA-B7, HLA-B51 molecule, and the like.

In preferred embodiments the polypeptide can be a housekeeping epitope. The epitope or polypeptide can correspond to an epitope displayed on a tumor cell, to an epitope displayed on a neovasculature cell, and the like. The epitope or polypeptide can be an immune epitope. The epitope, cluster and/or polypeptide can be a nucleic acid. The epitope, cluster and/or polypeptide can be encoded by a nucleic acid.

Other embodiments relate to compositions, including pharmaceutical or immunogenic compositions comprising the polypeptides, including an epitope from Tables 1A or 1B, a cluster, or a polypeptide comprising the same, and a pharmaceutically acceptable adjuvant, carrier, diluent, excipient, and the like. The adjuvant can be a polynucleotide. The polynucleotide can include a dinucleotide, which can be CpG, for example. The adjuvant can be encoded by a polynucleotide. The adjuvant can be a cytokine and the cytokine can be, for example, GM-CSF.

The compositions can further include a professional antigen-presenting cell (pAPC). The pAPC can be a dendritic cell, for example. The composition can further include a second epitope. The second epitope can be a polypeptide, a nucleic acid, a housekeeping epitope, an immune epitope, and the like.

Still further embodiments relate to compositions, including pharmaceutical and immunogenic compositions that include any of the nucleic acids discussed herein, including those that encode polypeptides that comprise epitopes or antigens from Tables 1A or 1B. Such compositions can include a pharmaceutically acceptable adjuvant, carrier, diluent, excipient, and the like.

Other embodiments relate to recombinant constructs that include such a nucleic acid as described herein, including those that encode polypeptides that comprise epitopes or antigens from Tables 1A or 1B. The constructs can further include a plasmid, a viral vector, an artificial chromosome, and the like. The construct can further include a sequence encoding at least one feature, such as for example, a second epitope, an IRES, an ISS, an NIS, a ubiquitin, and the like.

Further embodiments relate to purified antibodies that specifically bind to at least one of the epitopes in Tables 1A or 1B. Other embodiments relate to purified antibodies that specifically bind to a peptide-MHC protein complex comprising an epitope disclosed in Tables 1A or 1B or any other suitable epitope. The antibody from any embodiment can be a monoclonal antibody or a polyclonal antibody.

Still other embodiments relate to multimeric MHC-peptide complexes that include an epitope, such as, for example, an epitope disclosed in Tables 1A or 1B. Also, contemplated are antibodies specific for the complexes.

Embodiments relate to isolated T cells expressing a T cell receptor specific for an MHC-peptide complex. The complex can include an epitope, such as, for example, an epitope disclosed in Tables 1A or 1B. The T cell can be produced by an in vitro immunization and can be isolated from an immunized animal. Embodiments relate to T cell clones, including cloned T cells, such as those discussed above. Embodiments also relate to polyclonal population of T cells. Such populations can include a T cell, as described above, for example.

Still further embodiments relate to compositions, including pharmaceutical and immunogenic compositions that include a T cell, such as those described above, for example, and a pharmaceutically acceptable adjuvant, carrier, diluent, excipient, and the like.

Embodiments of the invention relate to isolated protein molecules comprising the binding domain of a T cell receptor specific for an MHC-peptide complex. The complex can include an epitope as disclosed in Tables 1A or 1B. The protein can be multivalent. Other embodiments relate to isolated nucleic acids encoding such proteins. Still further embodiments relate to recombinant constructs that include such nucleic acids.

Other embodiments of the invention relate to host cells expressing a recombinant construct as described above and elsewhere herein. The host cells can include constructs encoding an epitope, a cluster or a polypeptide comprising said epitope or said cluster. The epitope or epitope cluster can be one or more of those disclosed in Tables 1A or 1B, for example, and as otherwise defined. The host cell can be a dendritic cell, macrophage, tumor cell, tumor-derived cell, a bacterium, fungus, protozoan, and the like. Embodiments also relate to compositions, including pharmaceutical and immunogenic compositions that include a host cell, such as those discussed herein, and a pharmaceutically acceptable adjuvant, carrier, diluent, excipient, and the like.

Still other embodiments relate to compositions including immunogenic compositions, such as for example, vaccines or immunotherapeutic compositions. The compositions can include at least one component, such as, for example, an epitope disclosed in Tables 1A or 1B or otherwise described herein; a cluster that includes such an epitope, an antigen or polypeptide that includes such an epitope; a composition as described above and herein; a construct as described above and herein, a T cell, a construct comprising a nucleic acid encoding a T cell receptor binding domain specific for an MHC-peptide complex and compositions including the same, a host cell as described above and herein, and compositions comprising the same.

Further embodiments relate to methods of treating an animal. The methods can include administering to an animal a composition, including a pharmaceutical or an immunogenic composition, such as, a vaccine or immunotherapeutic composition, including those disclosed above and herein. The administering step can include a mode of delivery, such as, for example, transdermal, intranodal, perinodal, oral, intravenous, intradermal, intramuscular, intraperitoneal, mucosal, aerosol inhalation, instillation, and the like. The method can further include a step of assaying to determine a characteristic indicative of a state of a target cell or target cells. The method can include a first assaying step and a second assaying step, wherein the first assaying step precedes the administering step, and wherein the second assaying step follows the administering step. The method can further include a step of comparing the characteristic determined in the first assaying step with the characteristic determined in the second assaying step to obtain a result. The result can be for example, evidence of an immune response, a diminution in number of target cells, a loss of mass or size of a tumor comprising target cells, a decrease in number or concentration of an intracellular parasite infecting target cells, and the like.

Embodiments relate to methods of evaluating immunogenicity of a composition, including a vaccine or an immunotherapeutic composition. The methods can include administering to an animal a vaccine or immunotherapeutic, such as those described above and elsewhere herein, and evaluating immunogenicity based on a characteristic of the animal. The animal can be MHC-transgenic.

Other embodiments relate to methods of evaluating immunogenicity that include in vitro stimulation of a T cell with the vaccine or immunotherapeutic composition, such as those described above and elsewhere herein, and evaluating immunogenicity based on a characteristic of the T cell. The stimulation can be a primary stimulation.

Still further embodiments relate to methods of making a passive/adoptive immunotherapeutic. The methods can include combining a T cell or a host cell, such as those described above and elsewhere herein, with a pharmaceutically acceptable adjuvant, carrier, diluent, excipient, and the like.

Other embodiments relate to methods of determining specific T cell frequency, and can include the step of contacting T cells with a MHC-peptide complex comprising an epitope disclosed in Tables 1A or 1B, or a complex comprising a cluster or antigen comprising such an epitope. The contacting step can include at least one feature, such as, for example, immunization, restimulation, detection, enumeration, and the like. The method can further include ELISPOT analysis, limiting dilution analysis, flow cytometry, in situ hybridization, the polymerase chain reaction, any combination thereof, and the like.

Embodiments relate to methods of evaluating immunologic response. The methods can include the above-described methods of determining specific T cell frequency carried out prior to and subsequent to an immunization step.

Other embodiments relate to methods of evaluating immunologic response. The methods can include determining frequency, cytokine production, or cytolytic activity of T cells, prior to and subsequent to a step of stimulation with MHC-peptide complexes comprising an epitope, such as, for example an epitope from Tables 1A or 1B, a cluster or a polypeptide comprising such an epitope.

Further embodiments relate to methods of diagnosing a disease. The methods can include contacting a subject tissue with at least one component, including, for example, a T cell, a host cell, an antibody, a protein, including those described above and elsewhere herein; and diagnosing the disease based on a characteristic of the tissue or of the component. The contacting step can take place in vivo or in vitro, for example.

Still other embodiments relate to methods of making a composition, including for example, a vaccine. The methods can include combining at least one component. For example, the component can be an epitope, a composition, a construct, a T cell, a host cell; including any of those described above and elsewhere herein, and the like, with a pharmaceutically acceptable adjuvant, carrier, diluent, excipient, and the like.

Embodiments relate to computer readable media having recorded thereon the sequence of any one of SEQ ID NOS: 108-610, in a machine having a hardware or software that calculates the physical, biochemical, immunologic, molecular genetic properties of a molecule embodying said sequence, and the like.

Still other embodiments relate to methods of treating an animal. The methods can include combining the method of treating an animal that includes administering to the animal a vaccine or immunotherapeutic composition, such as described above and elsewhere herein, combined with at least one mode of treatment, including, for example, radiation therapy, chemotherapy, biochemotherapy, surgery, and the like.

Further embodiments relate to isolated polypeptides that include an epitope cluster. In preferred embodiments the cluster can be from a target-associated antigen having the sequence as disclosed in any one of Tables 68-73, wherein the amino acid sequence includes not more than about 80% of the amino acid sequence of the antigen.

Other embodiments relate to immunogenic compositions, including vaccines or immunotherapeutic products that include an isolated peptide as described above and elsewhere herein. Still other embodiments relate to isolated polynucleotides encoding a polypeptide as described above and elsewhere herein. Other embodiments relate vaccines or immunotherapeutic products that include these polynucleotides. The polynucleotide can be DNA, RNA, and the like.

Still further embodiments relate to kits comprising a delivery device and any of the embodiments mentioned above and elsewhere herein. The delivery device can be a catheter, a syringe, an internal or external pump, a reservoir, an inhaler, microinjector, a patch, and any other like device suitable for any route of delivery. As mentioned, the kit, in addition to the delivery device also includes any of the embodiments disclosed herein. For example, without limitations, the kit can include an isolated epitope, a polypeptide, a cluster, a nucleic acid, an antigen, a pharmaceutical composition that includes any of the foregoing, an antibody, a T cell, a T cell receptor, an epitope-MHC complex, a vaccine, an immunotherapeutic, and the like. The kit can also include items such as detailed instructions for use and any other like item.

### BRIEF DESCRIPTION OF THE DRAWINGS

FIGS. 1A-C is a sequence alignment of NY-ESO-1 and several similar protein sequences.

FIG. 2 graphically represents a plasmid vaccine backbone useful for delivering nucleic acid-encoded epitopes.

FIGS. 3A and 3B are FACS profiles showing results of HLA-A2 binding assays for tyrosinase₂₀₇₋₂₁₅ and tyrosinase_{208-216.}

FIG. 3C shows cytolytic activity against a tyrosinase epitope by human CTL induced by in vitro immunization.

FIG. 4 is a T=120 min. time point mass spectrum of the fragments produced by proteasomal cleavage of SSX-2_{31-168.}

FIG. 5 shows a binding curve for HLA-A2:SSX-2₄₁₋₄₉ with controls.

FIG. 6 shows specific lysis of SSX-2₄₁₋₄₉-pulsed targets by CTL from SSX-2₄₁₋₄₉-immunized HLA-A2 transgenic mice.

FIGS. 7A, B, and C show results of N-terminal pool sequencing of a T=60 min. time point aliquot of the PSMA₁₆₃₋₁₉₂ proteasomal digest.

FIG. 8 shows binding curves for HLA-A2:PSMA₁₆₈₋₁₇₇ and HLA-A2:PSMA₂₈₈₋₂₉₇ with controls.

FIG. 9 shows results of N-terminal pool sequencing of a T=60 min. time point aliquot of the PSMA₂₈₁₋₃₁₀ proteasomal digest.

FIG. 10 shows binding curves for HLA-A2:PSMA₄₆₁₋₄₆₉, HLA-A2:PSMA₄₆₀₋₄₆₉, and HLA-A2:PSMA₆₆₃₋₆₇₁, with controls.

FIG. 11 shows the results of a γ (gamma)-IFN-based ELISPOT assay detecting PSMA₄₆₃₋₄₇₁-reactive HLA-A1⁺ CD8⁺ T cells.

FIG. 12 shows blocking of reactivity of the T cells used in FIG. 10 by anti-HLA-A1 mAb, demonstrating HLA-A1-restricted recognition.

FIG. 13 shows a binding curve for HLA-A2:PSMA₆₆₃₋₆₇₁, with controls.

FIG. 14 shows a binding curve for HLA-A2:PSMA₆₆₂₋₆₇₁, with controls.

FIG. 15. Comparison of anti-peptide CTL responses following immunization with various doses of DNA by different routes of injection.

FIG. 16. Growth of transplanted gp33 expressing tumor in mice immunized by i.ln. injection of gp33 epitope-expressing, or control, plasmid.

FIG. 17. Amount of plasmid DNA detected by real-time PCR in injected or draining lymph nodes at various times after i.ln. of i.m. injection, respectively.

FIGS. 18-70 are proteasomal digestion maps depicting the mapping of mass spectrum peaks from the digest onto the sequence of the indicated substrate.

### DETAILED DESCRIPTION OF THE PREFERRED EMBODIMENT

Definitions

Unless otherwise clear from the context of the use of a term herein, the following listed terms shall generally have the indicated meanings for purposes of this description.

PROFESSIONAL ANTIGEN-PRESENTING CELL (pAPC)—a cell that possesses T cell costimulatory molecules and is able to induce a T cell response. Well characterized pAPCs include dendritic cells, B cells, and macrophages.

PERIPHERAL CELL—a cell that is not a pAPC.

HOUSEKEEPING PROTEASOME—a proteasome normally active in peripheral cells, and generally not present or not strongly active in pAPCs.

IMMUNE PROTEASOME—a proteasome normally active in pAPCs; the immune proteasome is also active in some peripheral cells in infected tissues.

EPITOPE—a molecule or substance capable of stimulating an immune response. In preferred embodiments, epitopes according to this definition include but are not necessarily limited to a polypeptide and a nucleic acid encoding a polypeptide, wherein the polypeptide is capable of stimulating an immune response. In other preferred embodiments, epitopes according to this definition include but are not necessarily limited to peptides presented on the surface of cells, the peptides being non-covalently bound to the binding cleft of class I MHC, such that they can interact with T cell receptors (TCR). Epitopes presented by class I MHC may be in immature or mature form. “Mature” refers to an MHC epitope in distinction to any precursor (“immature”) that may include or consist essentially of a housekeeping epitope, but also includes other sequences in a primary translation product that are removed by processing, including without limitation, alone or in any combination proteasomal digestion, N-terminal trimming, or the action of exogenous enzymatic activities. Thus, a mature epitope may be provided embedded in a somewhat longer polypeptide, the immunological potential of which is due, at least in part, to the embedded epitope; or in its ultimate form that can bind in the MHC binding cleft to be recognized by TCR, respectively.

MHC EPITOPE—a polypeptide having a known or predicted binding affinity for a mammalian class I or class II major histocompatibility complex (MHC) molecule.

HOUSEKEEPING EPITOPE—In a preferred embodiment, a housekeeping epitope is defined as a polypeptide fragment that is an MHC epitope, and that is displayed on a cell in which housekeeping proteasomes are predominantly active. In another preferred embodiment, a housekeeping epitope is defined as a polypeptide containing a housekeeping epitope according to the foregoing definition, that is flanked by one to several additional amino acids. In another preferred embodiment, a housekeeping epitope is defined as a nucleic acid that encodes a housekeeping epitope according to the foregoing definitions.

IMMUNE EPITOPE—In a preferred embodiment, an immune epitope is defined as a polypeptide fragment that is an MHC epitope, and that is displayed on a cell in which immune proteasomes are predominantly active. In another preferred embodiment, an immune epitope is defined as a polypeptide containing an immune epitope according to the foregoing definition, that is flanked by one to several additional amino acids. In another preferred embodiment, an immune epitope is defined as a polypeptide including an epitope cluster sequence, having at least two polypeptide sequences having a known or predicted affinity for a class I MHC. In yet another preferred embodiment, an immune epitope is defined as a nucleic acid that encodes an immune epitope according to any of the foregoing definitions.

TARGET CELL—a cell to be targeted by the vaccines and methods of the invention. Examples of target cells according to this definition include but are not necessarily limited to: a neoplastic cell and a cell harboring an intracellular parasite, such as, for example, a virus, a bacterium, or a protozoan.

TARGET-ASSOCIATED ANTIGEN (TAA)—a protein or polypeptide present in a target cell.

TUMOR-ASSOCIATED ANTIGENS (TuAA)—a TAA, wherein the target cell is a neoplastic cell.

HLA EPITOPE—a polypeptide having a known or predicted binding affinity for a human class I or class II HLA complex molecule.

ANTIBODY—a natural immunoglobulin (Ig), poly- or monoclonal, or any molecule composed in whole or in part of an Ig binding domain, whether derived biochemically or by use of recombinant DNA. Examples include inter alia, F(ab), single chain Fv, and Ig variable region-phage coat protein fusions.

ENCODE—an open-ended term such that a nucleic acid encoding a particular amino acid sequence can consist of codons specifying that (poly)peptide, but can also comprise additional sequences either translatable, or for the control of transcription, translation, or replication, or to facilitate manipulation of some host nucleic acid construct.

SUBSTANTIAL SIMILARITY—this term is used to refer to sequences that differ from a reference sequence in an inconsequential way as judged by examination of the sequence. Nucleic acid sequences encoding the same amino acid sequence are substantially similar despite differences in degenerate positions or modest differences in length or composition of any non-coding regions. Amino acid sequences differing only by conservative substitution or minor length variations are substantially similar. Additionally, amino acid sequences comprising housekeeping epitopes that differ in the number of N-terminal flanking residues, or immune epitopes and epitope clusters that differ in the number of flanking residues at either terminus, are substantially similar. Nucleic acids that encode substantially similar amino acid sequences are themselves also substantially similar.

FUNCTIONAL SIMILARITY—this term is used to refer to sequences that differ from a reference sequence in an inconsequential way as judged by examination of a biological or biochemical property, although the sequences may not be substantially similar. For example, two nucleic acids can be useful as hybridization probes for the same sequence but encode differing amino acid sequences. Two peptides that induce cross-reactive CTL responses are functionally similar even if they differ by non-conservative amino acid substitutions (and thus do not meet the substantial similarity definition). Pairs of antibodies, or TCRs, that recognize the same epitope can be functionally similar to each other despite whatever structural differences exist. In testing for functional similarity of immunogenicity one would generally immunize with the “altered” antigen and test the ability of the elicited response (Ab, CTL, cytokine production, etc.) to recognize the target antigen. Accordingly, two sequences may be designed to differ in certain respects while retaining the same function. Such designed sequence variants are among the embodiments of the present invention.

VACCINE—this term is used to refer to those immunogenic compositions that are capable of eliciting prophylactic and/or therapeutic responses that prevent, cure, or ameliorate disease.

IMMUNOGENIC COMPOSITION—this term is used to refer to compositions capable of inducing an immune response, a reaction, an effect, and/or an event. In some embodiments, such responses, reactions, effects, and/or events can be induced in vitro or in vivo, for example. Included among these embodiments are the induction, activation, or expansion of cells involved in cell mediated immunity, for example. One example of such cells is cytotoxic T lymphocytes (CTLs). A vaccine is one type of immunogenic composition. Another example of such a composition is one that induces, activates, or expands CTLs in vitro. Further examples include pharmaceutical compositions and the like.

TABLE 1A
SEQ ID NOS.* including epitopes in Examples 1-7, 13, 14.

| SEQ | | |
|---|---|---|
| ID NO | IDENTITY | SEQUENCE |
| 1 | Tyr 207-216 | FLPWHRLFLL |
| 2 | Tyrosinase protein | Accession number**: P14679 |
| 3 | SSX-2 protein | Accession number: NP_003138 |
| 4 | PSMA protein | Accession number: NP_004467 |
| 5 | Tyrosinase cDNA | Accession number: NM_000372 |
| 6 | SSX-2 cDNA | Accession number: NM_003147 |
| 7 | PSMA cDNA | Accession number: NM_004476 |
| 8 | Tyr 207-215 | FLPWHRLFL |
| 9 | Tyr 208-216 | LPWHRLFLL |
| 10 | SSX-2 31-68 | YFSKEEWEKMKASEKIFYVYMKRKYEAMTKLGFKATLP |
| 11 | SSX-2 32-40 | FSKEEWEKM |
| 12 | SSX-2 39-47 | KMKASEKIF |
| 13 | SSX-2 40-48 | MKASEKIFY |
| 14 | SSX-2 39-48 | KMKASEKIFY |
| 15 | SSX-2 41-49 | KASEKIFYV |
| 16 | SSX-2 40-49 | MKASEKIFYV |
| 17 | SSX-2 41-50 | KASEKIFYVY |
| 18 | SSX-2 42-49 | ASEKIFYVY |
| 19 | SSX-2 53-61 | RKYEAMTKL |
| 20 | SSX-2 52-61 | KRKYEAMTKL |
| 21 | SSX-2 54-63 | KYEAMTKLGF |
| 22 | SSX-2 55-63 | YEAMTKLGF |
| 23 | SSX-2 56-63 | EAMTKLGF |
| 24 | HBV18-27 | FLPSDYFPSV |
| 25 | HLA-B44 binder | AEMGKYSFY |
| 26 | SSX-1 41-49 | KYSEKISYV |
| 27 | SSX-3 41-49 | KVSEKIVYV |
| 28 | SSX-4 41-49 | KSSEKIVYV |
| 29 | SSX-5 41-49 | KASEKIIYV |
| 30 | PSMA163-192 | AFSPQGMPEGDLVYVNYARTEDFFKLERDM |
| 31 | PSMA 168-190 | GMPEGDLVYVNYARTEDFFKLER |
| 32 | PSMA 169-177 | MPEGDLVYV |
| 33 | PSMA 168-177 | GMPEGDLVYV |
| 34 | PSMA 168-176 | GMPEGDLVY |
| 35 | PSMA 167-176 | QGMPEGDLVY |
| 36 | PSMA 169-176 | MPEGDLVY |
| 37 | PSMA 171-179 | EGDLVYVNY |
| 38 | PSMA 170-179 | PEGDLVYVNY |
| 39 | PSMA 174-183 | LVYVNYARTE |
| 40 | PSMA 177-185 | VNYARTEDF |
| 41 | PSMA 176-185 | YVNYARTEDF |
| 42 | PSMA 178-186 | NYARTEDFF |
| 43 | PSMA 179-186 | YARTEDFF |
| 44 | PSMA 181-189 | RTEDFFKLE |
| 45 | PSMA 281-310 | RGIAEAVGLPSIPVHPIGYYDAQKLLEKMG |
| 46 | PSMA 283-307 | IAEAVGLPSIPVHPIGYYDAQKLLE |
| 47 | PSMA 289-297 | LPSIPVHPI |
| 48 | PSMA 288-297 | GLPSIPVHPI |
| 49 | PSMA 297-305 | IGYYDAQKL |
| 50 | PSMA 296-305 | PIGYYDAQKL |
| 51 | PSMA 291-299 | SIPVHPIGY |
| 52 | PSMA 290-299 | PSIPVHPIGY |
| 53 | PSMA 292-299 | IPVHPIGY |
| 54 | PSMA 299-307 | YYDAQKLLE |
| 55 | PSMA454-481 | SSIEGNYTLRVDCTPLMYSLVHLTKEL |
| 56 | PSMA 456-464 | IEGNYTLRV |
| 57 | PSMA 455-464 | SIEGNYTLRV |
| 58 | PSMA 457-464 | EGNYTLRV |
| 59 | PSMA 461-469 | TLRVDCTPL |
| 60 | PSMA 460-469 | YTLRVDCTPL |
| 61 | PSMA 462-470 | LRVDCTPLM |
| 62 | PSMA 463-471 | RVDCTPLMY |
| 63 | PSMA 462-471 | LRVDCTPLMY |
| 64 | PSMA653-687 | FDKSNPIVLRMMNDQLMFLERAFIDPLGLPDRPFY |
| 65 | PSMA 660-681 | VLRMMNDQLMFLERAFIDPLGL |
| 66 | PSMA 663-671 | MMNDQLMFL |
| 67 | PSMA 662-671 | RMMNDQLMFL |
| 68 | PSMA 662-670 | RMMNDQLMF |
| 69 | Tyr 1-17 | MLLAVLYCLLWSFQTSA |
| 70 | GP100 protein² | Accession number: P40967 |
| 71 | MAGE-1 protein | Accession number: P43355 |
| 72 | MAGE-2 protein | Accession number: P43356 |
| 73 | MAGE-3 protein | Accession number: P43357 |
| 74 | NY-ESO-1 protein | Accession number: P78358 |
| 75 | LAGE-1a protein | Accession number: CAA11116 |
| 76 | LAGE-1b protein | Accession number: CAA11117 |
| 77 | PRAME protein | Accession number: NP 006106 |
| 78 | PSA protein | Accession number: P07288 |
| 79 | PSCA protein | Accession number: O43653 |
| 80 | GP100 cds | Accession number: U20093 |
| 81 | MAGE-1 cds | Accession number: M77481 |
| 82 | MAGE-2 cds | Accession number: L18920 |
| 83 | MAGE-3 cds | Accession number: U03735 |
| 84 | NY-ESO-1 cDNA | Accession number: U87459 |
| 85 | PRAME cDNA | Accession number: NM_006115 |
| 86 | PSA cDNA | Accession number: NM_001648 |
| 87 | PSCA cDNA | Accession number: AF043498 |
| 88 | CEA protein | Accession number: P06731 |
| 89 | CEA cDNA | Accession number: NM_004363 |
| 90 | Her2/Neu protein | Accession number: P04626 |
| 91 | Her2/Neu cDNA | Accession number: M11730 |
| 92 | SCP-1 protein | Accession number: Q15431 |
| 93 | SCP-1 cDNA | Accession number: X95654 |
| 94 | SSX-4 protein | Accession number: O60224 |
| 95 | SSX-4 cDNA | Accession number: NM_005636 |
| 96 | GAGE-1 protein | Accession number: Q13065 |
| 97 | GAGE-1 cDNA | Accession number: U19142 |
| 98 | Suvivin protein | Accession number: O15392 |
| 99 | Survivin cDNA | Accession number: NM_001168 |
| 100 | Melan-A protein | Accession number: Q16655 |
| 101 | Melan-A cDNA | Accession number: U06452 |
| 102 | BAGE protein | Accession number: Q13072 |
| 103 | BAGE cDNA | Accession number: U19180 |
| 104 | PSA 59-67 | WVLTAAHCI |
| 105 | Glandular Kallikrein 1 | Accession number: P06870 |
| 106 | Elastase 2A | Accession number: P08217 |
| 107 | Pancreatic elastase IIB | Accession number: NP_056933 |

TABLE 1B
SEQ ID NOS.* including epitopes in Examples 15-67.

| SEQ | | |
|---|---|---|
| ID NO | IDENTITY | SEQUENCE |
| 108 | Tyr 171-179 | NIYDLFVWM |
| 109 | Tyr 173-182 | YDLFVWMHYY |
| 110 | Tyr 174-182 | DLFVWMHYY |
| 111 | Tyr 186-194 | DALLGGSEI |
| 112 | Tyr 191-200 | GSEIWRDIDF |
| 113 | Tyr 192-200 | SEIWRDIDF |
| 114 | Tyr 193-201 | EIWRDIDFA |
| 115 | Tyr 407-416 | LQEVYPEANA |
| 116 | Tyr 409-418 | EVYPEANAPI |
| 117 | Tyr 410-418 | VYPEANAPI |
| 118 | Tyr 411-418 | YPEANAPI |
| 119 | Tyr 411-420 | YPEANAPIGH |
| 120 | Tyr 416-425 | APIGHNRESY |
| 121 | Tyr 417-425 | PIGHNRESY |
| 122 | Tyr 417-426 | PIGHNRESYM |
| 123 | Tyr 416-425 | APIGHNRESY |
| 124 | Tyr 417-425 | PIGHNRESY |
| 125 | Tyr 423-430 | ESYMVPFI |
| 126 | Tyr 423-432 | ESYMVPFIPL |
| 127 | Tyr 424-432 | SYMVPFIPL |
| 128 | Tyr 424-433 | SYMVPFIPLY |
| 129 | Tyr 425-433 | YMVPFIPLY |
| 130 | Tyr 426-434 | MVPFIPLYR |
| 131 | Tyr 426-435 | MVPFIPLYRN |
| 132 | Tyr 427-434 | VPFIPLYR |
| 133 | Tyr 430-437 | IPLYRNGD |
| 134 | Tyr 430-439 | IPLYRNGDFF |
| 135 | Tyr 431-439 | PLYRNGDFF |
| 136 | Tyr 431-440 | PLYRNGDFFI |
| 137 | Tyr 434-443 | RNGDFFISSK |
| 138 | Tyr 435-443 | NGDFFISSK |
| 139 | Tyr 463-471 | YIKSYLEQA |
| 140 | Tyr 466-474 | SYLEQASRI |
| 141 | Tyr 469-478 | EQASRIWSWL |
| 142 | Tyr 470-478 | QASRIWSWL |
| 143 | Tyr 471-478 | ASRIWSWL |
| 144 | Tyr 471-479 | ASRIWSWLL |
| 145 | Tyr 473-481 | RIWSWLLGA |
| 146 | CEA 92-100 | GPAYSGREI |
| 147 | CEA 92-101 | GPAYSGREII |
| 148 | CEA 93-100 | PAYSGREI |
| 149 | CEA 93-101 | PAYSGREII |
| 150 | CEA 93-102 | PAYSGREIIY |
| 151 | CEA 94-102 | AYSGREIIY |
| 152 | CEA 97-105 | GREIIYPNA |
| 153 | CEA 98-107 | REIIYPNASL |
| 154 | CEA 99-107 | EIIYPNASL |
| 155 | CEA 99-108 | EIIYPNASLL |
| 156 | CEA 100-107 | IIYPNASL |
| 157 | CEA 100-108 | IIYPNASLL |
| 158 | CEA 100-109 | IIYPNASLLI |
| 159 | CEA 102-109 | YPNASLLI |
| 160 | CEA 107-116 | LLIQNIIQND |
| 161 | CEA 132-141 | EEATGQFRVY |
| 162 | CEA 133-141 | EATGQFRVY |
| 163 | CEA 141-149 | YPELPKPSI |
| 164 | CEA 142-149 | PELPKPSI |
| 165 | CEA 225-233 | RSDSVILNV |
| 166 | CEA 225-234 | RSDSVILNVL |
| 167 | CEA 226-234 | SDSVILNVL |
| 168 | CEA 226-235 | SDSVBLNVLY |
| 169 | CEA 227-235 | DSVILNVLY |
| 170 | CEA 233-242 | VLYGPDAPTI |
| 171 | CEA 234-242 | LYGPDAPTI |
| 172 | CEA 235-242 | YGPDAPTI |
| 173 | CEA 236-245 | GPDAPTISPL |
| 174 | CEA 237-245 | PDAPTISPL |
| 175 | CEA 238-245 | DAPTISPL |
| 176 | CEA 239-247 | APTISPLNT |
| 177 | CEA 240-249 | PTISPLNTSY |
| 178 | CEA 241-249 | TISPLNTSY |
| 179 | CEA 240-249 | PTISPLNTSY |
| 180 | CEA 241-249 | TISPLNTSY |
| 181 | CEA 246-255 | NTSYRSGENL |
| 182 | CEA 247-255 | TSYRSGENL |
| 183 | CEA 248-255 | SYRSGENL |
| 184 | CEA 248-257 | SYRSGENLNL |
| 185 | CEA 249-257 | YRSGENLNL |
| 186 | CEA 251-259 | SGENLNLSC |
| 187 | CEA 253-262 | ENLNLSCHAA |
| 188 | CEA 254-262 | NLNLSCHAA |
| 189 | CEA 260-269 | HAASNPPAQY |
| 190 | CEA 261-269 | AASNPPAQY |
| 191 | CEA 264-273 | NPPAQYSWFV |
| 192 | CEA 265-273 | PPAQYSWFV |
| 193 | CEA 266-273 | PAQYSWFV |
| 194 | CEA 272-280 | FVNGTFQQS |
| 195 | CEA 310-319 | RTTVTTITVY |
| 196 | CEA 311-319 | TTVTTITVY |
| 197 | CEA 319-327 | YAEPPKPFI |
| 198 | CEA 319-328 | YAEPPKPFIT |
| 199 | CEA 320-327 | AEPPKPFI |
| 200 | CEA 321-328 | EPPKPFIT |
| 201 | CEA 321-329 | EPPKPFITS |
| 202 | CEA 322-329 | PPKPFITS |
| 203 | CEA 382-391 | SVTRNDVGPY |
| 204 | CEA 383-391 | VTRNDVGPY |
| 205 | CEA 389-397 | GPYECGIQN |
| 206 | CEA 391-399 | YECGIQNEL |
| 207 | CEA 394-402 | GIQNELSVD |
| 208 | CEA 403-411 | HSDPVILNV |
| 209 | CEA 403-412 | HSDPVILNVL |
| 210 | CEA 404-412 | SDPVILNVL |
| 211 | CEA 404-413 | SDPVILNVLY |
| 212 | CEA 405-412 | DPVILNVL |
| 213 | CEA 405-413 | DPVILNVLY |
| 214 | CEA 408-417 | ILNVLYGPDD |
| 215 | CEA 411-420 | VLYGPDDPTI |
| 216 | CEA 412-420 | LYGPDDPTI |
| 217 | CEA 413-420 | YGPDDPTI |
| 218 | CEA 417-425 | DPTISPSYT |
| 219 | CEA 418-427 | PTISPSYTYY |
| 220 | CEA 419-427 | TISPSYTYY |
| 221 | CEA 418-427 | PTISPSYTYY |
| 222 | CEA 419-427 | TISPSYTYY |
| 223 | CEA 419-428 | TISPSYTYYR |
| 224 | CEA 424-433 | YTYYRPGVNL |
| 225 | CEA 425-433 | TYYRPGVNL |
| 226 | CEA 426-433 | YYRPGVNL |
| 227 | CEA 426-435 | YYRPGVNLSL |
| 228 | CEA 427-435 | YRPGVNLSL |
| 229 | CEA 428-435 | RPGVNLSL |
| 230 | CEA 428-437 | RPGVNLSLSC |
| 231 | CEA 430-438 | GVNLSLSCH |
| 232 | CEA 431-440 | VNLSLSCHAA |
| 233 | CEA 432-440 | NLSLSCHAA |
| 234 | CEA 438-447 | HAASNPPAQY |
| 235 | CEA 439-447 | AASNPPAQY |
| 236 | CEA 442-451 | NPPAQYSWLI |
| 237 | CEA 443-451 | PPAQYSWLI |
| 238 | CEA 444-451 | PAQYSWLI |
| 239 | CEA 449-458 | WLIDGNIQQH |
| 240 | CEA 450-458 | LIDGNIQQH |
| 241 | CEA 450-459 | LIDGNIQQHT |
| 242 | CEA 581-590 | RSDPVTLDVL |
| 243 | CEA 582-590 | SDPVTLDVL |
| 244 | CEA 582-591 | SDPVTLDVLY |
| 245 | CEA 583-590 | DPVTLDVL |
| 246 | CEA 583-591 | DPVTLDVLY |
| 247 | CEA 588-597 | DVLYGPDTPI |
| 248 | CEA 589-597 | VLYGPDTPI |
| 249 | CEA 596-605 | PIISPPDSSY |
| 250 | CEA 597-605 | IISPPDSSY |
| 251 | CEA 597-606 | IISPPDSSYL |
| 252 | CEA 599-606 | SPPDSSYL |
| 253 | CEA 600-608 | PPDSSYLSG |
| 254 | CEA 600-609 | PPDSSYLSGA |
| 255 | CEA 602-611 | DSSYLSGANL |
| 256 | CEA 603-611 | SSYLSGANL |
| 257 | CEA 604-613 | SYLSGANLNL |
| 258 | CEA 605-613 | YLSGANLNL |
| 259 | CEA 610-618 | NLNLSCHSA |
| 260 | CEA 620-629 | NPSPQYSWRI |
| 261 | CEA 622-629 | SPQYSWRI |
| 262 | CEA 627-635 | WRINGIPQQ |
| 263 | CEA 628-636 | RINGIPQQH |
| 264 | CEA 628-637 | RINGIPQQHT |
| 265 | CEA 631-639 | GIPQQHTQV |
| 266 | CEA 632-639 | IPQQHTQV |
| 267 | CEA 644-653 | KITPNNNGTY |
| 268 | CEA 645-653 | ITPNNNGTY |
| 269 | CEA 647-656 | PNNNGTYACF |
| 270 | CEA 648-656 | NNNGTYACF |
| 271 | CEA 650-657 | NGTYACFV |
| 272 | CEA 661-670 | ATGRNNSIVK |
| 273 | CEA 662-670 | TGRNNSIVK |
| 274 | CEA 664-672 | RNNSIVKSI |
| 275 | CEA 666-674 | NSIVKSITV |
| 276 | GAGE-1 7-16 | STYRPRPRRY |
| 277 | GAGE-1 8-16 | TYRPRPRRY |
| 278 | GAGE-1 10-18 | RPRPRRYVE |
| 279 | GAGE-1 16-23 | YVEPPEMI |
| 280 | GAGE-1 22-31 | MIGPMRPEQF |
| 281 | GAGE-1 23-31 | IGPMRPEQF |
| 282 | GAGE-1 24-31 | GPMRPEQF |
| 283 | GAGE-1 105-114 | KTPEEEMRSH |
| 284 | GAGE-1 106-115 | TPEEEMRSHY |
| 285 | GAGE-1 107-115 | PEEEMRSHY |
| 286 | GAGE-1 110-119 | EMRSHYVAQT |
| 287 | GAGE-1 113-121 | SHYVAQTGI |
| 288 | GAGE-1 115-124 | YVAQTGILWL |
| 289 | GAGE-1 116-124 | VAQTGILWL |
| 290 | GAGE-1 116-125 | VAQTGILWLL |
| 291 | GAGE-1 117-125 | AQTGILWLL |
| 292 | GAGE-1 118-126 | QTGILWLLM |
| 293 | GAGE-1 118-127 | QTGILWLLMN |
| 294 | GAGE-1 120-129 | GILWLLMNNC |
| 295 | GAGE-1 121-129 | ILWLLMNNC |
| 296 | GAGE-1 124-131 | LLMNNCFL |
| 297 | GAGE-1 123-131 | WLLMNNCFL |
| 298 | GAGE-1 122-130 | LWLLMNNCF |
| 299 | GAGE-1 121-130 | ILWLLMNNCF |
| 300 | GAGE-1 121-129 | ILWLLMNNC |
| 301 | GAGE-1 120-129 | GILWLLMNNC |
| 302 | GAGE-1 118-127 | QTGILWLLMN |
| 303 | GAGE-1 118-126 | QTGILWLLM |
| 304 | GAGE-1 117-125 | AQTGILWLL |
| 305 | GAGE-1 116-125 | VAQTGILWLL |
| 306 | GAGE-1 116-124 | VAQTGILWL |
| 307 | GAGE-1 115-124 | YVAQTGILWL |
| 308 | GAGE-1 113-121 | SHYVAQTGI |
| 309 | MAGE-1 62-70 | SAFPTTINF |
| 310 | MAGE-1 61-70 | ASAFPTTINF |
| 311 | MAGE-1 60-68 | GASAFPTTI |
| 312 | MAGE-1 57-66 | SPQGASAFPT |
| 313 | MAGE-1 144-151 | FGKASESL |
| 314 | MAGE-1 143-151 | IFGKASESL |
| 315 | MAGE-1 142-151 | EIFGKASESL |
| 316 | MAGE-1 142-149 | EIFGKASE |
| 317 | MAGE-1 133-140 | IKNYKHCF |
| 318 | MAGE-1 132-140 | VIKNYKHCF |
| 319 | MAGE-1 131-140 | SVIKNYKHCF |
| 320 | MAGE-1 132-139 | VIKNYKHC |
| 321 | MAGE-1 131-139 | SVIKNYKHC |
| 322 | MAGE-1 128-136 | MLESVIKNY |
| 323 | MAGE-1 127-136 | EMLESVIKNY |
| 324 | MAGE-1 126-134 | AEMLESVIK |
| 325 | MAGE-2 274-283 | GPRALIETSY |
| 326 | MAGE-2 275-283 | PRALIETSY |
| 327 | MAGE-2 276-284 | RALIETSYV |
| 328 | MAGE-2 277-286 | ALIETSYVKV |
| 329 | MAGE-2 278-286 | LIETSYVKV |
| 330 | MAGE-2 278-287 | LIETSYVKVL |
| 331 | MAGE-2 279-287 | IETSYVKVL |
| 332 | MAGE-2 280-289 | ETSYVKVLHH |
| 333 | MAGE-2 282-291 | SYVKVLHHTL |
| 334 | MAGE-2 283-291 | YVKVLHHTL |
| 335 | MAGE-2 285-293 | KVLHHTLKI |
| 336 | MAGE-2 303-311 | PLHERALRE |
| 337 | MAGE-2 302-309 | PPLHERAL |
| 338 | MAGE-2 301-309 | YPPLHERAL |
| 339 | MAGE-2 300-309 | SYPPLHERAL |
| 340 | MAGE-2 299-307 | ISYPPLHER |
| 341 | MAGE-2 298-307 | HISYPPLHER |
| 342 | MAGE-2 292-299 | KIGGEPHI |
| 343 | MAGE-2 291-299 | LKIGGEPHI |
| 344 | MAGE-2 290-299 | TLKIGGEPHI |
| 345 | MAGE-3 303-311 | PLHEWVLRE |
| 346 | MAGE-3 302-309 | PPLHEWVL |
| 347 | MAGE-3 301-309 | YPPLHEWVL |
| 348 | MAGE-3 301-308 | YPPLHEWV |
| 349 | MAGE-3 300-308 | SYPPLHEWV |
| 350 | MAGE-3 299-308 | ISYPPLHEWV |
| 351 | MAGE-3 298-307 | HISYPPLHEW |
| 352 | MAGE-3 293-301 | ISGGPHISY |
| 353 | MAGE-3 292-301 | KISGGPHISY |
| 354 | Melan-A 45-54 | CWYCRRRNGY |
| 355 | Melan-A 46-54 | WYCRRRNGY |
| 356 | Melan-A 47-55 | YCRRRNGYR |
| 357 | Melan-A 49-57 | RRRNGYRAL |
| 358 | Melan-A 51-60 | RNGYRALMDK |
| 359 | Melan-A 52-60 | NGYRALMDK |
| 360 | Melan-A 55-63 | RALMDKSLH |
| 361 | Melan-A 56-63 | ALMDKSLH |
| 362 | Melan-A 55-64 | RALMDKSLHV |
| 363 | Melan-A 56-64 | ALMDKSLHV |
| 364 | PRAME 275-284 | YISPEKEEQY |
| 365 | PRAME 276-284 | ISPEKEEQY |
| 366 | PRAME 277-285 | SPEKEEQYI |
| 367 | PRAME 278-285 | PEKEEQYI |
| 368 | PRAME 279-288 | EKEEQYIAQF |
| 369 | PRAME 280-288 | KEEQYIAQF |
| 370 | PRAME 283-292 | QYIAQFTSQF |
| 371 | PRAME 284-292 | YIAQFTSQF |
| 372 | PRAME 284-293 | YIAQFTSQFL |
| 373 | PRAME 285-293 | IAQFTSQFL |
| 374 | PRAME 286-295 | AQFTSQFLSL |
| 375 | PRAME 287-295 | QFTSQFLSL |
| 376 | PRAME 290-298 | SQFLSLQCL |
| 377 | PRAME 439-448 | VLYPVPLESY |
| 378 | PRAME 440-448 | LYPVPLESY |
| 379 | PRAME 446-455 | ESYEDIHGTL |
| 380 | PRAME 448-457 | YEDIHGTLHL |
| 381 | PRAME 449-457 | EDIHGTLHL |
| 382 | PRAME 451-460 | IHGTLHLERL |
| 383 | PRAME 454-463 | TLHLERLAYL |
| 384 | PRAME 455-463 | LHLERLAYL |
| 385 | PRAME 456-463 | HLERLAYL |
| 386 | PRAME 456-465 | HLERLAYLHA |
| 387 | PRAME 458-467 | ERLAYLHARL |
| 388 | PRAME 459-467 | RLAYLHARL |
| 389 | PRAME 459-468 | RLAYLHARLR |
| 390 | PRAME 460-467 | LAYLHARL |
| 391 | PRAME 460-468 | LAYLHARLR |
| 392 | PRAME 461-470 | AYLHARLREL |
| 393 | PRAME 462-470 | YLHARLREL |
| 394 | PRAME 462-471 | YLHARLRELL |
| 395 | PRAME 463-471 | LHARLRELL |
| 396 | PRAME 464-471 | HARLRELL |
| 397 | PRAME 464-472 | HARLRELLC |
| 398 | PRAME 469-478 | ELLCELGRPS |
| 399 | PRAME 470-478 | LLCELGRPS |
| 400 | PSA 144-153 | QEPALGTTCY |
| 401 | PSA 145-153 | EPALGTTCY |
| 402 | PSA 162-171 | PEEFLTPKKL |
| 403 | PSA 163-171 | EEFLTPKKL |
| 404 | PSA 165-173 | FLTPKKLQC |
| 405 | PSA 165-174 | FLTPKKLQCV |
| 406 | PSA 166-174 | LTPKKLQCV |
| 407 | PSA 167-174 | TPKKLQCV |
| 408 | PSA 167-175 | TPKKLQCVD |
| 409 | PSA 170-179 | KLQCVDLHVI |
| 410 | PSA 171-179 | LQCVDLHVI |
| 411 | PSCA 73-81 | DSQDYYVGK |
| 412 | PSCA 74-82 | SQDYYVGKK |
| 413 | PSCA 74-83 | SQDYYVGKKN |
| 414 | PSCA 76-84 | DYYVGKKNI |
| 415 | PSCA 77-84 | YYVGKKNI |
| 416 | PSCA 78-86 | YVGKKNITC |
| 417 | PSCA 78-87 | YVGKKNITCC |
| 418 | PSMA 381-390 | WVFGGIDPQS |
| 419 | PSMA 385-394 | GIDPQSGAAV |
| 420 | PSMA 386-394 | IDPQSGAAV |
| 421 | PSMA 387-394 | DPQSGAAV |
| 422 | PSMA 387-395 | DPQSGAAVV |
| 423 | PSMA 387-396 | DPQSGAAVVH |
| 424 | PSMA 388-396 | PQSGAAVVH |
| 425 | PSMA 389-398 | QSGAAVVHEI |
| 426 | PSMA 390-398 | SGAAVVHEI |
| 427 | PSMA 391-398 | GAAVVHEI |
| 428 | PSMA 391-399 | GAAVVHEIV |
| 429 | PSMA 392-399 | AAVVHEIV |
| 430 | PSMA 597-605 | CRDYAVVLR |
| 431 | PSMA 598-607 | RDYAVVLRKY |
| 432 | PSMA 599-607 | DYAVVLRKY |
| 433 | PSMA 600-607 | YAVVLRKY |
| 434 | PSMA 602-611 | VVLRKYADKI |
| 435 | PSMA 603-611 | VLRKYADKI |
| 436 | PSMA 603-612 | VLRKYADKIY |
| 437 | PSMA 604-611 | LRKYADKI |
| 438 | PSMA 604-612 | LRKYADKIY |
| 439 | PSMA 605-614 | RKYADKIYSI |
| 440 | PSMA 606-614 | KYADKIYSI |
| 441 | PSMA 607-614 | YADKIYSI |
| 442 | PSMA 616-625 | MKHPQEMKTY |
| 443 | PSMA 617-625 | KHPQEMKTY |
| 444 | PSMA 618-627 | HPQEMKTYSV |
| 445 | SCP-1 62-71 | IDSDPALQKV |
| 446 | SCP-1 63-71 | DSDPALQKV |
| 447 | SCP-1 67-76 | ALQKVNFLPV |
| 448 | SCP-1 70-78 | KVNFLPVLE |
| 449 | SCP-1 71-80 | VNFLPVLEQV |
| 450 | SCP-1 72-80 | NFLPVLEQV |
| 451 | SCP-1 75-84 | PVLEQVGNSD |
| 452 | SCP-1 76-84 | VLEQVGNSD |
| 453 | SCP-1 202-210 | YEREETRQV |
| 454 | SCP-1 202-211 | YEREETRQVY |
| 455 | SCP-1 203-211 | EREETRQVY |
| 456 | SCP-1 203-212 | EREETRQVYM |
| 457 | SCP-1 204-212 | REETRQVYM |
| 458 | SCP-1 211-220 | YMDLNSNIEK |
| 459 | SCP-1 213-221 | DLNSNIEKM |
| 460 | SCP-1 216-226 | SNIEKMITAF |
| 461 | SCP-1 217-225 | NIEKMITAF |
| 462 | SCP-1 218-225 | IEKMITAF |
| 463 | SCP-1 397-406 | RLENYEDQLI |
| 464 | SCP-1 398-406 | LENYEDQLI |
| 465 | SCP-1 398-407 | LENYEDQLII |
| 466 | SCP-1 399-407 | ENYEDQLII |
| 467 | SCP-1 399-408 | ENYEDQLIIL |
| 468 | SCP-1 400-408 | NYEDQLIIL |
| 469 | SCP-1 400-409 | NYEDQLIILT |
| 470 | SCP-1 401-409 | YEDQLIILT |
| 471 | SCP-1 401-410 | YEDQLIILTM |
| 472 | SCP-1 402-410 | EDQLIILTM |
| 473 | SCP-1 406-415 | IILTMELQKT |
| 474 | SCP-1 407-415 | ILTMELQKT |
| 475 | SCP-1 424-432 | KLTNNKEVE |
| 476 | SCP-1 424-433 | KLTNNKEVEL |
| 477 | SCP-1 425-433 | LTNNKEVEL |
| 478 | SCP-1 429-438 | KEVELEELKK |
| 479 | SCP-1 430-438 | EVELEELKK |
| 480 | SCP-1 430-439 | EVELEELKKV |
| 481 | SCP-1 431-439 | VELEELKKV |
| 482 | SCP-1 530-539 | ETSDMTLELK |
| 483 | SCP-1 531-539 | TSDMTLELK |
| 484 | SCP-1 548-556 | NKKQEERML |
| 485 | SCP-1 553-562 | ERMLTQIENL |
| 486 | SCP-1 554-562 | RMLTQIENL |
| 487 | SCP-1 555-562 | MLTQIENL |
| 488 | SCP-1 555-564 | MLTQIENLQE |
| 489 | SCP-1 560-569 | ENLQETETQL |
| 490 | SCP-1 561-569 | NLQETETQL |
| 491 | SCP-1 561-570 | NLQETETQLR |
| 492 | SCP-1 567-576 | TQLRNELEYV |
| 493 | SCP-1 568-576 | QLRNELEYV |
| 494 | SCP-1 571-580 | NELEYVREEL |
| 495 | SCP-1 572-580 | ELEYVREEL |
| 496 | SCP-1 573-580 | LEYVREEL |
| 497 | SCP-1 574-583 | EYVREELKQK |
| 498 | SCP-1 575-583 | YVREELKQK |
| 499 | SCP-1 675-684 | LLEEVEKAKV |
| 500 | SCP-1 676-684 | LEEVEKAKV |
| 501 | SCP-1 676-685 | LEEVEKAKVI |
| 502 | SCP-1 677-685 | EEVEKAKVI |
| 503 | SCP-1 681-690 | KAKVIADEAV |
| 504 | SCP-1 683-692 | KVIADEAVKL |
| 505 | SCP-1 684-692 | VIADEAVKL |
| 506 | SCP-1 685-692 | IADEAVKL |
| 507 | SCP-1 694-702 | KEIDKRCQH |
| 508 | SCP-1 694-703 | KEIDKRCQHK |
| 509 | SCP-1 695-703 | EIDKRCQHK |
| 510 | SCP-1 695-704 | EIDKRCQHKI |
| 511 | SCP-1 696-704 | IDKRCQHKI |
| 512 | SCP-1 697-704 | DKRCQHKI |
| 513 | SCP-1 698-706 | KRCQHKIAE |
| 514 | SCP-1 698-707 | KRCQHKIAEM |
| 515 | SCP-1 699-707 | RCQHKIAEM |
| 516 | SCP-1 701-710 | QHKIAEMVAL |
| 517 | SCP-1 702-710 | HKIAEMVAL |
| 518 | SCP-1 703-710 | KIAEMVAL |
| 519 | SCP-1 737-746 | QEQSSLRASL |
| 520 | SCP-1 738-746 | EQSSLRASL |
| 521 | SCP-1 739-746 | QSSLRASL |
| 522 | SCP-1 741-750 | SLRASLEIEL |
| 523 | SCP-1 742-750 | LRASLEIEL |
| 524 | SCP-1 743-750 | RASLEIEL |
| 525 | SCP-1 744-753 | ASLEIELSNL |
| 526 | SCP-1 745-753 | SLEIELSNL |
| 527 | SCP-1 745-754 | SLEIELSNLK |
| 528 | SCP-1 746-754 | LEIELSNLK |
| 529 | SCP-1 747-755 | EIELSNLKA |
| 530 | SCP-1 749-758 | ELSNLKAELL |
| 531 | SCP-1 750-758 | LSNLKAELL |
| 532 | SCP-1 751-760 | SNLKAELLSV |
| 533 | SCP-1 752-760 | NLKAELLSV |
| 534 | SCP-1 752-761 | NLKAELLSVK |
| 535 | SCP-1 753-761 | LKAELLSVK |
| 536 | SCP-1 753-762 | LKAELLSVKK |
| 537 | SCP-1 754-762 | KAELLSVKK |
| 538 | SCP-1 755-763 | AELLSVKKQ |
| 539 | SCP-1 787-796 | EKKDKKTQTF |
| 540 | SCP-1 788-796 | KKDKKTQTF |
| 541 | SCP-1 789-796 | KDKKTQTF |
| 542 | SCP-1 797-806 | LLETPDIYWK |
| 543 | SCP-1 798-806 | LETPDIYWK |
| 544 | SCP-1 798-807 | LETPDIYWKL |
| 545 | SCP-1 799-807 | ETPDIYWKL |
| 546 | SCP-1 800-807 | TPDIYWKL |
| 547 | SCP-1 809-817 | SKAVPSQTV |
| 548 | SCP-1 810-817 | KAVPSQTV |
| 549 | SCP-1 812-821 | VPSQTVSRNF |
| 550 | SCP-1 815-824 | QTVSRNFTSV |
| 551 | SCP-1 816-824 | TVSRNFTSV |
| 552 | SCP-1 816-825 | TVSRNFTSVD |
| 553 | SCP-1 823-832 | SVDHGISKDK |
| 554 | SCP-1 829-838 | SKDKRDYLWT |
| 555 | SCP-1 832-840 | KRDYLWTSA |
| 556 | SCP-1 832-841 | KRDYLWTSAK |
| 557 | SCP-1 833-841 | RDYLWTSAK |
| 558 | SCP-1 835-843 | YLWTSAKNT |
| 559 | SCP-1 835-844 | YLWTSAKNTL |
| 560 | SCP-1 837-844 | WTSAKNTL |
| 561 | SCP-1 841-850 | KNTLSTPLPK |
| 562 | SCP-1 842-850 | NTLSTPLPK |
| 563 | SCP-1 832-840 | KRDYLWTSA |
| 564 | SCP-1 832-841 | KRDYLWTSAK |
| 565 | SCP-1 833-841 | RDYLWTSAK |
| 566 | SCP-1 835-843 | YLWTSAKNT |
| 567 | SCP-1 839-846 | SAKNTLST |
| 568 | SCP-1 841-850 | KNTLSTPLPK |
| 569 | SCP-1 842-850 | NTLSTPLPK |
| 570 | SCP-1 843-852 | TLSTPLPKAY |
| 571 | SCP-1 844-852 | LSTPLPKAY |
| 572 | SSX-2 5-12 | DAFARRPT |
| 573 | SSX-2 7-15 | FARRPTVGA |
| 574 | SSX-2 8-17 | ARRPTVGAQI |
| 575 | SSX-2 9-17 | RRPTVGAQI |
| 576 | SSX-2 10-17 | RPTVGAQI |
| 577 | SSX-2 13-21 | VGAQIPEKI |
| 578 | SSX-2 14-21 | GAQIPEKI |
| 579 | SSX-2 15-24 | AQIPEKIQKA |
| 580 | SSX-2 16-24 | QIPEKIQKA |
| 581 | SSX-2 16-25 | QIPEKIQKAF |
| 582 | SSX-2 17-24 | IPEKIQKA |
| 583 | SSX-2 17-25 | IPEKIQKAF |
| 584 | SSX-2 18-25 | PEKIQKAF |
| 585 | Survivin 116-124 | ETNNKKKEF |
| 586 | Survivin 117-124 | TNNKKKEF |
| 587 | Survivin 122-131 | KEFEETAKKV |
| 588 | Survivin 123-131 | EFEETAKKV |
| 589 | Survivin 127-134 | TAKKVRRA |
| 590 | Survivin 126-134 | ETAKKVRRA |
| 591 | Survivin 128-136 | AKKVRRAIE |
| 592 | Survivin 129-138 | KKVRRAIEQL |
| 593 | Survivin 130-138 | KVRRAIEQL |
| 594 | Survivin 130-139 | KVRRAIEQLA |
| 595 | Survivin 131-138 | VRRAIEQL |
| 596 | BAGE 24-31 | SPVVSWRL |
| 597 | BAGE 21-29 | KEESPVVSW |
| 598 | BAGE 19-27 | LMKEESPVV |
| 599 | BAGE 18-27 | RLMKEESPVV |
| 600 | BAGE 18-26 | RLMKEESPV |
| 601 | BAGE 14-22 | LLQARLMKE |
| 602 | BAGE 13-22 | QLLQARLMKE |
| 603 | Survivin 13-28 | FLKDHRISTFKNWPFL |
| 604 | Survivin 79-111 | KHSSGCAFLSVKKQFEELTLGEFLKLDRERAKN |
| 605 | Survivin 130-141 | KVRRAIEQLAAM |
| 606 | GAGE-1 116-133 | VAQTGILWLLMNNCFLNL |
| 607 | BAGE 7-17 | FLALSAQLLQA |
| 608 | BAGE 18-27 | RLMKEESPVV |
| 609 | BAGE 2-27 | AARAVFLALSAQLLQARLMKEESPVV |
| 610 | BAGE 30-39 | RLEPEDGTAL |

*Any of SEQ ID NOS. 108-602 can be useful as epitopes in any of the various embodiments of the invention. Any of SEQ ID NOS. 603-610 can be useful as sequences containing epitopes or epitope clusters, as described in various embodiments of the invention.
**All accession numbers used here and throughout can be accessed through the NCBI databases, for example, through the Entrez seek and retreival system on the world wide web.

Note that the following discussion sets forth the inventors' understanding of the operation of the invention. However, it is not intended that this discussion limit the patent to any particular theory of operation not set forth in the claims.

In pursuing the development of epitope vaccines others have generated lists of predicted epitopes based on MHC binding motifs. Such peptides can be immunogenic, but may not correspond to any naturally produced antigenic fragment. Therefore, whole antigen will not elicit a similar response or sensitize a target cell to cytolysis by CTL. Therefore such lists do not differentiate between those sequences that can be useful as vaccines and those that cannot. Efforts to determine which of these predicted epitopes are in fact naturally produced have often relied on screening their reactivity with tumor infiltrating lymphocytes (TIL). However, TIL are strongly biased to recognize immune epitopes whereas tumors (and chronically infected cells) will generally present housekeeping epitopes. Thus, unless the epitope is produced by both the housekeeping and immuno-proteasomes, the target cell will generally not be recognized by CTL induced with TIL-identified epitopes. The epitopes of the present invention, in contrast, are generated by the action of a specified proteasome, indicating that they can be naturally produced, and enabling their appropriate use. The importance of the distinction between housekeeping and immune epitopes to vaccine design is more fully set forth in PCT publication WO 01/82963A2, which is hereby incorporated by reference in its entirety. The teachings and embodiments disclosed in said PCT publication are contemplated as supporting principals and embodiments related to and useful in connection with the present invention.

The epitopes of the invention include or encode polypeptide fragments of TAAs that are precursors or products of proteasomal cleavage by a housekeeping or immune proteasome, and that contain or consist of a sequence having a known or predicted affinity for at least one allele of MHC I. In some embodiments, the epitopes include or encode a polypeptide of about 6 to 25 amino acids in length, preferably about 7 to 20 amino acids in length, more preferably about 8 to 15 amino acids in length, and still more preferably 9 or 10 amino acids in length. However, it is understood that the polypeptides can be larger as long as N-terminal trimming can produce the MHC epitope or that they do not contain sequences that cause the polypeptides to be directed away from the proteasome or to be destroyed by the proteasome. For immune epitopes, if the larger peptides do not contain such sequences, they can be processed in the pAPC by the immune proteasome. Housekeeping epitopes may also be embedded in longer sequences provided that the sequence is adapted to facilitate liberation of the epitope's C-terminus by action of the immunoproteasome. The foregoing discussion has assumed that processing of longer epitopes proceeds through action of the immunoproteasome of the pAPC. However, processing can also be accomplished through the contrivance of some other mechanism, such as providing an exogenous protease activity and a sequence adapted so that action of the protease liberates the MHC epitope. The sequences of these epitopes can be subjected to computer analysis in order to calculate physical, biochemical, immunologic, or molecular genetic properties such as mass, isoelectric point, predicted mobility in electrophoresis, predicted binding to other MHC molecules, melting temperature of nucleic acid probes, reverse translations, similarity or homology to other sequences, and the like.

In constructing the polynucleotides encoding the polypeptide epitopes of the invention, the gene sequence of the associated TAA can be used, or the polynucleotide can be assembled from any of the corresponding codons. For a 10 amino acid epitope this can constitute on the order of 106 different sequences, depending on the particular amino acid composition. While large, this is a distinct and readily definable set representing a miniscule fraction of the >1018 possible polynucleotides of this length, and thus in some embodiments, equivalents of a particular sequence disclosed herein encompass such distinct and readily definable variations on the listed sequence. In choosing a particular one of these sequences to use in a vaccine, considerations such as codon usage, self-complementarity, restriction sites, chemical stability, etc. can be used as will be apparent to one skilled in the art.

The invention contemplates producing peptide epitopes. Specifically these epitopes are derived from the sequence of a TAA, and have known or predicted affinity for at least one allele of MHC I. Such epitopes are typically identical to those produced on target cells or pAPCs.

Compositions Containing Active Epitopes

Embodiments of the present invention provide polypeptide compositions, including vaccines, therapeutics, diagnostics, pharmacological and pharmaceutical compositions. The various compositions include newly identified epitopes of TAAs, as well as variants of these epitopes. Other embodiments of the invention provide polynucleotides encoding the polypeptide epitopes of the invention. The invention further provides vectors for expression of the polypeptide epitopes for purification. In addition, the invention provides vectors for the expression of the polypeptide epitopes in an APC for use as an anti-tumor vaccine. Any of the epitopes or antigens, or nucleic acids encoding the same, from Table 1 can be used. Other embodiments relate to methods of making and using the various compositions.

A general architecture for a class I MHC-binding epitope can be described, and has been reviewed more extensively in Madden, D. R. Annu. Rev. Immunol. 13:587-622, 1995, which is hereby incorporated by reference in its entirety. Much of the binding energy arises from main chain contacts between conserved residues in the MHC molecule and the N- and C-termini of the peptide. Additional main chain contacts are made but vary among MHC alleles. Sequence specificity is conferred by side chain contacts of so-called anchor residues with pockets that, again, vary among MHC alleles. Anchor residues can be divided into primary and secondary. Primary anchor positions exhibit strong preferences for relatively well-defined sets of amino acid residues. Secondary positions show weaker and/or less well-defined preferences that can often be better described in terms of less favored, rather than more favored, residues. Additionally, residues in some secondary anchor positions are not always positioned to contact the pocket on the MHC molecule at all. Thus, a subset of peptides exists that bind to a particular MHC molecule and have a side chain-pocket contact at the position in question and another subset exists that show binding to the same MHC molecule that does not depend on the conformation the peptide assumes in the peptide-binding groove of the MHC molecule. The C-terminal residue (PΩ; omega) is preferably a primary anchor residue. For many of the better studied HLA molecules (e.g. A2, A68, B27, B7, B35, and B53) the second position (P2) is also an anchor residue. However, central anchor residues have also been observed including P3 and P5 in HLA-B8, as well as P5 and PΩ(omega)-3 in the murine MHC molecules H-2D and H-2 Kb, respectively. Since more stable binding will generally improve immunogenicity, anchor residues are preferably conserved or optimized in the design of variants, regardless of their position.

Because the anchor residues are generally located near the ends of the epitope, the peptide can buckle upward out of the peptide-binding groove allowing some variation in length. Epitopes ranging from 8-11 amino acids have been found for HLA-A68, and up to 13 amino acids for HLA-A2. In addition to length variation between the anchor positions, single residue truncations and extensions have been reported and the N- and C-termini, respectively. Of the non-anchor residues, some point up out of the groove, making no contact with the MHC molecule but being available to contact the TCR, very often P1, P4, and PΩ(omega)-1 for HLA-A2. Others of the non-anchor residues can become interposed between the upper edges of the peptide-binding groove and the TCR, contacting both. The exact positioning of these side chain residues, and thus their effects on binding, MHC fine conformation, and ultimately immunogenicity, are highly sequence dependent. For an epitope to be highly immunogenic it must not only promote stable enough TCR binding for activation to occur, but the TCR must also have a high enough off-rate that multiple TCR molecules can interact sequentially with the same peptide-MHC complex (Kalergis, A. M. et al., Nature Immunol. 2:229-234, 2001, which is hereby incorporated by reference in its entirety). Thus, without further information about the ternary complex, both conservative and non-conservative substitutions at these positions merit consideration when designing variants.

The polypeptide epitope variants can be made, for example, using any of the techniques and guidelines for conservative and non-conservative mutations. Variants can be derived from substitution, deletion or insertion of one or more amino acids as compared with the native sequence. Amino acid substitutions can be the result of replacing one amino acid with another amino acid having similar structural and/or chemical properties, such as the replacement of a threonine with a serine, for example. Such replacements are referred to as conservative amino acid replacements, and all appropriate conservative amino acid replacements are considered to be embodiments of one invention. Insertions or deletions can optionally be in the range of about 1 to 4, preferably 1 to 2, amino acids. It is generally preferable to maintain the “anchor positions” of the peptide which are responsible for binding to the MHC molecule in question. Indeed, immunogenicity of peptides can be improved in many cases by substituting more preferred residues at the anchor positions (Franco, et al., Nature Immunology, 1(2):145-150, 2000, which is hereby incorporated by reference in its entirety). Immunogenicity of a peptide can also often be improved by substituting bulkier amino acids for small amino acids found in non-anchor positions while maintaining sufficient cross-reactivity with the original epitope to constitute a useful vaccine. The variation allowed can be determined by routine insertions, deletions or substitutions of amino acids in the sequence and testing the resulting variants for activity exhibited by the polypeptide epitope. Because the polypeptide epitope is often 9 amino acids, the substitutions preferably are made to the shortest active epitope, for example, an epitope of 9 amino acids.

Variants can also be made by adding any sequence onto the N-terminus of the polypeptide epitope variant. Such N-terminal additions can be from 1 amino acid up to at least 25 amino acids. Because peptide epitopes are often trimmed by N-terminal exopeptidases active in the pAPC, it is understood that variations in the added sequence can have no effect on the activity of the epitope. In preferred embodiments, the amino acid residues between the last upstream proteasomal cleavage site and the N-terminus of the MHC epitope do not include a proline residue. Serwold, T. at al., Nature Immunol. 2:644-651, 2001, which is hereby incorporated by reference in its entirety. Accordingly, effective epitopes can be generated from precursors larger than the preferred 9-mer class I motif.

Generally, peptides are useful to the extent that they correspond to epitopes actually displayed by MHC I on the surface of a target cell or a pACP. A single peptide can have varying affinities for different MHC molecules, binding some well, others adequately, and still others not appreciably (Table 2). MHC alleles have traditionally been grouped according to serologic reactivity which does not reflect the structure of the peptide-binding groove, which can differ among different alleles of the same type. Similarly, binding properties can be shared across types; groups based on shared binding properties have been termed supertypes. There are numerous alleles of MHC I in the human population; epitopes specific to certain alleles can be selected based on the genotype of the patient.

TABLE 2
Predicted Binding of Tyrosinase₂₀₇₋₂₁₆ (SEQ
ID NO. 1) to Various MHC types

| | *Half time of |
|---|---|
| MHC I type | dissociation (min) |
| A1 | 0.05 |
| A*0201 | 1311. |
| A*0205 | 50.4 |
| A3 | 2.7 |
| A*1101 (part of the A3 supertype) | 0.012 |
| A24 | 6.0 |
| B7 | 4.0 |
| B8 | 8.0 |
| B14 (part of the B27 supertype) | 60.0 |
| B*2702 | 0.9 |
| B*2705 | 30.0 |
| B*3501 (part of the B7 supertype) | 2.0 |
| B*4403 | 0.1 |
| B*5101 (part of the B7 supertype) | 26.0 |
| B*5102 | 55.0 |
| B*5801 | 0.20 |
| B60 | 0.40 |
| B62 | 2.0 |

*HLA Peptide Binding Predictions (world wide web hypertext transfer protocol “access at bimas.dcrt.nih.gov/molbio/hla_bin”).

In further embodiments of the invention, the epitope, as peptide or encoding polynucleotide, can be administered as a pharmaceutical composition, such as, for example, a vaccine or an immunogenic composition, alone or in combination with various adjuvants, carriers, or excipients. It should be noted that although the term vaccine may be used throughout the discussion herein, the concepts can be applied and used with any other pharmaceutical composition, including those mentioned herein. Particularly advantageous adjuvants include various cytokines and oligonucleotides containing immunostimulatory sequences (as set forth in greater detail in the co-pending applications referenced herein). Additionally the polynucleotide encoded epitope can be contained in a virus (e.g. vaccinia or adenovirus) or in a microbial host cell (e.g. Salmonella or Listeria monocytogenes) which is then used as a vector for the polynucleotide (Dietrich, G. et al. Nat. Biotech. 16:181-185, 1998, which is hereby incorporated by reference in its entirety). Alternatively a pAPC can be transformed, ex vivo, to express the epitope, or pulsed with peptide epitope, to be itself administered as a vaccine. To increase efficiency of these processes, the encoded epitope can be carried by a viral or bacterial vector, or complexed with a ligand of a receptor found on pAPC. Similarly the peptide epitope can be complexed with or conjugated to a pAPC ligand. A vaccine can be composed of more than a single epitope.

Particularly advantageous strategies for incorporating epitopes and/or epitope clusters, into a vaccine or pharmaceutical composition are disclosed in PCT Publication WO 01/82963 and U.S. patent application Ser. No. 09/560,465 entitled “EPITOPE SYNCHRONIZATION IN ANTIGEN PRESENTING CELLS,” filed on Apr. 28, 2000, which are hereby incorporated by reference in their entireties. The teaching and embodiments disclosed in said PCT publication are contemplated as supporting principals and embodiments related to and useful in connection with the present invention. Epitope clusters for use in connection with this invention are disclosed in PCT Publication WO 01/82963 and U.S. patent application Ser. No. 09/561,571 entitled “EPITOPE CLUSTERS,” filed on Apr. 28, 2000, which are hereby incorporated by reference in their entireties. The teaching and embodiments disclosed in said PCT publication are contemplated as supporting principals and embodiments related to and useful in connection with the present invention.

Preferred embodiments of the present invention are directed to vaccines and methods for causing a pAPC or population of pAPCs to present housekeeping epitopes that correspond to the epitopes displayed on a particular target cell. Any of the epitopes or antigens in Table 1, can be used for example. In one embodiment, the housekeeping epitope is a TuAA epitope processed by the housekeeping proteasome of a particular tumor type. In another embodiment, the housekeeping epitope is a virus-associated epitope processed by the housekeeping proteasome of a cell infected with a virus. This facilitates a specific T cell response to the target cells. Concurrent expression by the pAPCs of multiple epitopes, corresponding to different induction states (pre- and post-attack), can drive a CTL response effective against target cells as they display either housekeeping epitopes or immune epitopes.

By having both housekeeping and immune epitopes present on the pAPC, this embodiment can optimize the cytotoxic T cell response to a target cell. With dual epitope expression, the pAPCs can continue to sustain a CTL response to the immune-type epitope when the tumor cell switches from the housekeeping proteasome to the immune proteasome with induction by IFN, which, for example, may be produced by tumor-infiltrating CTLs.

In a preferred embodiment, immunization of a patient is with a vaccine that includes a housekeeping epitope. Many preferred TAAs are associated exclusively with a target cell, particularly in the case of infected cells. In another embodiment, many preferred TAAs are the result of deregulated gene expression in transformed cells, but are found also in tissues of the testis, ovaries and fetus. In another embodiment, useful TAAs are expressed at higher levels in the target cell than in other cells. In still other embodiments, TAAs are not differentially expressed in the target cell compare to other cells, but are still useful since they are involved in a particular function of the cell and differentiate the target cell from most other peripheral cells; in such embodiments, healthy cells also displaying the TAA may be collaterally attacked by the induced T cell response, but such collateral damage is considered to be far preferable to the condition caused by the target cell.

The vaccine contains a housekeeping epitope in a concentration effective to cause a pAPC or populations of pAPCs to display housekeeping epitopes. Advantageously, the vaccine can include a plurality of housekeeping epitopes or one or more housekeeping epitopes optionally in combination with one or more immune epitopes. Formulations of the vaccine contain peptides and/or nucleic acids in a concentration sufficient to cause pAPCs to present the epitopes. The formulations preferably contain epitopes in a total concentration of about 1 μg-1 mg/1001 of vaccine preparation. Conventional dosages and dosing for peptide vaccines and/or nucleic acid vaccines can be used with the present invention, and such dosing regimens are well understood in the art. In one embodiment, a single dosage for an adult human may advantageously be from about 1 to about 5000 μl of such a composition, administered one time or multiple times, e.g., in 2, 3, 4 or more dosages separated by 1 week, 2 weeks, 1 month, or more insulin pump delivers 1 ul per hour (lowest frequency) ref intranodal method patent.

The compositions and methods of the invention disclosed herein further contemplate incorporating adjuvants into the formulations in order to enhance the performance of the vaccines. Specifically, the addition of adjuvants to the formulations is designed to enhance the delivery or uptake of the epitopes by the pAPCs. The adjuvants contemplated by the present invention are known by those of skill in the art and include, for example, GMCSF, GCSF, IL-2, IL-12, BCG, tetanus toxoid, osteopontin, and ETA-1.

In some embodiments of the invention, the vaccines can include a recombinant organism, such as a virus, bacterium or parasite, genetically engineered to express an epitope in a host. For example, Listeria monocytogenes, a gram-positive, facultative intracellular bacterium, is a potent vector for targeting TuAAs to the immune system. In a preferred embodiment, this vector can be engineered to express a housekeeping epitope to induce therapeutic responses. The normal route of infection of this organism is through the gut and can be delivered orally. In another embodiment, an adenovirus (Ad) vector encoding a housekeeping epitope for a TuAA can be used to induce anti-virus or anti-tumor responses. Bone marrow-derived dendritic cells can be transduced with the virus construct and then injected, or the virus can be delivered directly via subcutaneous injection into an animal to induce potent T-cell responses. Another embodiment employs a recombinant vaccinia virus engineered to encode amino acid sequences corresponding to a housekeeping epitope for a TAA. Vaccinia viruses carrying constructs with the appropriate nucleotide substitutions in the form of a minigene construct can direct the expression of a housekeeping epitope, leading to a therapeutic T cell response against the epitope.

The immunization with DNA requires that APCs take up the DNA and express the encoded proteins or peptides. It is possible to encode a discrete class I peptide on the DNA. By immunizing with this construct, APCs can be caused to express a housekeeping epitope, which is then displayed on class I MHC on the surface of the cell for stimulating an appropriate CTL response. Constructs generally relying on termination of translation or non-proteasomal proteases for generation of proper termini of housekeeping epitopes have been described in PCT Publication WO 01/82963 and U.S. patent application Ser. No. 09/561,572 entitled EXPRESSION VECTORS ENCODING EPITOPES OF TARGET-ASSOCIATED ANTIGENS, filed on Apr. 28, 2000, which are hereby incorporated herein by reference in their entirety. The teaching and embodiments disclosed in said PCT publication are contemplated as supporting principals and embodiments related to and useful in connection with the present invention.

As mentioned, it can be desirable to express housekeeping peptides in the context of a larger protein. Processing can be detected even when a small number of amino acids are present beyond the terminus of an epitope. Small peptide hormones are usually proteolytically processed from longer translation products, often in the size range of approximately 60-120 amino acids. This fact has led some to assume that this is the minimum size that can be efficiently translated. In some embodiments, the housekeeping peptide can be embedded in a translation product of at least about 60 amino acids. In other embodiments the housekeeping peptide can be embedded in a translation product of at least about 50, 30, or 15 amino acids.

Due to differential proteasomal processing, the immune proteasome of the pAPC produces peptides that are different from those produced by the housekeeping proteasome in peripheral body cells. Thus, in expressing a housekeeping peptide in the context of a larger protein, it is preferably expressed in the APC in a context other than its full length native sequence, because, as a housekeeping epitope, it is generally only efficiently processed from the native protein by the housekeeping proteasome, which is not active in the APC. In order to encode the housekeeping epitope in a DNA sequence encoding a larger protein, it is useful to find flanking areas on either side of the sequence encoding the epitope that permit appropriate cleavage by the immune proteasome in order to liberate that housekeeping epitope. Altering flanking amino acid residues at the N-terminus and C-terminus of the desired housekeeping epitope can facilitate appropriate cleavage and generation of the housekeeping epitope in the APC. Sequences embedding housekeeping epitopes can be designed de novo and screened to determine which can be successfully processed by immune proteasomes to liberate housekeeping epitopes.

Alternatively, another strategy is very effective for identifying sequences allowing production of housekeeping epitopes in APC. A contiguous sequence of amino acids can be generated from head to tail arrangement of one or more housekeeping epitopes. A construct expressing this sequence is used to immunize an animal, and the resulting T cell response is evaluated to determine its specificity to one or more of the epitopes in the array. By definition, these immune responses indicate housekeeping epitopes that are processed in the pAPC effectively. The necessary flanking areas around this epitope are thereby defined. The use of flanking regions of about 4-6 amino acids on either side of the desired peptide can provide the necessary information to facilitate proteasome processing of the housekeeping epitope by the immune proteasome. Therefore, a sequence ensuring epitope synchronization of approximately 16-22 amino acids can be inserted into, or fused to, any protein sequence effectively to result in that housekeeping epitope being produced in an APC. In alternate embodiments the whole head-to-tail array of epitopes, or just the epitopes immediately adjacent to the correctly processed housekeeping epitope can be similarly transferred from a test construct to a vaccine vector.

In a preferred embodiment, the housekeeping epitopes can be embedded between known immune epitopes, or segments of such, thereby providing an appropriate context for processing. The abutment of housekeeping and immune epitopes can generate the necessary context to enable the immune proteasome to liberate the housekeeping epitope, or a larger fragment, preferably including a correct C-terminus. It can be useful to screen constructs to verify that the desired epitope is produced. The abutment of housekeeping epitopes can generate a site cleavable by the immune proteasome. Some embodiments of the invention employ known epitopes to flank housekeeping epitopes in test substrates; in others, screening as described below are used whether the flanking regions are arbitrary sequences or mutants of the natural flanking sequence, and whether or not knowledge of proteasomal cleavage preferences are used in designing the substrates.

Cleavage at the mature N-terminus of the epitope, while advantageous, is not required, since a variety of N-terminal trimming activities exist in the cell that can generate the mature N-terminus of the epitope subsequent to proteasomal processing. It is preferred that such N-terminal extension be less than about 25 amino acids in length and it is further preferred that the extension have few or no proline residues. Preferably, in screening, consideration is given not only to cleavage at the ends of the epitope (or at least at its C-terminus), but consideration also can be given to ensure limited cleavage within the epitope.

Shotgun approaches can be used in designing test substrates and can increase the efficiency of screening. In one embodiment multiple epitopes can be assembled one after the other, with individual epitopes possibly appearing more than once. The substrate can be screened to determine which epitopes can be produced. In the case where a particular epitope is of concern a substrate can be designed in which it appears in multiple different contexts. When a single epitope appearing in more than one context is liberated from the substrate additional secondary test substrates, in which individual instances of the epitope are removed, disabled, or are unique, can be used to determine which are being liberated and truly constitute sequences ensuring epitope synchronization.

Several readily practicable screens exist. A preferred in vitro screen utilizes proteasomal digestion analysis, using purified immune proteasomes, to determine if the desired housekeeping epitope can be liberated from a synthetic peptide embodying the sequence in question. The position of the cleavages obtained can be determined by techniques such as mass spectrometry, HPLC, and N-terminal pool sequencing; as described in greater detail in U.S. Patent Applications entitled METHOD OF EPITOPE DISCOVERY, EPITOPE SYNCHRONIZATION IN ANTIGEN PRESENTING CELLS, PCT Publication, U.S. applications and Provisional U.S. Patent Applications entitled EPITOPE SEQUENCES, which are all cited and incorporated by reference herein.

Alternatively, in vivo screens such as immunization or target sensitization can be employed. For immunization a nucleic acid construct capable of expressing the sequence in question is used. Harvested CTL can be tested for their ability to recognize target cells presenting the housekeeping epitope in question. Such targets cells are most readily obtained by pulsing cells expressing the appropriate MHC molecule with synthetic peptide embodying the mature housekeeping epitope. Alternatively, cells known to express housekeeping proteasome and the antigen from which the housekeeping epitope is derived, either endogenously or through genetic engineering, can be used. To use target sensitization as a screen, CTL, or preferably a CTL clone, that recognizes the housekeeping epitope can be used. In this case it is the target cell that expresses the embedded housekeeping epitope (instead of the pAPC during immunization) and it must express immune proteasome. Generally, the target cell can be transformed with an appropriate nucleic acid construct to confer expression of the embedded housekeeping epitope. Loading with a synthetic peptide embodying the embedded epitope using peptide loaded liposomes or a protein transfer reagent such as BIOPORTER™ (Gene Therapy Systems, San Diego, Calif.) represents an alternative.

Additional guidance on nucleic acid constructs useful as vaccines in accordance with the present invention are disclosed in WO 01/82963 and U.S. patent application Ser. No. 09/561,572 entitled “EXPRESSION VECTORS ENCODING EPITOPES OF TARGET-ASSOCIATED ANTIGENS,” filed on Apr. 28, 2000, both of which are hereby incorporated by reference in their entireties. Further, expression vectors and methods for their design, which are useful in accordance with the present invention are disclosed in PCT Publication WO 03/063770; U.S. patent application Ser. No. 10/292,413, filed on Nov. 7, 2002; and U.S. Provisional Application No. 60/336,968 (attorney docket number CTLIMM.022PR) entitled “EXPRESSION VECTORS ENCODING EPITOPES OF TARGET-ASSOCIATED ANTIGENS AND METHODS FOR THEIR DESIGN,” filed on Nov. 7, 2001; all of which are incorporated by reference in their entireties. The teaching and embodiments disclosed in said PCT publications are contemplated as supporting principals and embodiments related to and useful in connection with the present invention.

A preferred embodiment of the present invention includes a method of administering a vaccine including an epitope (or epitopes) to induce a therapeutic immune response. The vaccine is administered to a patient in a manner consistent with the standard vaccine delivery protocols that are known in the art. Methods of administering epitopes of TAAs including, without limitation, transdermal, intranodal, perinodal, oral, intravenous, intradermal, intramuscular, intraperitoneal, and mucosal administration, including delivery by injection, instillation or inhalation. A particularly useful method of vaccine delivery to elicit a CTL response is disclosed in Australian Patent No. 739189 issued Jan. 17, 2002; PCT Publication No. WO 099/02183; U.S. patent application Ser. No. 09/380,534, filed on Sep. 1, 1999; a Continuation-in-Part thereof U.S. patent application Ser. No. 09/776,232 both entitled “A METHOD OF INDUCING A CTL RESPONSE,” filed on Feb. 2, 2001, published as 20020007173; and PCT Publication No. WO 02/062368; all of which are incorporated herein by reference in their entireties. The teachings and embodiments disclosed in said publications and applications are contemplated as supporting principals and embodiments related to and useful in connection with the present invention.

Reagents Recognizing Epitopes

In another aspect of the invention, proteins with binding specificity for the epitope and/or the epitope-MHC molecule complex are contemplated, as well as the isolated cells by which they can be expressed. In one set of embodiments these reagents take the form of immunoglobulins: polyclonal sera or monoclonal antibodies (mAb), methods for the generation of which are well know in the art. Generation of mAb with specificity for peptide-MHC molecule complexes is known in the art. See, for example, Aharoni et al. Nature 351:147-150, 1991; Andersen et al. Proc. Natl. Acad. Sci. USA 93:1820-1824, 1996; Dadaglio et al. Immunity 6:727-738, 1997; Duc et al. Int. Immunol. 5:427-431,1993; Eastman et al. Eur. J. Immunol. 26:385-393, 1996; Engberg et al. Immunotechnology 4:273-278, 1999; Porgdor et al. Immunity 6:715-726, 1997; Puri et al. J. Immunol. 158:2471-2476, 1997; and Polakova, K., et al. J. Immunol. 165 342-348, 2000; all of which are hereby incorporated by reference in their entirety.

In other embodiments the compositions can be used to induce and generate, in vivo and in vitro, T-cells specific for the any of the epitopes and/or epitope-MHC complexes. In preferred embodiments the epitope can be any one or more of those listed in TABLE 1, for example. Thus, embodiments also relate to and include isolated T cells, T cell clones, T cell hybridomas, or a protein containing the T cell receptor (TCR) binding domain derived from the cloned gene, as well as a recombinant cell expressing such a protein. Such TCR derived proteins can be simply the extra-cellular domains of the TCR, or a fusion with portions of another protein to confer a desired property or function. One example of such a fusion is the attachment of TCR binding domains to the constant regions of an antibody molecule so as to create a divalent molecule. The construction and activity of molecules following this general pattern have been reported, for example, Plaksin, D. et al. J. Immunol. 158:2218-2227, 1997 and Lebowitz, M. S. et al. Cell Immunol. 192:175-184, 1999, which are hereby incorporated by reference in their entirety. The more general construction and use of such molecules is also treated in U.S. Pat. No. 5,830,755 entitled T CELL RECEPTORS AND THEIR USE IN THERAPEUTIC AND DIAGNOSTIC METHODS, which is hereby incorporated by reference in its entirety.

The generation of such T cells can be readily accomplished by standard immunization of laboratory animals, and reactivity to human target cells can be obtained by immunizing with human target cells or by immunizing HLA-transgenic animals with the antigen/epitope. For some therapeutic approaches T cells derived from the same species are desirable. While such a cell can be created by cloning, for example, a murine TCR into a human T cell as contemplated above, in vitro immunization of human cells offers a potentially faster option. Techniques for in vitro immunization, even using naive donors, are know in the field, for example, Stauss et al., Proc. Natl. Acad. Sci. USA 89:7871-7875, 1992; Salgaller et al. Cancer Res. 55:4972-4979, 1995; Tsai et al., J. Immunol. 158:1796-1802, 1997; and Chung et al., J. Immunother. 22:279-287, 1999; which are hereby incorporated by reference in their entirety.

Any of these molecules can be conjugated to enzymes, radiochemicals, fluorescent tags, and toxins, so as to be used in the diagnosis (imaging or other detection), monitoring, and treatment of the pathogenic condition associated with the epitope. Thus a toxin conjugate can be administered to kill tumor cells, radiolabeling can facilitate imaging of epitope positive tumor, an enzyme conjugate can be used in an ELISA-like assay to diagnose cancer and confirm epitope expression in biopsied tissue. In a further embodiment, such T cells as set forth above, following expansion accomplished through stimulation with the epitope and/or cytokines, can be administered to a patient as an adoptive immunotherapy.

Reagents Comprising Epitopes

A further aspect of the invention provides isolated epitope-MHC complexes. In a particularly advantageous embodiment of this aspect of the invention, the complexes can be soluble, multimeric proteins such as those described in U.S. Pat. No. 5,635,363 (tetramers) or U.S. Pat. No. 6,015,884 (Ig-dimers), both of which are hereby incorporated by reference in their entirety. Such reagents are useful in detecting and monitoring specific T cell responses, and in purifying such T cells.

Isolated MHC molecules complexed with epitopic peptides can also be incorporated into planar lipid bilayers or liposomes. Such compositions can be used to stimulate T cells in vitro or, in the case of liposomes, in vivo. Co-stimulatory molecules (e.g. B7, CD40, LFA-3) can be incorporated into the same compositions or, especially for in vitro work, co-stimulation can be provided by anti-co-receptor antibodies (e.g. anti-CD28, anti-CD154, anti-CD2) or cytokines (e.g. IL-2, IL-12). Such stimulation of T cells can constitute vaccination, drive expansion of T cells in vitro for subsequent infusion in an immuotherapy, or constitute a step in an assay of T cell function.

The epitope, or more directly its complex with an MHC molecule, can be an important constituent of functional assays of antigen-specific T cells at either an activation or readout step or both. Of the many assays of T cell function current in the art (detailed procedures can be found in standard immunological references such as Current Protocols in Immunology 1999 John Wiley & Sons Inc., N.Y., which is hereby incorporated by reference in its entirety) two broad classes can be defined, those that measure the response of a pool of cells and those that measure the response of individual cells. Whereas the former conveys a global measure of the strength of a response, the latter allows determination of the relative frequency of responding cells. Examples of assays measuring global response are cytotoxicity assays, ELISA, and proliferation assays detecting cytokine secretion. Assays measuring the responses of individual cells (or small clones derived from them) include limiting dilution analysis (LDA), ELISPOT, flow cytometric detection of unsecreted cytokine (described in U.S. Pat. No. 5,445,939, entitled “METHOD FOR ASSESSMENT OF THE MONONUCLEAR LEUKOCYTE IMMUNE SYSTEM” and U.S. Pat. Nos. 5,656,446; and 5,843,689, both entitled “METHOD FOR THE ASSESSMENT OF THE MONONUCLEAR LEUKOCYTE IMMUNE SYSTEM,” reagents for which are sold by Becton, Dickinson & Company under the tradename ‘FASTIMMUNE’, which patents are hereby incorporated by reference in their entirety) and detection of specific TCR with tetramers or Ig-dimers as stated and referenced above. The comparative virtues of these techniques have been reviewed in Yee, C. et al. Current Opinion in Immunology, 13:141-146, 2001, which is hereby incorporated by reference in its entirety. Additionally detection of a specific TCR rearrangement or expression can be accomplished through a variety of established nucleic acid based techniques, particularly in situ and single-cell PCR techniques, as will be apparent to one of skill in the art.

These functional assays are used to assess endogenous levels of immunity, response to an immunologic stimulus (e.g. a vaccine), and to monitor immune status through the course of a disease and treatment. Except when measuring endogenous levels of immunity, any of these assays presume a preliminary step of immunization, whether in vivo or in vitro depending on the nature of the issue being addressed. Such immunization can be carried out with the various embodiments of the invention described above or with other forms of immunogen (e.g., pAPC-tumor cell fusions) that can provoke similar immunity. With the exception of PCR and tetramer/Ig-dimer type analyses which can detect expression of the cognate TCR, these assays generally benefit from a step of in vitro antigenic stimulation which can advantageously use various embodiments of the invention as described above in order to detect the particular functional activity (highly cytolytic responses can sometimes be detected directly). Finally, detection of cytolytic activity requires epitope-displaying target cells, which can be generated using various embodiments of the invention. The particular embodiment chosen for any particular step depends on the question to be addressed, case of use, cost, and the like, but the advantages of one embodiment over another for any particular set of circumstances will be apparent to one of skill in the art.

The peptide MHC complexes described in this section have traditionally been understood to be non-covalent associations. However it is possible, and can be advantageous, to create a covalent linkages, for example by encoding the epitope and MHC heavy chain or the epitope, β2-microglobulin, and MHC heavy chain as a single protein (Yu, Y. L. Y., et al., J. Immunol. 168:3145-3149, 2002; Mottez, E., et at., J. Exp. Med. 181:493,1995; Dela Cruz, C. S., et al., Int. Immunol. 12:1293, 2000; Mage, M. G., et al., Proc. Natl. Acad. Sci. USA 89:10658,1992; Toshitani, K., et al., Proc. Natl. Acad. Sci. USA 93:236,1996; Lee, L., et al., Eur. J. Immunol. 24:2633,1994; Chung, D. H., et al., J. Immunol. 163:3699,1999; Uger, R. A. and B. H. Barber, J. Immunol. 160:1598, 1998; Uger, R. A., et al., J. Immunol. 162:6024,1999; and White, J., et al., J. Immunol. 162:2671, 1999; which are incorporated herein by reference in their entirety). Such constructs can have superior stability and overcome roadblocks in the processing-presentation pathway. They can be used in the already described vaccines, reagents, and assays in similar fashion.

Tumor Associated Antigens

Epitopes of the present invention are derived from the TuAAs tyrosinase (SEQ ID NO. 2), SSX-2, (SEQ ID NO. 3), PSMA (prostate-specific membrane antigen) (SEQ ID NO. 4), MAGE-1 (SEQ ID NO. 71), MAGE-2 (SEQ ID NO. 72), MAGE-3 (SEQ ID NO. 73), PRAME, (SEQ ID NO. 77), PSA, (SEQ ID NO. 78), PSCA, (SEQ ID NO. 79), CEA (carcinoembryonic antigen), (SEQ ID NO. 88), SCP-1 (SEQ ID NO. 92), GAGE-1, (SEQ ID NO. 96), survivin, (SEQ ID NO. 98), Melan-A/MART-1 (SEQ ID NO. 100), and BAGE (SEQ ID NO. 102). The natural coding sequences for these fifteen proteins, or any segments within them, can be determined from their cDNA or complete coding (cds) sequences, SEQ ID NOS. 5-7, 81-83, 85-87, 89, 93, 97, 99, 101, and 103, respectively.

Tyrosinase is a melanin biosynthetic enzyme that is considered one of the most specific markers of melanocytic differentiation. Tyrosinase is expressed in few cell types, primarily in melanocytes, and high levels are often found in melanomas. The usefulness of tyrosinase as a TuAA is taught in U.S. Pat. No. 5,747,271 entitled “METHOD FOR IDENTIFYING INDIVIDUALS SUFFERING FROM A CELLULAR ABNORMALITY SOME OF WHOSE ABNORMAL CELLS PRESENT COMPLEXES OF HLA-A2/TYROSINASE DERIVED PEPTIDES, AND METHODS FOR TREATING SAID INDIVIDUALS” which is hereby incorporated by reference in its entirety.

GP100, also known as PMel17, also is a melanin biosynthetic protein expressed at high levels in melanomas. GP100 as a TuAA is disclosed in U.S. Pat. No. 5,844,075 entitled “MELANOMA ANTIGENS AND THEIR USE IN DIAGNOSTIC AND THERAPEUTIC METHODS,” which is hereby incorporated by reference in its entirety.

Melan-A, also called MART-1 (Melanoma Antigen Recognized by T cells), is another melanin biosynthetic protein expressed at high levels in melanomas. The usefulness of Melan-A/MART-1 as a TuAA is taught in U.S. Pat. Nos. 5,874,560 and 5,994,523 both entitiled “MELANOMA ANTIGENS AND THEIR USE IN DIAGNOSTIC AND THERAPEUTIC METHODS,” as well as U.S. Pat. No. 5,620,886, entitled “ISOLATED NUCLEIC ACID SEQUENCE CODING FOR A TUMOR REJECTION ANTIGEN PRECURSOR PROCESSED TO AT LEAST ONE TUMOR REJECTION ANTIGEN PRESENTED BY HLA-A2”, all of which are hereby incorporated by reference in their entirety.

SSX-2, also know as Hom-MeI-40, is a member of a family of highly conserved cancer-testis antigens (Gure, A. O. et al. Int. J Cancer 72:965-971, 1997, which is hereby incorporated by reference in its entirety). Its identification as a TuAA is taught in U.S. Pat. No. 6,025,191 entitled “ISOLATED NUCLEIC ACID MOLECULES WHICH ENCODE A MELANOMA SPECIFIC ANTIGEN AND USES THEREOF,” which is hereby incorporated by reference in its entirety. Cancer-testis antigens are found in a variety of tumors, but are generally absent from normal adult tissues except testis. Expression of different members of the SSX family have been found variously in tumor cell lines. Due to the high degree of sequence identity among SSX family members, similar epitopes from more than one member of the family will be generated and able to bind to an MHC molecule, so that some vaccines directed against one member of this family can cross-react and be effective against other members of this family (see example 3 below).

MAGE-1, MAGE-2, and MAGE-3 are members of another family of cancer-testis antigens originally discovered in melanoma (MAGE is a contraction of melanoma-associated antigen) but found in a variety of tumors. The identification of MAGE proteins as TuAAs is taught in U.S. Pat. No. 5,342,774 entitled NUCLEOTIDE SEQUENCE ENCODING THE TUMOR REJECTION ANTIGEN PRECURSOR, MAGE-1, which is hereby incorporated by reference in its entirety, and in numerous subsequent patents. Currently there are 17 entries for (human) MAGE in the SWISS Protein database. There is extensive similarity among these proteins so in many cases, an epitope from one can induce a cross-reactive response to other members of the family. A few of these have not been observed in tumors, most notably MAGE-HI and MAGE-D1, which are expressed in testes and brain, and bone marrow stromal cells, respectively. The possibility of cross-reactivity on normal tissue is ameliorated by the fact that they are among the least similar to the other MAGE proteins.

GAGE-1 is a member of the GAGE family of cancer testis antigens (Van den Eynde, B., et al., J. Exp. Med. 182: 689-698, 1995; U.S. Pat. Nos. 5,610,013; 5,648,226; 5,858,689; 6,013,481; and 6,069,001). The PubGene database currently lists 12 distinct accessible members, some of which are synonymously known as PAGE or XAGE. GAGE-1 through GAGE-8 have a very high degree of sequence identity, so most epitopes can be shared among multiple members of the family.

BAGE is a cancer-testis antigen commonly expressed in melanoma, particularly metastatic melanoma, as well as in carcinomas of the lung, breast, bladder, and squamous cells of the head and neck. It's usefulness as a TuAA is taught in U.S. Pat. Nos. 5,683,881 entiltled “TUMOR REJECTION ANTIGENS WHICH CORRESPOND TO AMINO ACID SEQUENCES IN TUMOR REJECTION ANTIGEN PRECURSOR BAGE, AND USES THEREOF” and 5,571,711 entitled “ISOLATED NUCLEIC ACID MOLECULES CODING FOR BAGE TUMOR REJECTION ANTIGEN PRECURSORS”, both of which are hereby incorporated by reference in their entirety.

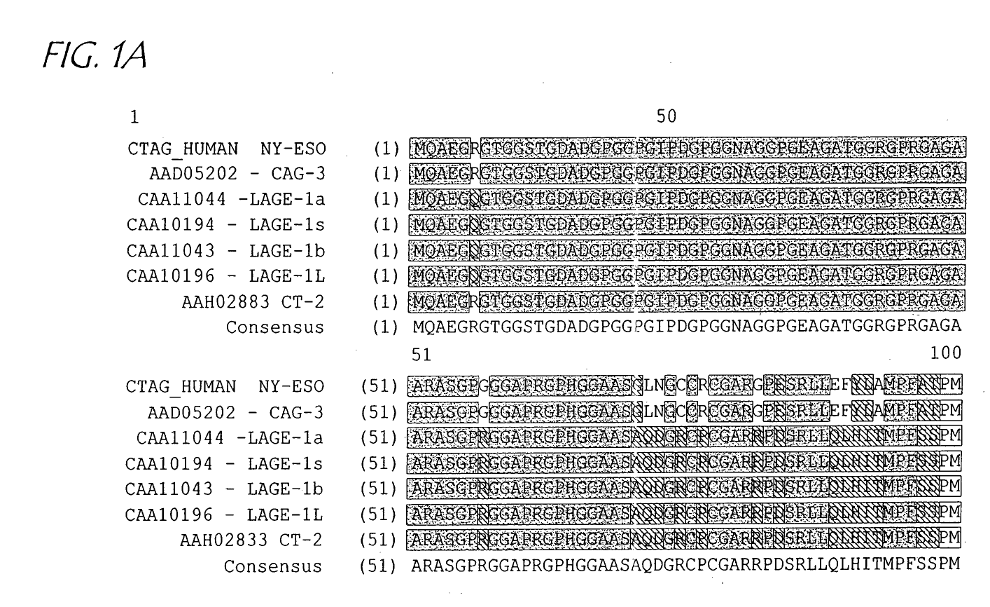

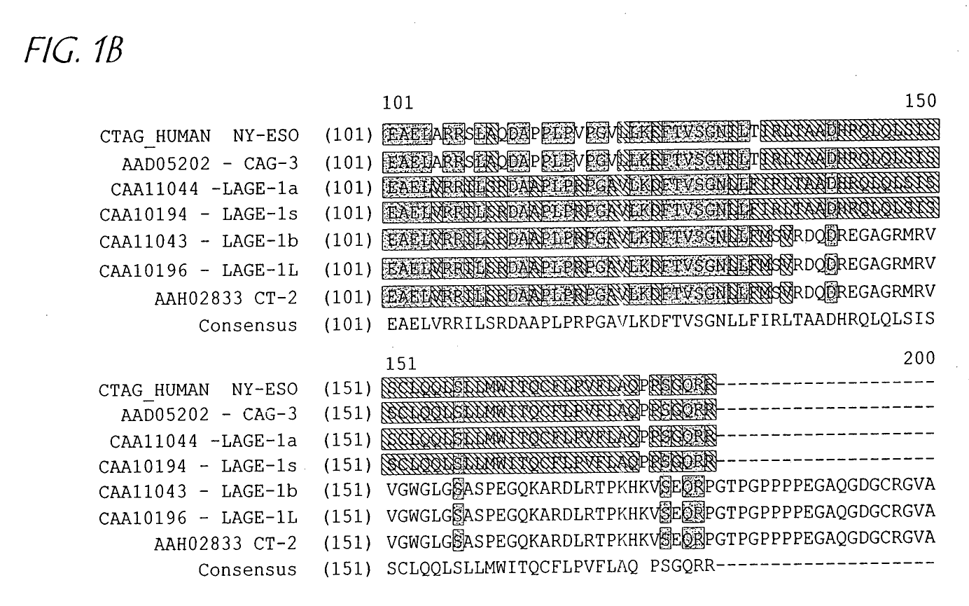

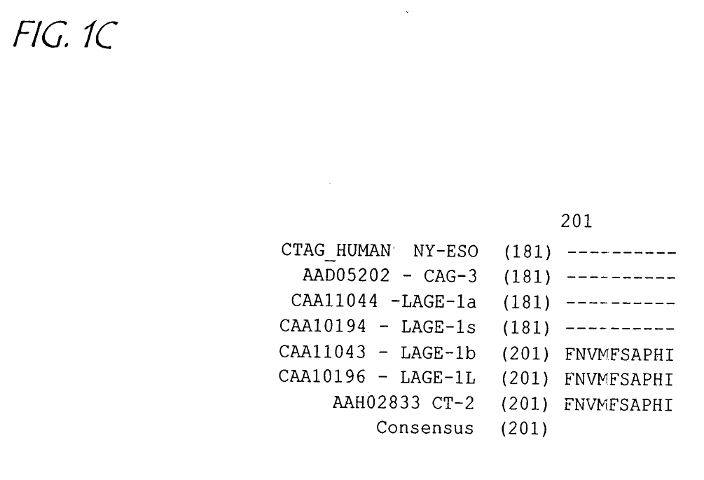

NY-ESO-1, is a cancer-testis antigen found in a wide variety of tumors, also known as CTAG-1 (Cancer-Testis Antigen-1) and CAG-3 (Cancer Antigen-3). NY-ESO-1 as a TuAA is disclosed in U.S. Pat. No. 5,804,381 entitled ISOLATED NUCLEIC ACID MOLECULE ENCODING AN ESOPHAGEAL CANCER ASSOCIATED ANTIGEN, THE ANTIGEN ITSELF, AND USES THEREOF which is hereby incorporated by reference in its entirety. A paralogous locus encoding antigens with extensive sequence identity, LAGE-1a/s (SEQ ID NO. 75) and LAGE-1b/L (SEQ ID NO. 76), have been disclosed in publicly available assemblies of the human genome, and have been concluded to arise through alternate splicing. Additionally, CT-2 (or CTAG-2, Cancer-Testis Antigen-2) appears to be either an allele, a mutant, or a sequencing discrepancy of LAGE-lb/L. Due to the extensive sequence identity, many epitopes from NY-ESO-1 can also induce immunity to tumors expressing these other antigens. See FIG. 1. The proteins are virtually identical through amino acid 70. From 71-134 the longest run of identities between NY-ESO-1 and LAGE is 6 residues, but potentially cross-reactive sequences are present. And from 135-180 NY-ESO and LAGE-1a/s are identical except for a single residue, but LAGE-lb/L is unrelated due to the alternate splice. The CAMEL and LAGE-2 antigens appear to derive from the LAGE-1 mRNA, but from alternate reading frames, thus giving rise to unrelated protein sequences. More recently, GenBank Accession AF277315.5, Homo sapiens chromosome X clone RP5-865E18, RP5-1087L19, complete sequence, reports three independent loci in this region which are labeled as LAGE1 (corresponding to CTAG-2 in the genome assemblies), plus LAGE2-A and LAGE2-B (both corresponding to CTAG-1 in the genome assemblies).

PSMA (prostate-specific membranes antigen), a TuAA described in U.S. Pat. No. 5,538,866 entitled “PROSTATE-SPECIFIC MEMBRANES ANTIGEN” which is hereby incorporated by reference in its entirety, is expressed by normal prostate epithelium and, at a higher level, in prostatic cancer. It has also been found in the neovasculature of non-prostatic tumors. PSMA can thus form the basis for vaccines directed to both prostate cancer and to the neovasculature of other tumors. This later concept is more fully described in U.S. Patent Publication No. 20030046714; PCT Publication No. WO 02/069907; and a provisional U.S. patent application No. 60/274,063 entitled ANTI-NEOVASCULAR VACCINES FOR CANCER, filed Mar. 7, 2001, and U.S. application Ser. No. 10/094,699, attorney docket number CTLIMM.015A, filed on Mar. 7, 2002, entitled “ANTI-NEOVASCULAR PREPARATIONS FOR CANCER,” all of which are hereby incorporated by reference in their entireties. The teachings and embodiments disclosed in said publications and applications are contemplated as supporting principals and embodiments related to and useful in connection with the present invention. Briefly, as tumors grow they recruit ingrowth of new blood vessels. This is understood to be necessary to sustain growth as the centers of unvascularized tumors are generally necrotic and angiogenesis inhibitors have been reported to cause tumor regression. Such new blood vessels, or neovasculature, express antigens not found in established vessels, and thus can be specifically targeted. By inducing CTL against neovascular antigens the vessels can be disrupted, interrupting the flow of nutrients to (and removal of wastes from) tumors, leading to regression.

Alternate splicing of the PSMA mRNA also leads to a protein with an apparent start at Met₅₈, thereby deleting the putative membrane anchor region of PSMA as described in U.S. Pat. No. 5,935,818 entitled “ISOLATED NUCLEIC ACID MOLECULE ENCODING ALTERNATIVELY SPLICED PROSTATE-SPECIFIC MEMBRANES ANTIGEN AND USES THEREOF” which is hereby incorporated by reference in its entirety. A protein termed PSMA-like protein, Genbank accession number AF261715, is nearly identical to amino acids 309-750 of PSMA and has a different expression profile. Thus the most preferred epitopes are those with an N-terminus located from amino acid 58 to 308.

PRAME, also know as MAPE, DAGE, and OIP4, was originally observed as a melanoma antigen. Subsequently, it has been recognized as a CT antigen, but unlike many CT antigens (e.g., MAGE, GAGE, and BAGE) it is expressed in acute myeloid leukemias. PRAME is a member of the MAPE family which consists largely of hypothetical proteins with which it shares limited sequence similarity. The usefulness of PRAME as a TuAA is taught in U.S. Pat. No. 5,830,753 entitled “ISOLATED NUCLEIC ACID MOLECULES CODING FOR TUMOR REJECTION ANTIGEN PRECURSOR DAGE AND USES THEREOF” which is hereby incorporated by reference in its entirety.

PSA, prostate specific antigen, is a peptidase of the kallikrein family and a differentiation antigen of the prostate. Expression in breast tissue has also been reported. Alternate names include gamma-seminoprotein, kallikrein 3, seminogelase, seminin, and P-30 antigen. PSA has a high degree of sequence identity with the various alternate splicing products prostatic/glandular kallikrein-1 and -2, as well as kallikrein 4, which is also expressed in prostate and breast tissue. Other kallikreins generally share less sequence identity and have different expression profiles. Nonetheless, cross-reactivity that might be provoked by any particular epitope, along with the likelihood that that epitope would be liberated by processing in non-target tissues (most generally by the housekeeping proteasome), should be considered in designing a vaccine.

PSCA, prostate stem cell antigen, and also known as SCAH-2, is a differentiation antigen preferentially expressed in prostate epithelial cells, and overexpresssed in prostate cancers. Lower level expression is seen in some normal tissues including neuroendocrine cells of the digestive tract and collecting ducts of the kidney. PSCA is described in U.S. Pat. No. 5,856,136 entitled “HUMAN STEM CELL ANTIGENS” which is hereby incorporated by reference in its entirety.

Synaptonemal complex protein 1 (SCP-1), also known as HOM-TES-14, is a meiosis-associated protein and also a cancer-testis antigen (Tureci, O., et al. Proc. Natl. Acad. Sci. USA 95:5211-5216, 1998). As a cancer antigen its expression is not cell-cycle regulated and it is found frequently in gliomas, breast, renal cell, and ovarian carcinomas. It has some similarity to myosins, but with few enough identities that cross-reactive epitopes are not an immediate prospect.

The ED-B domain of fibronectin is also a potential target. Fibronectin is subject to developmentally regulated alternative splicing, with the ED-B domain being encoded by a single exon that is used primarily in oncofetal tissues (Matsuura, H. and S. Hakomori Proc. Natl. Acad. Sci. USA 82:6517-6521, 1985; Carnemolla, B. et al. J. Cell Biol. 108:1139-1148, 1989; Loridon-Rosa, B. et al. Cancer Res. 50:1608-1612, 1990; Nicolo, G. et al. Cell Differ. Dev. 32:401-408, 1990; Borsi, L. et al. Exp. Cell Res. 199:98-105, 1992; Oyama, F. et al. Cancer Res. 53:2005-2011, 1993; Mandel, U. et al. APMIS 102:695-702, 1994; Farnoud, M. R. et al. Int. J. Cancer 61:27-34, 1995; Pujuguet, P. et al. Am. J. Pathol. 148:579-592, 1996; Gabler, U. et al. Heart 75:358-362, 1996; Chevalier, X. Br. J. Rheumatol. 35:407-415, 1996; Midulla, M. Cancer Res. 60:164-169, 2000).

The ED-B domain is also expressed in fibronectin of the neovasculature (Kaczmarek, J. et al. Int. J. Cancer 59:11-16, 1994; Castellani, P. et al. Int. J. Cancer 59:612-618, 1994; Neri, D. et al. Nat. Biotech. 15:1271-1275, 1997; Karelina, T. V. and A. Z. Eisen Cancer Detect. Prev. 22:438-444, 1998; Tarli, L. et al. Blood 94:192-198, 1999; Castellani, P. et al. Acta Neurochir. (Wien) 142:277-282, 2000). As an oncofetal domain, the ED-B domain is commonly found in the fibronectin expressed by neoplastic cells in addition to being expressed by the neovasculature. Thus, CTL-inducing vaccines targeting the ED-B domain can exhibit two mechanisms of action: direct lysis of tumor cells, and disruption of the tumor's blood supply through destruction of the tumor-associated neovasculature. As CTL activity can decay rapidly after withdrawal of vaccine, interference with normal angiogenesis can be minimal. The design and testing of vaccines targeted to neovasculature is described in Provisional U.S. patent Application No. 60/274,063 entitled “ANTI-NEOVASCULATURE VACCINES FOR CANCER” and in U.S. patent application Ser. No. 10/094,699, attorney docket number CTLIMM.015A, entitled “ANTI-NEOVASCULATURE PREPARATIONS FOR CANCER, filed on date even with this application (Mar. 7, 2002). A tumor cell line is disclosed in Provisional U.S. Application No. 60/363,131, filed on Mar. 7, 2002, attorney docket number CTLIMM.028PR, entitled “HLA-TRANSGENIC MURE TUMOR CELL LINE,” which is hereby incorporated by reference in its entirety.

Carcinoembryonic antigen (CEA) is a paradigmatic oncofetal protein first described in 1965 (Gold and Freedman, J. Exp. Med. 121: 439-462, 1965. Fuller references can be found in the Online Medelian Inheritance in Man; record *114890). It has officially been renamed carcinoembryonic antigen-related cell adhesion molecule 5 (CEACAM5). Its expression is most strongly associated with adenocarcinomas of the epithelial lining of the digestive tract and in fetal colon. CEA is a member of the immunoglobulin supergene family and the defining member of the CEA subfamily.

Survivin, also known as Baculoviral IAP Repeat-Containing Protein 5 (BIRC5), is another protein with an oncofetal pattern of expression. It is a member of the inhibitor of apoptosis protein (IAP) gene family. It is widely overexpressed in cancers (Ambrosini, G. et al., Nat. Med. 3:917-921, 1997; Velculiscu V. E. et al., Nat. Genet. 23:387-388, 1999) and it's function as an inhibitor of apoptosis is believed to contribute to the malignant phenotype.

HER2/NEU is an oncogene related to the epidermal growth factor receptor (van de Vijver, et al., New Eng. J. Med. 319:1239-1245, 1988), and apparently identical to the c-ERBB2 oncogene (Di Fiore, et al., Science 237: 178-182, 1987). The over-expression of ERBB2 has been implicated in the neoplastic transformation of prostate cancer. As HER2 it is amplified and over-expressed in 25-30% of breast cancers among other tumors where expression level is correlated with the aggressiveness of the tumor (Slamon, et al., New Eng. J. Med. 344:783-792, 2001). A more detailed description is available in the Online Medelian Inheritance in Man; record *164870.

All references mentioned herein are hereby incorporated by reference in their entirety. Further, incorporated by reference in its entirety is U.S. patent application Ser. No. 10/005,905 (attorney docket number CTLIMM.021CP1) entitled “EPITOPE SYNCHRONIZATION IN ANTIGEN PRESENTING CELLS,” filed on Nov. 7, 2001 and a continuation thereof, U.S. application Ser. No. 10/026,066, filed on Dec. 7, 2000, attorney docket number CTLIMM.21CP1C, also entitled “EPITOPE SYNCHRONIZATION IN ANTIGEN PRESENTING CELLS.”

Useful epitopes were identified and tested as described in the following examples. However, these examples are intended for illustration purposes only, and should not be construed as limiting the scope of the invention in any way.

### EXAMPLES

### Example 1

Manufacture of Epitopes.

A. Synthetic Production of Epitopes

Peptides having an amino acid sequence of any of SEQ ID NO: 1, 8, 9, 11-23, 26-29, 32-44, 47-54, 56-63, 66-68, or 108-602 are synthesized using either FMOC or tBOC solid phase synthesis methodologies. After synthesis, the peptides are cleaved from their supports with either trifluoroacetic acid or hydrogen fluoride, respectively, in the presence of appropriate protective scavengers. After removing the acid by evaporation, the peptides are extracted with ether to remove the scavengers and the crude, precipitated peptide is then lyophilized. Purity of the crude peptides is determined by HPLC, sequence analysis, amino acid analysis, counterion content analysis and other suitable means. If the crude peptides are pure enough (greater than or equal to about 90% pure), they can be used as is. If purification is required to meet drug substance specifications, the peptides are purified using one or a combination of the following: re-precipitation; reverse-phase, ion exchange, size exclusion or hydrophobic interaction chromatography; or counter-current distribution.

Drug Product Formulation

GMP-grade peptides are formulated in a parenterally acceptable aqueous, organic, or aqueous-organic buffer or solvent system in which they remain both physically and chemically stable and biologically potent. Generally, buffers or combinations of buffers or combinations of buffers and organic solvents are appropriate. The pH range is typically between 6 and 9. Organic modifiers or other excipients can be added to help solubilize and stabilize the peptides. These include detergents, lipids, co-solvents, antioxidants, chelators and reducing agents. In the case of a lyophilized product, sucrose or mannitol or other lyophilization aids can be added. Peptide solutions are sterilized by membrane filtration into their final container-closure system and either lyophilized for dissolution in the clinic, or stored until use.

B. Construction of Expression Vectors for Use as Nucleic Acid Vaccines

The construction of three generic epitope expression vectors is presented below. The particular advantages of these designs are set forth in PCT Publication No. WO 01/82963 and U.S. patent application Ser. No. 09/561,572 entitled “EXPRESSION VECTORS ENCODING EPITOPES OF TARGET-ASSOCIATED ANTIGENS,” filed on Apr. 28, 2000, which have been incorporated by reference in their entireties above. Additional vectors strategies for their design are disclosed in PCT Publication WO 03/063770; U.S. patent application Ser. No. 10/292,413, filed on Nov. 7, 2002; and Provisional U.S. patent application No. 60/336,968 entitled “EXPRESSION VECTORS ENCODING EPITOPES OF TARGET-ASSOCIATED ANTIGENS AND METHODS FOR THEIR DESIGN,” filed on Nov. 7, 2001, which were incorporated by reference in their entireties above. The teachings and embodiments disclosed in said PCT publications and applications are contemplated as supporting principals and embodiments related to and useful in connection with the present invention.

A suitable E. Coli strain was then transfected with the plasmid and plated out onto a selective medium. Several colonies were grown up in suspension culture and positive clones were identified by restriction mapping. The positive clone was then grown up and aliquotted into storage vials and stored at −70° C.

A mini-prep (QIAprep Spin Mini-prep: Qiagen, Valencia, Calif.) of the plasmid was then made from a sample of these cells and automated fluorescent dideoxy sequence analysis was used to confirm that the construct had the desired sequence.

B.1 Construction of pVAX-EP1-IRES-EP2

Overview:

The starting plasmid for this construct is pVAX1 purchased from Invitrogen (Carlsbad, Calif.). Epitopes EP1 and EP2 were synthesized by GIBCO BRL (Rockville, Md.). The IRES was excised from pIRES purchased from Clontech (Palo Alto, Calif.).

Procedure:

1. pIRES was digested with EcoRI and NotI. The digested fragments were separated by agarose gel electrophoresis, and the IRES fragment was purified from the excised band.

2. pVAX1 was digested with EcoRI and NotI, and the pVAX1 fragment was gel-purified.

3. The purified pVAX1 and IRES fragments were then ligated together.

4. Competent E. coli of strain DH5α were transformed with the ligation mixture.

5. Minipreps were made from 4 of the resultant colonies.

6. Restriction enzyme digestion analysis was performed on the miniprep DNA. One recombinant colony having the IRES insert was used for further insertion of EP1 and EP2. This intermediate construct was called pVAX-IRES.

7. Oligonucleotides encoding EP1 and EP2 were synthesized.

8. EP1 was subcloned into pVAX-IRES between AflII and EcoRI sites, to make pVAX-EP1-IRES;

9. EP2 was subcloned into pVAX-EP1-IRES between SalI and NotI sites, to make the final construct pVAX-EP1-IRES-EP2.

10. The sequence of the EP1-IRES-EP2 insert was confirmed by DNA sequencing.

B2. Construction of pVAX-EP 1-IRES-EP2-ISS-NIS

Overview:

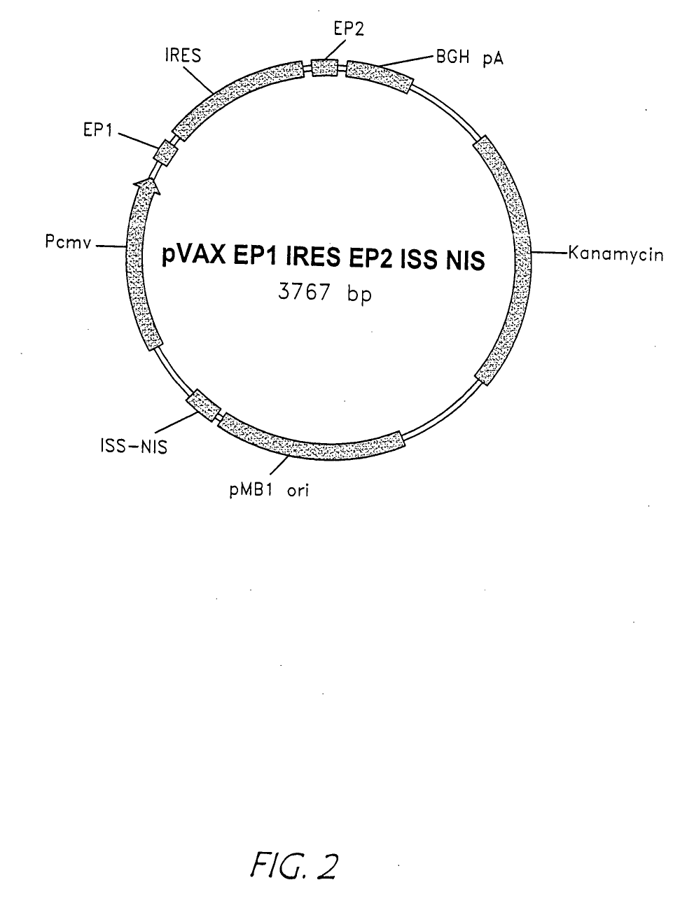

The starting plasmid for this construct was pVAX-EP1-IRES-EP2 (Example 1). The ISS (immunostimulatory sequence) introduced into this construct is AACGTT, and the NIS (standing for nuclear import sequence) used is the SV40 72 bp repeat sequence. ISS-NIS was synthesized by GIBCO BRL. See FIG. 2.

Procedure:

1. pVAX-EP1-IRES-EP2 was digested with NruI; the linearized plasmid was gel-purified.

2. ISS-NIS oligonucleotide was synthesized.

3. The purified linearized pVAX-EP1-IRES-EP2 and synthesized ISS-NIS were ligated together.

4. Competent E. coli of strain DH5α were transformed with the ligation product.

5. Minipreps were made from resultant colonies.

6. Restriction enzyme digestions of the minipreps were carried out.

7. The plasmid with the insert was sequenced.

B3. Construction of pVAX-EP2-UB-EP1

Overview:

The starting plasmid for this construct was pVAX1 (Invitrogen). EP2 and EP1 were synthesized by GIBCO BRL. Wild type Ubiquitin cDNA encoding the 76 amino acids in the construct was cloned from yeast.

Procedure:

1. RT-PCR was performed using yeast mRNA. Primers were designed to amplify the complete coding sequence of yeast Ubiquitin.

2. The RT-PCR products were analyzed using agarose gel electrophoresis. A band with the predicted size was gel-purified.

3. The purified DNA band was subcloned into pZERO1 at EcoRV site. The resulting clone was named pZERO-UB.

4. Several clones of pZERO-UB were sequenced to confirm the Ubiquitin sequence before further manipulations.

5. EP1 and EP2 were synthesized.

6. EP2, Ubiquitin and EP1 were ligated and the insert cloned into pVAX1 between BamHI and EcoRI, putting it under control of the CMV promoter.

7. The sequence of the insert EP2-UB-EP1 was confirmed by DNA sequencing.

### Example 2

Identification of Useful Epitope Variants.

The 10-mer FLPWHRLFLL (SEQ ID NO. 1) is identified as a useful epitope. Based on this sequence, numerous variants are made. Variants exhibiting activity in HLA binding assays (see Example 3, section 6) are identified as useful, and are subsequently incorporated into vaccines. Variants that increase the stability of binding, assayed can be particularly usefule, for example as described in WO 97/41440 entitled “Methods for Selecting and Producing T Cell Peptide Epitopes and Vaccines Incorporating Said Selected Epitopes,” which is incorporated herein by reference in its entirety. The teachings and embodiments disclosed in said PCT publication are contemplated as supporting principals and embodiments related to and useful in connection with the present invention.

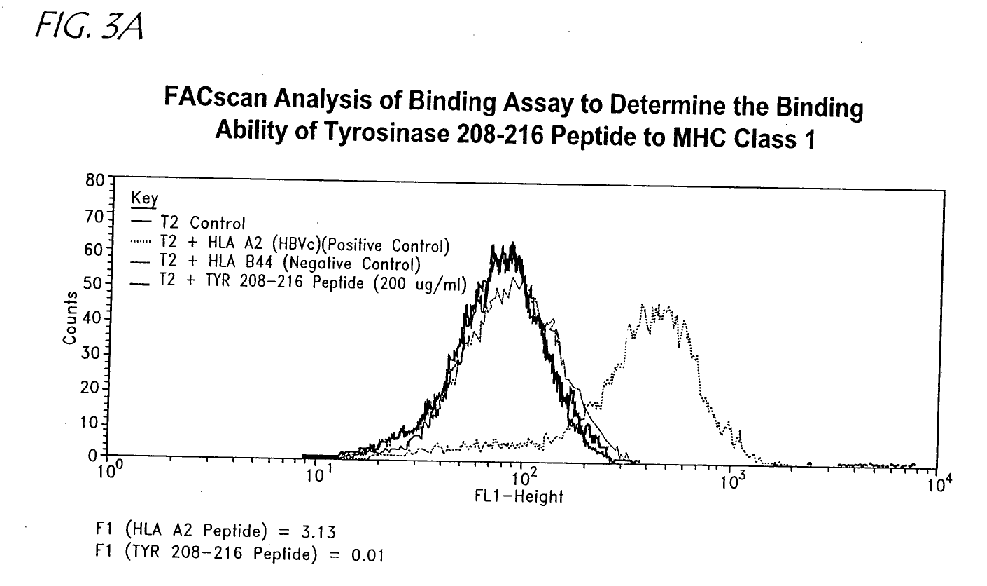

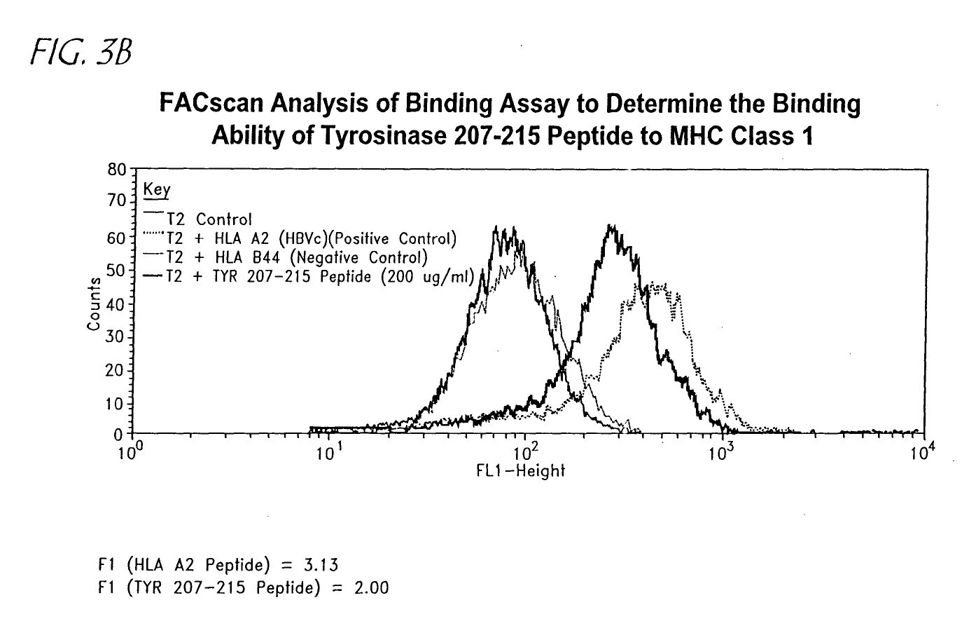

The HLA-A2 binding of length variants of FLPWHRLFLL have been evaluated. Proteasomal digestion analysis indicates that the C-terminus of the 9-mer FLPWHRLFL (SEQ ID NO. 8) is also produced. Additionally the 9-mer LPWHRLFLL (SEQ ID NO. 9) can result from N-terminal trimming of the 10-mer. Both are predicted to bind to the HLA-A*0201 molecule, however of these two 9-mers, FLPWHRLFL displayed more significant binding and is preferred (see FIGS. 3A and B).

In vitro proteasome digestion and N-terminal pool sequencing indicates that tyrosinase₂₀₇₋₂₁₆ (SEQ ID NO. 1) is produced more commonly than tyrosinase₂₀₇₋₂₁₅ (SEQ ID NO. 8), however the latter peptide displays superior immunogenicity, a potential concern in arriving at an optimal vaccine design. FLPWHRLFL, tyrosinase₂₀₇₋₂₁₅ (SEQ ID NO. 8) was used in an in vitro immunization of HLA-A2⁺ blood to generate CTL (see CTL Induction Cultures below). Using peptide pulsed T2 cells as targets in a standard chromium release assay it was found that the CTL induced by tyrosinase₂₀₇₋₂₁₅ (SEQ ID NO. 8) recognize tyrosinase₂₀₇₋₂₁₆ (SEQ ID NO. 1) targets equally well (see FIG. 3C). These CTL also recognize the HLA-A2⁺, tyrosinase⁺ tumor cell lines 624.38 and HTB64, but not 624.28 an HLA-A2 derivative of 624.38 (FIG. 3C). Thus the relative amounts of these two epitopes produced in vivo, does not become a concern in vaccine design.

CTL Induction Cultures

PBMCs from normal donors were purified by centrifugation in Ficoll-Hypaque from buffy coats. All cultures were carried out using the autologous plasma (AP) to avoid exposure to potential xenogeneic pathogens and recognition of FBS peptides. To favor the in vitro generation of peptide-specific CTL, we employed autologous dendritic cells (DC) as APCs. DC were generated and CTL were induced with DC and peptide from PBMCs as described (Keogh et al., 2001). Briefly, monocyte-enriched cell fractions were cultured for 5 days with GM-CSF and IL-4 and were cultured for 2 additional days in culture media with 2 μg/ml CD40 ligand to induce maturation. 2×10⁶ CD8+-enriched T lymphocytes/well and 2×10⁵ peptide-pulsed DC/well were co-cultured in 24-well plates in 2 ml RPMI supplemented with 10% AP, 10 ng/ml IL-7 and 20 IU/ml IL-2. Cultures were restimulated on days 7 and 14 with autologous irradiated peptide-pulsed DC.

Sequence variants of FLPWHRLFL are constructed as follow. Consistent with the binding coefficient table (see Table 3) from the NIH/BIMAS MHC binding prediction program (see reference in example 3 below), binding can be improved by changing the L at position 9, an anchor position, to V. Binding can also be altered, though generally to a lesser extent, by changes at non-anchor positions. Referring generally to Table 3, binding can be increased by employing residues with relatively larger coefficients. Changes in sequence can also alter immunogenicity independently of their effect on binding to MHC. Thus binding and/or immunogenicity can be improved as follows:

By substituting F,L,M,W, or Y for P at position 3; these are all bulkier residues that can also improve immunogenicity independent of the effect on binding. The amine and hydroxyl-bearing residues, Q and N; and S and T; respectively, can also provoke a stronger, cross-reactive response.

By substituting D or E for W at position 4 to improve binding; this addition of a negative charge can also make the epitope more immunogenic, while in some cases reducing cross-reactivity with the natural epitope. Alternatively the conservative substitutions of F or Y can provoke a cross-reactive response.

By substituting F for H at position 5 to improve binding. H can be viewed as partially charged, thus in some cases the loss of charge can hinder cross-reactivity. Substitution of the fully charged residues R or K at this position can enhance immunogenicity without disrupting charge-dependent cross-reactivity.

By substituting I, L, M, V, F, W, or Y for R at position 6. The same caveats and alternatives apply here as at position 5.

By substituting W or F for L at position 7 to improve binding. Substitution of V, I, S, T, Q, or N at this position are not generally predicted to reduce binding affinity by this model (the NIH algorithm), yet can be advantageous as discussed above.

Y and W, which are equally preferred as the Fs at positions 1 and 8, can provoke a useful cross-reactivity. Finally, while substitutions in the direction of bulkiness are generally favored to improve immunogenicity, the substitution of smaller residues such as A, S, and C, at positions 3-7 can be useful according to the theory that contrast in size, rather than bulkiness per se, is an important factor in immunogenicity. The reactivity of the thiol group in C can introduce other properties as discussed in Chen, J.-L., et al. J. Immunol. 165:948-955, 2000.

TABLE 3
9-mer Coefficient Table for HLA-A*0201*
HLA Coefficient table for file “A_0201_standard”

| Amino | | | | | | | | | |
|---|---|---|---|---|---|---|---|---|---|
| Acid | | | | | | | | | |
| Type | 1^{st} | 2^{nd} | 3rd | 4th | 5th | 6th | 7th | 8th | 9th |
| A | 1.000 | 1.000 | 1.000 | 1.000 | 1.000 | 1.000 | 1.000 | 1.000 | 1.000 |
| C | 1.000 | 0.470 | 1.000 | 1.000 | 1.000 | 1.000 | 1.000 | 1.000 | 1.000 |
| D | 0.075 | 0.100 | 0.400 | 4.100 | 1.000 | 1.000 | 0.490 | 1.000 | 0.003 |
| E | 0.075 | 1.400 | 0.064 | 4.100 | 1.000 | 1.000 | 0.490 | 1.000 | 0.003 |
| F | 4.600 | 0.050 | 3.700 | 1.000 | 3.800 | 1.900 | 5.800 | 5.500 | 0.015 |
| G | 1.000 | 0.470 | 1.000 | 1.000 | 1.000 | 1.000 | 0.130 | 1.000 | 0.015 |
| H | 0.034 | 0.050 | 1.000 | 1.000 | 1.000 | 1.000 | 1.000 | 1.000 | 0.015 |
| I | 1.700 | 9.900 | 1.000 | 1.000 | 1.000 | 2.300 | 1.000 | 0.410 | 2.100 |
| K | 3.500 | 0.100 | 0.035 | 1.000 | 1.000 | 1.000 | 1.000 | 1.000 | 0.003 |
| L | 1.700 | 72.000 | 3.700 | 1.000 | 1.000 | 2.300 | 1.000 | 1.000 | 4.300 |
| M | 1.700 | 52.000 | 3.700 | 1.000 | 1.000 | 2.300 | 1.000 | 1.000 | 1.000 |
| N | 1.000 | 0.470 | 1.000 | 1.000 | 1.000 | 1.000 | 1.000 | 1.000 | 0.015 |
| P | 0.022 | 0.470 | 1.000 | 1.000 | 1.000 | 1.000 | 1.000 | 1.000 | 0.003 |
| Q | 1.000 | 7.300 | 1.000 | 1.000 | 1.000 | 1.000 | 1.000 | 1.000 | 0.003 |
| R | 1.000 | 0.010 | 0.076 | 1.000 | 1.000 | 1.000 | 0.200 | 1.000 | 0.003 |
| S | 1.000 | 0.470 | 1.000 | 1.000 | 1.000 | 1.000 | 1.000 | 1.000 | 0.015 |
| T | 1.000 | 1.000 | 1.000 | 1.000 | 1.000 | 1.000 | 1.000 | 1.000 | 1.500 |
| V | 1.700 | 6.300 | 1.000 | 1.000 | 1.000 | 2.300 | 1.000 | 0.410 | 14.000 |
| W | 4.600 | 0.010 | 8.300 | 1.000 | 1.000 | 1.700 | 7.500 | 5.500 | 0.015 |
| Y | 4.600 | 0.010 | 3.200 | 1.000 | 1.000 | 1.500 | 1.000 | 5.500 | 0.015 |

*This table and other comparable data that are publicly available are useful in designing epitope variants and in determining whether a particular variant is substantially similar, or is functionally similar.

### Example 3

Cluster Analysis (SSX-2₃₁₋₆₈)

1. Epitope Cluster Region Prediction

The computer algorithms: SYFPEITHI (internet http://access at syfpeithi.bmi-heidelberg.com/Scripts/MHCServer.dll/EpPredict.htm), based on the book “MHC Ligands and Peptide Motifs” by H. G. Rammensee, J. Bachmann and S. Stevanovic; and HLA Peptide Binding Predictions (NIH) (internet http://access at bimas.dcrt.nih.gov/molbio/hla_bin), described in Parker, K. C., et al., J. Immunol. 152:163, 1994; were used to analyze the protein sequence of SSX-2 (GI:10337583). Epitope clusters (regions with higher than average density of peptide fragments with high predicted MHC affinity) were defined as described fully in U.S. patent application Ser. No. 09/561,571 entitled “EPITOPE CLUSTERS,” filed on Apr. 28, 2000. Using a epitope density ratio cutoff of 2, five and two clusters were defined using the SYFPETHI and NIH algorithms, respectively, and peptides score cutoffs of 16 (SYFPETHI) and 5 (NIH). The highest scoring peptide with the NIH algorithm, SSX-2₄₁₄₉, with an estimated halftime of dissociation of >1000 min., does not overlap any other predicted epitope but does cluster with SSX-2₅₇₋₆₅ in the NIH analysis.

2. Peptide Synthesis and Characterization:

SSX-2₃₁₋₆₈, YFSKEEWEKMKASEKIFYVYMKRKYEAMTKLGFKATLP (SEQ ID NO. 10) was synthesized by MPS (Multiple Peptide Systems, San Diego, Calif. 92121) using standard solid phase chemistry. According to the provided ‘Certificate of Analysis’, the purity of this peptide was 95%.

3. Proteasome digestion:

Proteasome was isolated from human red blood cells using the proteasome isolation protocol described in PCT Publication No. WO 01/82963 and U.S. patent application Ser. No. 09/561,074 entitled “METHOD OF EPITOPE DISCOVERY,” filed on Apr. 28, 2000; both of which are incorporated herein by reference in their entireties. The teachings and embodiments disclosed in said PCT publication and application are contemplated as supporting principals and embodiments related to and useful in connection with the present invention. SDS-PAGE, western-blotting, and ELISA were used as quality control assays. The final concentration of proteasome was 4 mg/ml, which was determined by non-interfering protein assay (Geno Technologies Inc.). Proteasomes were stored at −70° C. in 25 μl aliquots.

SSX-2₃₁₋₆₈ was dissolved in Milli-Q water, and a 2 mM stock solution prepared and 20 μL aliquots stored at −20° C.

1 tube of proteasome (25 μL) was removed from storage at −70° C. and thawed on ice. It was then mixed thoroughly with 12.5 μL of 2 mM peptide by repipetting (samples were kept on ice). A 5 μL sample was immediately removed after mixing and transferred to a tube containing 1.25 μL 10% TFA (final concentration of TFA was 2%); the T=0 min sample. The proteasome digestion reaction was then started and carried out at 37° C. in a programmable thermal controller. Additional 5 μL samples were taken out at 15, 30, 60, 120, 180 and 240 min respectively, the reaction was stopped by adding the sample to 1.25 μL 10% TFA as before. Samples were kept on ice or frozen until being analyzed by MALDI-MS. All samples were saved and stored at −20° C. for HPLC analysis and N-terminal sequencing. Peptide alone (without proteasome) was used as a blank control: 2 μL peptide+4 μL Tris buffer (20 mM, pH 7.6)+1.5 μL TFA.

4. MALDI-TOF MS Measurements:

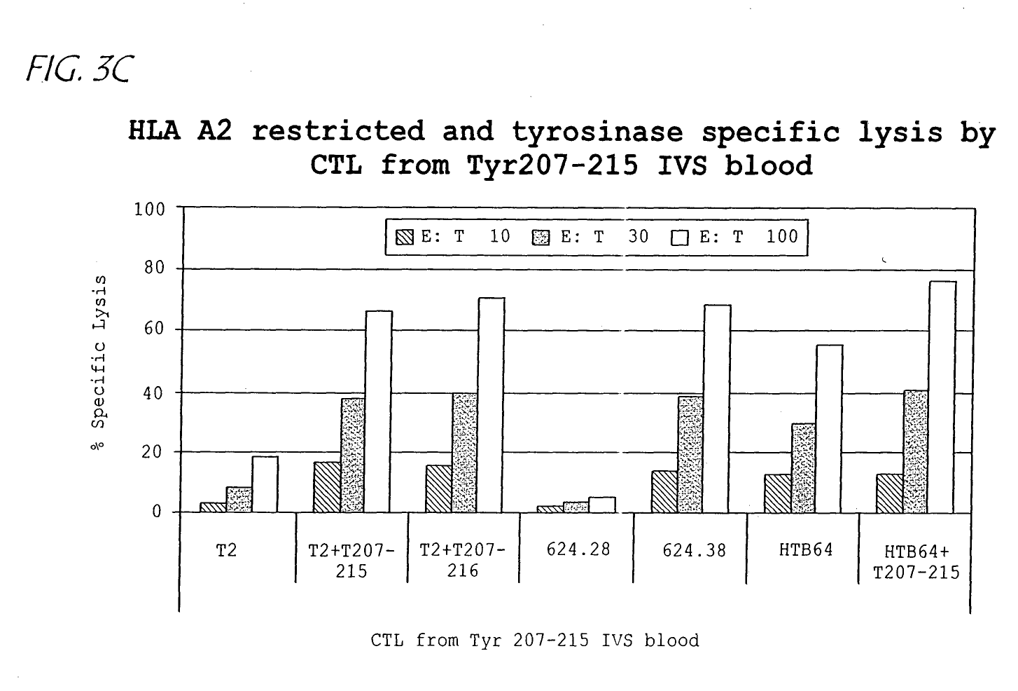

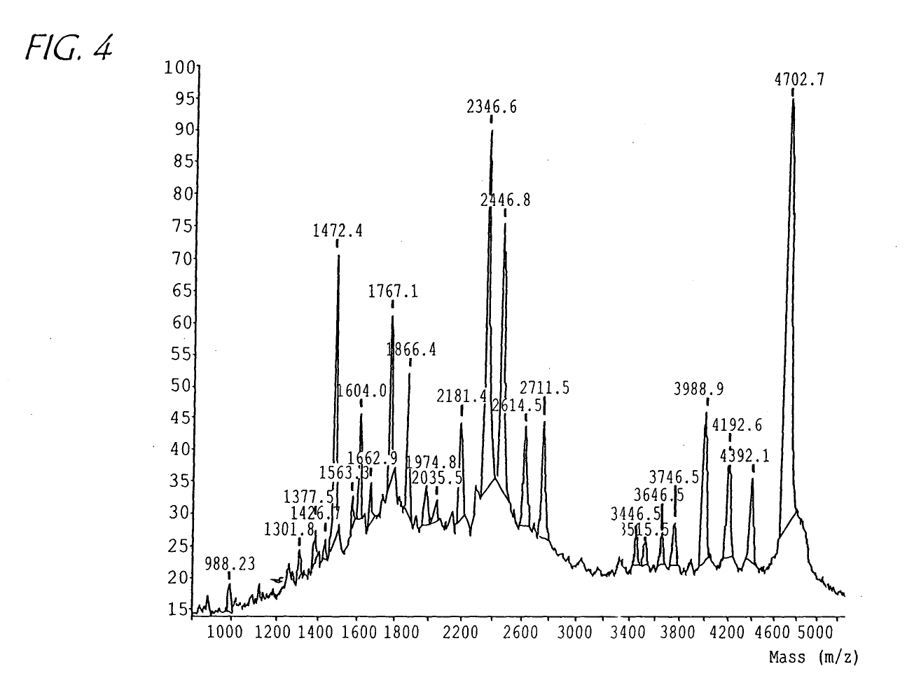

For each time point 0.3 μL of matrix solution (10 mg/ml α-cyano-4-hydroxycinnamic acid in AcCN/H₂O (70:30)) was first applied on a sample slide, and then an equal volume of digested sample was mixed gently with matrix solution on the slide. The slide was allowed to dry at ambient air for 3-5 min. before acquiring the mass spectra. MS was performed on a Lasermat 2000 MALDI-TOF mass spectrometer that was calibrated with peptide/protein standards. To improve the accuracy of measurement, the molecular ion weight (MH⁺) of the peptide substrate was used as an internal calibration standard. The mass spectrum of the T=120 min. digested sample is shown in FIG. 4.

5. MS Data Analysis and Epitope Identification:

To assign the measured mass peaks, the computer program MS-Product, a tool from the UCSF Mass Spectrometry Facility (http://accessible at prospector.ucsf.edu/ucsfhtml3.4/msprod.htm), was used to generate all possible fragments (N- and C-terminal ions, and internal fragments) and their corresponding molecular weights. Due to the sensitivity of the mass spectrometer, average molecular weight was used. The mass peaks observed over the course of the digestion were identified as summarized in Table 4.

Fragments co-C-terminal with 8-10 amino acid long sequences predicted to bind HLA by the SYFPEITHI or NIH algorithms were chosen for further study. The digestion and prediction steps of the procedure can be usefully practiced in any order. Although the substrate peptide used in proteasomal digest described here was specifically designed to include predicted HLA-A2.1 binding sequences, the actual products of digestion can be checked after the fact for actual or predicted binding to other MHC molecules. Selected results are shown in Table 5.

TABLE 4
SSX-2₃₁₋₆₈ Mass Peak Identification.

| | | | |
|---|---|---|---|
| MS PEAK | | | CALCULATED |
| (measured) | PEPTIDE | SEQUENCE | MASS (MH⁺) |

| | | | |
|---|---|---|---|
| 988.23 | 31-37 | YFSKEEW | 989.08 |
| 1377.68 ± 2.38 | 31-40 | YFSKEEWEKM | 1377.68 |
| 1662.45 ± 1.30 | 31-43 | YFSKEEWEKMKAS | 1663.90 |
| 2181.72 ± 0.85 | 31-47 | YFSKEEWEKMKASEKIF | 2181.52 |
| 2346.6 | 31-48 | YFSKEEWEKMKASEKIFY | 2344.71 |
| 1472.16 ± 1.54 | 38-49 | EKMKASEKIFYV | 1473.77 |
| 2445.78 ± 1.18 | 31-49* | YFSKEEWEKMKASEKIFYV | 2443.84 |
| 2607. | 31-50 | YFSKEEWEKMKASEKIFYVY | 2607.02 |
| 1563.3 | 50-61 | YMKRKYEAMTKL | 1562.93 |
| 3989.9 | 31-61 | YFSKEEWEKMKASEKIFYVYMKRKYEAMTKL | 3987.77 |
| 1603.74 ± 1.53 | 51-63 | MKRKYEAMTKLGF | 1603.98 |
| 1766.45 ± 1.5 | 50-63 | YMKRKYEAMTKLGF | 1767.16 |
| 1866.32 ± 1.22 | 49-63 | VYMKRKYEAMTKLGF | 1866.29 |
| 4192.6 | 31-63 | YFSKEEWEKMKASEKIFYVYMKRKYEAMTKLGF | 4192.00 |
| 4392.1 | 31-65** | YFSKEEWEKMKASEKIFYVYMKRKYEAMTKLGFKA | 4391.25 |

Boldface sequence correspond to peptides predicted to bind to MHC.
*On the basis of mass alone this peak could also have been assigned to the peptide 32-50, however proteasomal removal of just the N-terminal amino acid is unlikely. N-terminal sequencing (below) verifies the assignment to 31-49.
**On the basis of mass this fragment might also represent 33-68. N-terminal sequencing below is consistent with the assignment to 31-65.

TABLE 5
Predicted HLA binding by proteasomally generated fragments

| SEQ ID NO. | PEPTIDE | HLA | SYFPEITHI | NIH |
|---|---|---|---|---|
| 11 | FSKEEWEKM | B*3501 | NP† | 90 |
| 12 | KMKASEKIF | B*08 | 17 | <5 |
| 13 & (14) | (K) MKASEKIFY | A1 | 19 (19) | <5 |
| 15 & (16) | (M) KASEKIFYV | A*0201 | 22 (16) | 1017 |
| | | B*08 | 17 | <5 |
| | | B*5101 | 22 (13) | 60 |
| | | B*5102 | NP | 133 |
| | | B*5103 | NP | 121 |
| 17 & (18) | (K) ASEKIFYVY | A1 | 34 (19) | 14 |
| 19 & (20) | (K) RKYEAMTKL | A*0201 | 15 | <5 |
| | | A26 | 15 | NP |
| | | B14 | NP | 45 (60) |
| | | B*2705 | 21 | 15 |
| | | B*2709 | 16 | NP |
| | | B*5101 | 15 | <5 |
| 21 | KYEAMTKLGF | A1 | 16 | <5 |
| | | A24 | NP | 300 |
| 22 | YEAMTKLGF | B*4403 | NP | 80 |
| 23 | EAMTKLGF | B*08 | 22 | <5 |

†No prediction

As seen in Table 5, N-terminal addition of authentic sequence to epitopes can generate epitopes for the same or different MHC restriction elements. Note in particular the pairing of (K)RKYEAMTKL (SEQ ID NOS 19 and (20)) with HLA-B14, where the 10-mer has a longer predicted halftime of dissociation than the co-C-terminal 9mer. Also note the case of the 10-mer KYEAMTKLGF (SEQ ID NO. 21) which can be used as a vaccine useful with several MHC types by relying on N-terminal trimming to create the epitopes for HLA-B*4403 and -B*08.

6. LA-A0201 Binding Assay:

Binding of the candidate epitope KASEKIFYV, SSX-2₄₁₋₄₉, (SEQ ID NO. 15) to HLA-A2.1 was assayed using a modification of the method of Stauss et al., (Proc Natl Acad Sci USA 89(17):7871-5 (1992)). Specifically, T2 cells, which express empty or unstable MHC molecules on their surface, were washed twice with Iscove's modified Dulbecco's medium (IMDM) and cultured overnight in serum-free AIM-V medium (Life Technologies, Inc., Rockville, Md.) supplemented with human β2-microglobulinat 3 μg/ml (Sigma, St. Louis, Mo.) and added peptide, at 800, 400, 200, 100, 50, 25, 12.5, and 6.25 μg/ml.in a 96-well flat-bottom plate at 3×10⁵ cells/200 μl (microliter)/well. Peptide was mixed with the cells by repipeting before distributing to the plate (alternatively peptide can be added to individual wells), and the plate was rocked gently for 2 minutes. Incubation was in a 5% CO₂ incubator at 37° C. The next day the unbound peptide was removed by washing twice with serum free RPMI medium and a saturating amount of anti-class I HLA monoclonal antibody, fluorescein isothiocyanate (FITC)-conjugated anti-HLA A2, A28 (One Lambda, Canoga Park, Calif.) was added. After incubation for 30 minutes at 4° C., cells were washed 3 times with PBS supplemented with 0.5% BSA, 0.05%(w/v) sodium azide, pH 7.4-7.6 (staining buffer). (Alternatively W6/32 (Sigma) can be used as the anti-class I HLA monoclonal antibody the cells washed with staining buffer and then incubated with fluorescein isothiocyanate (FITC)-conjugated goat F(ab′) antimouse-IgG (Sigma) for 30 min at 4° C. and washed 3 times as before.) The cells were resuspended in 0.5 ml staining buffer. The analysis of surface HLA-A2.1 molecules stabilized by peptide binding was performed by flow cytometry using a FACScan (Becton Dickinson, San Jose, Calif.). If flow cytometry is not to be performed immediately the cells can be fixed by adding a quarter volume of 2% paraformaldehyde and storing in the dark at 4° C.

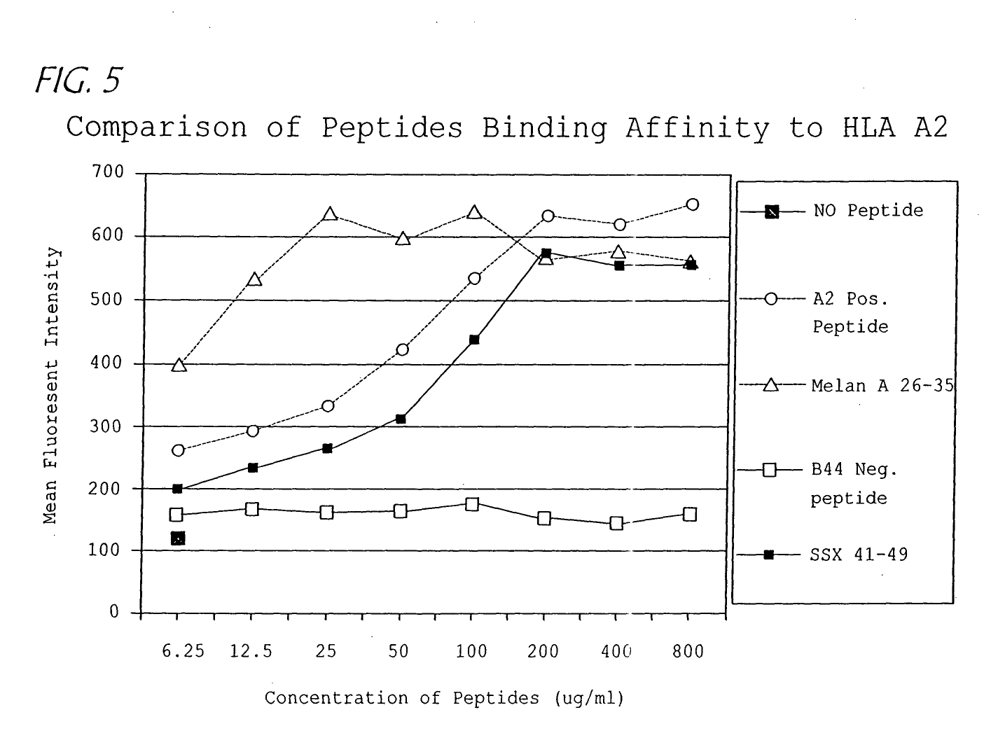

The results of the experiment are shown in FIG. 5. SSX-2₄₁₋₄₉ (SEQ ID NO. 15) was found to bind HLA-A2.1 to a similar extent as the known A2.1 binder FLPSDYFPSV (HBV₁₈₋₂₇; SEQ ID NO: 24) used as a positive control. An HLA-B44 binding peptide, AEMGKYSFY (SEQ ID NO: 25), was used as a negative control. The fluoresence obtained from the negative control was similar to the signal obtained when no peptide was used in the assay. Positive and negative control peptides were chosen from Table 18.3.1 in Current Protocols in Immunology p. 18.3.2, John Wiley and Sons, New York, 1998.

7. Immunogenicity:

A. In Vivo Immunization of Mice.

HHD1 transgenic A*0201 mice (Pascolo, S., et al. J. Exp. Med. 185:2043-2051, 1997) were anesthetized and injected subcutaneously at the base of the tail, avoiding lateral tail veins, using 100 μl containing 100 nmol of SSX-2₄₁₋₄₉ (SEQ ID NO. 15) and 20 μg of HTL epitope peptide in PBS emulsified with 50 μl of IFA (incomplete Freund's adjuvant).

B. Preparation of Stimulating Cells (LPS Blasts).

Using spleens from 2 naive mice for each group of immunized mice, un-immunized mice were sacrificed and the carcasses were placed in alcohol. Using sterile instruments, the top dermal layer of skin on the mouse's left side (lower mid-section) was cut through, exposing the peritoneum. The peritoneum was saturated with alcohol, and the spleen was aseptically extracted. The spleen was placed in a petri dish with serum-free media. Splenocytes were isolated by using sterile plungers from 3 ml syringes to mash the spleens. Cells were collected in a 50 ml conical tubes in serum-free media, rinsing dish well. Cells were centrifuged (12000 rpm, 7 min) and washed one time with RPMI. Fresh spleen cells were resuspended to a concentration of 1×10⁶ cells per ml in RPMI-10% FCS (fetal calf serum). 25 g/ml lipopolysaccharide and 7 μg/ml Dextran Sulfate were added. Cell were incubated for 3 days in T-75 flasks at 37° C., with 5% CO₂. Splenic blasts were collected in 50 ml tubes pelleted (12000 rpm, 7 min) and resuspended to 3×10⁷/ml in RPMI. The blasts were pulsed with the priming peptide at 50 μg/ml, RT 4 hr. mitomycin C-treated at 25 μg/ml, 37° C., 20 min and washed three times with DMEM. C. In vitro stimulation.

3 days after LPS stimulation of the blast cells and the same day as peptide loading, the primed mice were sacrificed (at 14 days post immunization) to remove spleens as above. 3×10⁶ splenocytes were co-cultured with 1×10⁶ LPS blasts/well in 24-well plates at 37° C., with 5% CO₂ in DMEM media supplemented with 10% FCS, 5×10⁻⁵ M β-mercaptoethanol, 100 μg/ml streptomycin and 100 IU/ml penicillin. Cultures were fed 5% (vol/vol) ConA supernatant on day 3 and assayed for cytolytic activity on day 7 in a ⁵¹Cr-release assay.

D. Chromium-Release Assay Measuring CTL Activity.

To assess peptide specific lysis, 2×10⁶ T2 cells were incubated with 100 μCi sodium chromate together with 50 μg/ml peptide at 37° C. for 1 hour. During incubation they were gently shaken every 15 minutes. After labeling and loading, cells were washed three times with 10 ml of DMEM-10% FCS, wiping each tube with a fresh Kimwipe after pouring off the supernatant. Target cells were resuspended in DMEM-10% FBS 1×10⁵/ml. Effector cells were adjusted to 1×10⁷/ml in DMEM-10% FCS and 100 μl serial 3-fold dilutions of effectors were prepared in U-bottom 96-well plates. 100 μl of target cells were added per well. In order to determine spontaneous release and maximum release, six additional wells containing 100 μl of target cells were prepared for each target. Spontaneous release was revealed by incubating the target cells with 100 μl medium; maximum release was revealed by incubating the target cells with 100 μl of 2% SDS. Plates were then centrifuged for 5 min at 600 rpm and incubated for 4 hours at 37° C. in 5% CO₂ and 80% humidity. After the incubation, plates were then centrifuged for 5 min at 1200 rpm. Supernatants were harvested and counted using a gamma counter. Specific lysis was determined as follows: % specific release=[(experimental release−spontaneous release)/(maximum release−spontaneous release)]×100.

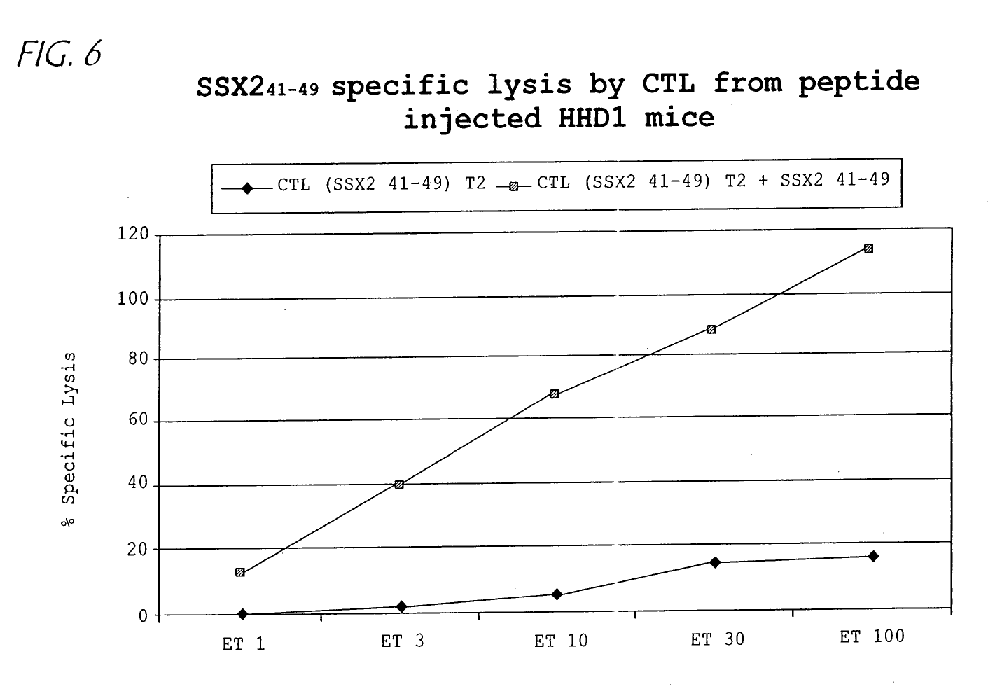

Results of the chromium release assay demonstrating specific lysis of peptide pulsed target cells are shown in FIG. 6.

8. Cross-Reactivity with Other SSX Proteins:

SSX-2₄₁₋₄₉ (SEQ ID NO. 15) shares a high degree of sequence identity with the same region of the other SSX proteins. The surrounding regions have also been generally well conserved. Thus the housekeeping proteasome can cleave following V₄₉ in all five sequences. Moreover, SSX₄₁₋₄₉ is predicted to bind HLA-A*0201 (see Table 6). CTL generated by immunization with SSX-2₄₁₋₄₉ cross-react with tumor cells expressing other SSX proteins.

TABLE 6
SSX₄₁₋₄₉ - A*0201 Predicted Binding

| | Family | | SYFPEITHI | NIH |
|---|---|---|---|---|
| SEQ ID NO. | Member | Sequence | Score | Score |
| 15 | SSX-2 | KASEKIFYV | 22 | 1017 |
| 26 | SSX-1 | KYSEKISYV | 18 | 1.7 |
| 27 | SSX-3 | KVSEKIVYV | 24 | 1105 |
| 28 | SSX-4 | KSSEKIVYV | 20 | 82 |
| 29 | SSX-5 | KASEKIIYV | 22 | 175 |

### Example 4

Cluster Analysis (PSMA₁₆₃₋₁₉₂).

A peptide, AFSPQGMPEGDLVYVNYARTEDFFKLERDM, PSMA₁₆₃₋₁₉₂, (SEQ ID NO. 30), containing an A1 epitope cluster from prostate specific membrane antigen, PSMA₁₆₈₋₁₉₀ (SEQ ID NO. 31) was synthesized using standard solid-phase F-moc chemistry on a 433A ABI Peptide synthesizer. After side chain deprotection and cleavage from the resin, peptide first dissolved in formic acid and then diluted into 30% Acetic acid, was run on a reverse-phase preparative HPLC C4 column at following conditions: linear AB gradient (5% B/min) at a flow rate of 4 ml/min, where eluent A is 0.1% aqueous TFA and eluent B is 0.1% TFA in acetonitrile. A fraction at time 16.642 min containing the expected peptide, as judged by mass spectrometry, was pooled and lyophilized. The peptide was then subjected to proteasome digestion and mass spectrum analysis essentially as described above. Prominent peaks from the mass spectra are summarized in Table 7.

TABLE 7
PSMA₁₆₃₋₁₉₂ Mass Peak Identification.

| | | |
|---|---|---|
| | | CALCULATE |
| | | D MASS |
| PEPTIDE | SEQUENCE | (MH⁺) |

| | | |
|---|---|---|
| 163-177 | AFSPQGMPEGDLVYV | 1610.0 |
| 178-189 | NYARTEDFFKLE | 1533.68 |
| 170-189 | PEGDLVYVNYARTEDFFKLE | 2406.66 |
| 178-191 | NYARTEDFFKLERD | 1804.95 |
| 170-191 | PEGDLVYVNYARTEDFFKLERD | 2677.93 |
| 178-192 | NYARTEDFFKLERDM | 1936.17 |
| 163-176 | AFSPQGMPEGDLVY | 1511.70 |
| 177-192 | VNYARTEDFFKLERDM | 2035.30 |
| 163-179 | AFSPQGMPEGDLVYVNY | 1888.12 |
| 180-192 | ARTEDFFKLERDM | 1658.89 |
| 163-183 | AFSPQGMPEGDLVYVNYARTE | 2345.61 |
| 184-192 | DFFKLERDM | 1201.40 |
| 176-192 | YVNYARTEDFFKLERDM | 2198.48 |
| 167-185 | QGMPEGDLVYVNYARTEDF | 2205.41 |
| 178-186 | NYARTEDFF | 1163.22 |

Boldface sequences correspond to peptides predicted to bind to MHC, see Table 8.

Boldface sequences correspond to peptides predicted to bind to MHC, see Table 8.

N-Terminal Pool Sequence Analysis

One aliquot at one hour of the proteasomal digestion (see Example 3 part 3 above) was subjected to N-terminal amino acid sequence analysis by an ABI 473A Protein Sequencer (Applied Biosystems, Foster City, Calif.). Determination of the sites and efficiencies of cleavage was based on consideration of the sequence cycle, the repetitive yield of the protein sequencer, and the relative yields of amino acids unique in the analyzed sequence. That is if the unique (in the analyzed sequence) residue X appears only in the nth cycle a cleavage site exists n−1 residues before it in the N-terminal direction. In addition to helping resolve any ambiguity in the assignment of mass to sequences, these data also provide a more reliable indication of the relative yield of the various fragments than does mass spectrometry.

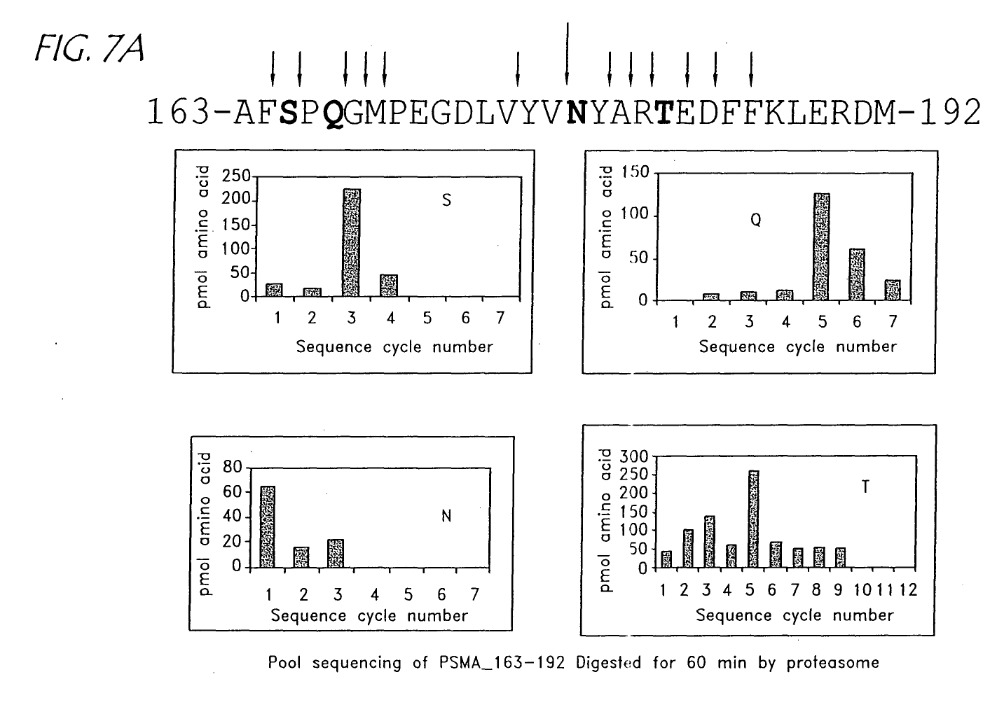

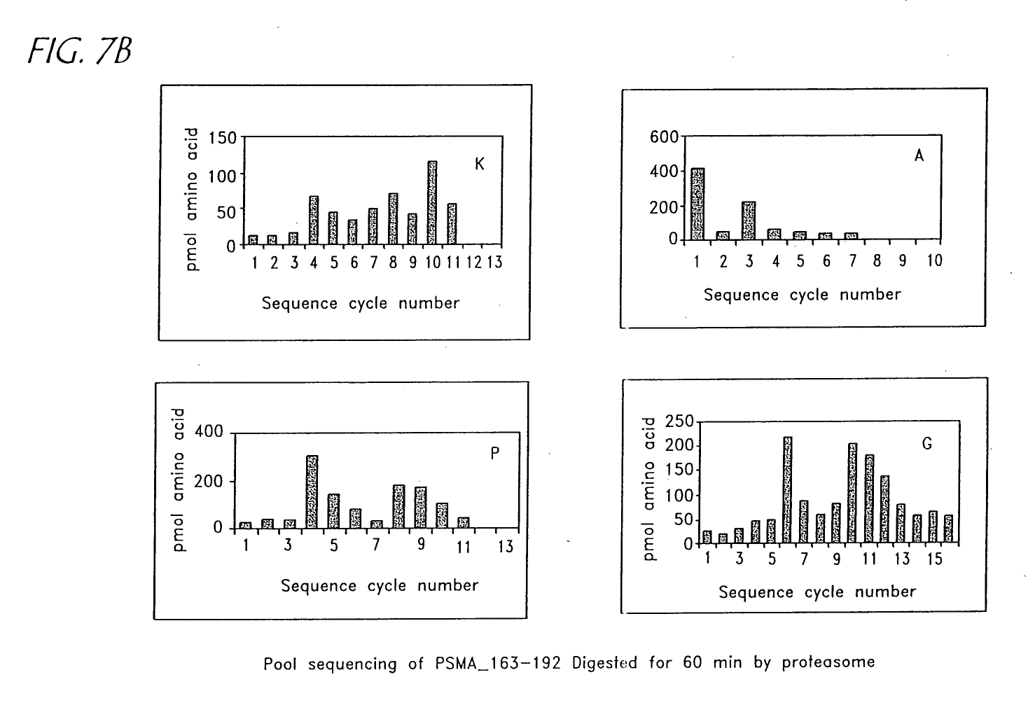

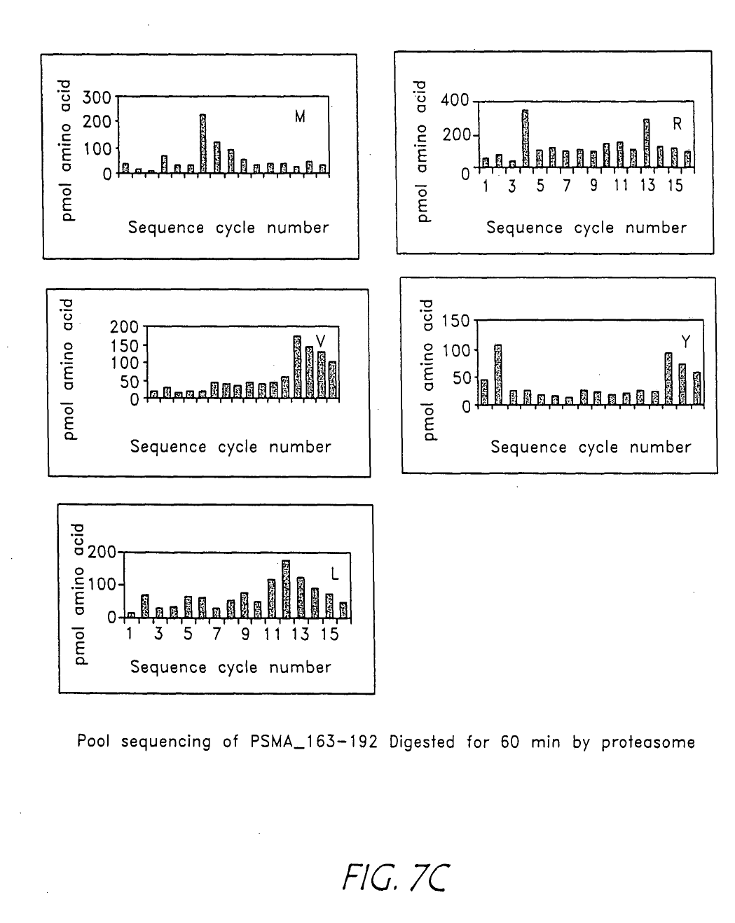

For PSMA₁₆₃₋₁₉₂ (SEQ ID NO. 30) this pool sequencing supports a single major cleavage site after V₁₇₇ and several minor cleavage sites, particularly one after Y₁₇₉. Reviewing the results presented in FIGS. 7A-C reveals the following:

S at the 3^{rd} cycle indicating presence of the N-terminus of the substrate.

Q at the 5^{th} cycle indicating presence of the N-terminus of the substrate.

N at the 1^{st} cycle indicating cleavage after V 77.

N at the 3^{rd} cycle indicating cleavage after V₁₇₅. Note the fragment 176-192 in Table 7.

T at the 5^{th} cycle indicating cleavage after V₁77.

T at the 1^{st}-3^{rd} cycles, indicating increasingly common cleavages after R₁₈₁, A₁₈₀ and Y₁₇₉. Only the last of these correspond to peaks detected by mass spectrometry; 163-179 and 180-192, see Table 7. The absence of the others can indicate that they are on fragments smaller than were examined in the mass spectrum.

K at the 4^{th}, 8^{th}, and 10^{th} cycles indicating cleavages after E₁₈₃, Y₁₇₉, and V₁₇₇, respectively, all of which correspond to fragments observed by mass spectroscopy. See Table 7.

A at the 11 and 3^{rd} cycles indicating presence of the N-terminus of the substrate and cleavage after V 77, respectively.

P at the 4^{th} and 8^{th} cycles indicating presence of the N-terminus of the substrate.

G at the 6^{th} and 10^{th} cycles indicating presence of the N-terminus of the substrate.

M at the 7^{th} cycle indicating presence of the N-terminus of the substrate and/or cleavage after F₁₈₅.

M at the 15^{th} cycle indicating cleavage after V₁₇₇.

The 1^{st} cycle can indicate cleavage after D₁₉₁, see Table 7.

R at the 4^{th} and 13^{th} cycle indicating cleavage after V₁ 77

R at the 2^{nd} and 11^{th} cycle indicating cleavage after Y₁₇₉.

V at the 2^{nd}, 6^{th}, and 13^{th} cycle indicating cleavage after V₁₇₅, M₁₆₉ and presence of the N-terminus of the substrate, respectively. Note fragments beginning at 176 and 170 in Table 7.

Y at the 1^{st}, 2^{nd}, and 14^{th} cycles indicating cleavage after V₁₇₅, V₁₇₇, and presence of the N-terminus of the substrate, respectively.

L at the 11^{th} and 12^{th} cycles indicating cleavage after V₁₇₇, and presence of the N-terminus of the substrate, respectively, is the interpretation most consistent with the other data. Comparing to the mass spectrometry results we see that L at the 2^{nd}, 5^{th}, and 9^{th} cycles is consistent with cleavage after F₁₈₆, E₁₈₃ or M₁₆₉, and Y₁₇₉, respectively. See Table 7.

Epitope Identification

Fragments co-C-terminal with 8-10 amino acid long sequences predicted to bind HLA by the SYFPEITHI or NIH algorithms were chosen for further analysis. The digestion and prediction steps of the procedure can be usefully practiced in any order. Although the substrate peptide used in proteasomal digest described here was specifically designed to include a predicted HLA-A1 binding sequence, the actual products of digestion can be checked after the fact for actual or predicted binding to other MHC molecules. Selected results are shown in Table 8.

TABLE 8
Predicted HLA binding by proteasomally generated fragments

| SEQ | | | | |
|---|---|---|---|---|
| ID NO | PEPTIDE | HLA | SYFPEITHI | NIH |
| 32 & (33) | (G)MPEGDLVYV | A*0201 | 17 (27) | (2605) |
| | | B*0702 | 20 | <5 |
| | | B*5101 | 22 | 314 |
| 34 & (35) | (Q)GMPEGDLVY | A1 | 24 (26) | <5 |
| | | A3 | 16 (18) | 36 |
| | | B*2705 | 17 | 25 |
| 36 | MPEGDLVY | B*5101 | 15 | NP† |
| 37 & (38) | (P)EGDLVYVNY | A1 | 27 (15) | 12 |
| | | A26 | 23 (17) | NP |
| 39 | LVYVNYARTE | A3 | 21 | <5 |
| 40 & (41) | (Y)VNYARTEDF | A26 | (20) | NP |
| | | B*08 | 15 | <5 |
| | | B*2705 | 12 | 50 |
| 42 | NYARTEDFF | A24 | NP† | 100 |
| | | Cw*0401 | NP | 120 |
| 43 | YARTEDFF | B*08 | 16 | <5 |
| 44 | RTEDFFKLE | A1 | 21 | <5 |
| | | A26 | 15 | NP |

†No prediction

HLA-A*0201 Binding Assay:

HLA-A*0201 binding studies were preformed with PSMA₁₆₈₋₁₇₇, GMPEGDLVYV, (SEQ ID NO. 33) essentially as described in Example 3 above. As seen in FIG. 8, this epitope exhibits significant binding at even lower concentrations than the positive control peptides. The Melan-A peptide used as a control in this assay (and throughout this disclosure), ELAGIGILTV, is actually a variant of the natural sequence (EAAGIGILTV) and exhibits a high affinity in this assay.

### Example 5

Cluster Analysis (PSMA₂₈₁₋₃₁₀).

Another peptide, RGIAEAVGLPSIPVHPIGYYDAQKLLEKMG, PSMA₂₈₁₋₃₁₀, (SEQ ID NO. 45), containing an A1 epitope cluster from prostate specific membrane antigen, PSMA₂₈₃₋₃₀₇ (SEQ ID NO. 46), was synthesized using standard solid-phase F-moc chemistry on a 433A ABI Peptide synthesizer. After side chain deprotection and cleavage from the resin, peptide in ddH2O was run on a reverse-phase preparative HPLC C18 column at following conditions: linear AB gradient (5% B/min) at a flow rate of 4 ml/min, where eluent A is 0.1% aqueous TFA and eluent B is 0.1% TFA in acetonitrile. A fraction at time 17.061 min containing the expected peptide as judged by mass spectrometry, was pooled and lyophilized. The peptide was then subjected to proteasome digestion and mass spectrum analysis essentially as described above. Prominent peaks from the mass spectra are summarized in Table 9.

TABLE 9
PSMA₂₈₁₋₃₁₀ Mass Peak Identification.

| | | |
|---|---|---|
| | | CALCULATE |
| PEPTIDE | SEQUENCE | D MASS (MH⁺) |

| | | |
|---|---|---|
| 281-297 | RGIAEAVGLPSIPVHPI* | 1727.07 |
| 286-297 | AVGLPSIPVHPI** | 1200.46 |
| 287-297 | VGLPSIPVHPI | 1129.38 |
| 288-297 | GLPSIPVHPI^{†} | 1030.25 |
| 298-310 | GYYDAQKLLEKMG‡ | 1516.5 |
| 298-305 | GYYDAQKL§ | 958.05 |
| 281-305 | RGIAEAVGLPSIPVHPIGYYDAQKL | 2666.12 |
| 281-307 | RGIAEAVGLPSIPVHPIGYYDAQKLLE | 2908.39 |
| 286-307 | AVGLPSIPVHPIGYYDAQKLLE¶ | 2381.78 |
| 287-307 | VGLPSIPVHPIGYYDAQKLLE | 2310.70 |
| 288-307 | GLPSIPVHPIGYYDAQKLLE# | 2211.57 |
| 281-299 | RGIAEAVGLPSIPVHPIGY | 1947 |
| 286-299 | AVGLPSIPVHPIGY | 1420.69 |
| 287-299 | VGLPSIPVHPIGY | 1349.61 |
| 288-299 | GLPSIPVHPIGY | 1250.48 |
| 287-310 | VGLPSIPVHPIGYYDAQKLLEKMG | 2627.14 |
| 288-310 | GLPSIPVHPIGYYDAQKLLEKMG | 2528.01 |

Boldface sequences correspond to peptides predicted to bind to MHC, see Table 10.
*By mass alone this peak could also have been 296-310 or 288-303.
**By mass alone this peak could also have been 298-307. Combination of HPLC and mass spectrometry show that at some later time points this peak is a mixture of both species.
^{†}By mass alone this peak could also have been 289-298.
≠By mass alone this peak could also have been 281-295 or 294-306.
§By mass alone this peak could also have been 297-303.
¶By mass alone this peak could also have been 285-306.
#By mass alone this peak could also have been 288-303.

None of these alternate assignments are supported N-terminal pool sequence analysis.

N-terminal Pool Sequence Analysis

One aliquot at one hour of the proteasomal digestion (see Example 3 part 3 above) was subjected to N-terminal amino acid sequence analysis by an ABI 473A Protein Sequencer (Applied Biosystems, Foster City, Calif.). Determination of the sites and efficiencies of cleavage was based on consideration of the sequence cycle, the repetitive yield of the protein sequencer, and the relative yields of amino acids unique in the analyzed sequence. That is if the unique (in the analyzed sequence) residue X appears only in the nth cycle a cleavage site exists n-1 residues before it in the N-terminal direction. In addition to helping resolve any ambiguity in the assignment of mass to sequences, these data also provide a more reliable indication of the relative yield of the various fragments than does mass spectrometry.

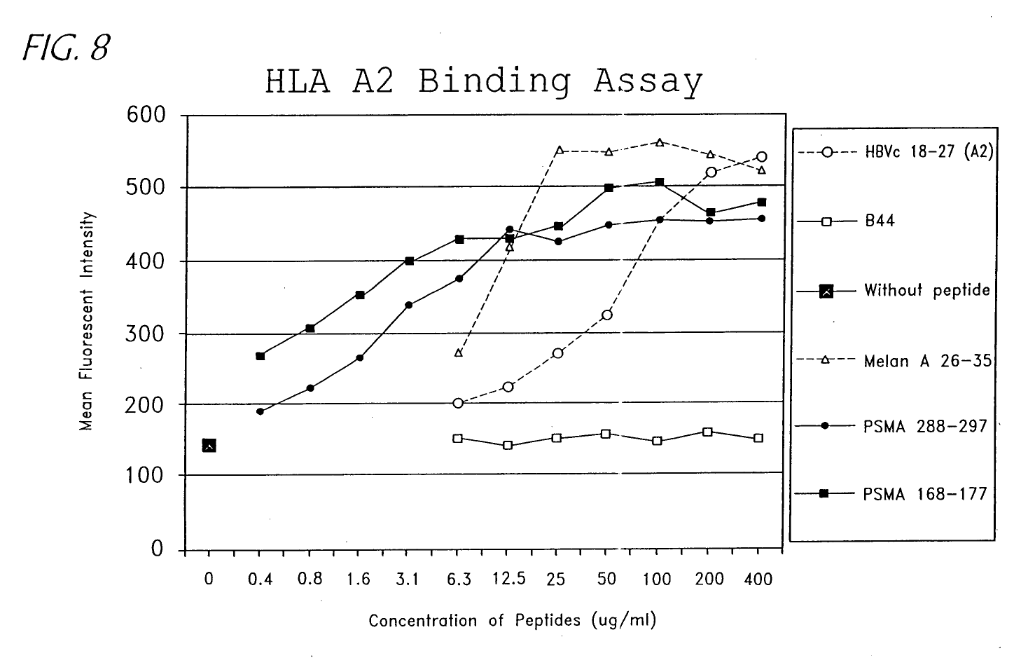

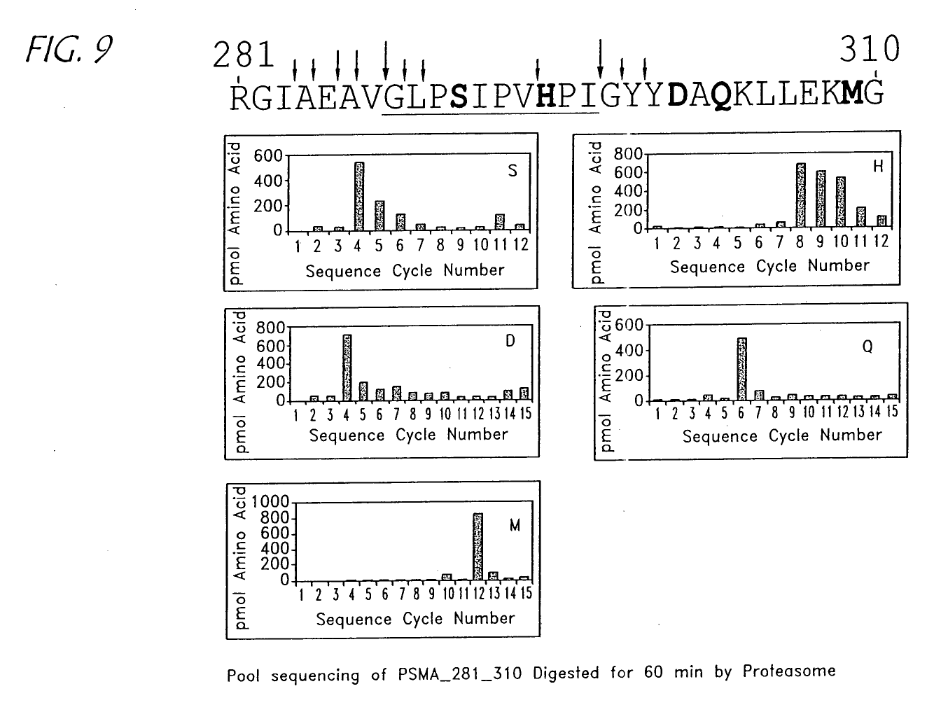

For PSMA₂₈₁₋₃₁₀ (SEQ ID NO. 45) this pool sequencing supports two major cleavage sites after V₂₈₇ and I₂₉₇ among other minor cleavage sites. Reviewing the results presented in FIG. 9 reveals the following:

S at the 4^{th} and 11^{th} cycles indicating cleavage after V₂₈₇ and presence of the N-terminus of the substrate, respectively.

H at the 8^{th} cycle indicating cleavage after V₂₈₇. The lack of decay in peak height at positions 9 and 10 versus the drop in height present going from 10 to 11 can suggest cleavage after A₂₈₆ and E₂₈₅ as well, rather than the peaks representing latency in the sequencing reaction.

D at the 2^{nd}, 4^{th}, and 7^{th} cycles indicating cleavages after Y₂₉₉, 1297, and V₂₉₄, respectively. This last cleavage is not observed in any of the fragments in Table 10 or in the alternate assignments in the notes below.

Q at the 6^{th} cycle indicating cleavage after 1297.

M at the 10^{th} and 12^{th} cycle indicating cleavages after Y₂₉₉ and I₂₉₇, respectively.

Epitope Identification

Fragments co-C-terminal with 8-10 amino acid long sequences predicted to bind HLA by the SYFPEITHI or NIH algorithms were chosen for further study. The digestion and prediction steps of the procedure can be usefully practiced in any order. Although the substrate peptide used in proteasomal digest described here was specifically designed to include a predicted HLA-A1 binding sequence, the actual products of digestion can be checked after the fact for actual or predicted binding to other MHC molecules. Selected results are shown in Table 10.

TABLE 10
Predicted HLA binding by proteasomally
generated fragments: PSMA₂₈₁₋₃₁₀

| SEQ | | | | |
|---|---|---|---|---|
| ID NO. | PEPTIDE | HLA | SYFPEITHI | NIH |
| 47 & (48) | (G)LPSIPVHPI | A*0201 | 16 (24) | (24) |
| | | B*0702/B7 | 23 | 12 |
| | | B*5101 | 24 | 572 |
| | | Cw*0401 | NP† | 20 |
| 49 & (50) | (P)IGYYDAQKL | A*0201 | (16) | <5 |
| | | A26 | (20) | NP |
| | | B*2705 | 16 | 25 |
| | | B*2709 | 15 | NP |
| | | B*5101 | 21 | 57 |
| | | Cw*0301 | NP | 24 |
| 51 & (52) | (P)SIPVHPIGY | A1 | 21 (27) | <5 |
| | | A26 | 22 | NP |
| | | A3 | 16 | <5 |
| 53 | | B*5101 | 16 | NP |
| | IPVHPIGY | | | |
| 54 | YYDAQKLLE | A1 | 22 | <5 |

†No prediction

As seen in Table 10, N-terminal addition of authentic sequence to epitopes can often generate still useful, even better epitopes, for the same or different MHC restriction elements. Note for example the pairing of (G)LPSIPVHPI with HLA-A*0201, where the 10-mer can be used as a vaccine useful with several MHC types by relying on N-terminal trimming to create the epitopes for HLA-B7, -B*5101, and Cw*0401.

HLA-A*0201 Binding Assay:

HLA-A*0201 binding studies were preformed with PSMA₂₈₈₋₂₉₇, GLPSIPVHPI, (SEQ ID NO. 48) essentially as described in Examples 3 and 4 above. As seen in FIG. 8, this epitope exhibits significant binding at even lower concentrations than the positive control peptides.

### Example 6

Cluster Analysis (PSMA₄₅₄₋₄₈₁).

Another peptide, SSIEGNYTLRVDCTPLMYSLVHLTKEL, PSMA₄₅₄₋₄₈₁, (SEQ ID NO. 55) containing an epitope cluster from prostate specific membrane antigen, was synthesized by MPS (purity >95%) and subjected to proteasome digestion and mass spectrum analysis as described above. Prominent peaks from the mass spectra are summarized in Table 11.

TABLE 11
PSMA₄₅₄₋₄₈₁ Mass Peak Identification.

| | | | |
|---|---|---|---|
| MS PEAK | | | CALCULATED |
| (measured) | PEPTIDE | SEQUENCE | MASS (MH⁺) |

| | | | |
|---|---|---|---|
| 1238.5 | 454-464 | SSIEGNYTLRV | 1239.78 |
| 1768.38 ± 0.60 | 454-469 | SSIEGNYTLRVDCTPL | 1768.99 |
| 1899.8 | 454-470 | SSIEGNYTLRVDCTPLM | 1900.19 |
| 1097.63 ± 0.91 | 463-471 | RVDCTPLMY | 1098.32 |
| 2062.87 ± 0.68 | 454-471* | SSIEGNYTLRVDCTPLMY | 2063.36 |
| 1153 | 472-481** | SLVHNLTKEL | 1154.36 |
| 1449.93 ± 1.79 | 470-481 | MYSLVHNLTKEL | 1448.73 |

Boldface sequence correspond to peptides predicted to bind to MHC, see Table 12.
*On the basis of mass alone this peak could equally well be assigned to the peptide 455-472 however proteasomal removal of just the N-terminal amino acid is considered unlikely. If the issue were important it could be resolved by N-terminal sequencing.
**On the basis of mass this fragment might also represent 455-464.

Epitope Identification

Fragments co-C-terminal with 8-10 amino acid long sequences predicted to bind HLA by the SYFPEITHI or NIH algorithms were chosen for further study. The digestion and prediction steps of the procedure can be usefully practiced in any order. Although the substrate peptide used in proteasomal digest described here was specifically designed to include predicted HLA-A2.1 binding sequences, the actual products of digestion can be checked after the fact for actual or predicted binding to other MHC molecules. Selected results are shown in Table 12.

TABLE 12
Predicted HLA binding by
proteasomally generated fragments

| | | | | |
|---|---|---|---|---|
| SEQ ID NO | PEPTIDE | HLA | SYFPEITHI | NIH |

| | | | | |
|---|---|---|---|---|
| 56 & (57) | (S) IEGNYTLRV | A1 | (19) | <5 |
| 58 | EGNYTLRV | A*0201 | 16 (22) | <5 |
| | | B*5101 | 15 | NP† |
| 59 & (60) | (Y) TLRVDCTPL | A*0201 | 20 (18) | (5) |
| | | A26 | 16 (18) | NP |
| | | B7 | 14 | 40 |
| | | B8 | 23 | <5 |
| | | B*2705 | 12 | 30 |
| | | Cw*0301 | NP | (30) |
| 61 | LRVDCTPLM | B*2705 | 20 | 600 |
| | | B*2709 | 20 | NP |
| 62 & (63) | (L) RVDCTPLMY | A1 | 32 (22) | 125 (13.5) |
| | | A3 | 25 | <5 |
| | | A26 | 22 | NP |
| | | B*2702 | NP | (200) |
| | | B*2705 | 13 (NP) | (1000) |

†No prediction

As seen in Table 12, N-terminal addition of authentic sequence to epitopes can often generate still useful, even better epitopes, for the same or different MHC restriction elements. Note for example the pairing of (L)RVDCTPLMY (SEQ ID NOS 62 and (63)) with HLA-B*2702/5, where the 10-mer has substantial predicted halftimes of dissociation and the co-C-terminal 9-mer does not. Also note the case of SIEGNYTLRV (SEQ ID NO 57) a predicted HLA-A*0201 epitope which can be used as a vaccine useful with HLA-B*5101 by relying on N-terminal trimming to create the epitope.

HLA-A*0201 Binding Assay

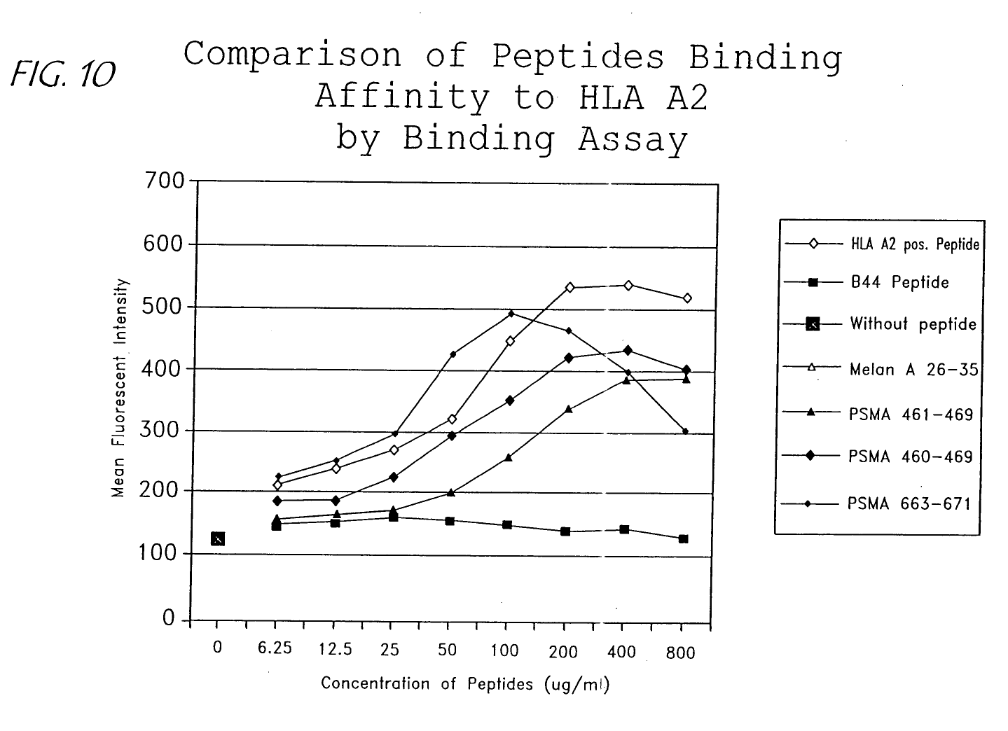

HLA-A*0201 binding studies were preformed, essentially as described in Example 3 above, with PSMA₄₆₀₋₄₆₉, TLRVDCTPL, (SEQ ID NO. 60). As seen in FIG. 10, this epitope was found to bind HLA-A2.1 to a similar extent as the known A2.1 binder FLPSDYFPSV (HBV₁₈₋₂₇; SEQ ID NO: 24) used as a positive control. Additionally, PSMA₄₆₁₋₄₆₉, (SEQ ID NO. 59) binds nearly as well.

ELISPOT analysis: PSMA₄₆₃₋₄₇₁ (SEQ ID NO. 62

The wells of a nitrocellulose-backed microtiter plate were coated with capture antibody by incubating overnight at 4° C. using 50 μl (microliter)/well of 4 μg/ml murine anti-human γ (gamma)-IFN monoclonal antibody in coating buffer (35 mM sodium bicarbonate, 15 mM sodium carbonate, pH 9.5). Unbound antibody was removed by washing 4 times 5 min. with PBS. Unbound sites on the membrane then were blocked by adding 20011 (microliter)/well of RPMI medium with 10% serum and incubating 1 hr. at room temperature. Antigen stimulated CD8⁺ T cells, in 1:3 serial dilutions, were seeded into the wells of the microtiter plate using 100 μl (microliter)/well, starting at 2×10⁵ cells/well. (Prior antigen stimulation was essentially as described in Scheibenbogen, C. et al. Int. J. Cancer 71:932-936, 1997. PSMA₄₆₂₋₄₇₁ (SEQ ID NO. 62) was added to a final concentration of 10 μg/ml and IL-2 to 100 U/ml and the cells cultured at 37° C. in a 5% CO₂, water-saturated atmosphere for 40 hrs. Following this incubation the plates Were washed with 6 times 200 μl (microliter)/well of PBS containing 0.05% Tween-20 (PBS-Tween). Detection antibody, 50 μl (microliter)/well of 2 g/ml biotinylated murine anti-human γ (gamma)-IFN monoclonal antibody in PBS+10% fetal calf serum, was added and the plate incubated at room temperature for 2 hrs. Unbound detection antibody was removed by washing with 4 times 200 μl of PBS-Tween. 100 μl of avidin-conjugated horseradish peroxidase (Pharmingen, San Diego, Calif.) was added to each well and incubated at room temperature for 1 hr. Unbound enzyme was removed by washing with 6 times 200 μl of PBS-Tween. Substrate was prepared by dissolving a 20 mg tablet of 3-amino 9-ethylcoarbasole in 2.5 ml of N,N-dimethylformamide and adding that solution to 47,5 ml of 0.05 M phosphate-citrate buffer (pH 5.0). 25 μl of 30% H₂O₂ was added to the substrate solution immediately before distributing substrate at 100 μl (microliter)/well and incubating the plate at room temperature. After color development (generally 15-30 min.), the reaction was stopped by washing the plate with water. The plate was air dried and the spots counted using a stereomicroscope.

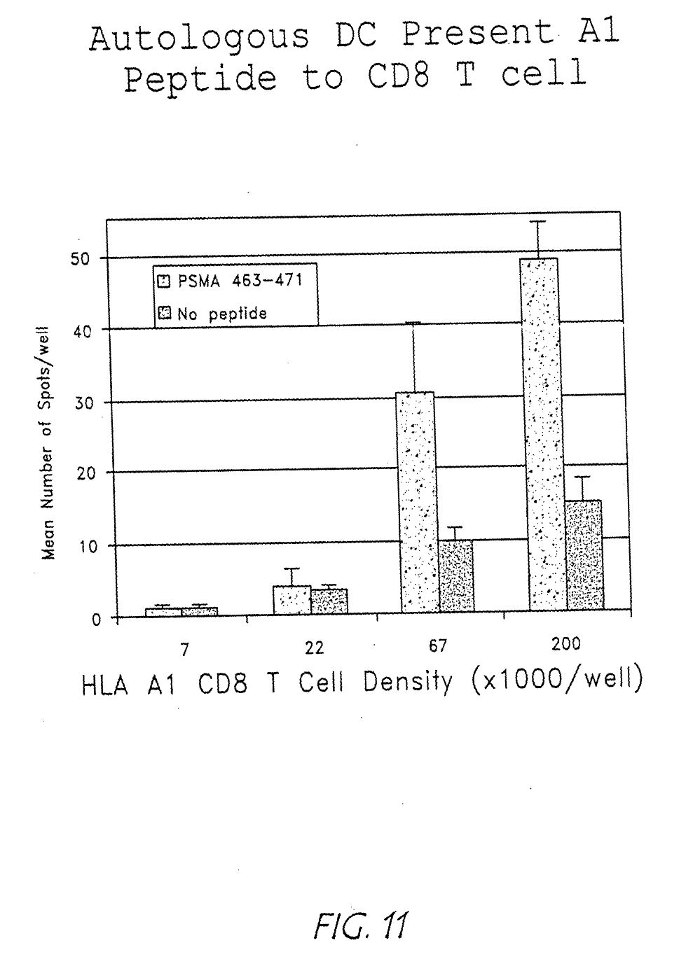

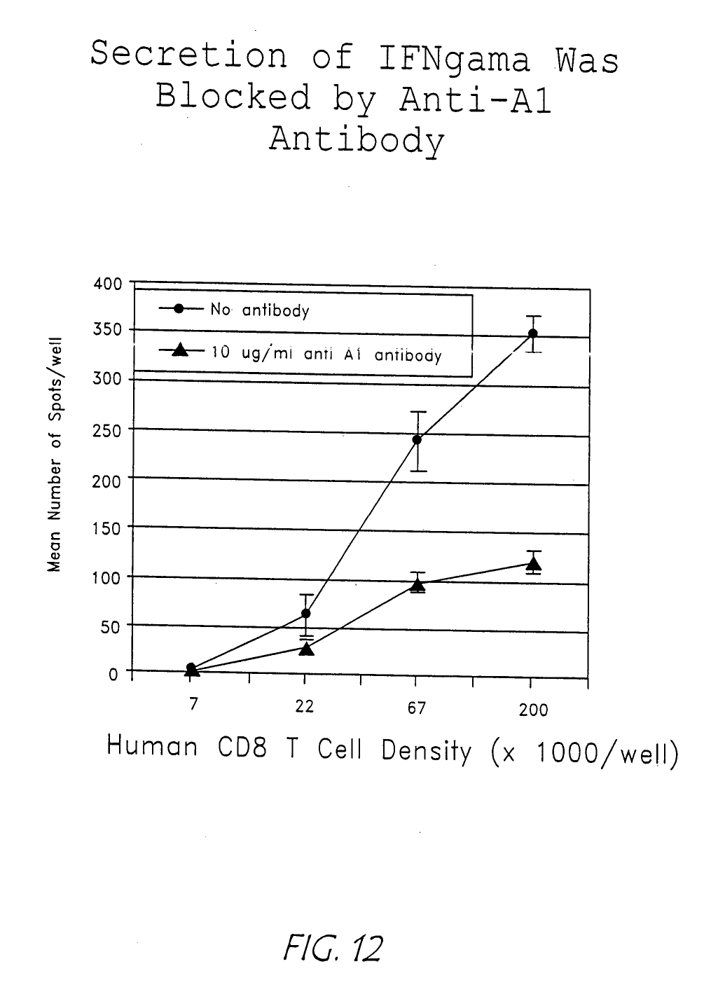

FIG. 11 shows the detection of PSMA₄₆₃₋₄₇₁ (SEQ ID NO. 62)-reactive HLA-A1⁺ CD8⁺ T cells previously generated in cultures of HLA-A1⁺ CD8⁺ T cells with autologous dendritic cells plus the peptide. No reactivity is detected from cultures without peptide (data not shown). In this case it can be seen that the peptide reactive T cells are present in the culture at a frequency between 1 in 2.2×10⁴ and 1 in 6.7×10⁴. That this is truly an HLA-A1-restricted response is demonstrated by the ability of anti-HLA-A1 monoclonal antibody to block γ (gamma) IFN production; see FIG. 12.

### Example 7

Cluster Analysis (PSMA₆₅₃₋₆₉₇).

Another peptide, FDKSNPIVLRMMNDQLMFLERAFIDPLGLPDRP FY PSMA₆₅₃₋₆₈₇, (SEQ ID NO. 64) containing an A2 epitope cluster from prostate specific membrane antigen, PSMA₆₆₀₋₆₈₁ (SEQ ID NO 65), was synthesized by MPS (purity >95%) and subjected to proteasome digestion and mass spectrum analysis as described above. Prominent peaks from the mass spectra are summarized in Table 13.

TABLE 13
PSMA₆₅₃₋₆₈₇ Mass Peak Identification.

| MS PEAK | | | CALCULATED |
|---|---|---|---|
| (measured) | PEPTIDE | SEQUENCE | MASS (MH⁺) |
| 906.17 ± 0.65 | 681-687** | LPDRPFY | 908.05 |
| 1287.73 ± 0.76 | 677-687** | DPLGLPDRPFY | 1290.47 |
| 1400.3 ± 1.79 | 676-687 | IDPLGLPDRPFY | 1403.63 |
| 1548.0 ± 1.37 | 675-687 | FIDPLGLPDRPFY | 1550.80 |
| 1619.5 ± 1.51 | 674-687** | AFIDPLGLPDRPFY | 1621.88 |
| 1775.48 ± 1.32 | 673-687* | RAFIDPLGLPDRPFY | 1778.07 |
| 2440.2 ± 1.3 | 653-672 | FDKSNPIVLRMMNDQLMFLE | 2442.932 |
| 1904.63 ± 1.56 | 672-687* | ERAFIDPLGLPDRPFY | 1907.19 |
| 2310.6 ± 2.5 | 653-671 | FDKSNPIVLRMMNDQLMFL | 2313.82 |
| 2017.4 ± 1.94 | 671-687 | LERAFIDPLGLPDRPFY | 2020.35 |
| 2197.43 ± 1.78 | 653-670 | FDKSNPIVLRMMNDQLMF | 2200.66 |

Boldface sequence correspond to peptides predicted to bind to MHC, see Table 13.
*On the basis of mass alone this peak could equally well be assigned to a peptide beginning at 654, however proteasomal removal of just the N-terminal amino acid is considered unlikely. If the issue were important it could be resolved by N-terminal sequencing.
**On the basis of mass alone these peaks could have been assigned to internal fragments, but given the overall pattern of digestion it was considered unlikely.

Epitope Identification

Fragments co-C-terminal with 8-10 amino acid long sequences predicted to bind HLA by the SYFPEITHI or NIH algorithms were chosen for further study. The digestion and prediction steps of the procedure can be usefully practiced in any order. Although the substrate peptide used in proteasomal digest described here was specifically designed to include predicted HLA-A2.1 binding sequences, the actual products of digestion can be checked after the fact for actual or predicted binding to other MHC molecules. Selected results are shown in Table 14.

TABLE 14
Predicted HLA binding by proteasomally generated fragments

| SEQ ID NO | PEPTIDE | HLA | SYFPEITHI | NIH |
|---|---|---|---|---|
| 66 & (67) | (R)MMNDQLMFL | A*0201 | 24 (23) | 1360 (722) |
| | | A*0205 | NP† | 71 (42) |
| | | A26 | 15 | NP |
| | | B*2705 | 12 | 50 |
| 68 | RMMNDQLMF | B*2705 | 17 | 75 |

†No prediction

As seen in Table 14, N-terminal addition of authentic sequence to epitopes can generate still useful, even better epitopes, for the same or different MHC restriction elements. Note for example the pairing of (R)MMNDQLMFL (SEQ ID NOS. 66 and (67)) with HLA-A*02, where the 10-mer retains substantial predicted binding potential.

HLA-A*0201 Binding Assay

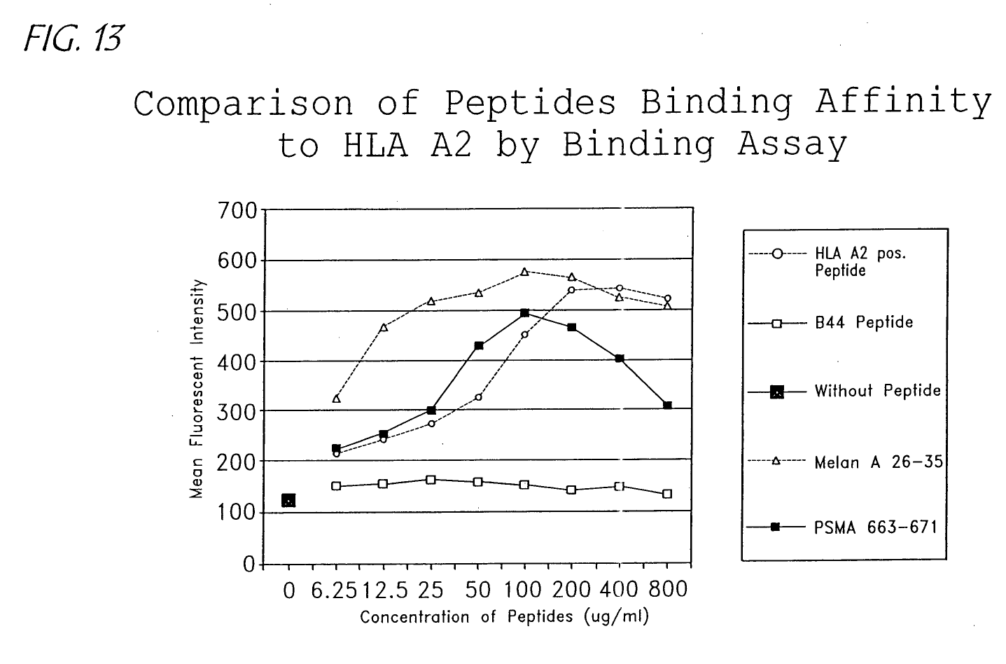

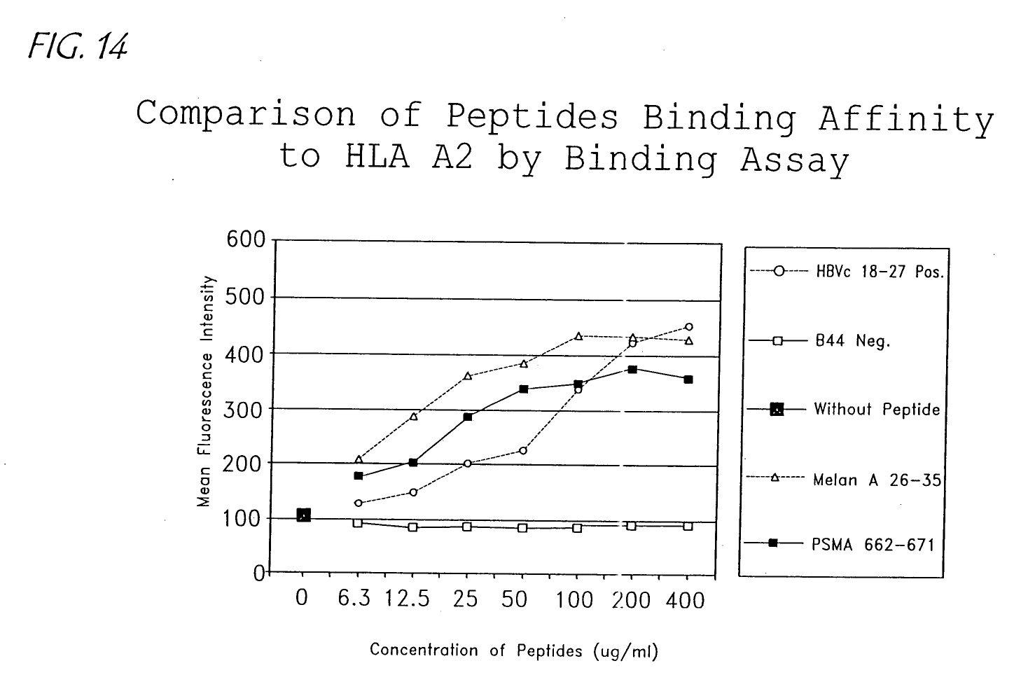

HLA-A*0201 binding studies were preformed, essentially as described in Example 3 above, with PSMA₆₆₃₋₆₇₁, (SEQ ID NO. 66) and PSMA₆₆₂₋₆₇₁, RMMNDQLMFL (SEQ NO. 67). As seen in FIGS. 10, 13 and 14, this epitope exhibits significant binding at even lower concentrations than the positive control peptide (FLPSDYFPSV (HBV₁₈₋₂₇); SEQ ID NO: 24). Though not run in parallel, comparison to the controls suggests that PSMA₆₆₂₋₆₇₁ (which approaches the Melan A peptide in affinity) has the superior binding activity of these two PSMA peptides.

### Example 8

Vaccinating with Epitope Vaccines.

1. Vaccination with Peptide Vaccines:

A. Intranodal Delivery

A formulation containing peptide in aqueous buffer with an antimicrobial agent, an antioxidant, and an immunomodulating cytokine, was injected continuously over several days into the inguinal lymph node using a miniature pumping system developed for insulin delivery (MiniMed; Northridge, Calif.). This infusion cycle was selected in order to mimic the kinetics of antigen presentation during a natural infection.

B. Controlled Release

A peptide formulation is delivered using controlled PLGA microspheres as is known in the art, which alter the pharmacokinetics of the peptide and improve immunogenicity. This formulation is injected or taken orally.

C. Gene Gun Delivery

A peptide formulation is prepared wherein the peptide is adhered to gold microparticles as is known in the art. The particles are delivered in a gene gun, being accelerated at high speed so as to penetrate the skin, carrying the particles into dermal tissues that contain pAPCs.

D. Aerosol Delivery

A peptide formulation is inhaled as an aerosol as is known in the art, for uptake into appropriate vascular or lymphatic tissue in the lungs.

2. Vaccination with Nucleic Acid Vaccines:

A nucleic acid vaccine is injected into a lymph node using a miniature pumping system, such as the MiniMed insulin pump. A nucleic acid construct formulated in an aqueous buffered solution containing an antimicrobial agent, an antioxidant, and an immunomodulating cytokine, is delivered over a several day infusion cycle in order to mimic the kinetics of antigen presentation during a natural infection.

Optionally, the nucleic acid construct is delivered using controlled release substances, such as PLGA microspheres or other biodegradable substances. These substances are injected or taken orally. Nucleic acid vaccines are given using oral delivery, priming the immune response through uptake into GALT tissues. Alternatively, the nucleic acid vaccines are delivered using a gene gun, wherein the nucleic acid vaccine is adhered to minute gold particles. Nucleic acid constructs can also be inhaled as an aerosol, for uptake into appropriate vascular or lymphatic tissue in the lungs.

### Example 9

Assays for the Effectiveness of Epitope Vaccines.

1. Tetramer Analysis:

Class I tetramer analysis is used to determine T cell frequency in an animal before and after administration of a housekeeping epitope. Clonal expansion of T cells in response to an epitope indicates that the epitope is presented to T cells by pAPCs. The specific T cell frequency is measured against the housekeeping epitope before and after administration of the epitope to an animal, to determine if the epitope is present on pAPCs. An increase in frequency of T cells specific to the epitope after administration indicates that the epitope was presented on pAPC.

2. Proliferation Assay:

Approximately 24 hours after vaccination of an animal with housekeeping epitope, pAPCs are harvested from PBMCs, splenocytes, or lymph node cells, using monoclonal antibodies against specific markers present on pAPCs, fixed to magnetic beads for affinity purification. Crude blood or splenoctye preparation is enriched for pAPCs using this technique. The enriched pAPCs are then used in a proliferation assay against a T cell clone that has been generated and is specific for the housekeeping epitope of interest. The pAPCs are coincubated with the T cell clone and the T cells are monitored for proliferation activity by measuring the incorporation of radiolabeled thymidine by T cells. Proliferation indicates that T cells specific for the housekeeping epitope are being stimulated by that epitope on the pAPCs.

3. Chromium Release Assay:

A human patient, or non-human animal genetically engineered to express human class I MHC, is immunized using a housekeeping epitope. T cells from the immunized subject are used in a standard chromium release assay using human tumor targets or targets engineered to express the same class I MHC. T cell killing of the targets indicates that stimulation of T cells in a patient would be effective at killing a tumor expressing a similar TuAA.

### Example 10

Induction of CTL Response with Naked DNA is Efficient by Intra-Lymph Node Immunization.

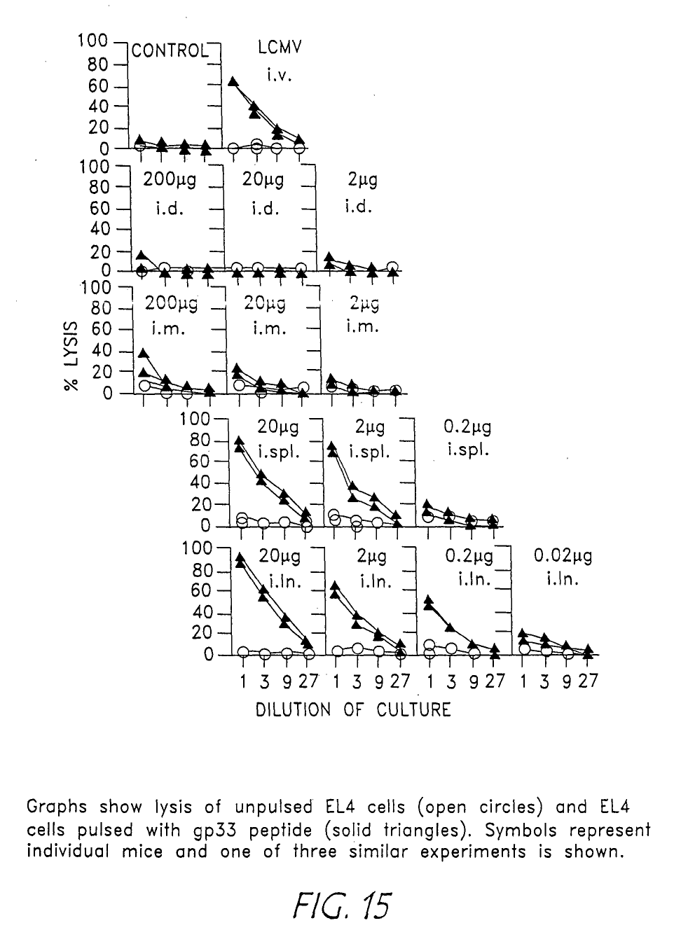

In order to quantitatively compare the CD8⁺ CTL responses induced by different routes of immunization a plasmid DNA vaccine (pEGFPL33A) containing a well-characterized immunodominant CTL epitope from the LCMV-glycoprotein (G) (gp33; amino acids 33-41) (Oehen, S., et al. Immunology 99, 163-169 2000) was used, as this system allows a comprehensive assessment of antiviral CTL responses. Groups of 2 C57BL/6 mice were immunized once with titrated doses (200-0.02 μg) of pEGFPL33A DNA or of control plasmid pEGFP-N3, administered i.m. (intramuscular), i.d. (intradermal), i.spl. (intrasplenic), or i.ln. (intra-lymph node). Positive control mice received 500 pfu LCMV i.v. (intravenous). Ten days after immunization spleen cells were isolated and gp33-specific CTL activity was determined after secondary in vitro restimulation. As shown in FIG. 15, i.m. or i.d. immunization induced weakly detectable CTL responses when high doses of pEFGPL33A DNA (200 μg) were administered. In contrast, potent gp33-specific CTL responses were elicited by immunization with only 2 μg pEFGPL33A DNA i.spl. and with as little as 0.2 μg pEFGPL33A DNA given i.ln. (FIG. 15; symbols represent individual mice and one of three similar experiments is shown). Immunization with the control pEGFP—N3 DNA did not elicit any detectable gp33-specific CTL responses (data not shown).

### Example 11

Intra-Lymph Node DNA Immunization Elicits Anti-Tumor Immunity.

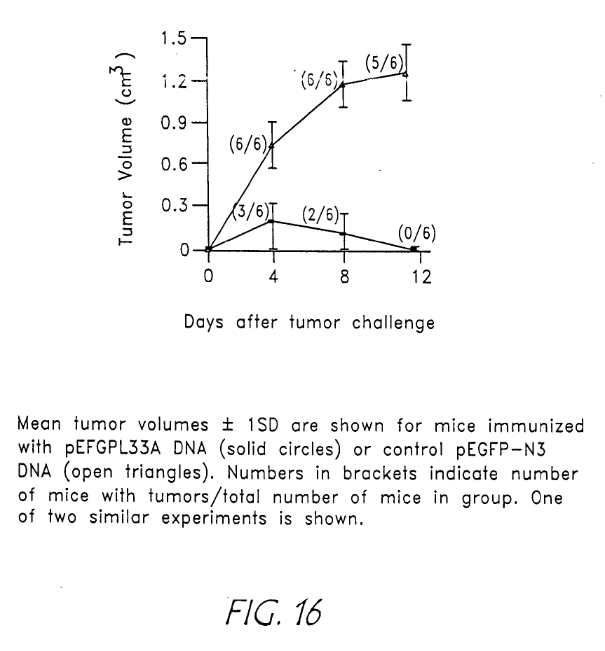

To examine whether the potent CTL responses elicited following i.ln. immunization were able to confer protection against peripheral tumors, groups of 6 C57BL/6mice were immunized three times at 6-day intervals with 10 μg of pEFGPL33A DNA or control pEGFP-N3 DNA. Five days after the last immunization small pieces of solid tumors expressing the gp33 epitope (EL4-33) were transplanted s.c. into both flanks and tumor growth was measured every 3-4d. Although the EL4-33 tumors grew well in mice that had been repetitively immunized with control pEGFP—N3 DNA (FIG. 16), mice which were immunized with pEFGPL33A DNA i.ln. rapidly eradicated the peripheral EL4-33 tumors (FIG. 16).

### Example 12

Differences in Lymph Node DNA Content Mirrors Differences in CTL Response Following Intra-Lymph Node and Intramuscular Injection.

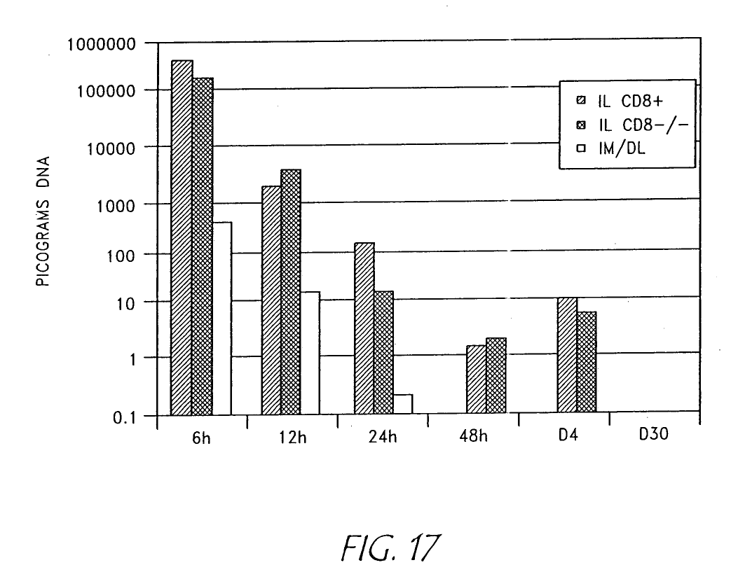

pEFGPL33A DNA was injected i.ln. or i.m. and plasmid content of the injected or draining lymph node was assessed by real time PCR after 6, 12, 24, 48 hours, and 4 and 30 days. At 6, 12, and 24 hours the plasmid DNA content of the injected lymph nodes was approximately three orders of magnitude greater than that of the draining lymph nodes following i.m. injection. No plasmid DNA was detectable in the draining lymph node at subsequent time points (FIG. 17). This is consonant with the three orders of magnitude greater dose needed using i.m. as compared to i.ln. injections to achieve a similar levels of CTL activity. CD8^{-/-} knockout mice, which do not develop a CTL response to this epitope, were also injected i.ln. showing clearance of DNA from the lymph node is not due to CD8⁺ CTL killing of cells in the lymph node. This observation also supports the conclusion that i.ln. administration will not provoke immunopathological damage to the lymph node.

### Example 13

Administration of a DNA Plasmid Formulation of a Therapeutic Vaccine for Melanoma to Humans.

A SYNCHROTOPE™ TA2M melanoma vaccine encoding the HLA-A2-restricted tyrosinase epitope SEQ ID NO. 1 and epitope cluster SEQ ID NO. 69, was formulated in 1% Benzyl alcohol, 1% ethyl alcohol, 0.5 mM EDTA, citrate-phosphate, pH 7.6. Aliquots of 80, 160, and 320 μg DNA/ml were prepared for loading into MINIMED 407C infusion pumps. The catheter of a SILHOUETTE infusion set was placed into an inguinal lymph node visualized by ultrasound imaging. The assembly of pump and infusion set was originally designed for the delivery of insulin to diabetics and the usual 17 mm catheter was substituted with a 31 mm catheter for this application. The infusion set was kept patent for 4 days (approximately 96 hours) with an infusion rate of about 25 μl (microliter)/hour resulting in a total infused volume of approximately 2.4 ml. Thus the total administered dose per infusion was approximately 200, and 400 μg; and can be 800 μg, respectively, for the three concentrations described above. Following an infusion subjects were given a 10 day rest period before starting a subsequent infusion. Given the continued residency of plasmid DNA in the lymph node after administration (as in example 12) and the usual kinetics of CTL response following disappearance of antigen, this schedule will be sufficient to maintain the immunologic CTL response.

### Example 14

Evaluating Likelihood of Epitope Cross-Reactivity on Non-Target Tissues.

As noted above PSA is a member of the kallikrein family of proteases, which is itself a subset of the serine protease family. While the members of this family sharing the greatest degree of sequence identity with PSA also share similar expression profiles, it remains possible that individual epitope sequences might be shared with proteins having distinctly different expression profiles. A first step in evaluating the likelihood of undesirable cross-reactivity is the identification of shared sequences. One way to accomplish this is to conduct a BLAST search of an epitope sequence against the SWISSPROT or Entrez non-redundant peptide sequence databases using the “Search for short nearly exact matches” option; hypertext transfer protocol accessible on the world wide web (http://www) at “ncbi.nlm.nih.gov/blast/index.html”. Thus searching SEQ ID NO. 104, WVLTAAHCI, against SWISSPROT (limited to entries for homo sapiens) one finds four exact matches, including PSA. The other three are from kallikrein 1 (tissue kallikrein), and elastase 2A and 2B. While these nine amino acid segments are identical, the flanking sequences are quite distinct, particularly on the C-terminal side, suggesting that processing may proceed differently and that thus the same epitope may not be liberated from these other proteins. (Please note that kallikrein naming is confused. Thus, the kallikrein 1 [accession number P06870] is a different protein than the one [accession number AAD13817] mentioned in the paragraph on PSA above in the section on tumor-associated antigens).

This possibility can be tested in several ways. Synthetic peptides containing the epitope sequence embedded in the context of each of these proteins can be subjected to in vitro proteasomal digestion and analysis as described above. Alternatively, cells expressing these other proteins, whether by natural or recombinant expression, can be used as targets in a cytotoxicity (or similar) assay using CD8⁺ T cells that recognize the epitope, in order to determine if the epitope is processed and presented.

### Examples 15-67

Epitopes.

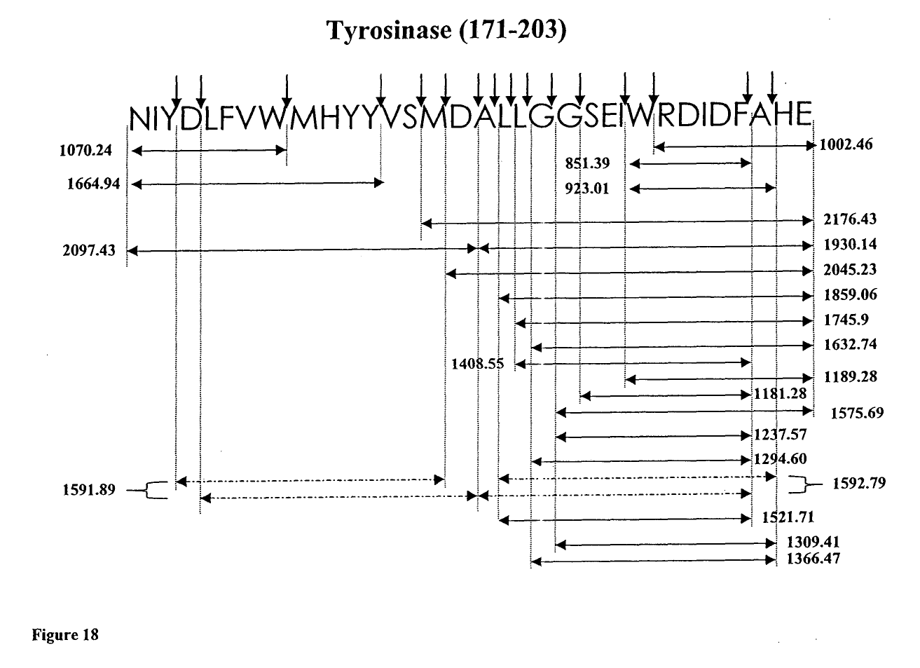

The methodologies described above, and in particular in examples 3-7, have been applied to additional synthetic peptide substrates, as summarized in FIGS. 18-70 leading to the identification of further epitopes as set forth the in tables 15-67 below. The substrates used here were generally designed to identify products of housekeeping proteasomal processing that give rise to HLA-A*0201 binding epitopes, but additional MHC-binding reactivities can be predicted, as discussed above. Many such reactivities are disclosed, however, these listings are meant to be exemplary, not exhaustive or limiting. As also discussed above, individual components of the analyses can be used in varying combinations and orders. N-terminal pool sequencing which allows quantitation of various cleavages and can resolve ambiguities in the mass spectrum where necessary, can also be used to identify cleavage sites when digests of substrate yield fragments that do not fly well in MALDI-TOF mass spectrometry. Due to these advantages it was routinely used. Although it is preferred to identify epitopes on the basis of the C-terminus of an observed fragment, epitopes can also be identified on the basis of the N-terminus of an observed fragment adjacent to the epitope.

Not all of the substrates necessarily meet the formal definition of an epitope cluster as referenced in example 3. Some clusters are so large that it was more convenient to use substrates spanning only a portion of the cluster. In other cases, substrates were extended beyond clusters meeting the formal definition to include neighboring predicted epitopes or were designed around predicted epitopes with no association with any cluster. In some instances, actual binding activity dictated what substrate was made when HLA binding activity was determined for a selection of peptides with predicted affinity, before synthetic substrates were designed.

FIGS. 18-70 show the results of proteasomal digestion analysis as a mapping of mass spectrum peaks onto the substrate sequence. Each figure presents an individual timepoint from the digestion judged to be respresentative of the overall data, however some epitopes listed in Tables 15-67 were identified based on fragments not observed at the particular timepoints illustrated. The mapping of peaks onto the sequence was informed by N-terminal pool sequencing of the digests, as noted above. Peaks possibly corresponding to more than one fragment are represented by broken lines. Nonetheless, epitope identifications are supported by unambiguous occurrence of the associated cleavage.

### Example 15

### Tyrosinase 171-203

TABLE 15
Preferred Epitopes Revealed by Housekeeping Proteasome Digestion

| | | Sequence | | HLA binding predictions† | |
|---|---|---|---|---|---|
| Epitope | Sequence | ID No. | HLA type | SYFPEITHI | NIH |
| 171-179 | NIYDLFVWM | 108 | A0201 | 17 | 93.656 |
| | | | A26 | 25 | N/A |
| | | | A3 | 18 | <5 |
| 173-182 | YDLFVWMHYY | 109 | A1 | 17 | <5 |
| 174-182 | DLFVWMHYY | 110 | A1 | 16 | <5 |
| | | | A26 | 30 | N/A |
| | | | A3 | 16 | 27 |
| 186-194 | DALLGGSEI | 111 | A0201 | 17 | <5 |
| | | | B5101 | 26 | 440 |
| 191-200 | GSEIWRDIDF | 112 | A1 | 18 | 67.5 |
| 192-200 | SEIWRDIDF | 113 | B08 | 16 | <5 |
| 193-201 | EIWRDIDFA | 114 | A26 | 20 | N/A |

†Scores are given from the two binding prediction programs referenced above (see example 3)
See also FIG. 18.

### Example 16

### Tyrosinase 401-427

TABLE 16
Preferred Epitopes Revealed by Housekeeping Proteasome Digestion

| | | Sequence | | HLA binding predictions† | |
|---|---|---|---|---|---|
| Epitope | Sequence | ID No. | HLA type | SYFPEITHI | NIH |
| 407-416 | LQEVYPEANA | 115 | A0203 | 18 | N/A |
| 409-418 | EVYPEANAPI | 116 | A26 | 19 | N/A |
| | | | A3 | 20 | <5 |
| 410-418 | VYPEANAPI | 117 | B5101 | 15 | 6.921 |
| 411-418 | YPEANAPI | 118 | B5101 | 22 | N/A |
| 411-420 | YPEANAPIGH | 119 | A1 | 16 | <5 |
| 416-425 | APIGHNRESY | 120 | A1 | 18 | <5 |
| | | | A26 | 15 | N/A |
| 417-425 | PIGHNRESY | 121 | A1 | 16 | <5 |
| | | | A26 | 21 | N/A |
| | | | A3 | 17 | <5 |
| 417-426 | PIGHNRESYM | 122 | A26 | 19 | N/A |

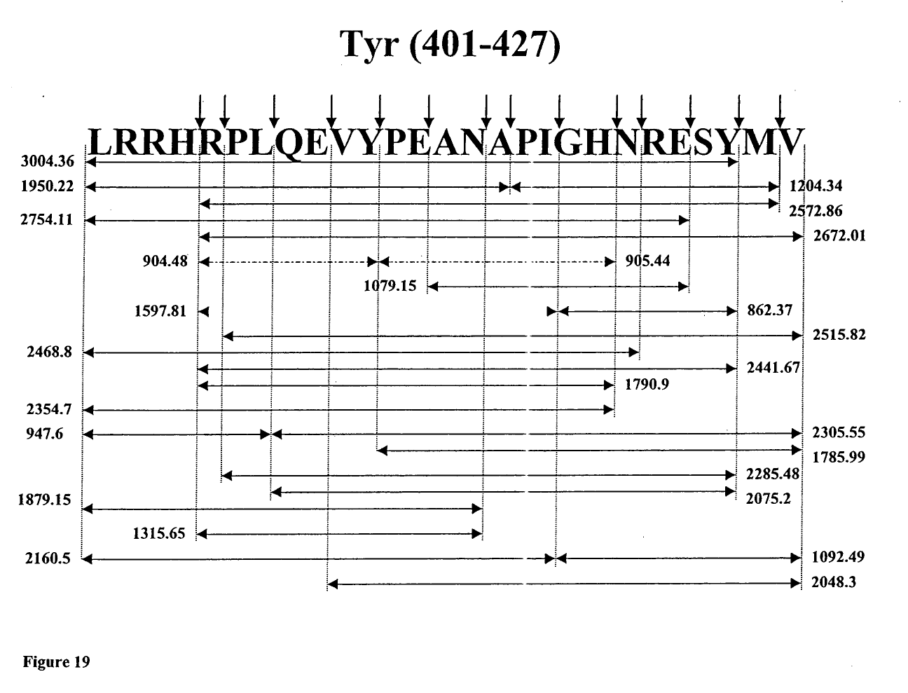

†Scores are given from the two binding prediction programs referenced above (see example 3)
See also FIG. 19.

### Example 17

### Tyrosinase 415-449

TABLE 17
Preferred Epitopes Revealed by Housekeeping Proteasome Digestion

| | | Sequence | | HLA binding predictions† | |
|---|---|---|---|---|---|
| Epitope | Sequence | ID No. | HLA type | SYFPEITHI | NIH |
| 416-425 | APIGHNRESY | 120 | A1 | 18 | <5 |
| | | | A26 | 15 | N/A |
| | | | A3 | 17 | <5 |
| | | | B0702 | 15 | N/A |
| 417-425 | PIGHNRESY | 124 | A1 | 16 | <5 |
| | | | A26 | 21 | N/A |
| | | | A3 | 17 | <5 |
| 423-430 | ESYMVPFI | 125 | B5101 | 17 | N/A |
| 423-432 | ESYMVPFIPL | 126 | A26 | 18 | N/A |
| 424-432 | SYMVPFIPL | 127 | B0702 | 16 | N/A |
| 424-433 | SYMVPFIPLY | 128 | A1 | 19 | <5 |
| | | | A26 | 15 | N/A |
| 425-433 | YMVPFIPLY | 129 | A0201 | 18 | <5 |
| | | | A1 | 23 | 5 |
| | | | A26 | 17 | N/A |
| 426-434 | MVPFIPLYR | 130 | A3 | 18 | <5 |
| 426-435 | MVPFIPLYRN | 131 | A26 | 16 | N/A |
| 427-434 | VPFIPLYR | 132 | B5101 | 18 | N/A |
| 430-437 | IPLYRNGD | 133 | B08 | 16 | <5 |
| 430-439 | IPLYRNGDFF | 134 | B0702 | 18 | N/A |
| 431-439 | PLYRNGDFF | 135 | A26 | 18 | N/A |
| | | | A3 | 24 | <5 |
| 431-440 | PLYRNGDFFI | 136 | A0201 | 16 | 23.43 |
| | | | A3 | 17 | <5 |
| 434-443 | RNGDFFISSK | 137 | A3 | 20 | <5 |
| 435-443 | NGDFFISSK | 138 | A3 | 15 | <5 |
| | | | B2705 | 15 | 5 |

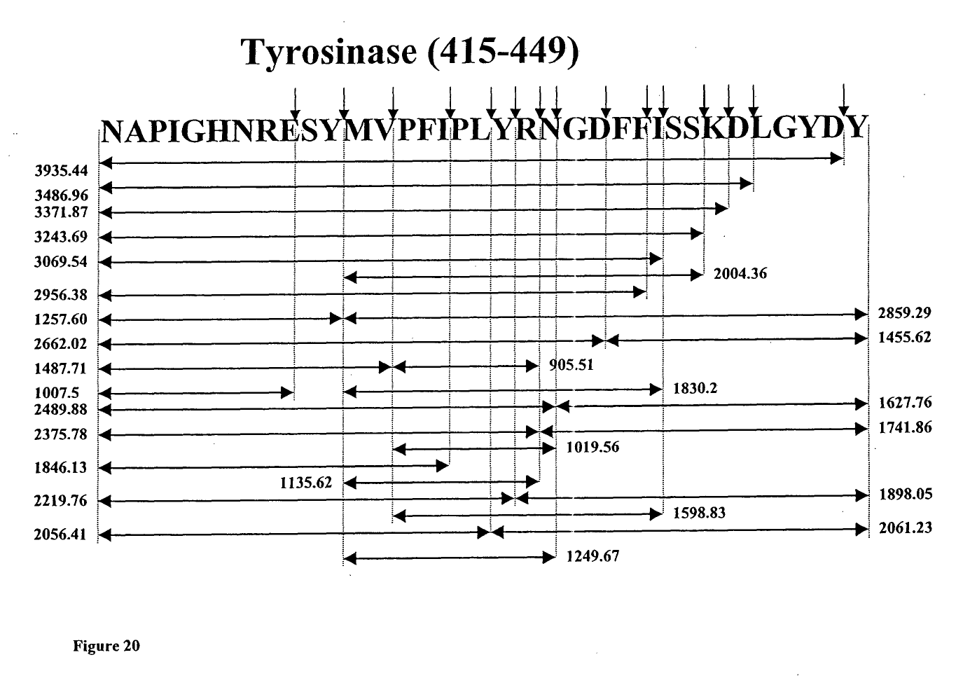

†Scores are given from the two binding prediction programs referenced above (see example 3)
See also FIG. 20.

### Example 18

### Tyrosinase 457-484

TABLE 18
Preferred Epitopes Revealed by Housekeeping Proteasome Digestion

| | | Sequence | | HLA binding predictions† | |
|---|---|---|---|---|---|
| Epitope | Sequence | ID No. | HLA type | SYFPEITHI | NIH |
| 463-471 | YIKSYLEQA | 139 | A0201 | 18 | <5 |
| | | | A26 | 17 | N/A |
| 466-474 | SYLEQASRI | 140 | B5101 | 16 | <5 |
| 469-478 | EQASRIWSWL | 141 | A26 | 17 | N/A |
| 470-478 | QASRIWSWL | 142 | B5101 | 16 | 55 |
| 471-478 | ASRIWSWL | 143 | B08 | 16 | <5 |
| 471-479 | ASRIWSWLL | 144 | B08 | 16 | <5 |
| 473-481 | RIWSWLLGA | 145 | A0201 | 19 | 13.04 |
| | | | A26 | 16 | N/A |
| | | | A3 | 15 | <5 |

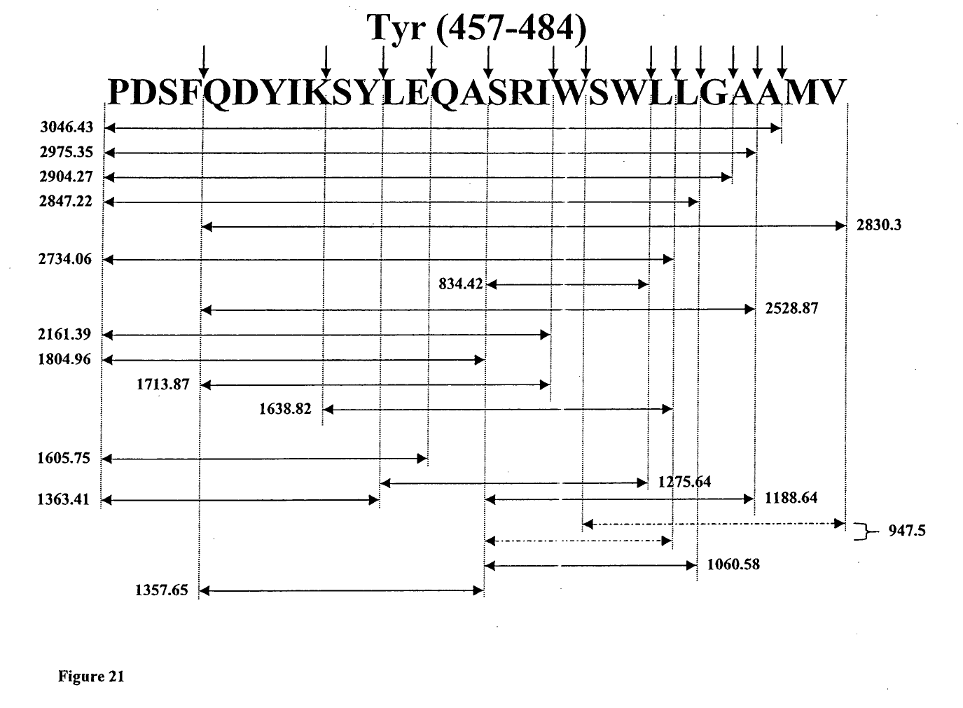

†Scores are given from the two binding prediction programs referenced above (see example 3)
See also FIG. 21.

### Example 19

### CEA 92-118

TABLE 19
Preferred Epitopes Revealed by Housekeeping Proteasome Digestion

| | | Sequence | | HLA binding predictions† | |
|---|---|---|---|---|---|
| Epitope | Sequence | ID No. | HLA type | SYFPEITHI | NIH |
| 92-100 | GPAYSGREI | 146 | B0702 | 18 | 8 |
| | | | B08 | 15 | <5 |
| | | | B5101 | 22 | 484 |
| 92-101 | GPAYSGREII | 147 | B0702 | 18 | 12 |
| 93-100 | PAYSGREI | 148 | B5101 | 22 | N.A. |
| 93-101 | PAYSGREII | 149 | B5101 | 24 | 48.4 |
| 93-102 | PAYSGREIIY | 150 | A1 | 19 | <5 |
| 94-102 | AYSGREIIY | 151 | A1 | 21 | <5 |
| 97-105 | GREIIYPNA | 152 | B2705 | 17 | 200 |
| | | | B2709 | 16 | |
| 98-107 | REIIYPNASL | 153 | A0201 | 16 | <5 |
| 99-107 | EIIYPNASL | 154 | A0201 | 21 | <5 |
| | | | A26 | 28 | N.A. |
| | | | A3 | 16 | <5 |
| | | | B0702 | 15 | 6 |
| | | | B08 | 18 | <5 |
| | | | B2705 | 16 | <5 |
| 99-108 | EIIYPNASLL | 155 | A0201 | 16 | <5 |
| | | | A26 | 27 | N.A. |
| | | | A3 | 17 | <5 |
| 100-107 | IIYPNASL | 156 | B08 | 15 | <5 |
| 100-108 | IIYPNASLL | 157 | A0201 | 23 | 15.979 |
| | | | A26 | 21 | N.A. |
| | | | A24 | N.A. | <5 |
| | | | A3 | 23 | <5 |
| | | | B08 | 15 | <5 |
| | | | B1510 | 15 | N.A. |
| | | | B2705 | 16 | 50 |
| | | | B2709 | 15 | |
| 100-109 | IIYPNASLLI | 158 | A0201 | 22 | 7.804 |
| | | | A3 | 20 | <5 |
| 102-109 | YPNASLLI | 159 | B5101 | 23 | N.A. |
| 107-116 | LLIQNIIQND | 160 | A0201 | 18 | <5 |
| | | | A26 | 17 | N.A. |

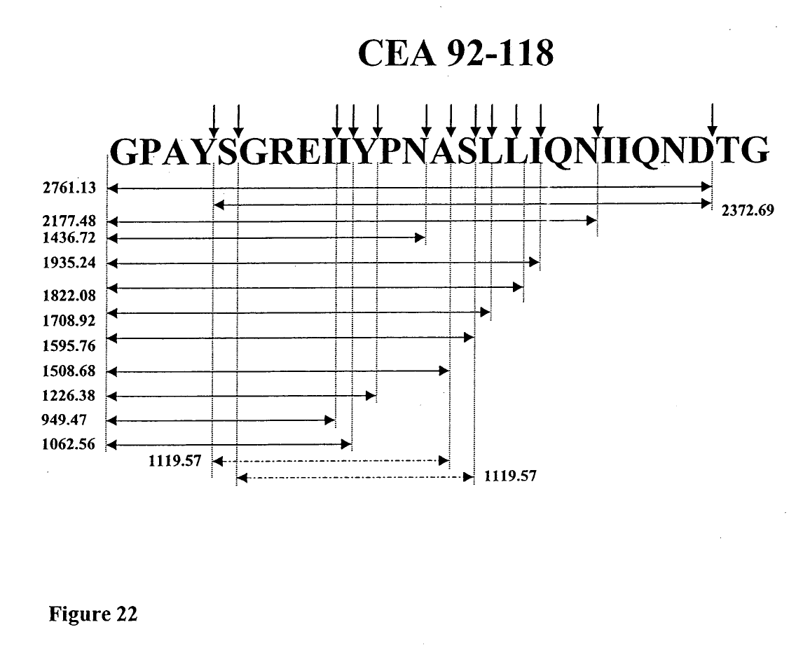

†Scores are given from the two binding prediction programs referenced above (see example 3)
See also FIG. 22.

### Example 20

### CEA 131-159

TABLE 20
Preferred Epitopes Revealed by Housekeeping Proteasome Digestion

| | | Sequence | | HLA binding predictions† | |
|---|---|---|---|---|---|
| Epitope | Sequence | ID No. | HLA type | SYFPEITHI | NIH |
| 132-141 | EEATGQFRVY | 161 | A1 | 19 | <5 |
| | | | A26 | 21 | N.A. |
| 133-141 | EATGQFRVY | 162 | A1 | 22 | <5 |
| | | | A26 | 23 | N.A. |
| | | | B5101 | 16 | <5 |
| 141-149 | YPELPKPSI | 163 | B0702 | 20 | <5 |
| | | | B5101 | 22 | 572 |
| 142-149 | PELPKPSI | 164 | B08 | 16 | <5 |

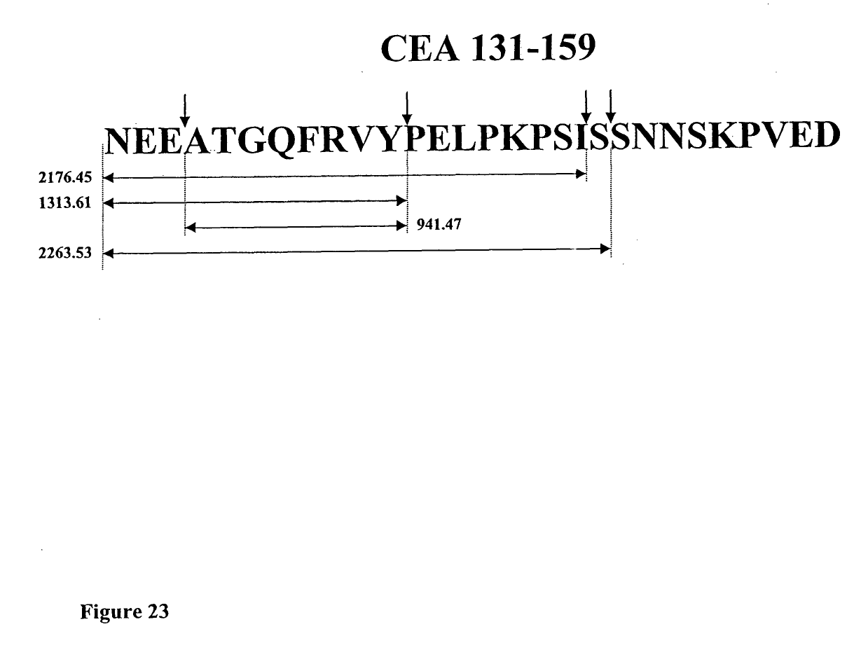

†Scores are given from the two binding prediction programs referenced above (see example 3)
See also FIG. 23.

### Example 21

### CEA 225-251

TABLE 21
Preferred Epitopes Revealed by Housekeeping Proteasome Digestion

| | | Sequence | | HLA binding predictions† | |
|---|---|---|---|---|---|
| Epitope | Sequence | ID No. | HLA type | SYFPEITHI | NIH |
| 225-233 | RSDSVILNV | 165 | A0201 | 15 | <5 |
| | | | A1 | 22 | <5 |
| | | | B2709 | 15 | N.A. |
| 225-234 | RSDSVILNVL | 166 | A0201 | 15 | <5 |
| 226-234 | SDSVILNVL | 167 | A0201 | 17 | <5 |
| 226-235 | SDSVILNVLY | 168 | A1 | 20 | <5 |
| 227-235 | DSVILNVLY | 169 | A1 | 22 | <5 |
| | | | A26 | 18 | N.A. |
| 233-242 | VLYGPDAPTI | 170 | A0201 | 25 | 56.754 |
| | | | A3 | 23 | <5 |
| 234-242 | LYGPDAPTI | 171 | A0201 | 15 | <5 |
| | | | B5101 | 15 | 5.72 |
| 235-242 | YGPDAPTI | 172 | B5101 | 22 | N.A. |
| 236-245 | GPDAPTISPL | 173 | A0201 | 15 | <5 |
| | | | B0702 | 23 | 24 |
| 237-245 | PDAPTISPL | 174 | A0201 | 15 | <5 |
| | | | A26 | 16 | N.A. |
| | | | B2705 | 15 | <5 |
| 238-245 | DAPTISPL | 175 | B5101 | 25 | N.A. |
| 239-247 | APTISPLNT | 176 | B0702 | 20 | 6 |
| 240-249 | PTISPLNTSY | 177 | A1 | 22 | <5 |
| | | | A26 | 24 | N.A. |
| 241-249 | TISPLNTSY | 178 | A1 | 20 | 5 |
| | | | A26 | 24 | N.A. |
| | | | A3 | 20 | <5 |

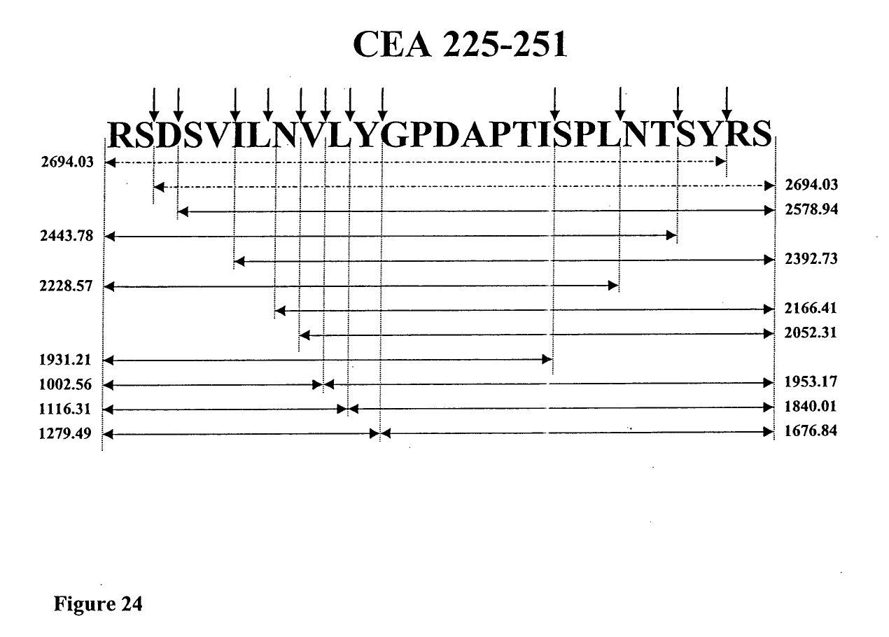

†Scores are given from the two binding prediction programs referenced above (see example 3)
See also FIG. 24.

### Example 22

### CEA 239-270

TABLE 22
Preferred Epitopes Revealed by Housekeeping Proteasome Digestion

| | | Sequence | | HLA binding predictions† | |
|---|---|---|---|---|---|
| Epitope | Sequence | ID No. | HLA type | SYFPEITHI | NIH |
| 240-249 | PTISPLNTSY | 179 | A1 | 22 | <5 |
| | | | A26 | 24 | N.A. |
| 241-249 | TISPLNTSY | 180 | A1 | 20 | 5 |
| | | | A26 | 24 | N.A. |
| | | | A3 | 20 | <5 |
| 246-255 | NTSYRSGENL | 181 | A26 | 19 | N.A. |
| 247-255 | TSYRSGENL | 182 | B2705 | 15 | 50 |
| 248-255 | SYRSGENL | 183 | B08 | 18 | <5 |
| 248-257 | SYRSGENLNL | 184 | B0702 | 14 | <5 |
| 249-257 | YRSGENLNL | 185 | A0201 | 15 | <5 |
| | | | B0702 | 16 | <5 |
| | | | B2705 | 27 | 2000 |
| | | | B2709 | 22 | N.A. |
| 251-259 | SGENLNLSC | 186 | A1 | 19 | <5 |
| 253-262 | ENLNLSCHAA | 187 | A0203 | 19 | <5 |
| 254-262 | NLNLSCHAA | 188 | A0201 | 17 | <5 |

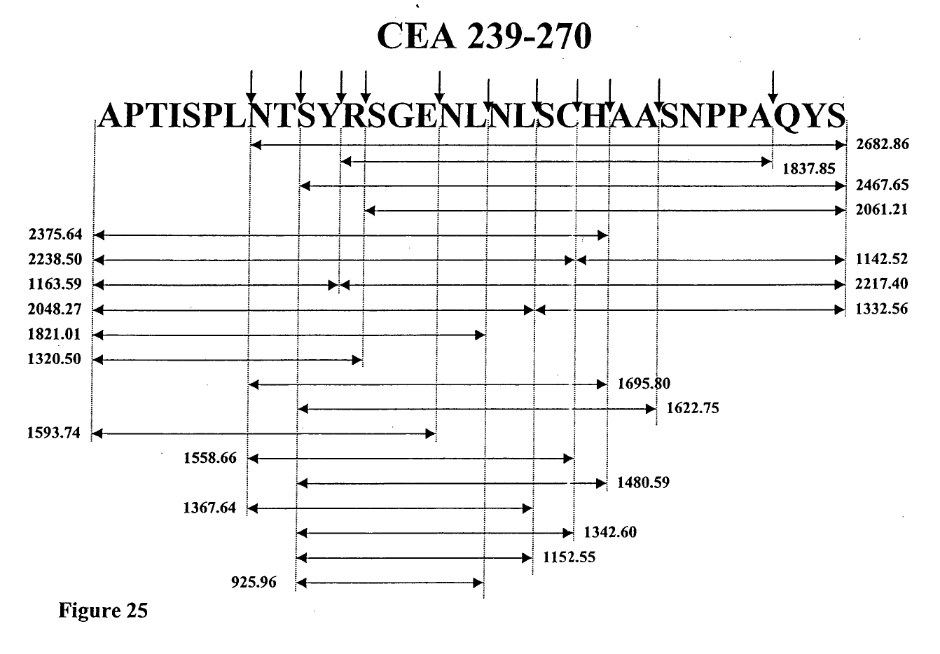

†Scores are given from the two binding prediction programs referenced above (see example 3)
See also FIG. 25.

### Example 23

### CEA 259-286

TABLE 23
Preferred Epitopes Revealed by Housekeeping Proteasome Digestion

| | | Sequence | | HLA binding predictions† | |
|---|---|---|---|---|---|
| Epitope | Sequence | ID No. | HLA type | SYFPEITHI | NIH |
| 260-269 | HAASNPPAQY | 189 | A1 | 15 | <5 |
| 261-269 | AASNPPAQY | 190 | A1 | 17 | <5 |
| | | | A3 | 17 | <5 |
| 264-273 | NPPAQYSWFV | 191 | B0702 | 18 | <5 |
| 265-273 | PPAQYSWFV | 192 | B0702 | 18 | <5 |
| | | | B5101 | 19 | 20 |
| 266-273 | PAQYSWFV | 193 | B5101 | 18 | N.A. |
| 272-280 | FVNGTFQQS | 194 | A26 | 18 | N.A. |
| | | | A3 | 15 | <5 |

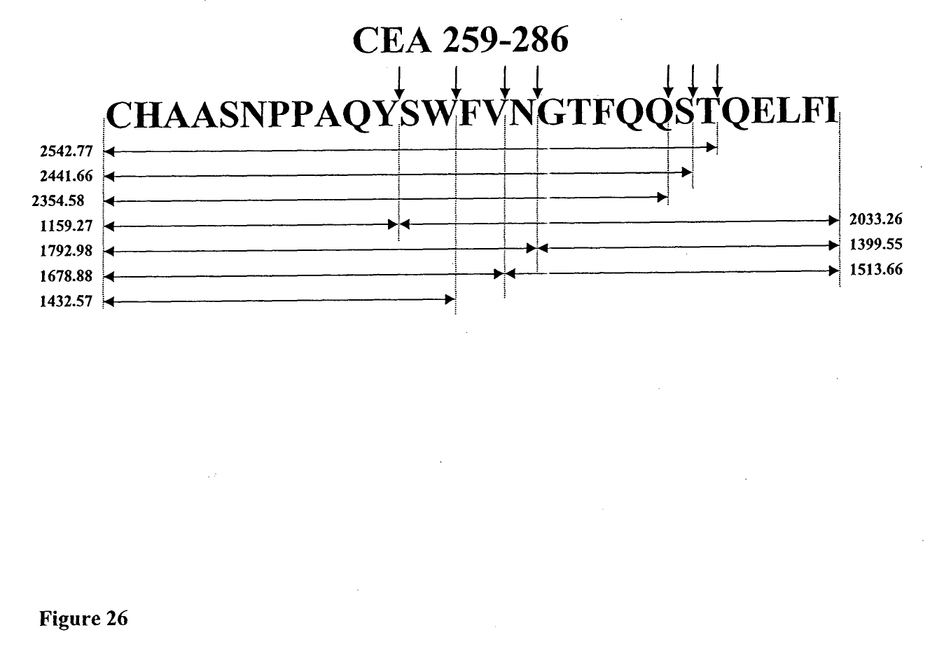

†Scores are given from the two binding prediction programs referenced above (see example 3)
See also FIG. 26.

### Example 24

### CEA 309-336

TABLE 24
Preferred Epitopes Revealed by Housekeeping
Proteasome Digestion

| | | | | HLA binding | |
|---|---|---|---|---|---|
| | | Sequence | HLA | predictions† | |
| Epitope | Sequence | ID No. | type | SYFPEITHI | NIH |
| 310-319 | RTTVTTITVY | 195 | A1 | 22 | <5 |
| | | | A26 | 24 | N.A. |
| | | | A3 | 15 | <5 |
| 311-319 | TTVTTITVY | 196 | A1 | 22 | <5 |
| | | | A26 | 24 | N.A. |
| | | | B2705 | 15 | 5 |
| 319-327 | YAEPPKPFI | 197 | A0201 | 17 | <5 |
| | | | A1 | 17 | 18 |
| | | | B5101 | 22 | 286 |
| 319-328 | YAEPPKPFIT | 198 | A1 | 16 | 45 |
| 320-327 | AEPPKPFI | 199 | B08 | 16 | <5 |
| 321-328 | EPPKPFIT | 200 | B5101 | 16 | N.A. |
| 321-329 | EPPKPFITS | 201 | B0702 | 16 | <5 |
| | | | B5101 | 16 | 12.1 |
| 322-329 | PPKPFITS | 202 | B08 | 16 | <5 |

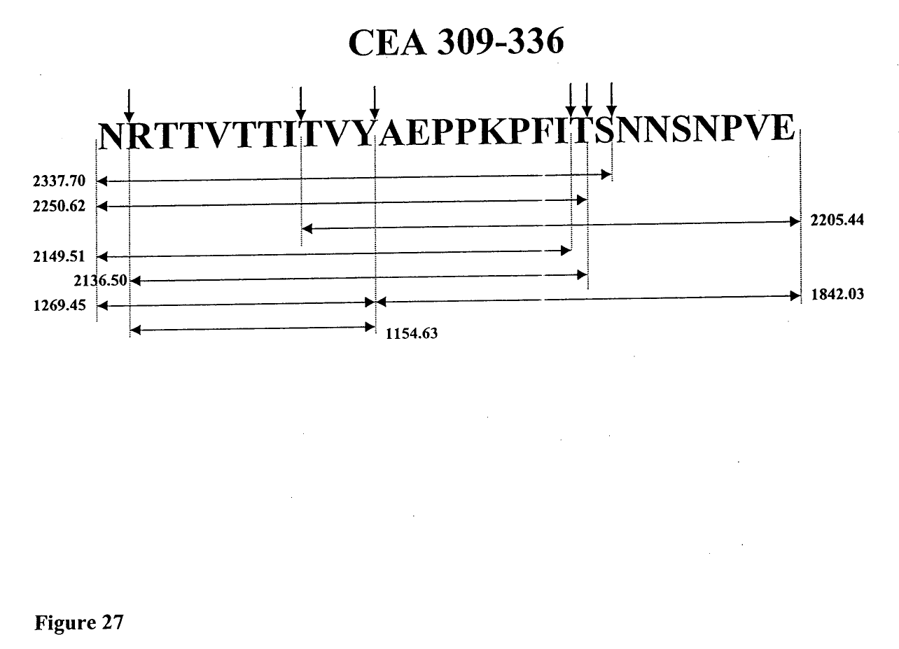

†Scores are given from the two binding prediction programs referenced above (see example 3)
See also FIG. 27.

### Example 25

### CEA 381-408

TABLE 25
Preferred Epitopes Revealed by Housekeeping
Proteasome Digestion

| | | | | HLA binding | |
|---|---|---|---|---|---|
| | | Sequence | HLA | predictions† | |
| Epitope | Sequence | ID No. | type | SYFPEITHI | NIH |
| 382-391 | SVTRNDVGPY | 203 | A1 | 18 | <5 |
| | | | A26 | 24 | N.A. |
| | | | A3 | 21 | <5 |
| 383-391 | VTRNDVGPY | 204 | A1 | 23 | <5 |
| | | | A26 | 24 | N.A. |
| 389-397 | GPYECGIQN | 205 | B5101 | 17 | 11 |
| 391-399 | YECGIQNEL | 206 | A0201 | 17 | <5 |
| | | | B2705 | 17 | 30 |
| 394-402 | GIQNELSVD | 207 | A26 | 15 | N.A. |
| | | | A3 | 16 | <5 |

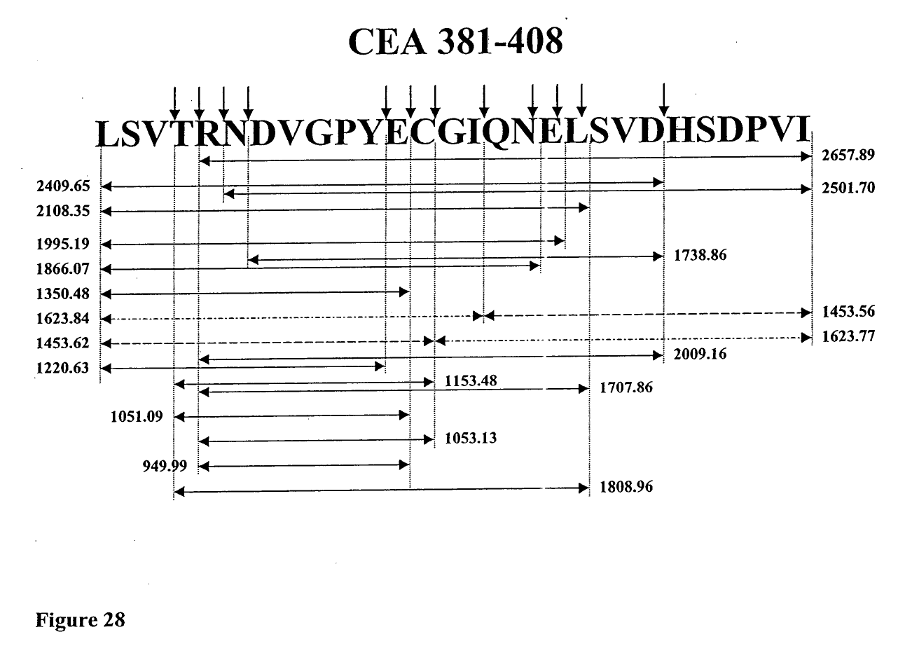

†Scores are given from the two binding prediction programs referenced above (see example 3)
See also FIG. 28.

### Example 26

### CEA 403-429

TABLE 26
Preferred Epitopes Revealed by Housekeeping
Proteasome Digestion

| | | Se- | | HLA binding | |
|---|---|---|---|---|---|
| | | quence | HLA | predictions† | |
| Epitope | Sequence | ID No. | type | SYFPEITHI | NIH |
| 403-411 | HSDPVILNV | 208 | A0201 | 17 | <5 |
| | | | A1 | 26 | 37.5 |
| 403-412 | HSDPVILNVL | 209 | A0201 | 17 | <5 |
| | | | A1 | 19 | 7.5 |
| | | | A26 | 15 | N.A. |
| | | | A24 | N.A. | 8.064 |
| | | | B4402 | 17 | N.A. |
| 404-412 | SDPVILNVL | 210 | A0201 | 17 | <5 |
| | | | B4402 | 16 | N.A. |
| 404-413 | SDPVILNVLY | 211 | A1 | 20 | <5 |
| 405-412 | DPVILNVL | 212 | B08 | 16 | <5 |
| | | | B5101 | 24 | N.A. |
| 405-413 | DPVILNVLY | 213 | A1 | 18 | <5 |
| | | | A26 | 18 | N.A. |
| | | | B5101 | 16 | 7.26 |
| 408-417 | ILNVLYGPDD | 214 | A3 | 15 | <5 |
| 411-420 | VLYGPDDPTI | 215 | A0201 | 25 | 56.754 |
| | | | A3 | 20 | <5 |
| 412-420 | LYGPDDPTI | 216 | A0201 | 15 | <5 |
| | | | A24 | N.A. | 60 |
| 413-420 | YGPDDPTI | 217 | B5101 | 22 | N.A. |
| 417-425 | DPTISPSYT | 218 | B0702 | 16 | <5 |
| 418-427 | PTISPSYTYY | 219 | A1 | 21 | <5 |
| | | | A26 | 27 | N.A. |
| 419-427 | TISPSYTYY | 220 | A1 | 19 | 5 |
| | | | A26 | 27 | N.A. |

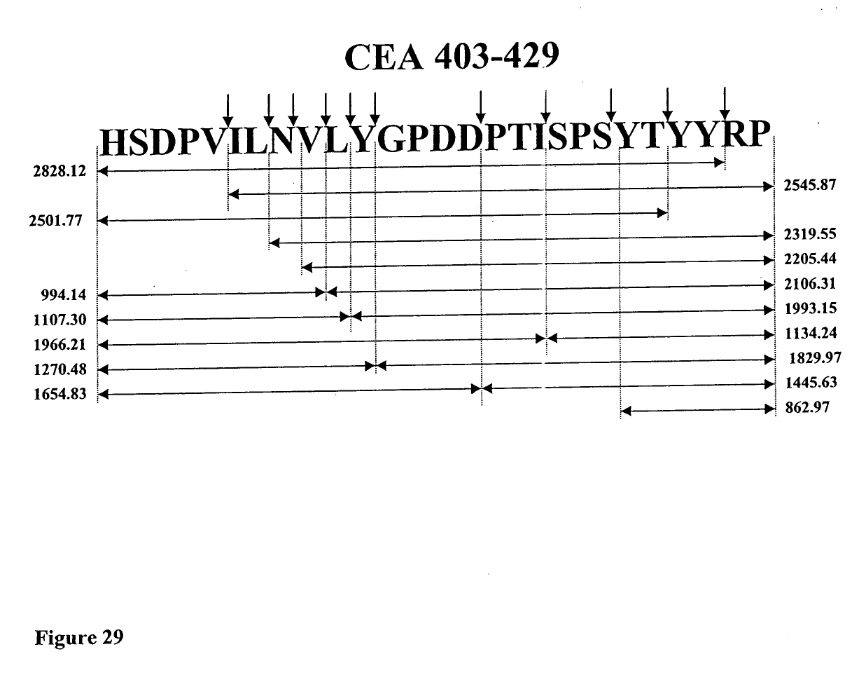

†Scores are given from the two binding prediction programs referenced above (see example 3)
See also FIG. 29.

### Example 27

### CEA 416-448

TABLE 27
Preferred Epitopes Revealed by Housekeeping
Proteasome Digestion

| | | Se- | | HLA binding | |
|---|---|---|---|---|---|
| | | quence | HLA | predictions† | |
| Epitope | Sequence | ID No. | type | SYFPEITHI | NIH |
| 418-427 | PTISPSYTYY | 221 | A1 | 21 | <5 |
| | | | A26 | 27 | N.A. |
| 419-427 | TISPSYTYY | 222 | A1 | 19 | 5 |
| | | | A26 | 27 | N.A. |
| | | | A3 | 18 | <5 |
| 419-428 | TISPSYTYYR | 223 | A3 | 15 | 5.4 |
| 424-433 | YTYYRPGVNL | 224 | A0201 | 18 | <5 |
| | | | A24 | N.A. | <5 |
| | | | A26 | 20 | N.A. |
| 425-433 | TYYRPGVNL | 225 | A0201 | 14 | <5 |
| | | | A24 | N.A. | 200 |
| | | | B0702 | 16 | <5 |
| | | | B2705 | 16 | 5 |
| 426-433 | YYRPGVNL | 226 | B08 | 16 | <5 |
| 426-435 | YYRPGVNLSL | 227 | A0201 | 17 | <5 |
| | | | B0702 | 15 | <5 |
| 427-435 | YRPGVNLSL | 228 | A0201 | 17 | <5 |
| | | | B2705 | 26 | 2000 |
| | | | B2709 | 21 | N.A. |
| 428-435 | RPGVNLSL | 229 | B08 | 17 | <5 |
| | | | B5101 | 17 | N.A. |
| 428-437 | RPGVNLSLSC | 230 | B0702 | 14 | <5 |
| 430-438 | GVNLSLSCH | 231 | A26 | 16 | N.A. |
| | | | B2705 | 15 | <5 |
| 431-440 | VNLSLSCHAA | 232 | A0203 | 19 | N.A. |
| 432-440 | NLSLSCHAA | 233 | A0201 | 16 | <5 |

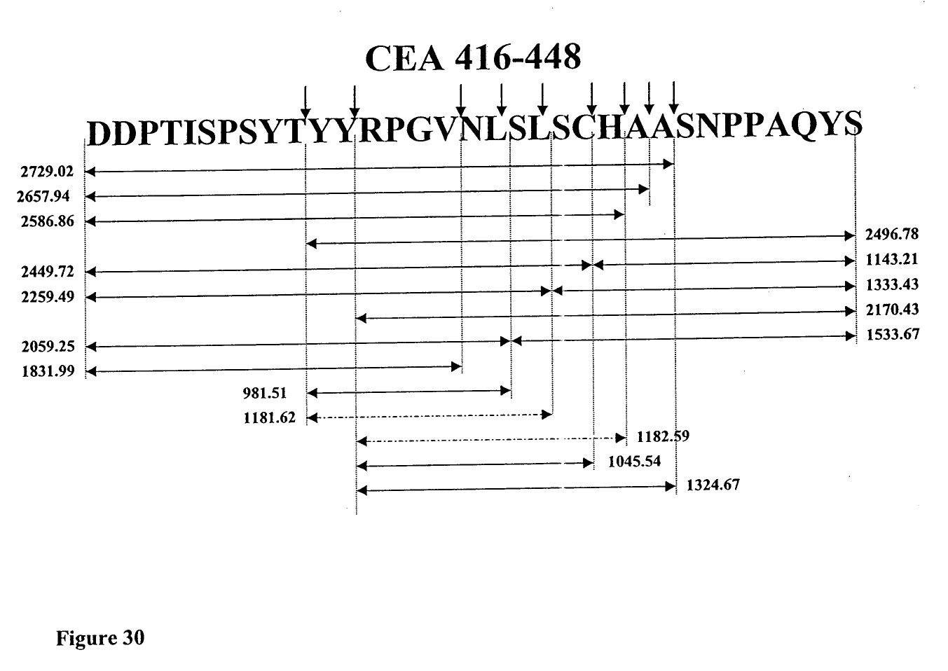

†Scores are given from the two binding prediction programs referenced above (see example 3)
See also FIG. 30.

### Example 28

### CEA 437-464

TABLE 28
Preferred Epitopes Revealed by Housekeeping
Proteasome Digestion

| | | | | HLA binding | |
|---|---|---|---|---|---|
| | | Sequence | HLA | predictions† | |
| Epitope | Sequence | ID No. | type | SYFPEITHI | NIH |
| 438-447 | HAASNPPAQY | 234 | A1 | 15 | <5 |
| 439-447 | AASNPPAQY | 235 | A1 | 17 | <5 |
| | | | A3 | 17 | <5 |
| 442-451 | NPPAQYSWLI | 236 | B0702 | 17 | 8 |
| 443-451 | PPAQYSWLI | 237 | B0702 | 17 | <5 |
| | | | B5101 | 21 | 40 |
| 444-451 | PAQYSWLI | 238 | B5101 | 20 | N.A. |
| 449-458 | WLIDGNIQQH | 239 | A0201 | 17 | <5 |
| | | | A26 | 17 | N.A. |
| | | | A3 | 21 | <5 |
| 450-458 | LIDGNIQQH | 240 | A0201 | 16 | <5 |
| | | | A26 | 19 | N.A. |
| | | | A3 | 17 | <5 |
| 450-459 | LIDGNIQQHT | 241 | A0201 | 16 | <5 |
| | | | A26 | 15 | N.A. |

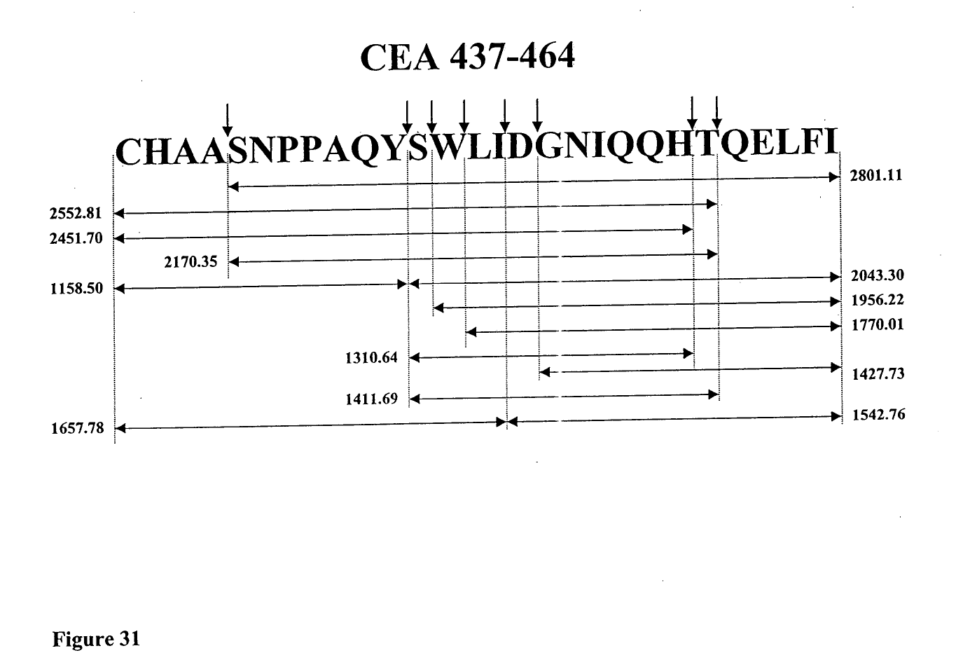

†Scores are given from the two binding prediction programs referenced above (see example 3)
See also FIG. 31.

### Example 29

### CEA 581-607

TABLE 29
Preferred Epitopes Revealed by Housekeeping
Proteasome Digestion

| | | Se- | | HLA binding | |
|---|---|---|---|---|---|
| | | quence | HLA | predictions† | |
| Epitope | Sequence | ID No. | type | SYFPEITHI | NIH |
| 581-590 | RSDPVTLDVL | 242 | A0201 | 16 | <5 |
| | | | A1 | 19 | 7.5 |
| | | | A26 | 15 | N.A. |
| | | | A24 | N.A. | 9.6 |
| 582-590 | SDPVTLDVL | 243 | A0201 | 16 | <5 |
| 582-591 | SDPVTLDVLY | 244 | A1 | 19 | <5 |
| 583-590 | DPVTLDVL | 245 | B08 | 16 | <5 |
| | | | B5101 | 25 | N.A. |
| 583-591 | DPVTLDVLY | 246 | A1 | 17 | <5 |
| | | | A26 | 18 | N.A. |
| | | | B5101 | 16 | 6 |
| 588-597 | DVLYGPDTPI | 247 | A26 | 16 | N.A. |
| 589-597 | VLYGPDTPI | 248 | A0201 | 25 | 56.754 |
| | | | A3 | 17 | 6.75 |
| | | | B5101 | 17 | 11.44 |
| 596-605 | PIISPPDSSY | 249 | A1 | 15 | <5 |
| | | | A26 | 25 | N.A. |
| | | | A3 | 22 | <5 |
| 597-605 | IISPPDSSY | 250 | A1 | 20 | 5 |
| | | | A26 | 24 | N.A. |
| | | | A3 | 24 | <5 |

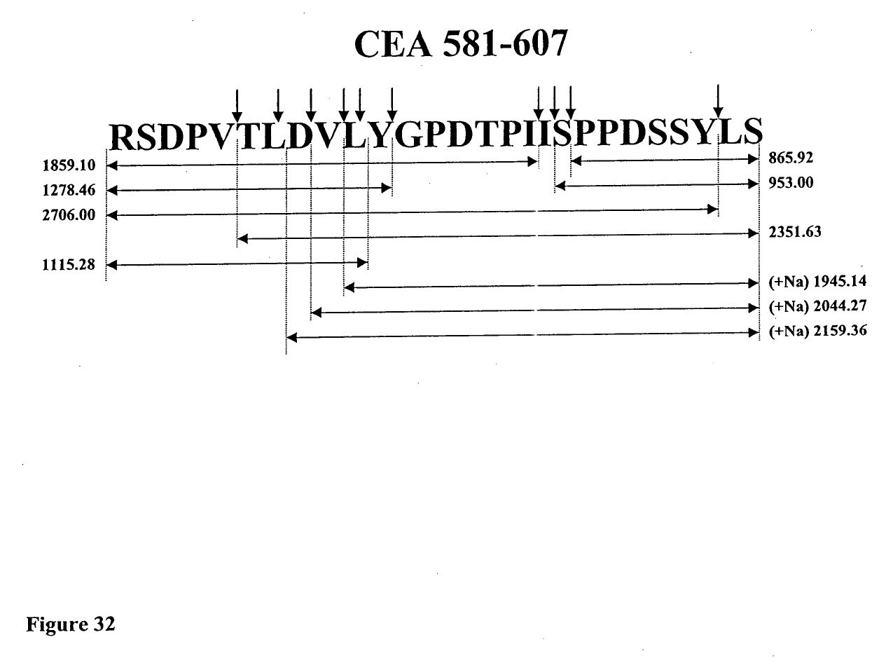

†Scores are given from the two binding prediction programs referenced above (see example 3)
See also FIG. 32.

### Example 30

### CEA 595-622

TABLE 30
Preferred Epitopes Revealed by Housekeeping
Proteasome Digestion

| | | Se- | | HLA binding | |
|---|---|---|---|---|---|
| | | quence | HLA | predictions† | |
| Epitope | Sequence | ID No. | type | SYFPEITHI | NIH |
| 597-606 | IISPPDSSYL | 251 | A0201 | 22 | 27.464 |
| | | | A26 | 21 | N.A. |
| | | | A3 | 16 | <5 |
| | | | B0702 | 14 | <5 |
| 599-606 | SPPDSSYL | 252 | B08 | 18 | <5 |
| | | | B5101 | 17 | N.A. |
| 600-608 | PPDSSYLSG | 253 | A1 | 16 | <5 |
| 600-609 | PPDSSYLSGA | 254 | B0702 | 17 | <5 |
| 602-611 | DSSYLSGANL | 255 | A26 | 16 | N.A. |
| 603-611 | SSYLSGANL | 256 | A0201 | 15 | <5 |
| | | | B2705 | 17 | 50 |
| 604-613 | SYLSGANLNL | 257 | A0201 | 15 | <5 |
| | | | A24 | N.A. | 300 |
| 605-613 | YLSGANLNL | 258 | A0201 | 25 | 98.267 |
| | | | A26 | 19 | N.A. |
| | | | A3 | 15 | <5 |
| | | | B0702 | 16 | <5 |
| | | | B08 | 17 | <5 |
| | | | B2705 | 16 | 30 |
| 610-618 | NLNLSCHSA | 259 | A0201 | 18 | <5 |

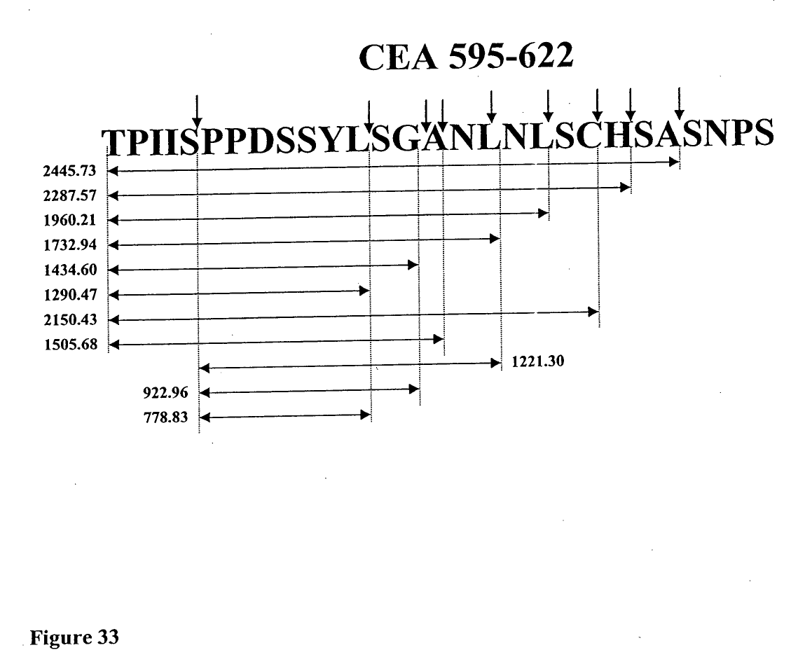

†Scores are given from the two binding prediction programs referenced above (see example 3)
See also FIG. 33.

### Example 31

### CEA 615-641

TABLE 31
Preferred Epitopes Revealed by Housekeeping
Proteasome Digestion

| | | | | HLA binding | |
|---|---|---|---|---|---|
| | | Sequence | HLA | predictions† | |
| Epitope | Sequence | ID No. | type | SYFPEITHI | NIH |
| 620-629 | NPSPQYSWRI | 260 | B0702 | 19 | 8 |
| 622-629 | SPQYSWRI | 261 | B08 | 15 | <5 |
| | | | B5101 | 20 | N.A. |
| 627-635 | WRINGIPQQ | 262 | B2705 | 19 | 20 |
| 628-636 | RINGIPQQH | 263 | A3 | 22 | <5 |
| | | | B2705 | 16 | <5 |
| 628-637 | RINGIPQQHT | 264 | A0201 | 15 | <5 |
| 631-639 | GIPQQHTQV | 265 | A0201 | 19 | 9.563 |
| 632-639 | IPQQHTQV | 266 | B5101 | 20 | N.A. |

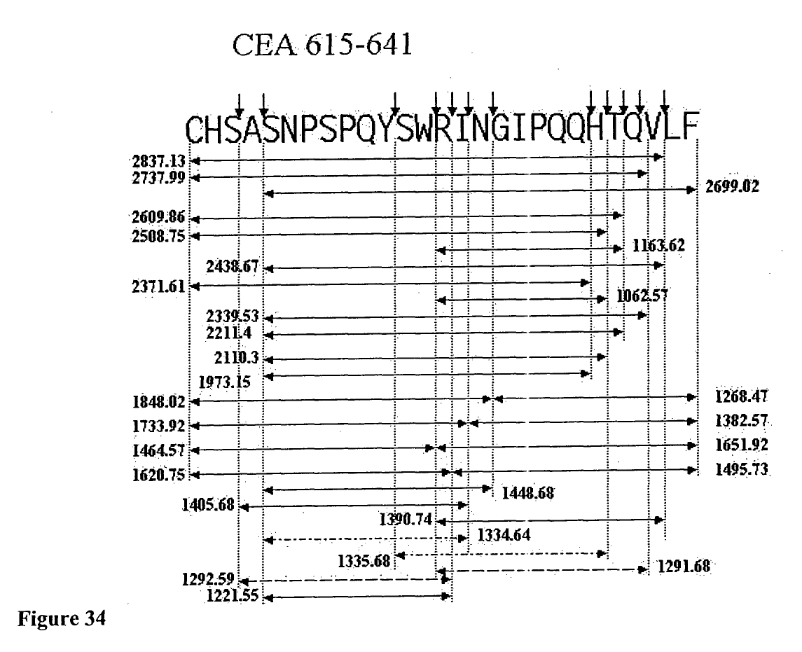

†Scores are given from the two binding prediction programs referenced above (see example 3)
See also FIG. 34.

### Example 32

### CEA 643-677

TABLE 32
Preferred Epitopes Revealed by Housekeeping
Proteasome Digestion

| | | | | HLA binding | |
|---|---|---|---|---|---|
| | | Sequence | HLA | predictions† | |
| Epitope | Sequence | ID No. | type | SYFPEITHI | NIH |
| 644-653 | KITPNNNGTY | 267 | A1 | 20 | 5 |
| | | | A26 | 22 | N.A. |
| | | | A3 | 25 | <5 |
| 645-653 | ITPNNNGTY | 268 | A1 | 22 | <5 |
| | | | A26 | 21 | N.A. |
| | | | A3 | 14 | <5 |
| 647-656 | PNNNGTYACF | 269 | A26 | 15 | N.A. |
| 648-656 | NNNGTYACF | 270 | A26 | 17 | N.A. |
| 650-657 | NGTYACFV | 271 | B5101 | 15 | N.A. |
| 661-670 | ATGRNNSIVK | 272 | A3 | 20 | <5 |
| 662-670 | TGRNNSIVK | 273 | A3 | 18 | <5 |
| 664-672 | RNNSIVKSI | 274 | B2709 | 15 | N.A. |
| 666-674 | NSIVKSITV | 275 | A0201 | 16 | <5 |

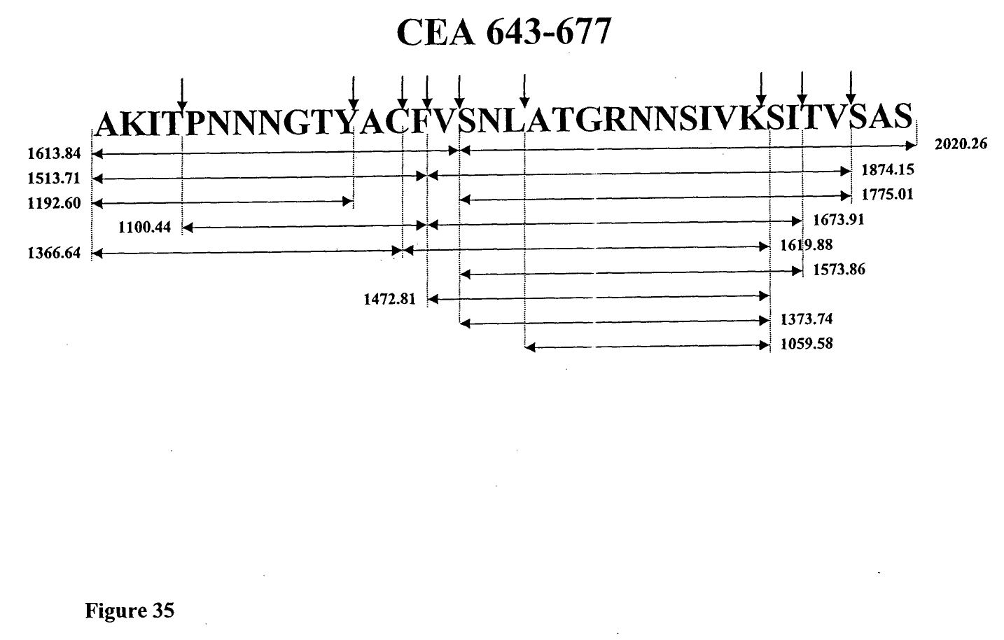

†Scores are given from the two binding prediction programs referenced above (see example 3)
See also FIG. 35.

### Example 33

### GAGE-1 6-32

TABLE 33
Preferred Epitopes Revealed by Housekeeping
Proteasome Digestion

| | | | | HLA binding | |
|---|---|---|---|---|---|
| | | Sequence | HLA | predictions† | |
| Epitope | Sequence | ID No. | type | SYFPEITHI | NIH |
| 7-16 | STYRPRPRRY | 276 | A1 | 23 | <5 |
| | | | A26 | 21 | N/A |
| | | | A3 | 15 | <5 |
| 8-16 | TYRPRPRRY | 277 | A1 | 19 | <5 |
| | | | A3 | 15 | <5 |
| 10-18 | RPRPRRYVE | 278 | A3 | 17 | <5 |
| | | | B0702 | 16 | N/A |
| | | | B08 | 20 | <5 |
| 16-23 | YVEPPEMI | 279 | B5101 | 15 | N/A |
| 22-31 | MIGPMRPEQF | 280 | A26 | 23 | N/A |
| | | | A3 | 19 | <5 |
| 23-31 | IGPMRPEQF | 281 | B08 | 15 | <5 |
| 24-31 | GPMRPEQF | 282 | B5101 | 16 | N/A |

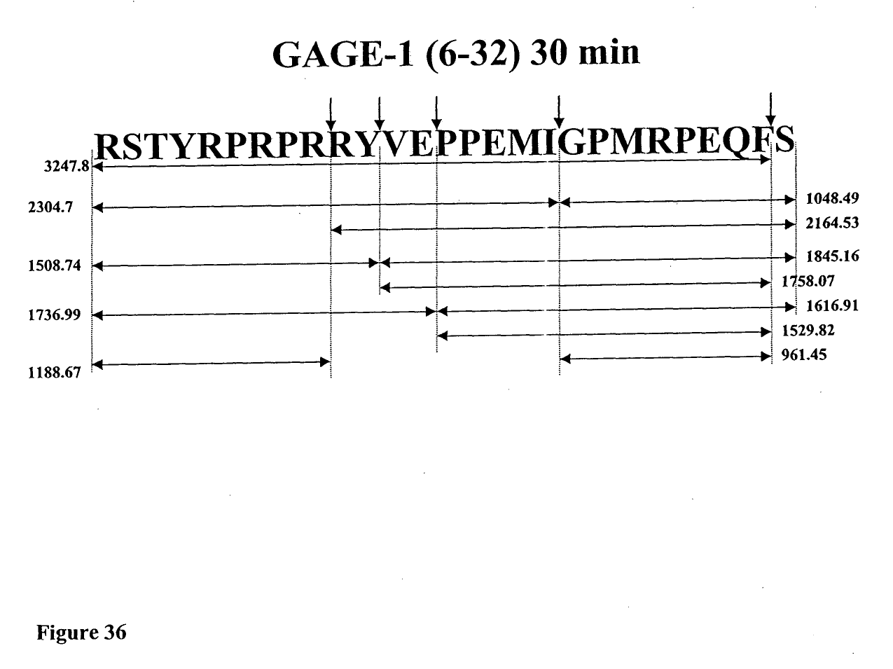

†Scores are given from the two binding prediction programs referenced above (see example 3)
See also FIG. 36.

### Example 34

### GAGE-1 105-131

TABLE 34
Preferred Epitopes Revealed by Housekeeping
Proteasome Digestion

| | | Se- | | HLA binding | |
|---|---|---|---|---|---|
| | | quence | HLA | predictions† | |
| Epitope | Sequence | ID No. | type | SYFPEITHI | NIH |
| 105-114 | KTPEEEMRSH | 283 | A26 | 18 | N/A |
| 106-115 | TPEEEMRSHY | 284 | A1 | 26 | 11.25 |
| 107-115 | PEEEMRSHY | 285 | A1 | 26 | <5 |
| 110-119 | EMRSHYVAQT | 286 | A0201 | 15 | <5 |
| 113-121 | SHYVAQTGI | 287 | B5101 | 15 | <5 |
| 115-124 | YVAQTGILWL | 288 | A0201 | 23 | 108.769 |
| | | | A26 | 24 | N/A |
| | | | A3 | 15 | <5 |
| 116-124 | VAQTGILWL | 289 | A0201 | 22 | 6.381 |
| | | | B08 | 16 | <5 |
| | | | B2705 | 16 | 10 |
| | | | B5101 | 20 | 78.65 |
| 116-125 | VAQTGILWLL | 290 | A0201 | 19 | 8.701 |
| 117-125 | AQTGILWLL | 291 | A0201 | 17 | 37.362 |
| | | | B2705 | 16 | 200 |
| 118-126 | QTGILWLLM | 292 | A26 | 19 | N/A |
| 118-127 | QTGILWLLMN | 293 | A26 | 15 | N/A |
| 120-129 | GILWLLMNNC | 294 | A26 | 15 | N/A |
| 121-129 | ILWLLMNNC | 295 | A0201 | 15 | 161.227 |

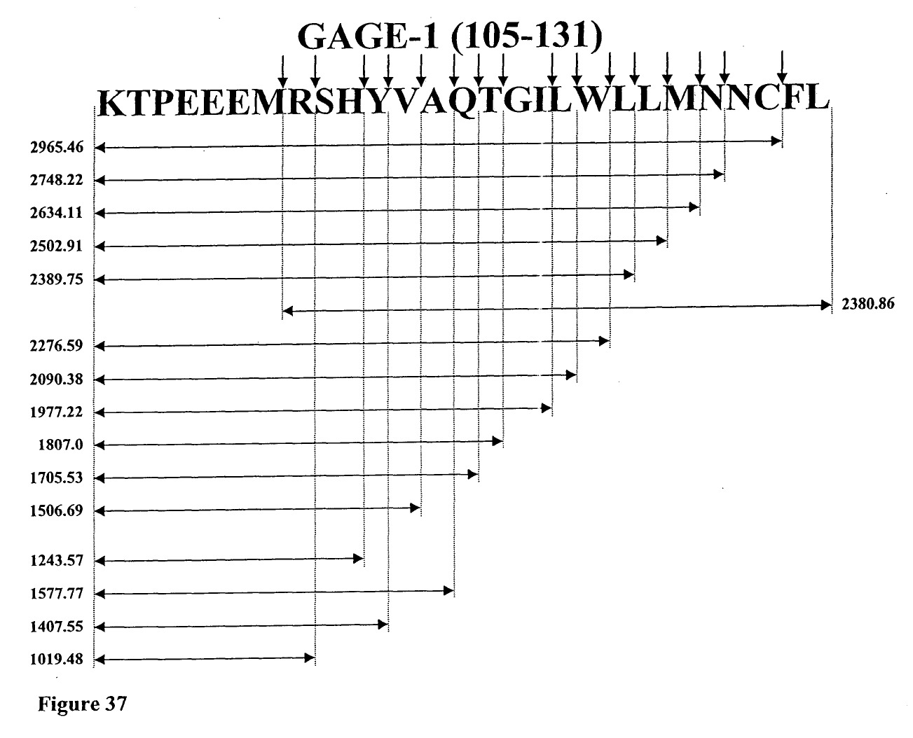

†Scores are given from the two binding prediction programs referenced above (see example 3)
See also FIG. 37.

### Example 35

### GAGE-1 112-137

TABLE 35
Preferred Epitopes Revealed by Housekeeping Proteasome Digestion

| | | | | HLA binding | |
|---|---|---|---|---|---|
| | | Sequence | HLA | predictions† | |
| Epitope | Sequence | ID No. | type | SYFPEITHI | NIH |
| 124-131 | LLMNNCFL | 296 | B08 | 16 | <5 |
| 123-131 | WLLMNNCFL | 297 | A0201 | 22 | 1999.734 |
| | | | A26 | 16 | N/A |
| | | | B08 | 17 | <5 |
| 122-130 | LWLLMNNCF | 298 | B2705 | 15 | <5 |
| 121-130 | ILWLLMNNCF | 299 | A26 | 18 | N/A |
| | | | A3 | 17 | 10 |
| 121-129 | ILWLLMNNC | 295 | A0201 | 15 | 161.227 |
| 120-129 | GILWLLMNNC | 294 | A26 | 15 | N/A |
| 118-127 | QTGILWLLMN | 293 | A26 | 15 | N/A |
| 118-126 | QTGILWLLM | 292 | A26 | 19 | N/A |
| 117-125 | AQTGILWLL | 291 | A0201 | 17 | 37.362 |
| | | | B2705 | 16 | 200 |
| | | | B4402 | 17 | N/A |
| 116-125 | VAQTGILWLL | 290 | A0201 | 19 | 8.701 |
| 116-124 | VAQTGILWL | 289 | A0201 | 22 | 6.381 |
| | | | B08 | 16 | <5 |
| | | | B2705 | 16 | 10 |
| | | | B4402 | 15 | N/A |
| | | | B5101 | 20 | 78.65 |
| 115-124 | YVAQTGILWL | 288 | A0201 | 23 | 108.769 |
| | | | A26 | 24 | N/A |
| | | | A3 | 15 | <5 |
| 113-121 | SHYVAQTGI | 287 | B5101 | 15 | <5 |

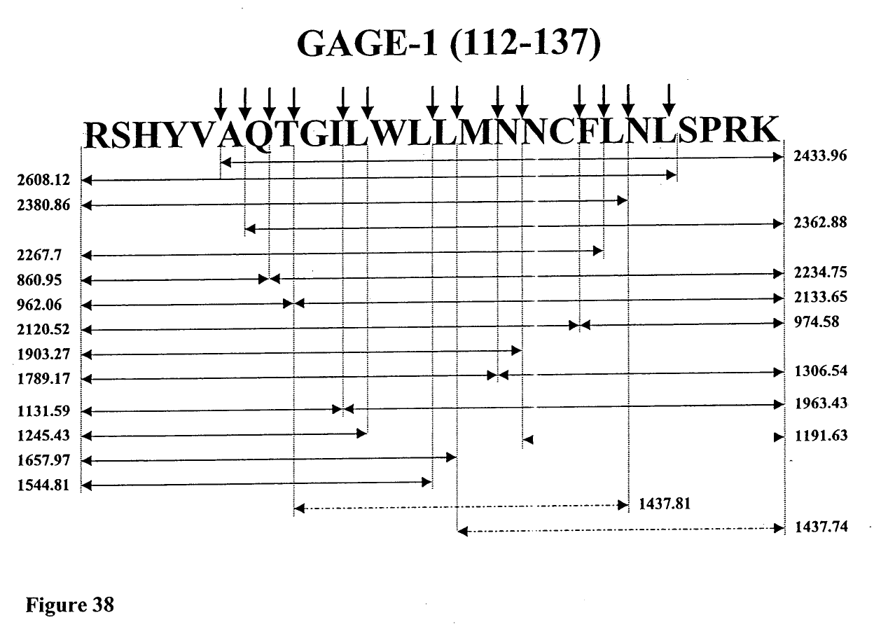

†Scores are given from the two binding prediction programs referenced above (see example 3)
See also FIG. 38.

### Example 36

### MAGE-1 51-77

TABLE 36
Preferred Epitopes Revealed by Housekeeping
Proteasome Digestion

| | | Se- | | HLA binding | |
|---|---|---|---|---|---|
| Epi- | | quence | HLA | predictions† | |
| tope | Sequence | ID No. | type | SYFPEITHI | NIH |
| 62-70 | SAFPTTINF | 309 | A26 | 15 | N/A |
| | | | B4402 | 18 | N/A |
| | | | B2705 | 17 | 25 |
| 61-70 | ASAFPTTINF | 310 | B4402 | 15 | N/A |
| 60-68 | GASAFPTTI | 311 | A0201 | 16 | <5 |
| | | | B5101 | 25 | 220 |
| 57-66 | SPQGASAFPT | 312 | B0702 | 19 | N/A |

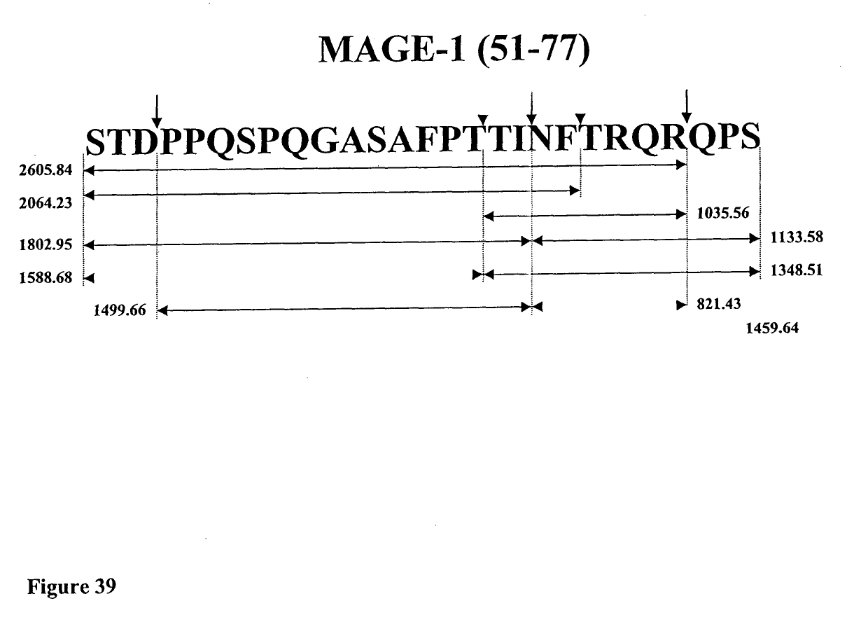

†Scores are given from the two binding prediction programs referenced above
See also FIG. 39.

### Example 37

### MAGE-1 126-153

TABLE 37
Preferred Epitopes Revealed by Housekeeping
Proteasome Digestion

| | | | | HLA binding | |
|---|---|---|---|---|---|
| | | Sequence | HLA | predictions† | |
| Epitope | Sequence | ID No. | type | SYFPEITHI | NIH |
| 144-151 | FGKASESL | 313 | B08 | 21 | <5 |
| 143-151 | IFGKASESL | 314 | A26 | 16 | N/A |
| | | | B2705 | 15 | <5 |
| 142-151 | EIFGKASESL | 315 | A0201 | 20 | <5 |
| | | | A26 | 29 | N/A |
| | | | B4402 | 15 | N/A |
| 142-149 | EIFGKASE | 316 | B08 | 16 | <5 |
| 133-140 | IKNYKHCF | 317 | B08 | 18 | <5 |
| 132-140 | VIKNYKHCF | 318 | A26 | 21 | N/A |
| | | | B08 | 21 | <5 |
| 131-140 | SVIKNYKHCF | 319 | A26 | 23 | N/A |
| | | | A3 | 18 | <5 |
| | | | B4402 | 15 | N/A |
| 132-139 | VIKNYKHC | 320 | B08 | 15 | <5 |
| 131-139 | SVIKNYKHC | 321 | A26 | 18 | N/A |
| 128-136 | MLESVIKNY | 322 | A1 | 28 | 45 |
| | | | A26 | 24 | N/A |
| | | | A3 | 17 | <5 |
| | | | B4402 | 15 | N/A |
| 127-136 | EMLESVIKNY | 323 | A1 | 15 | <5 |
| | | | A26 | 23 | N/A |
| | | | B4402 | 18 | N/A |
| 126-134 | AEMLESVIK | 324 | A3 | 18 | <5 |
| | | | B2705 | 15 | 30 |
| | | | B4402 | 16 | N/A |

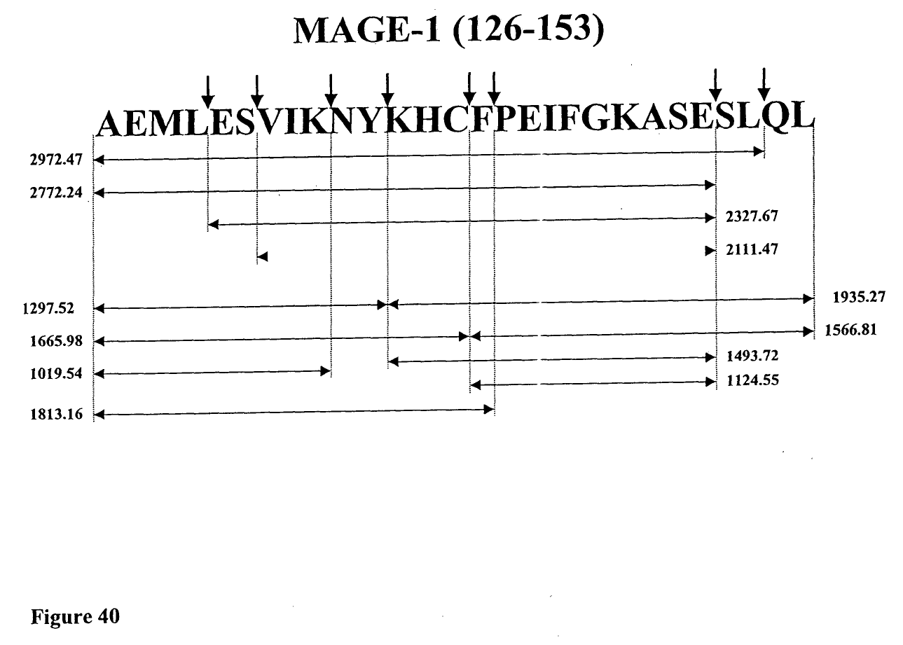

†Scores are given from the two binding prediction programs referenced above (see example 3).
See also FIG. 40.

### Example 38

### MAGE-2 272-299

TABLE 38
Preferred Epitopes Revealed by Housekeeping
Proteasome Digestion

| | | Se- | | HLA binding | |
|---|---|---|---|---|---|
| Epi- | | quence | HLA | predictions† | |
| tope | Sequence | ID No. | type | SYFPEITHI | NIH |
| 274-283 | GPRALIETSY | 325 | A1 | 15 | <5 |
| 275-283 | PRALIETSY | 326 | A1 | 15 | <5 |
| | | | B2705 | 23 | 100 |
| 276-284 | RALIETSYV | 327 | A0201 | 18 | 19.658 |
| | | | B5101 | 20 | 55 |
| 277-286 | ALIETSYVKV | 328 | A0201 | 30 | 427.745 |
| | | | A26 | 18 | N/A |
| | | | A3 | 21 | <5 |
| 278-286 | LIETSYVKV | 329 | A0201 | 23 | <5 |
| | | | A26 | 17 | N/A |
| | | | B5101 | 15 | <5 |
| 278-287 | LIETSYVKVL | 330 | A0201 | 22 | <5 |
| | | | A26 | 22 | N/A |
| 279-287 | IETSYVKVL | 331 | A0201 | 15 | <5 |
| | | | B1510 | 15 | N/A |
| | | | B5101 | 15 | <5 |
| 280-289 | ETSYVKVLHH | 332 | A26 | 21 | N/A |
| 282-291 | SYVKVLHHTL | 333 | A0201 | 15 | <5 |
| 283-291 | YVKVLHHTL | 334 | A0201 | 19 | <5 |
| | | | A26 | 20 | N/A |
| | | | A3 | 15 | <5 |
| | | | B08 | 21 | <5 |
| 285-293 | KVLHHTLKI | 335 | A0201 | 20 | 11.822 |
| | | | A3 | 18 | <5 |
| | | | B5101 | 15 | <5 |

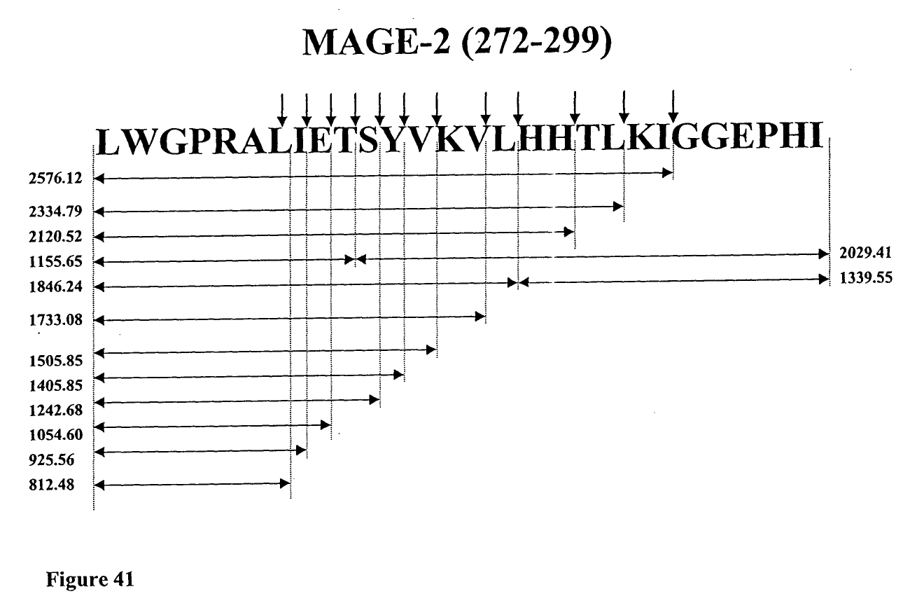

†Scores are given from the two binding prediction programs referenced above (see example 3)
See also FIG. 41.

### Example 39

### MAGE-2 287-314

TABLE 39
Preferred Epitopes Revealed by Housekeeping
Proteasome Digestion

| | | | | HLA binding | |
|---|---|---|---|---|---|
| | | Sequence | HLA | predictions† | |
| Epitope | Sequence | ID No. | type | SYFPEITHI | NIH |
| 303-311 | PLHERALRE | 336 | A3 | 19 | <5 |
| | | | B08 | 16 | <5 |
| 302-309 | PPLHERAL | 337 | B08 | 16 | <5 |
| | | | B5101 | 18 | N/A |
| 301-309 | YPPLHERAL | 338 | B0702 | 21 | N/A |
| | | | B08 | 18 | <5 |
| | | | B4402 | 15 | N/A |
| | | | B5101 | 20 | 143 |
| 300-309 | SYPPLHERAL | 339 | A0201 | 15 | <5 |
| | | | B4402 | 18 | N/A |
| 299-307 | ISYPPLHER | 340 | B2705 | 17 | 25 |
| 298-307 | HISYPPLHER | 341 | A26 | 15 | N/A |
| 292-299 | KIGGEPHI | 342 | B5101 | 15 | N/A |
| 291-299 | LKIGGEPHI | 343 | A0201 | 17 | <5 |
| 290-299 | TLKIGGEPHI | 344 | A0201 | 18 | <5 |

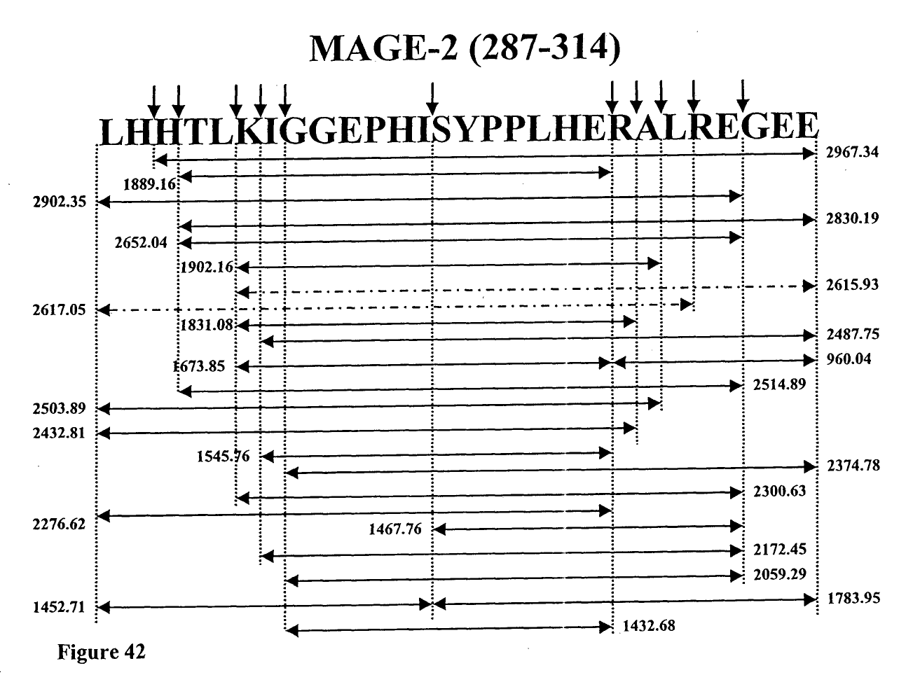

†Scores are given from the two binding prediction programs referenced above (see example 3)
See also FIG. 42.

### Example 40

### Mage-3 287-314

TABLE 40
Preferred Epitopes Revealed by Housekeeping
Proteasome Digestion

| | | | | HLA binding | |
|---|---|---|---|---|---|
| | | Sequence | HLA | predictions† | |
| Epitope | Sequence | ID No. | type | SYFPEITHI | NIH |
| 303-311 | PLHEWVLRE | 345 | A26 | 15 | N/A |
| 302-309 | PPLHEWVL | 346 | B08 | 16 | <5 |
| | | | B5101 | 19 | N/A |
| 301-309 | YPPLHEWVL | 347 | B0702 | 21 | N/A |
| | | | B08 | 17 | <5 |
| | | | B5101 | 22 | 130 |
| 301-308 | YPPLHEWV | 348 | B5101 | 22 | N/A |
| 300-308 | SYPPLHEWV | 349 | A0201 | 15 | <5 |
| 299-308 | ISYPPLHEWV | 350 | A0201 | 15 | 6.656 |
| 298-307 | HISYPPLHEW | 351 | A26 | 15 | N/A |
| 293-301 | ISGGPHISY | 352 | A1 | 25 | <5 |
| 292-301 | KISGGPHISY | 353 | A1 | 20 | <5 |
| | | | A26 | 23 | N/A |
| | | | A3 | 21 | 5.4 |

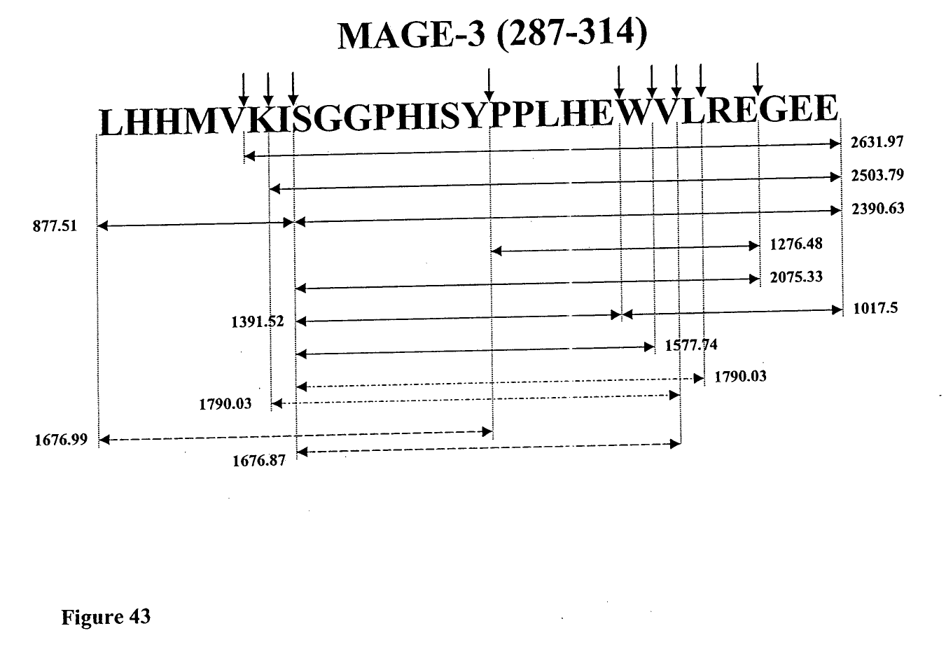

†Scores are given from the two binding prediction programs referenced above (see example 3)
See also FIG. 43.

### Example 41

### Melan-A 44-71

TABLE 41
Preferred Epitopes Revealed by Housekeeping
Proteasome Digestion

| | | Se- | | HLA binding | |
|---|---|---|---|---|---|
| Epi- | | quence | HLA | predictions† | |
| tope | Sequence | ID No. | type | SYFPEITHI | NIH |
| 45-54 | CWYCRRRNGY | 354 | A1 | 16 | <5 |
| 46-54 | WYCRRRNGY | 355 | A1 | 16 | <5 |
| 47-55 | YCRRRNGYR | 356 | B08 | 15 | <5 |
| 49-57 | RRRNGYRAL | 357 | B08 | 17 | <5 |
| | | | B2705 | 26 | 1800 |
| | | | B2709 | 24 | N/A |
| 51-60 | RNGYRALMDK | 358 | A3 | 15 | <5 |
| 52-60 | NGYRALMDK | 359 | A3 | 18 | <5 |
| 55-63 | RALMDKSLH | 360 | B2705 | 16 | <5 |
| 56-63 | ALMDKSLH | 361 | B08 | 16 | <5 |
| 55-64 | RALMDKSLHV | 362 | A0201 | 17 | <5 |
| 56-64 | ALMDKSLHV | 363 | A0201 | 26 | 1055.104 |
| | | | A3 | 18 | <5 |
| | | | B08 | 16 | <5 |

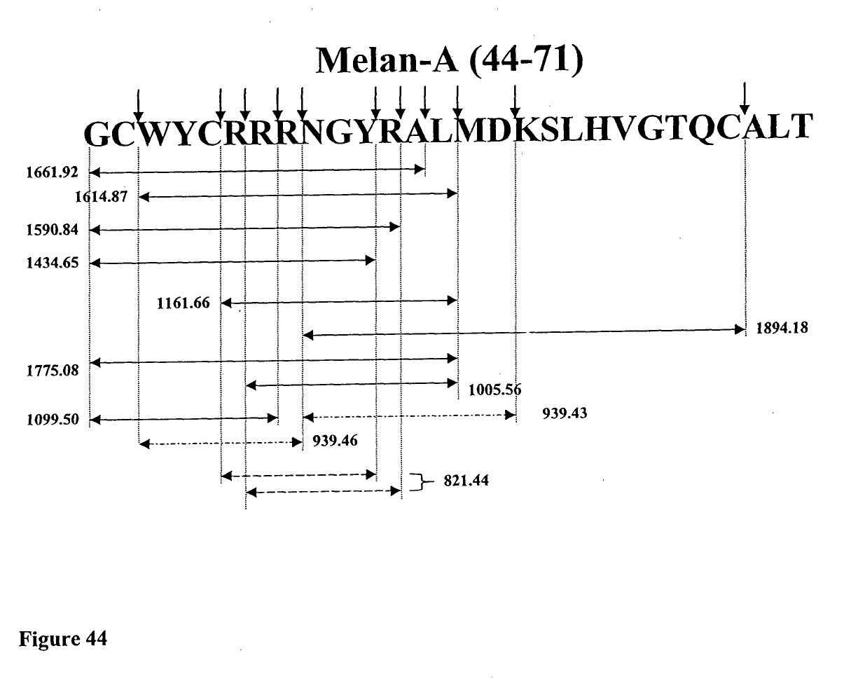

†Scores are given from the two binding prediction programs referenced above (see example 3)
See also FIG. 44.

### Example 42

### PRAME 274-301

TABLE 42
Preferred Epitopes Revealed by Housekeeping
Proteasome Digestion

| | | Se- | | HLA binding | |
|---|---|---|---|---|---|
| | | quence | HLA | predictions† | |
| Epitope | Sequence | ID No. | type | SYFPEITHI | NIH |
| 275-284 | YISPEKEEQY | 364 | A1 | 21 | 5 |
| | | | A26 | 23 | N/A |
| | | | A3 | 20 | <5 |
| | | | B4402 | 15 | N/A |
| 276-284 | ISPEKEEQY | 365 | A1 | 19 | <5 |
| | | | A26 | 15 | N/A |
| 277-285 | SPEKEEQYI | 366 | B0702 | 17 | N/A |
| | | | B5101 | 21 | 484 |
| 278-285 | PEKEEQYI | 367 | B08 | 18 | <5 |
| 279-288 | EKEEQYIAQF | 368 | A26 | 24 | N/A |
| | | | B4402 | 16 | N/A |
| 280-288 | KEEQYIAQF | 369 | A26 | 17 | N/A |
| | | | B2705 | 19 | 45 |
| | | | B4402 | 25 | N/A |
| 283-292 | QYIAQFTSQF | 370 | A3 | 17 | <5 |
| | | | B4402 | 15 | N/A |
| 284-292 | YIAQFTSQF | 371 | A0201 | 15 | <5 |
| | | | A26 | 24 | N/A |
| | | | A3 | 19 | <5 |
| 284-293 | YIAQFTSQFL | 372 | A0201 | 22 | 74.314 |
| | | | A26 | 21 | N/A |
| 285-293 | IAQFTSQFL | 373 | A0201 | 15 | <5 |
| | | | B08 | 15 | <5 |
| | | | B5101 | 19 | 78.65 |
| 286-295 | AQFTSQFLSL | 374 | A0201 | 16 | 15.226 |
| | | | A26 | 15 | N/A |
| | | | B0702 | 15 | N/A |
| | | | A4402 | 18 | N/A |
| 287-295 | QFTSQFLSL | 375 | A26 | 21 | N/A |
| 290-298 | SQFLSLQCL | 376 | A0201 | 17 | 18.432 |
| | | | A26 | 16 | N/A |
| | | | B2705 | 16 | 1000 |
| | | | B4402 | 15 | N/A |

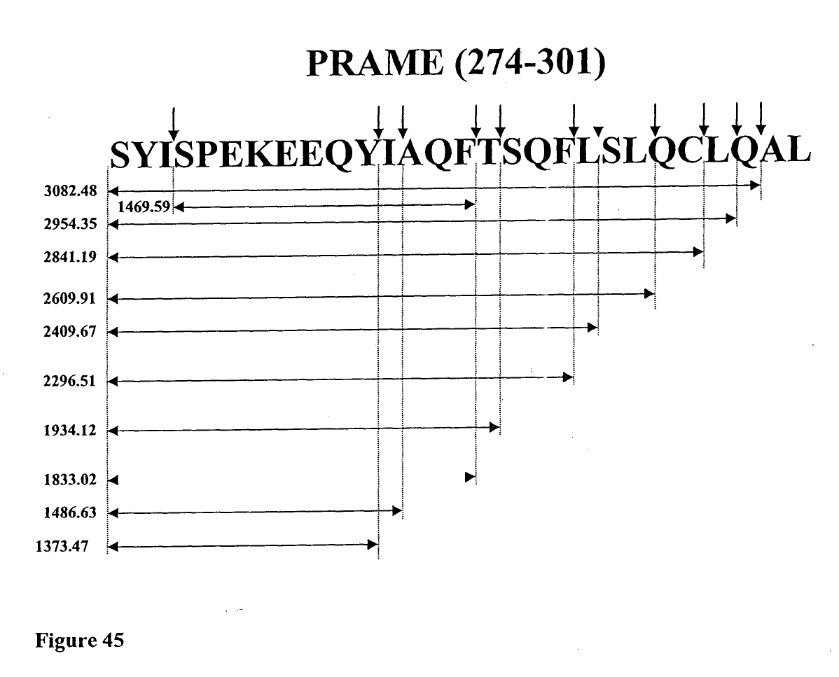

†Scores are given from the two binding prediction programs referenced above (see example 3)
See also FIG. 45.

### Example 43

### PRAME 434-463

TABLE 43
Preferred Epitopes Revealed by Housekeeping
Proteasome Digestion

| | | | | HLA binding | |
|---|---|---|---|---|---|
| | | Sequence | HLA | predictions† | |
| Epitope | Sequence | ID No. | type | SYFPEITHI | NIH |
| 439-448 | VLYPVPLESY | 377 | A0201 | 20 | <5 |
| | | | A1 | 21 | 5 |
| | | | A26 | 25 | N/A |
| | | | A3 | 25 | 67.5 |
| 440-448 | LYPVPLESY | 378 | A1 | 16 | <5 |
| 446-455 | ESYEDIHGTL | 379 | A26 | 16 | N/A |
| 448-457 | YEDIHGTLHL | 380 | A1 | 18 | <5 |
| 449-457 | EDIHGTLHL | 381 | B2705 | 15 | <5 |
| 451-460 | IHGTLHLERL | 382 | A0201 | 16 | <5 |

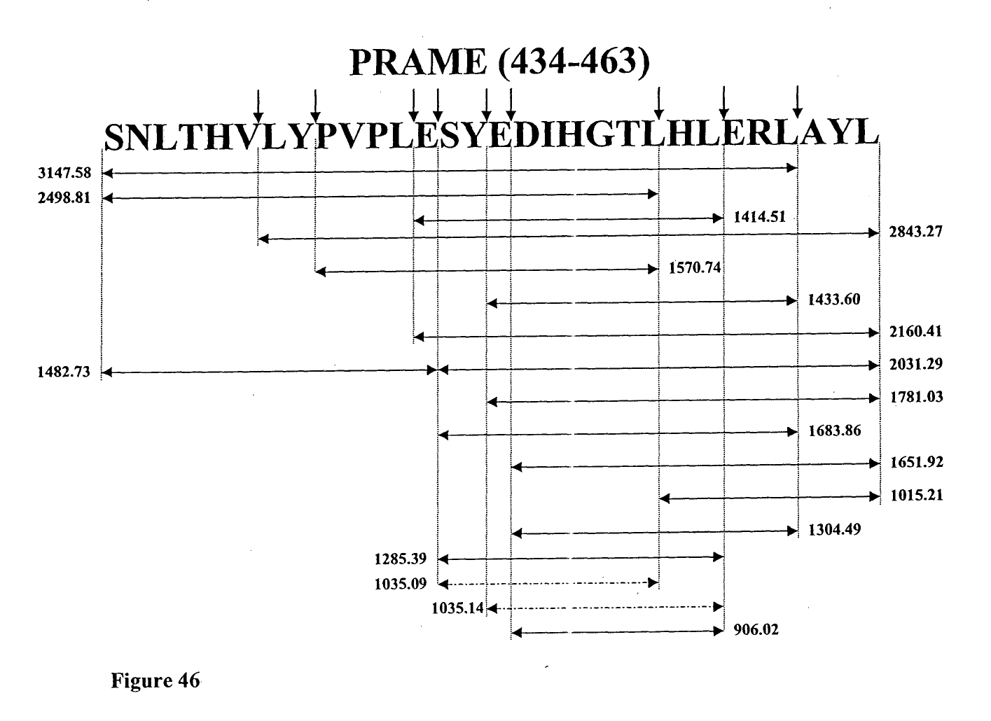

†Scores are given from the two binding prediction programs referenced above (see example 3)
See also FIG. 46.

### Example 44

### PRAME 452-480

TABLE 44
Preferred Epitopes Revealed by Housekeeping
Proteasome Digestion

| | | Se- | | HLA binding | |
|---|---|---|---|---|---|
| | | quence | HLA | predictions† | |
| Epitope | Sequence | ID No. | type | SYFPEITHI | NIH |
| 454-463 | TLHLERLAYL | 383 | A0201 | 26 | 270.234 |
| | | | A26 | 21 | N/A |
| 455-463 | LHLERLAYL | 384 | A0201 | 22 | <5 |
| | | | B08 | 20 | <5 |
| | | | B1510 | 21 | N/A |
| | | | B2705 | 15 | <5 |
| 456-463 | HLERLAYL | 385 | B08 | 17 | <5 |
| 456-465 | HLERLAYLHA | 386 | A3 | 16 | <5 |
| | | | A1 | 17 | <5 |
| 458-467 | ERLAYLHARL | 387 | A26 | 16 | N/A |
| 459-467 | RLAYLHARL | 388 | A0201 | 24 | 21.362 |
| | | | B08 | 17 | <5 |
| | | | B2705 | 18 | 90 |
| | | | B2709 | 15 | N/A |
| 459-468 | RLAYLHARLR | 389 | A3 | 22 | <5 |
| 460-467 | LAYLHARL | 390 | B08 | 15 | <5 |
| | | | B5101 | 20 | N/A |
| 460-468 | LAYLHARLR | 391 | B5101 | 18 | <5 |
| 461-470 | AYLHARLREL | 392 | A0201 | 20 | <5 |
| | | | B4402 | 16 | N/A |
| 462-470 | YLHARLREL | 393 | A0201 | 28 | 45.203 |
| | | | B08 | 25 | 8 |
| 462-471 | YLHARLRELL | 394 | A0201 | 22 | 48.151 |
| | | | A26 | 16 | N/A |
| 463-471 | LHARLRELL | 395 | A0201 | 15 | <5 |
| | | | B1510 | 22 | N/A |
| 464-471 | HARLRELL | 396 | B08 | 30 | 320 |
| | | | B5101 | 17 | N/A |
| 464-472 | HARLRELLC | 397 | B08 | 20 | 16 |
| 469-478 | ELLCELGRPS | 398 | A3 | 15 | <5 |
| 470-478 | LLCELGRPS | 399 | A0201 | 15 | <5 |

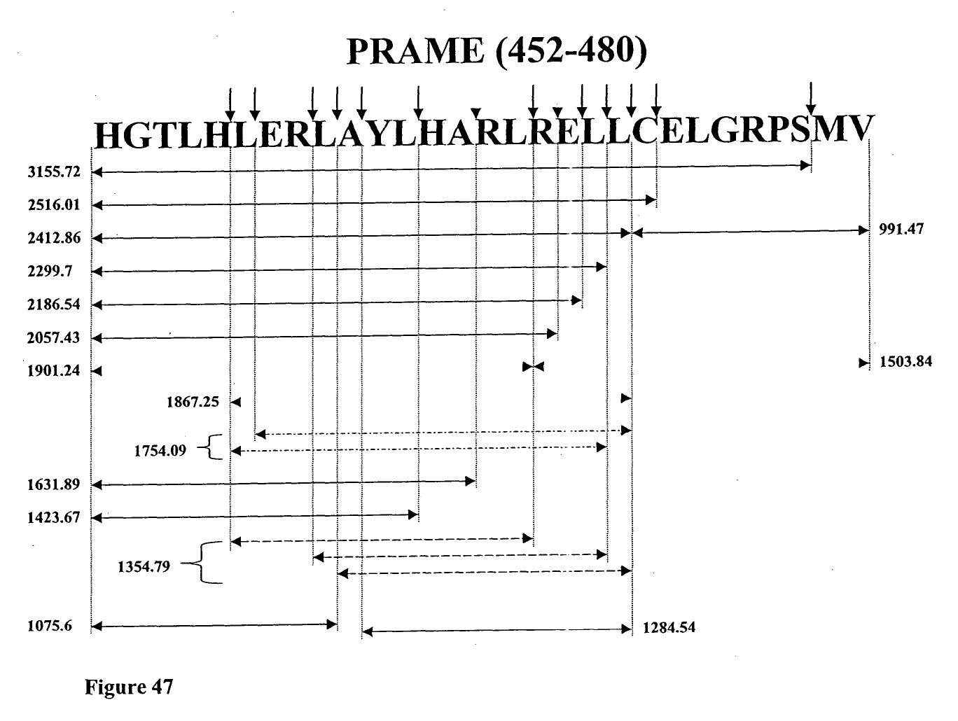

†Scores are given from the two binding prediction programs referenced above (see example 3)
See also FIG. 47.

### Example 45

### PSA 143-169

TABLE 45
Preferred Epitopes Revealed by Housekeeping
Proteasome Digestion

| | | Se- | | HLA binding | |
|---|---|---|---|---|---|
| | | quence | HLA | predictions† | |
| Epitope | Sequence | ID No. | type | SYFPEITHI | NIH |
| 144-153 | QEPALGTTCY | 400 | A1 | 15 | <5 |
| 145-153 | EPALGTTCY | 401 | A1 | 17 | <5 |
| | | | A26 | 17 | N/A |

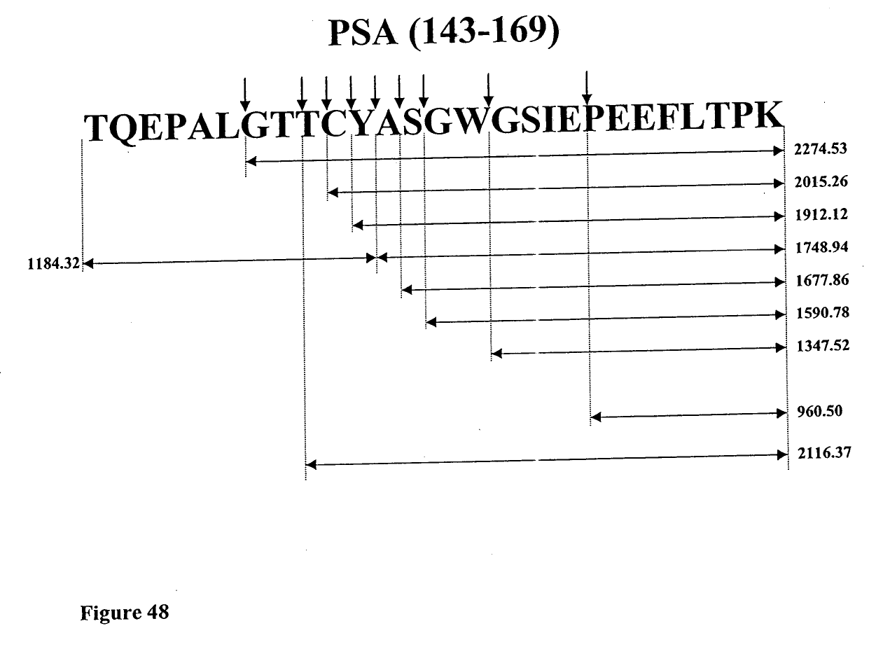

†Scores are given from the two binding prediction programs referenced above (see example 3)
See also FIG. 48.

### Example 46

### PSA 156-1883

TABLE 46
Preferred Epitopes Revealed by Housekeeping
Proteasome Digestion

| | | Se- | | HLA binding | |
|---|---|---|---|---|---|
| | | quence | HLA | predictions† | |
| Epitope | Sequence | ID No. | type | SYFPEITHI | NIH |
| 162-171 | PEEFLTPKKL | 402 | B4402 | 24 | N.A. |
| 163-171 | EEFLTPKKL | 403 | A26 | 17 | N.A. |
| | | | B4402 | 29 | N.A. |
| 165-173 | FLTPKKLQC | 404 | A3 | 20 | <5 |
| | | | B08 | 17 | <5 |
| 165-174 | FLTPKKLQCV | 405 | A0201 | 26 | 735.86 |
| | | | A26 | 15 | N.A. |
| 166-174 | LTPKKLQCV | 406 | A0201 | 21 | <5 |
| | | | A26 | 18 | N.A. |
| 167-174 | TPKKLQCV | 407 | B08 | 16 | <5 |
| | | | B5101 | 22 | N.A. |
| 167-175 | TPKKLQCVD | 408 | B5101 | 15 | <5 |
| 170-179 | KLQCVDLHVI | 409 | A0201 | 24 | 34.433 |
| | | | A3 | 17 | <5 |
| 171-179 | LQCVDLHVI | 410 | A0201 | 15 | <5 |
| | | | B5101 | 16 | 6.292 |

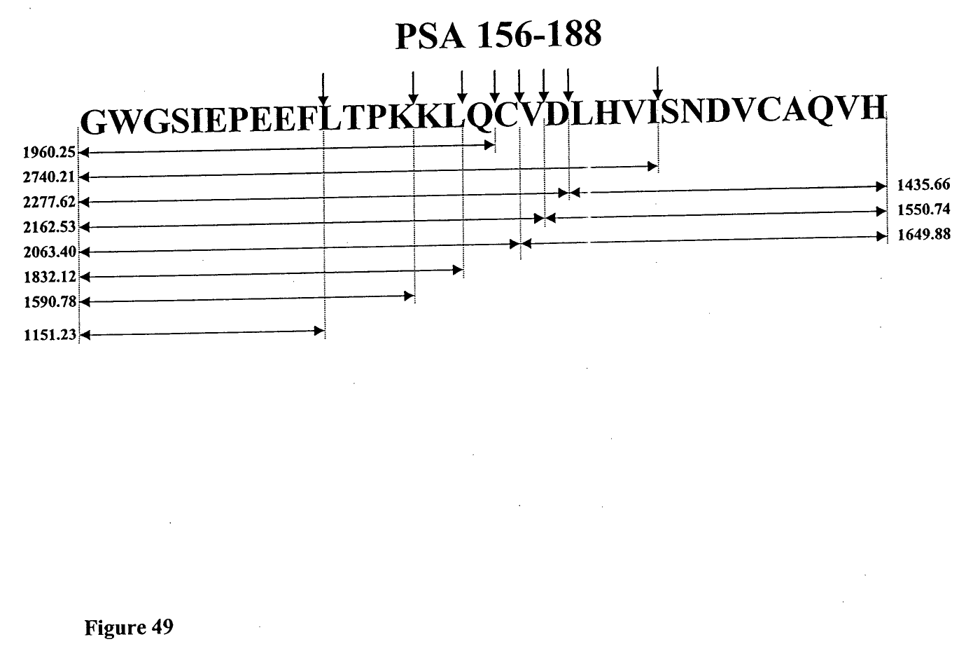

†Scores are given from the two binding prediction programs referenced above (see example 3)
See also FIG. 49.

### Example 47

### PSCA 67-94

TABLE 47
Preferred Epitopes Revealed by Housekeeping
Proteasome Digestion

| | | Se- | | HLA binding | |
|---|---|---|---|---|---|
| Epi- | | quence | HLA | predictions† | |
| tope | Sequence | ID No. | type | SYFPEITHI | NIH |
| 73-81 | DSQDYYVGK | 411 | A3 | 15 | <5 |
| 74-82 | SQDYYVGKK | 412 | A1 | 16 | <5 |
| 74-83 | SQDYYVGKKN | 413 | A1 | 15 | <5 |
| 76-84 | DYYVGKKNI | 414 | B5101 | 19 | 23.426 |
| 77-84 | YYVGKKNI | 415 | B08 | 16 | <5 |
| 78-86 | YVGKKNITC | 416 | A3 | 15 | <5 |
| 78-87 | YVGKKNITCC | 417 | A26 | 15 | N/A |

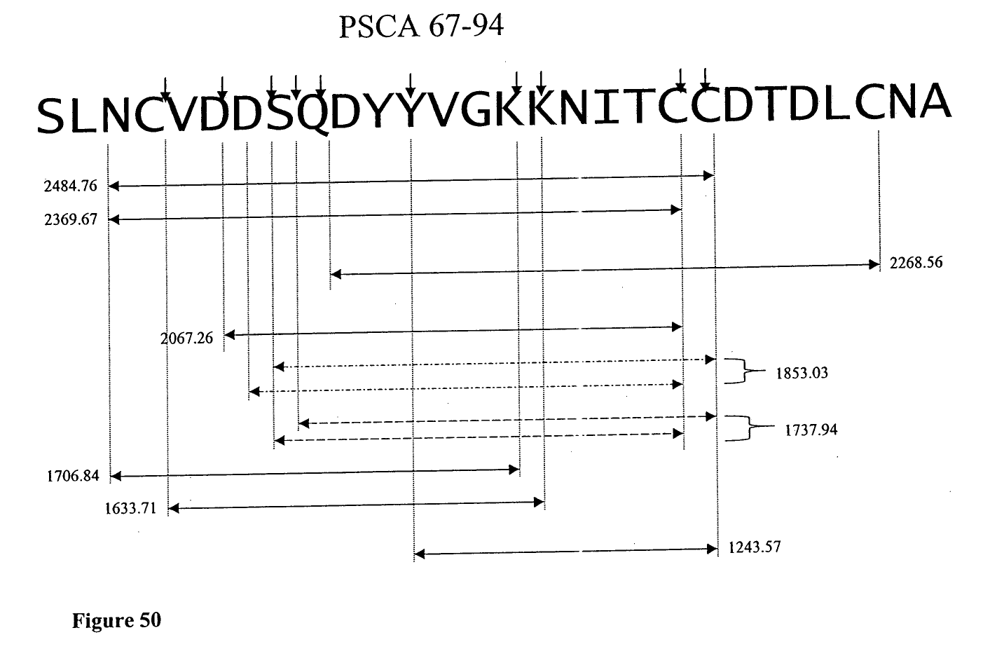

†Scores are given from the two binding prediction programs referenced above (see example 3)
See also FIG. 50.

### Example 48

### PSMA 378-405

TABLE 48
Preferred Epitopes Revealed by Housekeeping
Proteasome Digestion

| | | Se- | | HLA binding | |
|---|---|---|---|---|---|
| | | quence | HLA | predictions† | |
| Epitope | Sequence | ID No. | type | SYFPEITHI | NIH |
| 381-390 | WVFGGIDPQS | 418 | A26 | 16 | N/A |
| | | | A3 | 15 | <5 |
| 385-394 | GIDPQSGAAV | 419 | A0201 | 24 | <5 |
| | | | A0203 | 17 | N/A |
| | | | A1 | 15 | 10 |
| | | | A26 | 15 | N/A |
| | | | A3 | 18 | <5 |
| 386-394 | IDPQSGAAV | 420 | A0201 | 15 | <5 |
| 387-394 | DPQSGAAV | 421 | B5101 | 22 | N/A |
| 387-395 | DPQSGAAVV | 422 | B0702 | 18 | N/A |
| | | | B5101 | 26 | 440 |
| 387-396 | DPQSGAAVVH | 423 | A3 | 15 | <5 |
| 388-396 | PQSGAAVVH | 424 | A3 | 17 | <5 |
| 389-398 | QSGAAVVHEI | 425 | A0201 | 15 | <5 |
| 390-398 | SGAAVVHEI | 426 | A0201 | 19 | <5 |
| | | | B5101 | 21 | 88 |
| 391-398 | GAAVVHEI | 427 | B5101 | 23 | N/A |
| 391-399 | GAAVVHEIV | 428 | A0201 | 17 | <5 |
| | | | B5101 | 20 | 133.1 |
| 392-399 | AAVVHEIV | 429 | B5101 | 19 | N/A |

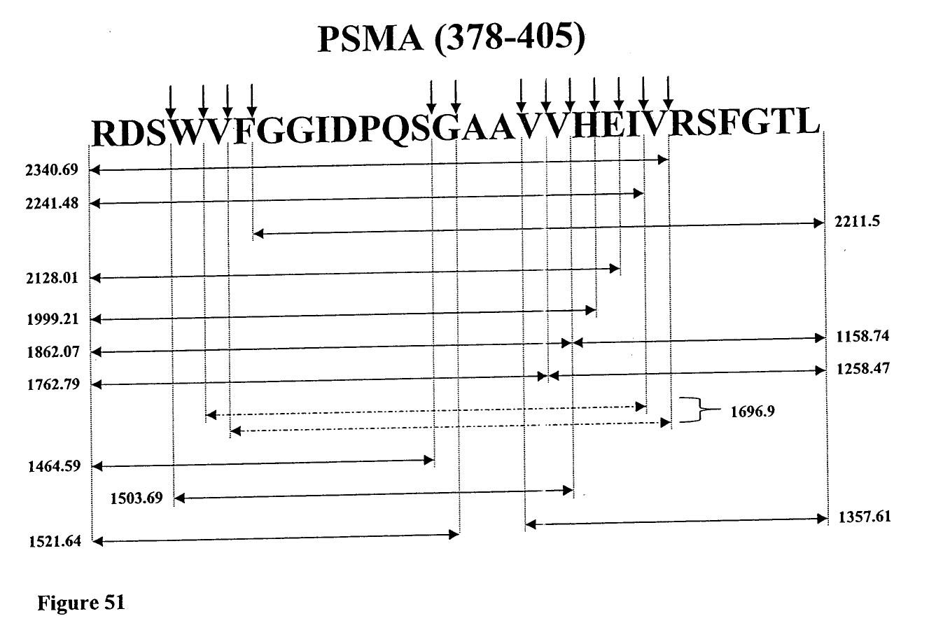

†Scores are given from the two binding prediction programs referenced above (see example 3)
See also FIG. 51.

### Example 49

### PSMA 597-623

TABLE 49
Preferred Epitopes Revealed by Housekeeping
Proteasome Digestion

| | | Se- | | HLA binding | |
|---|---|---|---|---|---|
| | | quence | HLA | predictions† | |
| Epitope | Sequence | ID No. | type | SYFPEITHI | NIH |
| 597-605 | CRDYAVVLR | 430 | B2705 | 22 | N/A |
| 598-607 | RDYAVVLRKY | 431 | A1 | 17 | <5 |
| | | | A26 | 15 | N/A |
| | | | A3 | 16 | <5 |
| 599-607 | DYAVVLRKY | 432 | A1 | 19 | <5 |
| | | | A26 | 22 | N/A |
| 600-607 | YAVVLRKY | 433 | B5101 | 17 | N/A |
| 602-611 | VVLRKYADKI | 434 | A0201 | 17 | <5 |
| | | | A3 | 18 | <5 |
| 603-611 | VLRKYADKI | 435 | A0201 | 22 | <5 |
| | | | A3 | 16 | <5 |
| | | | B08 | 19 | <5 |
| | | | B5101 | 16 | 5.72 |
| 603-612 | VLRKYADKIY | 436 | A1 | 17 | <5 |
| | | | A26 | 19 | N/A |
| | | | A3 | 19 | <5 |
| 604-611 | LRKYADKI | 437 | B08 | 17 | <5 |
| 604-612 | LRKYADKIY | 438 | A1 | 15 | <5 |
| | | | B2705 | 19 | N/A |
| 605-614 | RKYADKIYSI | 439 | A0201 | 16 | <5 |
| 606-614 | KYADKIYSI | 440 | A0201 | 20 | <5 |
| | | | B08 | 17 | <5 |
| 607-614 | YADKIYSI | 441 | B5101 | 27 | N/A |

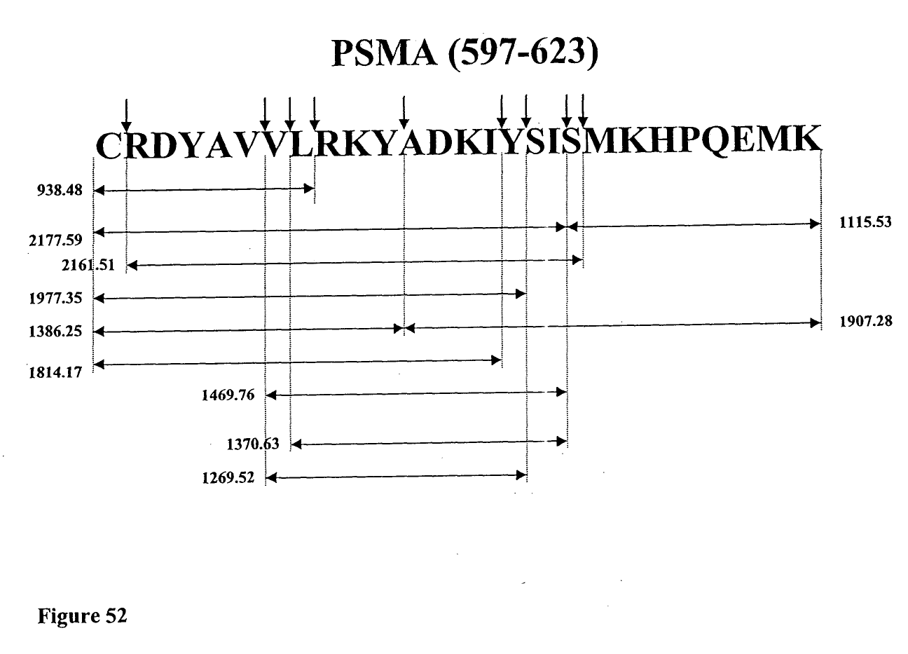

†Scores are given from the two binding prediction programs referenced above (see example 3)
See also FIG. 52.

### Example 50

### PSMA 615-642

TABLE 50
Preferred Epitopes Revealed by Housekeeping
Proteasome Digestion

| | | Se- | | HLA binding | |
|---|---|---|---|---|---|
| | | quence | HLA | predictions† | |
| Epitope | Sequence | ID No. | type | SYFPEITHI | NIH |
| 616-625 | MKHPQEMKTY | 442 | A1 | 19 | <5 |
| | | | A26 | 16 | N/A |
| 617-625 | KHPQEMKTY | 443 | A1 | 15 | <5 |
| | | | A26 | 16 | N/A |
| 618-627 | HPQEMKTYSV | 444 | A0201 | 15 | <5 |
| | | | B0702 | 17 | N/A |

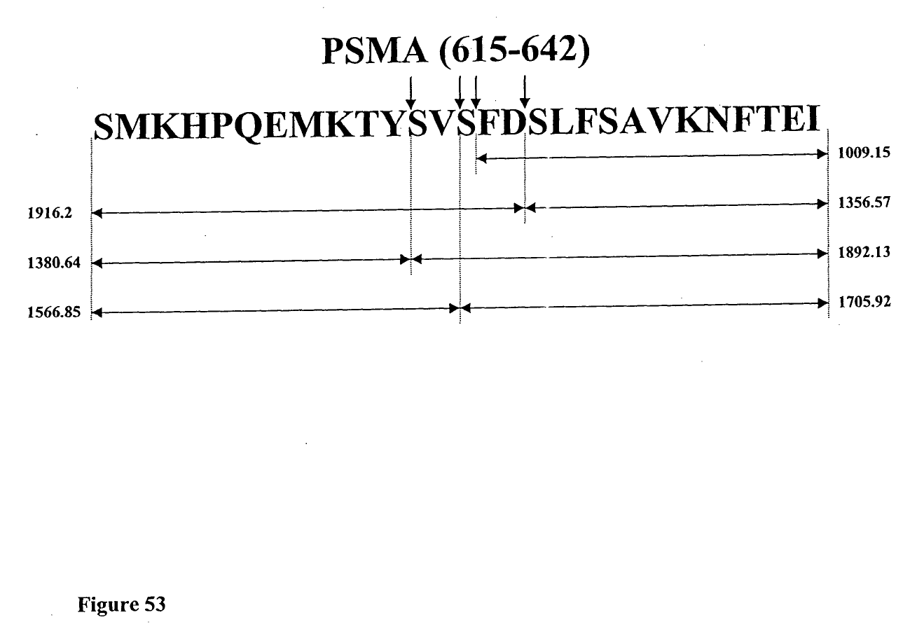

†Scores are given from the two binding prediction programs referenced above (see example 3)
See also FIG. 53.

### Example 51

### SCP-1 57-86

TABLE 51
Preferred Epitopes Revealed by Housekeeping
Proteasome Digestion

| | | Se- | | HLA binding | |
|---|---|---|---|---|---|
| | | quence | HLA | predictions† | |
| Epitope | Sequence | ID No. | type | SYFPEITHI | NIH |
| 62-71 | IDSDPALQKV | 445 | A0201 | 19 | <5 |
| 63-71 | DSDPALQKV | 446 | A0201 | 17 | <5 |
| | | | A1 | 20 | 7.5 |
| | | | A26 | 15 | N/A |
| | | | B5101 | 15 | 5.324 |
| 67-76 | ALQKVNFLPV | 447 | A0201 | 23 | 132.149 |
| | | | A3 | 16 | <5 |
| 70-78 | KVNFLPVLE | 448 | A3 | 18 | <5 |
| 71-80 | VNFLPVLEQV | 449 | A0201 | 16 | <5 |
| 72-80 | NFLPVLEQV | 450 | A0201 | 18 | <5 |
| 75-84 | PVLEQVGNSD | 451 | A3 | 18 | <5 |
| 76-84 | VLEQVGNSD | 452 | A1 | 15 | <5 |
| | | | A3 | 16 | <5 |

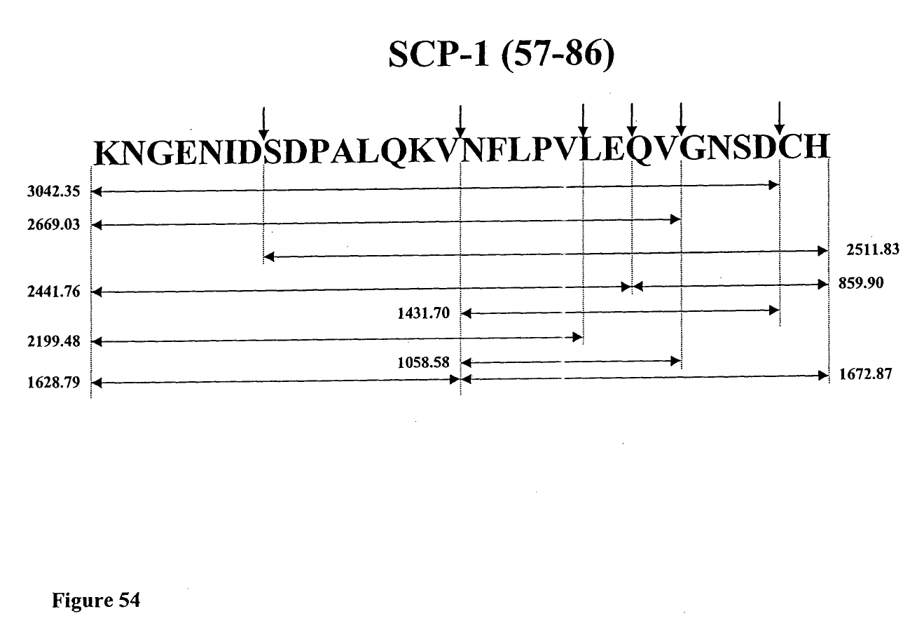

†Scores are given from the two binding prediction programs referenced above (see example 3)
See also FIG. 54.

### Example 52

### SCP-1 201-227

TABLE 52
Preferred Epitopes Revealed by Housekeeping
Proteasome Digestion

| | | Se- | | HLA binding | |
|---|---|---|---|---|---|
| | | quence | HLA | predictions† | |
| Epitope | Sequence | ID No. | type | SYFPEITHI | NIH |
| 202-210 | YEREETRQV | 453 | A0201 | 16 | <5 |
| 202-211 | YEREETRQVY | 454 | A1 | 19 | <5 |
| | | | A3 | 15 | <5 |
| | | | A4402 | 22 | N/A |
| 203-211 | EREETRQVY | 455 | A1 | 27 | <5 |
| | | | A26 | 19 | N/A |
| | | | B2705 | 20 | N/A |
| 203-212 | EREETRQVYM | 456 | A26 | 17 | N/A |
| 204-212 | REETRQVYM | 457 | B2705 | 15 | N/A |
| 211-220 | YMDLNSNIEK | 458 | A1 | 17 | 25 |
| 213-221 | DLNSNIEKM | 459 | A0201 | 20 | <5 |
| | | | A26 | 28 | N/A |
| 216-226 | SNIEKMITAF | 460 | A26 | 19 | N/A |
| | | | B4402 | 19 | N/A |
| 217-225 | NIEKMITAF | 461 | A26 | 26 | N/A |
| | | | B2705 | 17 | N/A |
| | | | B4402 | 16 | N/A |
| 218-225 | IEKMITAF | 462 | B08 | 17 | <5 |

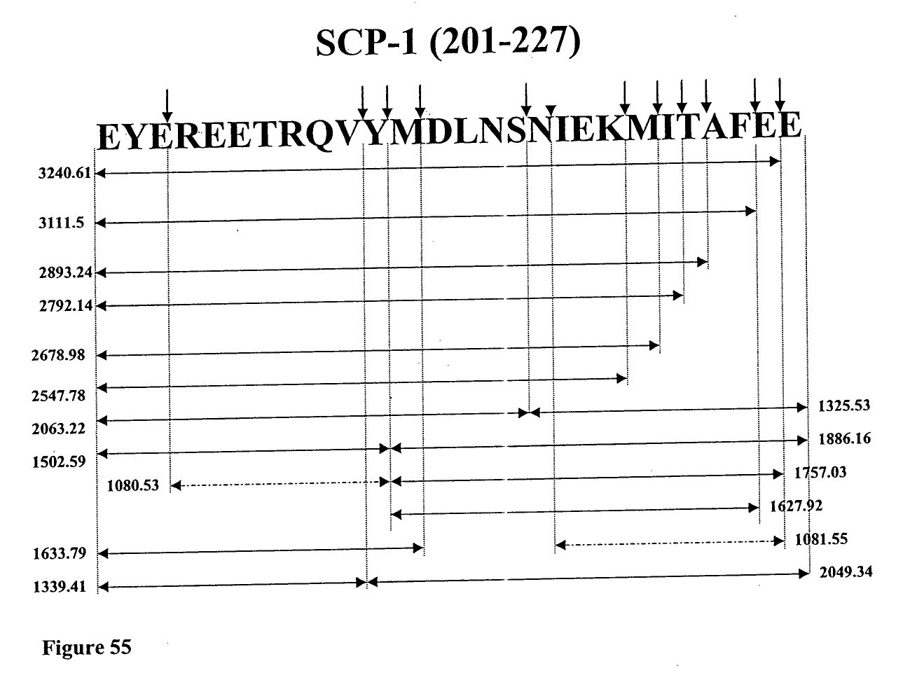

†Scores are given from the two binding prediction programs referenced above (see example 3)
See also FIG. 55.

### Example 53

### SCP-1 395-424

TABLE 53
Preferred Epitopes Revealed by Housekeeping
Proteasome Digestion

| | | Se- | | HLA binding | |
|---|---|---|---|---|---|
| | | quence | HLA | predictions† | |
| Epitope | Sequence | ID No. | type | SYFPEITHI | NIH |
| 397-406 | RLENYEDQLI | 463 | A0201 | 17 | <5 |
| | | | A3 | 15 | <5 |
| 398-406 | LENYEDQLI | 464 | B4402 | 19 | N/A |
| 398-407 | LENYEDQLII | 465 | B4402 | 19 | N/A |
| 399-407 | ENYEDQLII | 466 | B5101 | 17 | 19.36 |
| 399-408 | ENYEDQLIIL | 467 | A26 | 20 | N/A |
| 400-408 | NYEDQLIIL | 468 | A1 | 16 | <5 |
| 400-409 | NYEDQLIILT | 469 | A1 | 16 | <5 |
| 401-409 | YEDQLIILT | 470 | A1 | 18 | <5 |
| | | | B4402 | 16 | N/A |
| 401-410 | YEDQLIILTM | 471 | A1 | 18 | <5 |
| | | | B4402 | 16 | N/A |
| 402-410 | EDQLIILTM | 472 | A26 | 18 | N/A |
| | | | B2705 | 15 | <5 |
| 406-415 | IILTMELQKT | 473 | A0201 | 22 | 14.824 |
| | | | A26 | 16 | N/A |
| 407-415 | ILTMELQKT | 474 | A0201 | 21 | 29.137 |

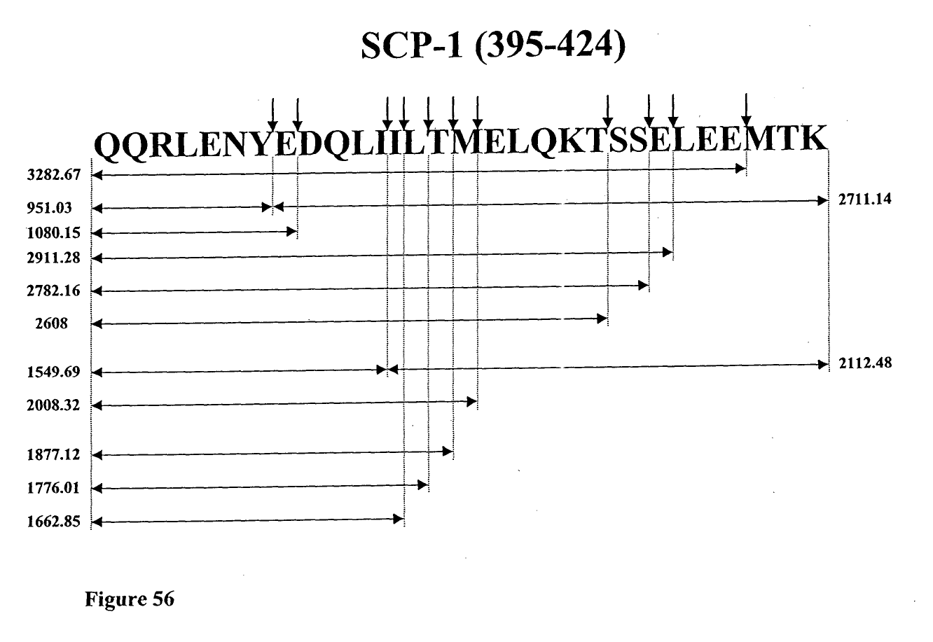

†Scores are given from the two binding prediction programs referenced above (see example 3).
See also FIG. 56.

### Example 54

### SCP-1 416-442

TABLE 54
Preferred Epitopes Revealed by Housekeeping
Proteasome Digestion

| | | Se- | | HLA binding | |
|---|---|---|---|---|---|
| | | quence | HLA | predictions† | |
| Epitope | Sequence | ID No. | type | SYFPEITHI | NIH |
| 424-432 | KLTNNKEVE | 475 | A3 | 18 | <5 |
| 424-433 | KLTNNKEVEL | 476 | A0201 | 24 | 74.768 |
| | | | A26 | 18 | N/A |
| | | | A3 | 18 | <5 |
| 425-433 | LTNNKEVEL | 477 | A0201 | 22 | <5 |
| | | | A26 | 21 | N/A |
| | | | B08 | 22 | <5 |
| 429-438 | KEVELEELKK | 478 | A3 | 17 | <5 |
| 430-438 | EVELEELKK | 479 | A1 | 18 | 90 |
| | | | A26 | 17 | N/A |
| | | | A3 | 24 | <5 |
| | | | B2705 | 15 | <5 |
| 430-439 | EVELEELKKV | 480 | A0201 | 15 | <5 |
| | | | A26 | 21 | N/A |
| 431-439 | VELEELKKV | 481 | A0201 | 20 | 80.217 |
| | | | A4402 | 15 | N/A |
| | | | B5101 | 17 | <5 |

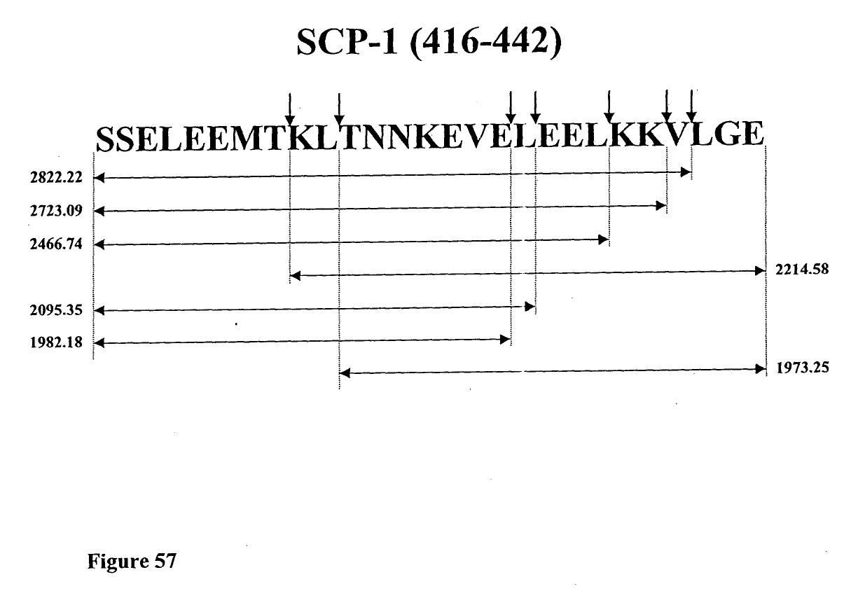

†Scores are given from the two binding prediction programs referenced above (see example 3)
See also FIG. 57.

### Example 55

### SCP-1 518-545

TABLE 55
Preferred Epitopes Revealed by Housekeeping
Proteasome Digestion

| | | Se- | | HLA binding | |
|---|---|---|---|---|---|
| | | quence | HLA | predictions† | |
| Epitope | Sequence | ID No. | type | SYFPEITHI | NIH |
| 530-539 | ETSDMTLELK | 482 | A26 | 21 | N/A |
| 531-539 | TSDMTLELK | 483 | A1 | 16 | 15 |

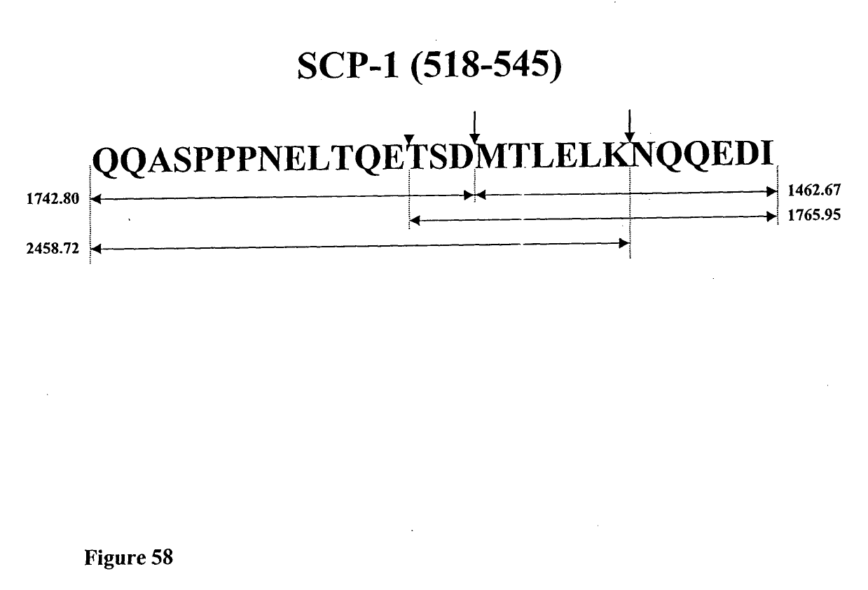

†Scores are given from the two binding prediction programs referenced above (see example 3)
See also FIG. 58.

### Example 56

### SCP-1 545-578

TABLE 56
Preferred Epitopes Revealed by Housekeeping
Proteasome Digestion

| | | Se- | | HLA binding | |
|---|---|---|---|---|---|
| | | quence | HLA | predictions† | |
| Epitope | Sequence | ID No. | type | SYFPEITHI | NIH |
| 548-556 | NKKQEERML | 484 | B08 | 20 | <5 |
| 553-562 | ERMLTQIENL | 485 | A26 | 19 | N/A |
| | | | B4402 | 17 | N/A |
| 554-562 | RMLTQIENL | 486 | A0201 | 24 | 64.335 |
| | | | B2705 | 21 | 150 |
| | | | B2709 | 17 | N/A |
| | | | B4402 | 15 | N/A |
| 555-562 | MLTQIENL | 487 | B08 | 16 | <5 |
| 555-564 | MLTQIENLQE | 488 | A3 | 16 | <5 |
| 560-569 | ENLQETETQL | 489 | A26 | 16 | N/A |
| 561-569 | NLQETETQL | 490 | A0201 | 22 | 87.586 |
| | | | A26 | 19 | N/A |
| | | | A3 | 15 | <5 |
| | | | B08 | 18 | <5 |
| 561-570 | NLQETETQLR | 491 | A3 | 15 | 6 |

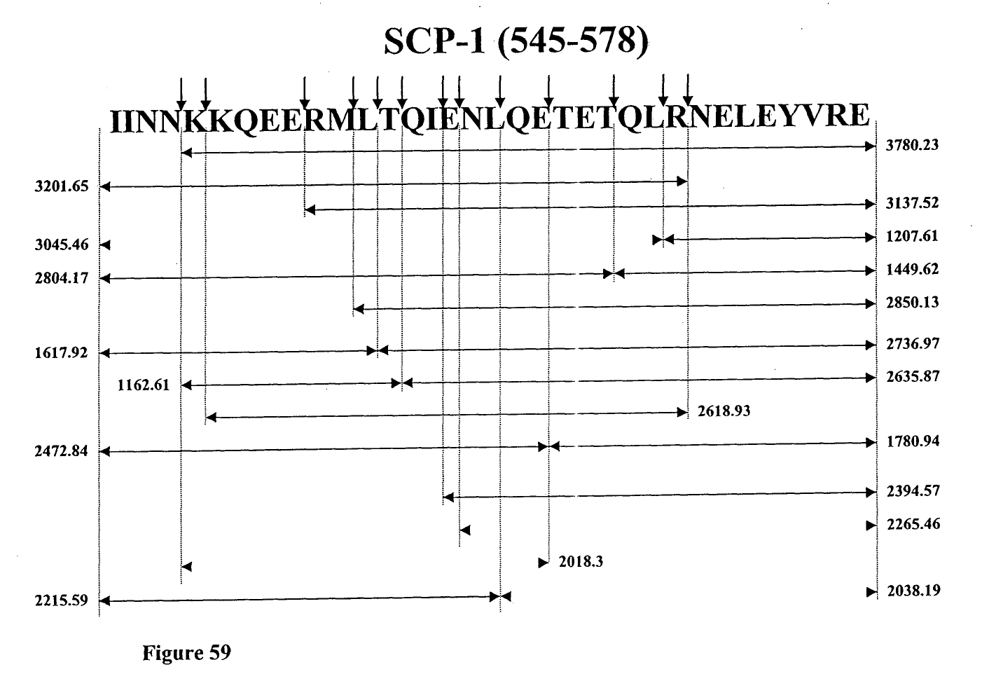

†Scores are given from the two binding prediction programs referenced above (see example 3).
See also FIG. 59.

### Example 57

### SCP-1 559-585

TABLE 57
Preferred Epitopes Revealed by Housekeeping
Proteasome Digestion

| | | Se- | | HLA binding | |
|---|---|---|---|---|---|
| | | quence | HLA | predictions† | |
| Epitope | Sequence | ID No. | type | SYFPEITHI | NIH |
| 567-576 | TQLRNELEYV | 492 | A0201 | 16 | 161.729 |
| 568-576 | QLRNELEYV | 493 | A0201 | 24 | 32.765 |
| | | | A3 | 16 | <5 |
| 571-580 | NELEYVREEL | 494 | A0201 | 16 | <5 |
| | | | B4402 | 23 | N/A |
| 572-580 | ELEYVREEL | 495 | A0201 | 17 | <5 |
| | | | A26 | 23 | N/A |
| | | | B08 | 20 | <5 |
| 573-580 | LEYVREEL | 496 | B08 | 19 | <5 |
| 574-583 | EYVREELKQK | 497 | A3 | 16 | <5 |
| 575-583 | YVREELKQK | 498 | A26 | 17 | N/A |
| | | | A3 | 27 | <5 |

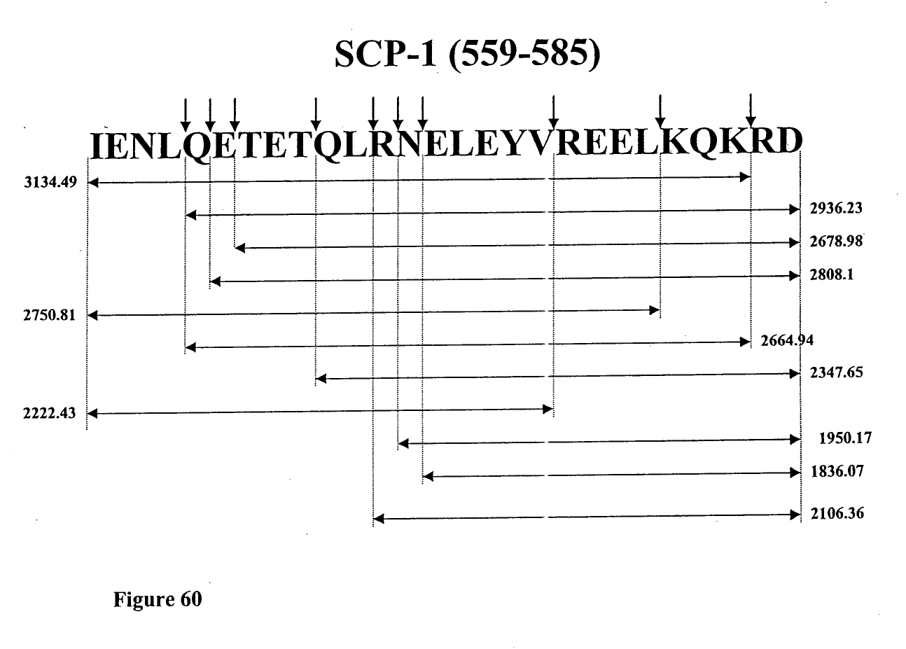

†Scores are given from the two binding prediction programs referenced above (see example 3)
See also FIG. 60.

### Example 58

### SCP-1 665-701

TABLE 58
Preferred Epitopes Revealed by Housekeeping
Proteasome Digestion

| | | Se- | | HLA binding | |
|---|---|---|---|---|---|
| | | quence | HLA | predictions† | |
| Epitope | Sequence | ID No. | type | SYFPEITHI | NIH |
| 675-684 | LLEEVEKAKV | 499 | A0201 | 27 | 31.026 |
| 676-684 | LEEVEKAKV | 500 | A0201 | 15 | <5 |
| 676-685 | LEEVEKAKVI | 501 | A4402 | 22 | N/A |
| 677-685 | EEVEKAKVI | 502 | B08 | 21 | <5 |
| | | | B4402 | 24 | N/A |
| | | | B5101 | 18 | <5 |
| 681-690 | KAKVIADEAV | 503 | A0201 | 15 | <5 |
| 683-692 | KVIADEAVKL | 504 | A0201 | 21 | 6.542 |
| | | | A26 | 22 | N/A |
| | | | A3 | 25 | <5 |
| | | | B4402 | 17 | N/A |
| 684-692 | VIADEAVKL | 505 | A0201 | 26 | 20.473 |
| | | | A26 | 22 | N/A |
| | | | A3 | 17 | <5 |
| | | | B08 | 16 | <5 |
| | | | B2705 | 15 | N/A |
| 685-692 | IADEAVKL | 506 | B08 | 17 | <5 |
| | | | B5101 | 21 | N/A |

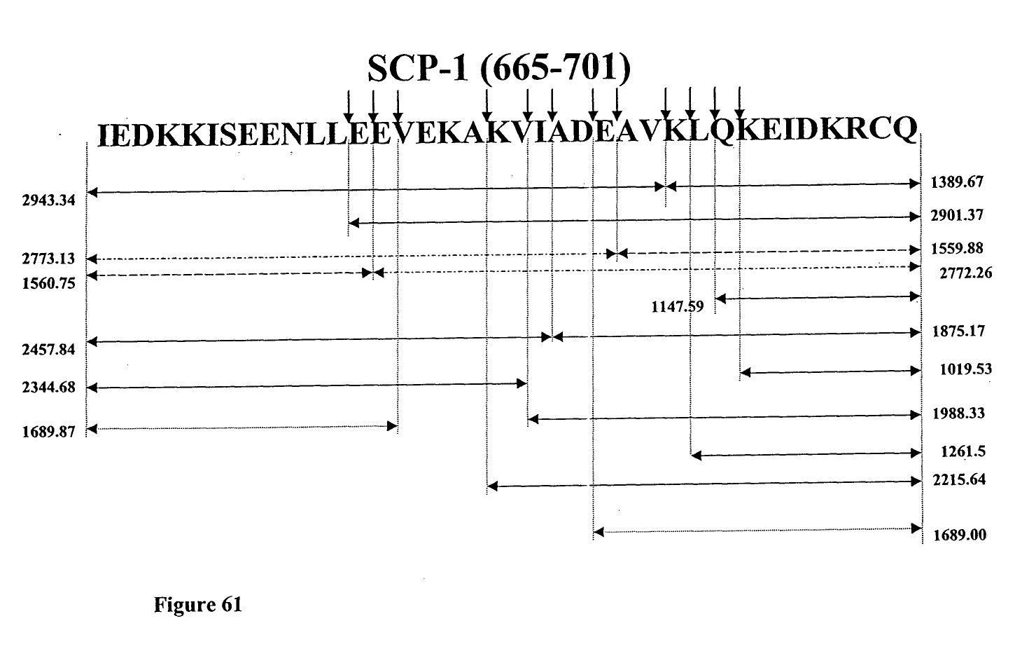

†Scores are given from the two binding prediction programs referenced above (see example 3)
See also FIG. 61.

### Example 59

### SCP-1 694-720

TABLE 59
Preferred Epitopes Revealed by Housekeeping
Proteasome Digestion

| | | Se- | | HLA binding | |
|---|---|---|---|---|---|
| | | quence | HLA | predictions† | |
| Epitope | Sequence | ID No. | type | SYFPEITHI | NIH |
| 694-702 | KEIDKRCQH | 507 | A3 | 16 | <5 |
| | | | A4402 | 17 | N/A |
| 694-703 | KEIDKRCQHK | 508 | A3 | 17 | <5 |
| | | | B4402 | 15 | N/A |
| 695-703 | EIDKRCQHK | 509 | A26 | 20 | N/A |
| | | | A3 | 20 | <5 |
| 695-704 | EIDKRCQHKI | 510 | A0201 | 16 | <5 |
| | | | A26 | 19 | N/A |
| 696-704 | IDKRCQHKI | 511 | B08 | 17 | <5 |
| 697-704 | DKRCQHKI | 512 | B5101 | 16 | N/A |
| 698-706 | KRCQHKIAE | 513 | B2705 | 16 | 60 |
| 698-707 | KRCQHKIAEM | 514 | A26 | 15 | N/A |
| 699-707 | RCQHKIAEM | 515 | A26 | 15 | N/A |
| | | | B2705 | 18 | 9 |
| 701-710 | QHKIAEMVAL | 516 | A26 | 15 | N/A |
| 702-710 | HKIAEMVAL | 517 | A0201 | 15 | <5 |
| | | | A26 | 16 | N/A |
| | | | B4402 | 16 | N/A |
| 703-710 | KIAEMVAL | 518 | B08 | 16 | <5 |

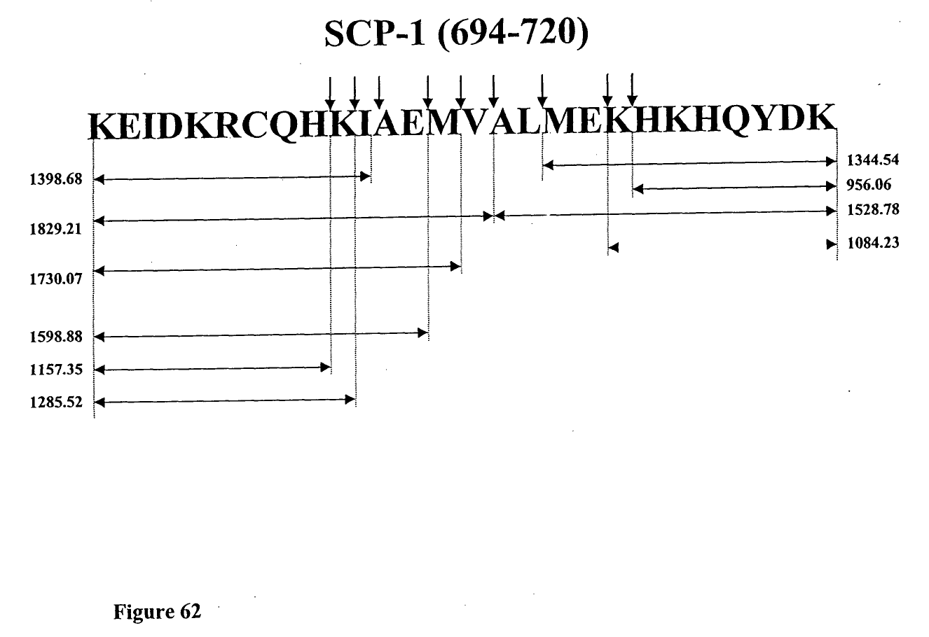

†Scores are given from the two binding prediction programs referenced above (see example 3)
See also FIG. 62.

### Example 60

### SCP-1 735-769

TABLE 60
Preferred Epitopes Revealed by Housekeeping Proteasome Digestion

| | | Se- | | HLA binding | |
|---|---|---|---|---|---|
| | | quence | HLA | predictions† | |
| Epitope | Sequence | ID No. | type | SYFPEITHI | NIH |
| 737-746 | QEQSSLRASL | 519 | B4402 | 21 | N.A. |
| 738-746 | EQSSLRASL | 520 | A26 | 22 | N.A. |
| | | | B0702 | 15 | 6 |
| 739-746 | QSSLRASL | 521 | B08 | 19 | <5 |
| 741-750 | SLRASLEIEL | 522 | A0201 | 24 | <5 |
| | | | A26 | 17 | N.A. |
| | | | A3 | 16 | <5 |
| 742-750 | LRASLEIEL | 523 | A0201 | 17 | <5 |
| | | | B2705 | 23 | 2000 |
| | | | B2709 | 21 | N.A. |
| 743-750 | RASLEIEL | 524 | B5101 | 17 | N.A. |
| 744-753 | ASLEIELSNL | 525 | A0201 | 20 | <5 |
| | | | A26 | 16 | N.A. |
| 745-753 | SLEIELSNL | 526 | A0201 | 25 | <5 |
| | | | A26 | 22 | N.A. |
| | | | A3 | 15 | <5 |
| | | | B08 | 18 | <5 |
| 745-754 | SLEIELSNLK | 527 | A1 | 15 | 18 |
| | | | A3 | 22 | 20 |
| 746-754 | LEIELSNLK | 528 | B2705 | 16 | 30 |
| | | | B4402 | 15 | N.A. |
| 747-755 | EIELSNLKA | 529 | A1 | 19 | <5 |
| | | | A26 | 18 | N.A. |
| 749-758 | ELSNLKAELL | 530 | A0201 | 17 | <5 |
| | | | A26 | 22 | N.A. |
| 750-758 | LSNLKAELL | 531 | B08 | 21 | <5 |
| 751-760 | SNLKAELLSV | 532 | A0201 | 21 | <5 |
| 752-760 | NLKAELLSV | 533 | A0201 | 26 | 5.599 |
| | | | A3 | 18 | <5 |
| | | | B08 | 16 | <5 |
| 752-761 | NLKAELLSVK | 534 | A3 | 30 | 30 |
| 753-761 | LKAELLSVK | 535 | A3 | 19 | <5 |
| 753-762 | LKAELLSVKK | 536 | A3 | 16 | <5 |
| 754-762 | KAELLSVKK | 537 | A3 | 18 | <5 |
| | | | B2705 | 18 | 30 |
| 755-763 | AELLSVKKQ | 538 | B4402 | 19 | N.A. |

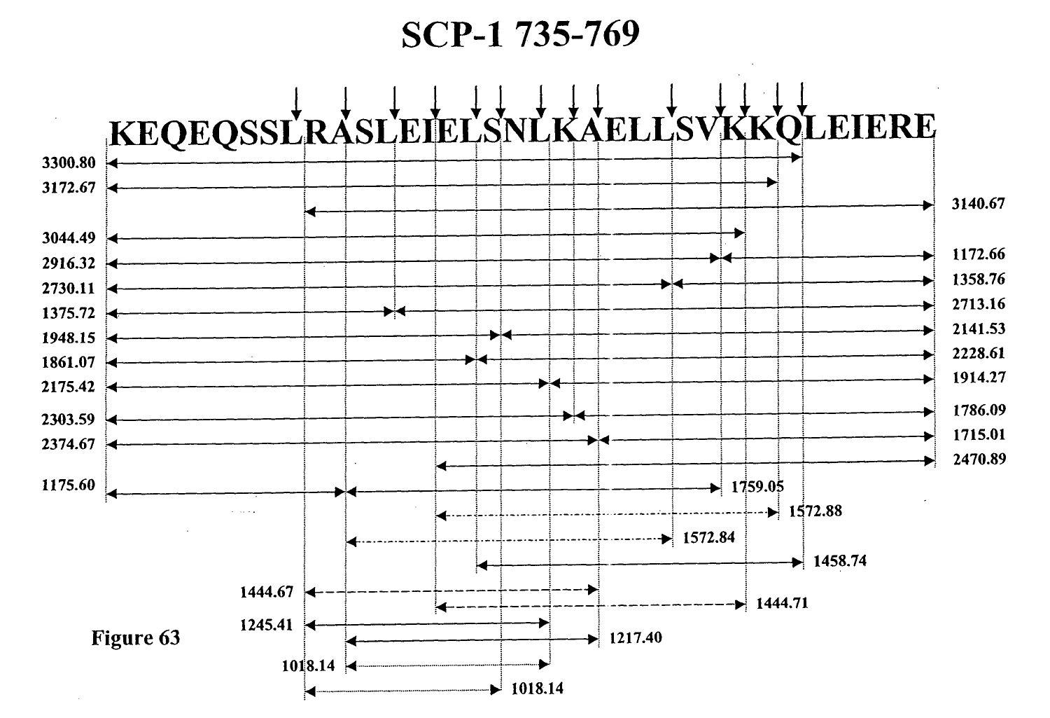

†Scores are given from the two binding prediction programs referenced above (see example 3)
See also FIG. 63.

### Example 61

### SCP-1 786-816

TABLE 61
Preferred Epitopes Revealed by Housekeeping
Proteasome Digestion

| | | Se- | | HLA binding | |
|---|---|---|---|---|---|
| | | quence | HLA | predictions† | |
| Epitope | Sequence | ID No. | type | SYFPEITHI | NIH |
| 787-796 | EKKDKKTQTF | 539 | A26 | 19 | N/A |
| | | | B4402 | 15 | N/A |
| 788-796 | KKDKKTQTF | 540 | B08 | 16 | <5 |
| | | | B2705 | 16 | <5 |
| 789-796 | KDKKTQTF | 541 | B08 | 16 | <5 |
| 797-806 | LLETPDIYWK | 542 | A0201 | 16 | <5 |
| | | | A3 | 21 | 90 |
| 798-806 | LETPDIYWK | 543 | B2705 | 15 | 30 |
| | | | B4402 | 16 | N/A |
| 798-807 | LETPDIYWKL | 544 | A0201 | 15 | 7.944 |
| | | | A26 | 15 | N/A |
| | | | A4402 | 24 | N/A |
| 799-807 | ETPDIYWKL | 545 | A26 | 31 | N/A |
| | | | B4402 | 16 | N/A |
| 800-807 | TPDIYWKL | 546 | B08 | 16 | <5 |
| | | | B5101 | 19 | N/A |

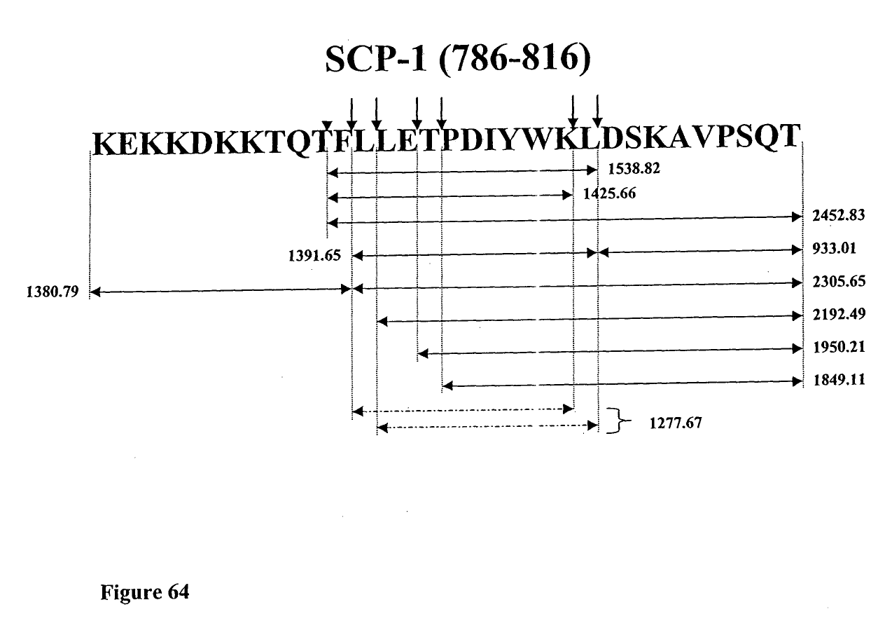

†Scores are given from the two binding prediction programs referenced above (see example 3)
See also FIG. 64.

### Example 62

### SCP-1 806-833

TABLE 62
Preferred Epitopes Revealed by Housekeeping
Proteasome Digestion

| | | Se- | | HLA binding | |
|---|---|---|---|---|---|
| | | quence | HLA | predictions† | |
| Epitope | Sequence | ID No. | type | SYFPEITHI | NIH |
| 809-817 | SKAVPSQTV | 547 | A0201 | 17 | <5 |
| 810-817 | KAVPSQTV | 548 | B5101 | 19 | N/A |
| 812-821 | VPSQTVSRNF | 549 | B0702 | 18 | N/A |
| 815-824 | QTVSRNFTSV | 550 | A0201 | 16 | <5 |
| | | | A26 | 16 | N/A |
| 816-824 | TVSRNFTSV | 551 | A0201 | 16 | 11.426 |
| | | | A26 | 15 | N/A |
| | | | A3 | 16 | <5 |
| 816-825 | TVSRNFTSVD | 552 | A3 | 20 | <5 |
| 823-832 | SVDHGISKDK | 553 | A3 | 21 | <5 |

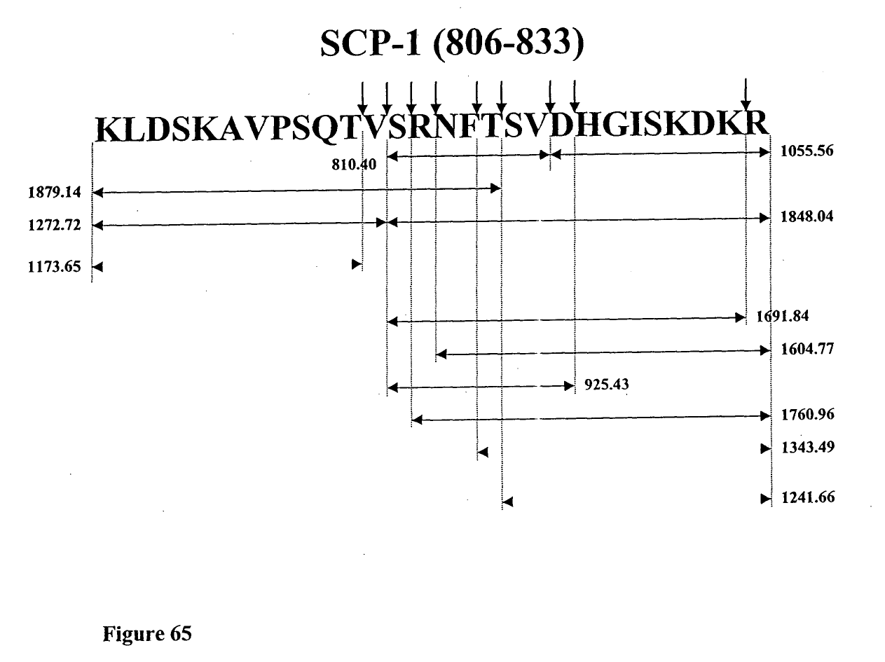

†Scores are given from the two binding prediction programs referenced above (see example 3)
See also FIG. 65.

### Example 63

### SCP-1 826-853

TABLE 63
Preferred Epitopes Revealed by Housekeeping
Proteasome Digestion

| | | Se- | | HLA binding | |
|---|---|---|---|---|---|
| | | quence | HLA | predictions† | |
| Epitope | Sequence | ID No. | type | SYFPEITHI | NIH |
| 829-838 | SKDKRDYLWT | 554 | A1 | 18 | <5 |
| 832-840 | KRDYLWTSA | 555 | B2705 | 16 | 600 |
| 832-841 | KRDYLWTSAK | 556 | A3 | 17 | <5 |
| 833-841 | RDYLWTSAK | 557 | A3 | 23 | <5 |
| | | | B2705 | 18 | 15 |
| 835-843 | YLWTSAKNT | 558 | A0201 | 16 | 284.517 |
| 835-844 | YLWTSAKNTL | 559 | A0201 | 26 | 815.616 |
| | | | A26 | 16 | N/A |
| 837-844 | WTSAKNTL | 560 | B08 | 20 | <5 |
| 841-850 | KNTLSTPLPK | 561 | A3 | 18 | <5 |
| 842-850 | NTLSTPLPK | 562 | A3 | 16 | <5 |

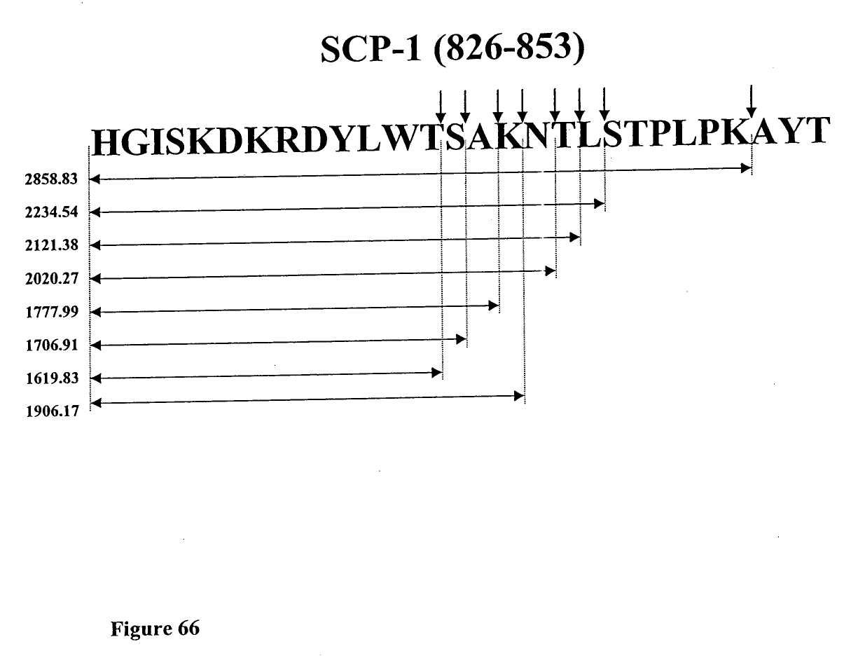

†Scores are given from the two binding prediction programs referenced above (see example 3)
See also FIG. 66.

### Example 64

### SCP-1 832-859

TABLE 64
Preferred Epitopes Revealed by Housekeeping
Proteasome Digestion

| | | Se- | | HLA binding | |
|---|---|---|---|---|---|
| | | quence | HLA | predictions† | |
| Epitope | Sequence | ID No. | type | SYFPEITHI | NIH |
| 832-840 | KRDYLWTSA | 563 | B2705 | 16 | 600 |
| 832-841 | KKDYLWTSAK | 564 | A3 | 17 | <5 |
| 833-841 | RDYLWTSAK | 565 | A3 | 23 | <5 |
| | | | B2705 | 18 | 15 |
| 835-843 | YLWTSAKNT | 566 | A0201 | 16 | 284.517 |
| 839-846 | SAKNTLST | 567 | B08 | 16 | <5 |
| 841-850 | KNTLSTPLPK | 568 | A3 | 18 | <5 |
| 842-850 | NTLSTPLPK | 569 | A3 | 16 | <5 |
| 843-852 | TLSTPLPKAY | 570 | A1 | 16 | <5 |
| | | | A26 | 19 | N/A |
| | | | A3 | 18 | <5 |
| | | | B4402 | 17 | N/A |
| 844-852 | LSTPLPKAY | 571 | A1 | 23 | 7.5 |
| | | | A4402 | 18 | N/A |

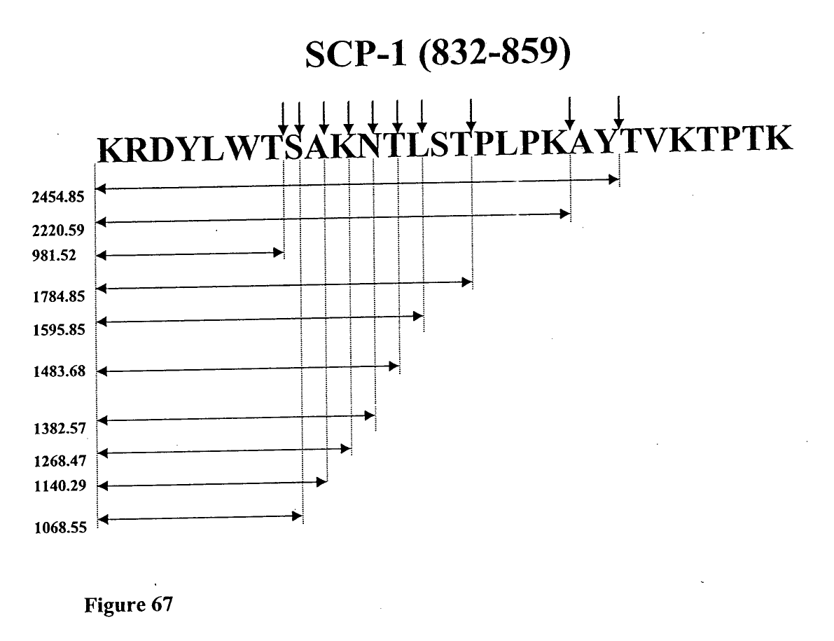

†Scores are given from the two binding prediction programs referenced above (see example 3)
See also FIG. 67.

### Example 65

### SSX-2 1-27

TABLE 65
Preferred Epitopes Revealed by Housekeeping
Proteasome Digestion

| | | Se- | | HLA binding | |
|---|---|---|---|---|---|
| | | quence | HLA | predictions† | |
| Epitope | Sequence | ID No. | type | SYFPEITHI | NIH |
| 5-12 | DAFARRPT | 572 | B5101 | 18 | N/A |
| 7-15 | FARRPTVGA | 573 | A0201 | 15 | <5 |
| 8-17 | ARRPTVGAQI | 574 | A3 | 18 | <5 |
| 9-17 | RRPTVGAQI | 575 | B2705 | 23 | 1800 |
| | | | B2709 | 23 | N/A |
| 10-17 | RPTVGAQI | 576 | B5101 | 20 | N/A |
| 13-21 | VGAQIPEKI | 577 | B5101 | 20 | 125.84 |
| 14-21 | GAQIPEKI | 578 | B5101 | 25 | N/A |
| 15-24 | AQIPEKIQKA | 579 | A0201 | 16 | <5 |
| 16-24 | QIPEKIQKA | 580 | A0201 | 21 | 6.442 |
| | | | A26 | 20 | N/A |
| | | | B08 | 17 | <5 |
| 16-25 | QIPEKIQKAF | 581 | A26 | 24 | N/A |
| | | | A3 | 16 | <5 |
| 17-24 | IPEKIQKA | 582 | B5101 | 19 | N/A |
| 17-25 | IPEKIQKAF | 583 | B0702 | 19 | N/A |
| | | | B08 | 15 | <5 |
| | | | B2705 | 16 | <5 |
| 18-25 | PEKIQKAF | 584 | B08 | 16 | <5 |

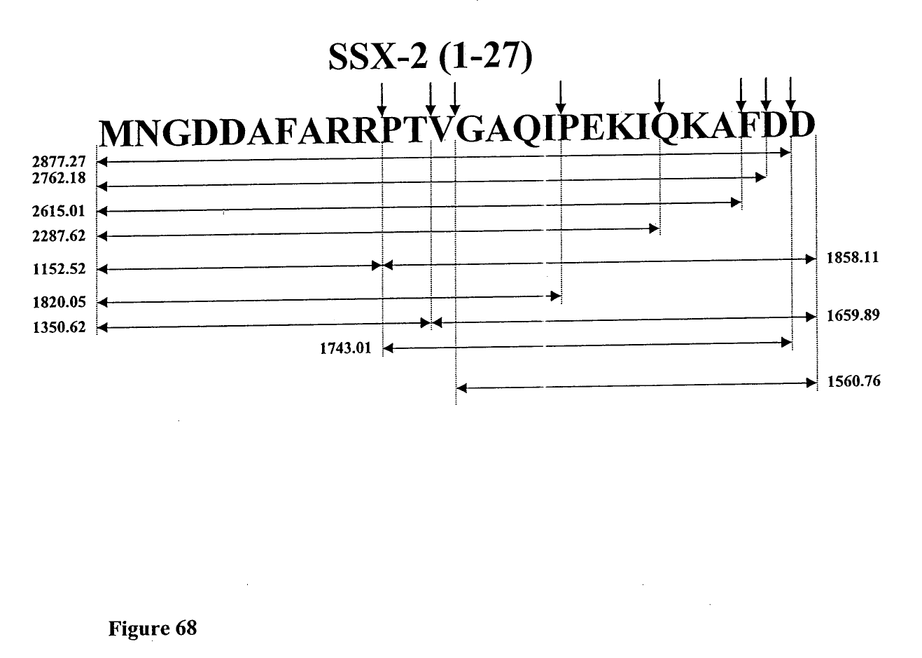

†Scores are given from the two binding prediction programs referenced above (see example 3)
See also FIG. 68.

### Example 66

### Survivin 116-142

TABLE 66
Preferred Epitopes Revealed by Housekeeping
Proteasome Digestion

| | | Se- | | HLA binding | |
|---|---|---|---|---|---|
| | | quence | HLA | predictions† | |
| Epitope | Sequence | ID No. | type | SYFPEITHI | NIH |
| 116-124 | ETNNKKKEF | 585 | A26 | 28 | N/A |
| | | | B08 | 20 | <5 |
| 117-124 | TNNKKKEF | 586 | B08 | 16 | <5 |
| 122-131 | KEFEETAKKV | 587 | A0201 | 15 | 71.806 |
| 123-131 | EFEETAKKV | 588 | A26 | 15 | N/A |
| | | | B5101 | 15 | 5.324 |
| 127-134 | TAKKVRRA | 589 | B5101 | 17 | N/A |
| 126-134 | ETAKKVRRA | 590 | A26 | 24 | N/A |
| 128-136 | AKKVRRAIE | 591 | B08 | 19 | <5 |
| 129-138 | KKVRRAIEQL | 592 | A0201 | 15 | <5 |
| 130-138 | KVRRAIEQL | 593 | A0201 | 19 | <5 |
| | | | A26 | 23 | N/A |
| | | | A3 | 22 | <5 |
| | | | B08 | 17 | <5 |
| | | | B2705 | 16 | 30 |
| 130-139 | KVRRAIEQLA | 594 | A3 | 19 | <5 |
| 131-138 | VRRAIEQL | 595 | B08 | 17 | <5 |

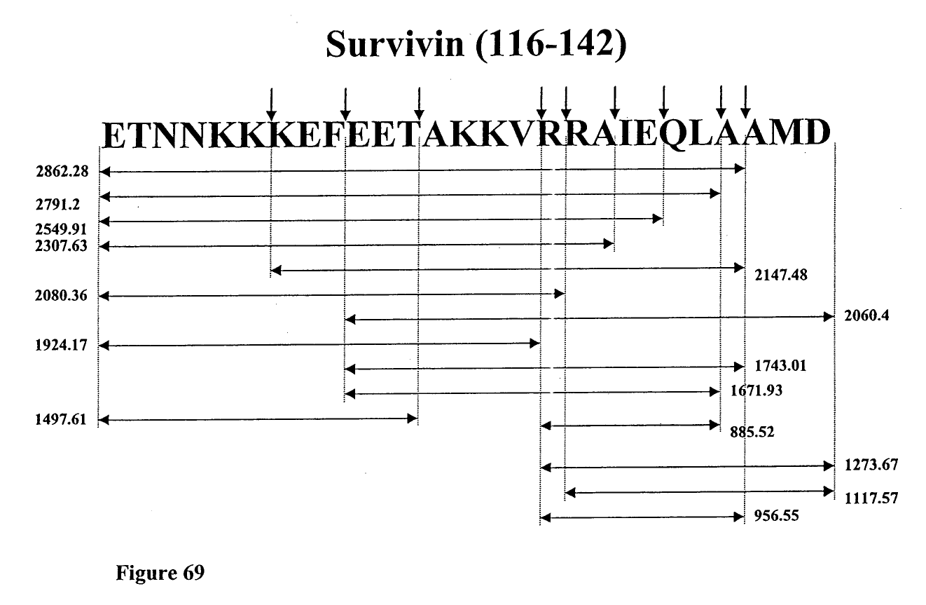

†Scores are given from the two binding prediction programs referenced above (see example 3)
See also FIG. 69.

### Example 67

### BAGE 1-35

TABLE 67
Preferred Epitopes Revealed by Housekeeping
Proteasome Digestion

| | | Se- | | HLA binding | |
|---|---|---|---|---|---|
| | | quence | HLA | predictions† | |
| Epitope | Sequence | ID No. | type | SYFPEITHI | NIH |
| 24-31 | SPVVSWRL | 596 | B08 | 19 | <5 |
| | | | B5101 | 17 | N/A |
| 21-29 | KEESPVVSW | 597 | B4402 | 23 | N/A |
| 19-27 | LMKEESPVV | 598 | A0201 | 22 | 5.024 |
| | | | B5101 | 15 | <5 |
| 18-27 | RLMKEESPVV | 599 | A0201 | 22 | 105.51 |
| | | | A3 | 18 | <5 |
| 18-26 | RLMKEESPV | 600 | A0201 | 21 | 257.342 |
| | | | A3 | 17 | <5 |
| 14-22 | LLQARLMKE | 601 | A0201 | 18 | <5 |
| | | | A3 | 15 | <5 |
| 13-22 | QLLQARLMKE | 602 | A0201 | 18 | <5 |
| | | | A26 | 15 | N/A |
| | | | A3 | 15 | <5 |

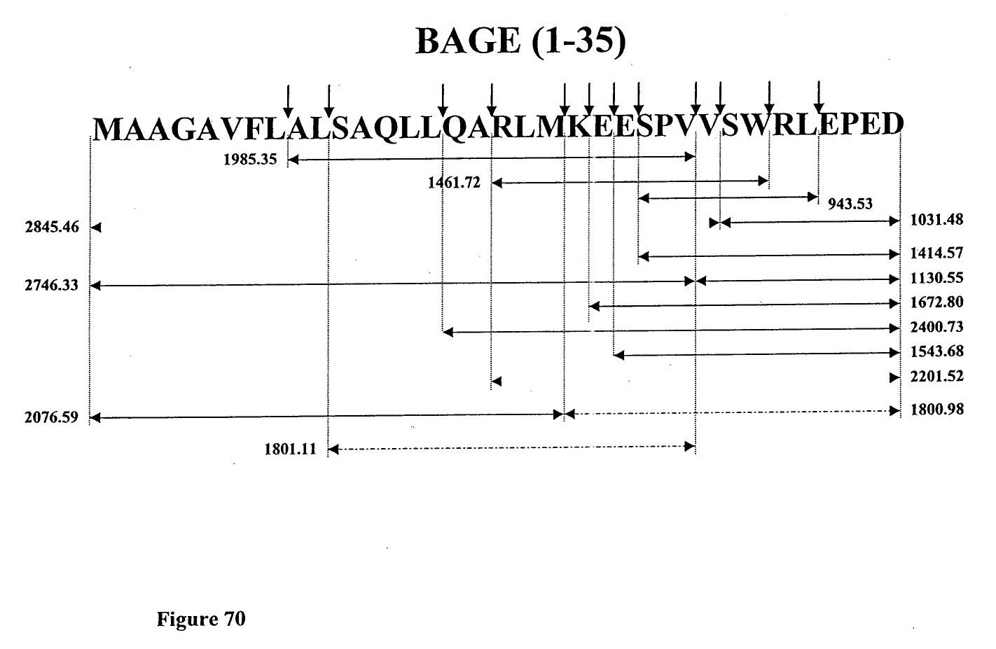

†Scores are given from the two binding prediction programs referenced above (see example 3)
See also FIG. 70.

### Example 68

Epitope Clusters.

Known and predicted epitopes are generally not evenly distributed across the sequences of protein antigens. As referred to above, we have defined segments of sequence containing a higher than average density of (known or predicted) epitopes as epitope clusters. Among the uses of epitope clusters is the incorporation of their sequence into substrate peptides used in proteasomal digestion analysis as described herein, or to otherwise inform the selection and design of such substrates. Epitope clusters can also be useful as vaccine components. Fuller discussions of the definition and uses of epitope clusters is found in PCT Publication No. WO 01/82963; PCT Publication No. WO 03/057823; and U.S. patent application Ser. No. 09/561,571 entitled EPITOPE CLUSTERS, which all are or were previously incorporated by reference in their entireties and in U.S. patent application Ser. No. 10/026,066 entitled “EPITOPE SYNCHRONIZATION IN ANTIGEN PRESENTING CELLS”, which is hereby incorporated by reference in its entirety. Epitopes and epitope clusters for many of the TAA mentioned herein have been previously disclosed in PCT Publication No. WO 02/081646; in patent application Ser. No. 09/561,571; in U.S. patent application Ser. No. 10/117,937; U.S. Provisional Application No. 60/337,017 filed on Nov. 7, 2001, and 60/363,210 filed on Mar. 7, 2002, all entitled EPITOPE SEQUENCES, which are all incorporated by reference in their entirety. The teachings and embodiments disclosed in said publications and applications are contemplated as supporting principals and embodiments related to and useful in connection with the present invention.

For the TuAAs survivin (SEQ ID NO. 98) and GAGE-1 (SEQ ID NO.

96) the following tables (68-73) present 9-mer epitopes predicted for HLA-A2 binding using both the SYFPEITHI and NIH algorithms and the epitope density of regions of overlapping epitopes, and of epitopes in the whole protein, and the ratio of these two densities. (The ratio must exceed one for there to be a cluster by the above definition; requiring higher values of this ratio reflect preferred embodiments). Individual 9-mers are ranked by score and identified by the position of their first amino in the complete protein sequence. Each potential cluster from a protein is numbered. The range of amino acid positions within the complete sequence that the cluster covers is indicated, as are the rankings of the individual predicted epitopes it is made up of.

TABLE 68
HLA-A2 Epitope cluster analysis for Survivin (NIH algorithm)

Length of protein sequence: 142 amino acids
Number of 9-mers: 134
Number of 9-mers with NIH score ≧ 5:2

| | | Peptide | Start | | Peptides/AAs | | |
|---|---|---|---|---|---|---|---|
| Cluster | AA | Rank | Position | Score | Cluster | Whole Pro. | Ratio |
| 1 | 13-28 | 1 | 13 | 10.26 | 0.125 | 0.014 | 8.875 |
| SEQ ID NO: 603 | | 2 | 20 | 4.919 | | | |

TABLE 69
HLA-A2 Epitope cluster analysis for Survivin (SYFPEITHI algorithm)

Length of protein sequence: 142 amino acids
Number of 9-mers: 134
Number of 9-mers with SYFPEITHI score ≧ 15:10

| | | Peptide | Start | | Peptides/AAs | | |
|---|---|---|---|---|---|---|---|
| Cluster | AA | Rank | Position | Score | Cluster | Whole Pro. | Ratio |
| 1 | 13-28 | 5 | 13 | 17 | 0.125 | 0.070 | 1.775 |
| SEQ ID NO: 603 | | 4 | 20 | 18 | | | |
| 2 | 79-111 | 8 | 79 | 15 | 0.182 | 0.070 | 2.597 |
| SEQ ID NO: 604 | | 9 | 81 | 15 | | | |
| | | 6 | 88 | 17 | | | |
| | | 1 | 96 | 23 | | | |
| | | 7 | 97 | 16 | | | |
| | | 10 | 103 | 15 | | | |
| 3 | 130-141 | 2 | 130 | 19 | 0.167 | 0.070 | 2.381 |
| SEQ ID NO: 605 | | 3 | 133 | 19 | | | |

TABLE 70
HLA-A2 Epitope cluster analysis for GAGE-1 (NIH algorithm)

Length of protein sequence: 138 amino acids
Number of 9-mers: 130
Number of 9-mers with NIH score ≧ 5:5

| | | Peptide | Start | | Peptides/AAs | | |
|---|---|---|---|---|---|---|---|
| Cluster | AA | Rank | Position | Score | Cluster | Whole Pro. | Ratio |
| 1 | 116-133 | 1 | 123 | 1999.734 | 0.278 | 0.036 | 7.667 |
| SEQ ID NO: 606 | | 2 | 121 | 161.227 | | | |
| | | 3 | 125 | 49.834 | | | |
| | | 4 | 117 | 37.362 | | | |
| | | 5 | 116 | 6.381 | | | |

TABLE 71
HLA-A2 Epitope cluster analysis for GAGE-1 (SYFPEITHI algorithm)

Length of protein sequence: 138 amino acids
Number of 9-mers: 130
Number of 9-mers with SYFPEITHI score ≧ 5:6

| | | Peptide | Start | | Peptides/AAs | | |
|---|---|---|---|---|---|---|---|
| Cluster | AA | Rank | Position | Score | Cluster | Whole Pro. | Ratio |
| 1 | 116-133 | 1 | 116 | 22 | 0.333 | 0.043 | 7.667 |
| SEQ ID NO: 606 | | 2 | 123 | 22 | | | |
| | | 3 | 125 | 22 | | | |
| | | 4 | 117 | 17 | | | |
| | | 5 | 120 | 16 | | | |
| | | 6 | 121 | 15 | | | |

TABLE 72
HLA-A2 Epitope cluster analysis for BAGE (NIH algorithm)

Length of protein sequence: 43 amino acids
Number of 9-mers included: 35
Number of 9-mers with NIH score ≧ 5:4

| | | Peptide | Start | | Peptides/AAs | | |
|---|---|---|---|---|---|---|---|
| Cluster | AA | Rank | Position | Score | Cluster | Whole Pro. | Ratio |
| 1 | 7-17 | 2 | 7 | 98.267 | 0.182 | 0.093 | 1.955 |
| SEQ ID NO: 607 | | 3 | 9 | 11.426 | | | |
| 2 | 18-27 | 1 | 18 | 257.342 | 0.200 | 0.093 | 2.151 |
| SEQ ID NO: 608 | | 4 | 19 | 5.024 | | | |

TABLE 73
HLA-A2 Epitope cluster analysis for BAGE (SYFPEITHI algorithm)

Length of protein sequence: 43 amino acids
Number of 9-mers included: 35
Number of 9-mers with SYFPEITHI score ≧ 15:10

| | | Peptide | Start | | Peptides/AAs | | |
|---|---|---|---|---|---|---|---|
| Cluster | AA | Rank | Position | Score | Cluster | Whole Pro. | Ratio |
| 1 | 2-27 | 6 | 2 | 18 | 0.308 | 0.233 | 1.323 |
| SEQ ID NO: 609 | | 9 | 6 | 16 | | | |
| | | 1 | 7 | 23 | | | |
| | | 3 | 9 | 21 | | | |
| | | 5 | 11 | 19 | | | |
| | | 7 | 14 | 18 | | | |
| | | 4 | 18 | 21 | | | |
| | | 2 | 19 | 22 | | | |
| 2 | 30-39 | 8 | 30 | 17 | 0.200 | 0.233 | 0.858 |
| SEQ ID NO: 610 | | 10 | 31 | 15 | | | |

The embodiments of the invention are applicable to and contemplate variations in the sequences of the target antigens provided herein, including those disclosed in the various databases that are accessible by the world wide web. Specifically for the specific sequences disclosed herein, variation in sequences can be found by using the provided accession numbers to access information for each antigen.

| TYROSINASE PROTEIN; SEQ ID NO 2 | |
|---|---|
| 1 | MLLAVLYCLL WSFQTSAGHF PRACVSSKNL MEKECCPPWS GDRSPCGQLS GRGSCQNILL |
| 61 | SNAPLGPQFP FTGVDDRESW PSVFYNRTCQ CSGNFMGFNC GNCKFGFWGP NCTERRLLVR |
| 121 | RNIFDLSAPE KDKFFAYLTL AKHTISSDYV IPIGTYGQMK NGSTPMFNDI NIYDLFVWMH |
| 181 | YYVSMDALLG GSEIWRDIDF AHEAPAFLPW HRLFLLRWEQ EIQKLTGDEN FTIPYWDWRD |
| 241 | AEKCDICTDE YMGGQHPTNP NLLSPASFFS SWQIVCSRLE EYNSHQSLCN GTPEGPLRRN |
| 301 | PGNHDKSRTP RLPSSADVEF CLSLTQYESG SMDKAANFSF RNTLEGFASP LTGIADASQS |
| 361 | SMHNALHIYM NGTMSQVQGS ANDPIFLLHH AFVDSIFEQW LRRHRPLQEV YPEANAPIGH |
| 421 | NRESYMVPFI PLYRNGDFFI SSKDLGYDYS YLQDSDPDSF QDYIKSYLEQ ASRIWSWLLG |
| 481 | AAMVGAVLTA LLAGLVSLLC RHKRKQLPEE KQPLLMEKED YHSLYQSHL |
| SSX-2 PROTEIN; SEQ ID NO 3 | |
| 1 | MNGDDAFARR PTVGAQIPEK IQKAFDDIAK YFSKEEWEKM KASEKIFYVY MKRKYEAMTK |
| 61 | LGFKATLPPF MCNKRAEDFQ GNDLDNDPNR GNQVERPQMT FGRLQGISPK IMPKKPAEEG |
| 121 | NDSEEVPEAS GPQNDGKELC PPGKPTTSEK IHERSGPKRG EHAWTHRLRE RKQLVIYEEI |
| 181 | SDPEEDDE |
| PSMA PROTEIN; SEQ ID NO 4 | |
| 1 | MWNLLHETDS AVATARRPRW LCAGALVLAG GFFLLGFLFG WFIKSSNEAT NITPKHNMKA |
| 61 | FLDELKAENI KKFLYNFTQI PHLAGTEQNF QLAKQIQSQW KEFGLDSVEL AHYDVLLSYP |
| 121 | NKTHPNYISI INEDGNEIFN TSLFEPPPPG YENVSDIVPP FSAFSPQGMP EGDLVYVNYA |
| 181 | RTEDFFKLER DMKINCSGKI VIARYGKVFR GNKVKNAQLA GAKGVILYSD PADYFAPGVK |
| 241 | SYPDGWNLPG GGVQRGNILN LNGAGDPLTP GYPANEYAYR RGIAEAVGLP SIPVHPIGYY |
| 301 | DAQKLLEKMG GSAPPDSSWR GSLKVPYNVG PGFTGNFSTQ KVKMHIHSTN EVTRIYNVIG |
| 361 | TLRGAVEPDR YVILGGHRDS WVFGGIDPQS GAAVVHEIVR SFGTLKKEGW RPRRTILFAS |
| 421 | WDAEEFGLLG STEWAEENSR LLQERGVAYI NADSSIEGNY TLRVDCTPLM YSLVHNLTKE |
| 481 | LKSPDEGFEG KSLYESWTKK SPSPEFSGMP RISKLGSGND FEVFFQRLGI ASGRARYTKN |
| 541 | WETNKFSGYP LYHSVYETYE LVEKFYDPMF KYHLTVAQVR GGMVFELANS IVLPFDCRDY |
| 601 | AVVLRKYADK IYSISMKHPQ EMKTYSVSFD SLFSAVKNFT EIASKFSERL QDFDKSNPIV |
| 661 | LRNMNDQLMF LERAFIDPLG LPDRPFYRHV IYAPSSHNKY AGESFPGIYD ALFDIESKVD |
| 721 | PSKAWGEVKR QIYVAAFTVQ AAAETLSEVA |
| Homo sapiens tyrosinase (oculocutaneous albinism IA) (TYR), mRNA.; | |
| ACCESSION NM_000372 | |
| VERSION NM_000372.1 GI:4507752 | |
| SEQ ID NO 2 | |
| /translation = “MLLAVLYCLLWSFQTSAGHFPRACVSSKNLMEKECCPPWSGDRSPCGQLSGRG | |
| SCQNILLSNAPLGPQFPFTGVDDRESWPSVFYNRTCQCSGNFMGFNCGNCKFGFWGPNCTERRLLVRRN | |
| IFDLSAPEKDKFFAYLTLAKHTISSDYVIPIGTYGQMKNGSTPMFNDINIYDLFVWMHYYVSMDALLGG | |
| SEIWRDIDFAHEAPAFLPWHRLFLLRWEQEIQKLTGDENFTIPYWDWRDAEKCDICTDEYMGGQHPTNP | |
| NLLSPASFFSSWQIVCSRLEEYNSHQSLCNGTPEGPLRRNPGNHDKSRTPRLPSSADVEFCLSLTQYES | |
| GSMDKAANFSFRNTLEGFASPLTGIADASQSSMHNALHIYMNGTMSQVQGSANDPIFLLHHAFVDSIFE | |
| QWLRRHRPLQEVYPEANAPIGHNRESYMVPFIPLYRNGDFFISSKDLGYDYSYLQDSDPDSFQDYIKSY | |
| LEQASRIWSWLLGAAMVGAVLTALLAGLVSLLCRHKRKQLPEEKQPLLMEKEDYHSLYQSHL” | |
| SEQ ID NO 5 | |
| ORIGIN | |
| 1 | atcactgtag tagtagctgg aaagagaaat ctgtgactcc aattagccag ttcctgcaga |
| 61 | ccttgtgagg actagaggaa gaatgctcct qgctgttttg tactgcctgc tgtggagttt |
| 121 | ccagacctcc gctggccatt tccctagagc ctgtgtctcc tctaagaacc tgatggagaa |
| 181 | ggaatgctgt ccaccgtgga gcggggacag gagtccctgt ggccagcttt caggcagagg |
| 241 | ttcctgtcag aatatccttc tgtccaatgc accacttggg cctcaatttc ccttcacagg |
| 301 | ggtqgatgac cgggagtcgt ggccttccgt cttttataat aggacctgcc agtgctctgg |
| 361 | caacttcatg ggattcaact gtggaaactg caagtttggc ttttggggac caaactgcac |
| 421 | agagagacga ctcttggtga gaagaaacat cttcgatttg agtgccccag agaaggacaa |
| 481 | attttttgcc tacctcactt tagcaaagca taccatcagc tcagactatg tcatccccat |
| 541 | agggacctat ggccaaatga aaaatggatc aacacccatg tttaacgaca tcaatattta |
| 601 | tgacctcttt gtctggatgc attattatgt gtcaatggat gcactgcttg ggggatctga |
| 661 | aattctggaga gacattgatt ttgcccatga agcaccagct tttctgcctt ggcatagact |
| 721 | cttcttgttg cggtgggaac aagaaatcca gaagctgaca ggagatgaaa acttcactat |
| 781 | tccatattgg gactggcggg atgcagaaaa gtgtgacatt tgcacagatg agtacatggg |
| 841 | aggtcagcac cccacaaatc ctaacttact cagcccagca tcattcttct cctcttggca |
| 901 | gattgtctgt agccgattgg aggagtacaa cagccatcag tctttatgca atggaacgcc |
| 961 | cgagggacct ttacggcgta atcctggaaa ccatgacaaa tccagaaccc caaggctccc |
| 1021 | ctcttcagct gatgtagaat tttgcctgag tttgacccaa tatgaatctg gttccatgga |
| 1081 | taaagctqcc aatttcagct ttagaaatac actggaagga tttgctagtc cacttactgg |
| 1141 | gatagcggat gcctctcaaa gcagcatgca caatgccttg cacatctata tgaatggaac |
| 1201 | aatgtcccag gtacagggat ctgccaacga tcctatcttc cttcttcacc atgcatttgt |
| 1261 | tgacagtatt tttgagcagt ggctccgaag gcaccgtcct cttcaagaag tttatccaga |
| 1321 | agccaatgca cccattggac ataaccggga atcctacatg gttcctttta taccactgta |
| 1381 | cagaaatggt gatttcttta tttcatccaa agatctgggc tatgactata gctatctaca |
| 1441 | agattcagac ccagactctt ttcaagacta cattaagtcc tatttggaac aagcgagtcg |
| 1501 | gatctggtca tggctccttg ggqcggcgat ggtaggggcc gtcctcactg ccctgctggc |
| 1561 | agggcttgtg agcttgctgt gtcgtcacaa gagaaagcag cttcctgaag aaaagcagcc |
| 1621 | actcctcatg gagaaagagg attaccacag cttgtatcag agccatttat aaaaggctta |
| 1681 | ggcaatagag tagggccaaa aagcctgacc tcactctaac tcaaagtaat gtccaggttc |
| 1741 | ccagagaata tctgctggta tttttctgta aagaccattt gcaaaattgt aacctaatac |
| 1801 | aaagtgtagc cttcttccaa ctcaggtaga acacacctgt ctttgtcttg ctgttttcac |
| 1861 | tcagcccttt taacattttc ccctaagccc atatgtctaa ggaaaggatg ctatttggta |
| 1921 | atgaggaact gttatttgta tgtgaattaa agtgctctta tttt |
| Homo sapiens synovial sarcoma, X breakpoint 2 (SSX2), mRNA. | |
| ACCESSION NM_003147 | |
| VERSION NM_003147.1 GI:10337582 | |
| SEQ ID NO 3 | |
| /translation = “MNGDDAFARRPTVGAQIPEKIQKAFDDIAKYFSKEEWEKMKASEKIFYVYMKR | |
| KYEAMTKLGFKATLPPFMCNKRAEDFQGNDLDNDPNRGNQVERPQMTFGRLQGISPKIMPKKPAEEGND | |
| SEEVPEASGPQNDGKELCPPGKPTTSEKIHERSGPKRGEHAWTHRLRERKQLVIYEEISDPEEDDE” | |
| SEQ ID NO 6 | |
| ORIGIN | |
| 1 | ctctctttcg attcttccat actcagagta cgcacggtct gattttctct ttggattctt |
| 61 | ccaaaatcag agtcagactg ctcccggtgc catgaacgga gacgacgcct ttgcaaggag |
| 121 | acccacggtt ggtgctcaaa taccagagaa gatccaaaag gccttcgatg atattgccaa |
| 181 | atacttctct aaggaagagt gggaaaagat gaaagcctcg gagaaaatct tctatgtgta |
| 241 | tatgaagaga aagtatgagg ctatgactaa actaggtttc aaggccaccc tcccaccttt |
| 301 | catgtgtaat aaacgggccg aagacttcca ggggaatgat ttggataatg accctaaccg |
| 361 | tgggaatcag gttgaacgtc ctcagatgac tttcggcagg ctccagggaa tctccccgaa |
| 421 | gatcatgccc aagaagccag cagaggaagg aaatgattcg gaggaagtgc cagaagcatc |
| 481 | tggcccacaa aatgatggga aagagctgtg ccccccggga aaaccaacta cctctgagaa |
| 541 | gattcacgag agatctggac ccaaaagggg ggaacatgcc tggacccaca gactgcgtga |
| 601 | gagaaaacag ctggtgattt atgaagagat cagcgaccct gaggaagatg acgagtaact |
| 661 | cccctcaggg atacgacaca tgcccatgat gagaagcaga acgtggtgac ctttcacgaa |
| 721 | catgggcatg gctgcggacc cctcgtcatc aggtgcatag caagtg |
| Homo sapiens folate hydrolase (prostate-specific membrane antigen) | |
| 1 (FOLH1), mRNA. | |
| ACCESSION NM_004476 | |
| VERSION NM_004476.1 GI:4758397 | |
| SEQ ID No. 4 | |
| /translation = “MWNLLHETDSAVATARRPRWLCAGALVLAGGFFLLGFLFGWFIKSSNEATNIT | |
| PKHNMKAFLDELKAENIKKFLYNFTQIPHLAGTEQNFQLAKQIQSQWKEFGLDSVELAHYDVLLSYPNK | |
| THPNYISIINEDGNEIFNTSLFEPPPPGYENVSDIVPPFSAFSPQGMPEGDLVYVNYARTEDFFKLERD | |
| MKINCSGKIVIARYGKVFRGNKVKNAQLAGAKGVILYSDPADYFAPGVKSYPDGWNLPGGGVQRGNILN | |
| LNGAGDPLTPGYPANEYAYRRGIAEAVGLPSIPVHPIGYYDAQKLLEKMGGSAPPDSSWRGSLKVPYNV | |
| GPGFTGNFSTQKVKMHIHSTNEVTRIYNVIGTLRGAVEPDRYVILGGHRDSWVFGGIDPQSGAAVVHEI | |
| VRSFGTLKKEGWRPRRTILFASWDAEEFGLLGSTEWAEENSRLLQERGVAYINADSSIEGNYTLRVDCT | |
| PLMYSLVHNLTKELKSPDEGFEGKSLYESWTKKSPSPEFSGMPRISKLGSGNDFEVFFQRLGIASGRAR | |
| YTKNWETNKFSGYPLYHSVYETYELVEKFYDPMFKYHLTVAQVRGGMVFELANSIVLPFDCRDYAVVLR | |
| KYADKIYSISMKHPQEMKTYSVSFDSLFSAVKNFTEIASKFSERLQDFDKSNPIVLRMMNDQLMFLERA | |
| FIDPLGLPDRPFYRHVIYAPSSHNKYAGESFPGIYDALFDIESKVDPSKAWGEVKRQIYVAAFTVQAAA | |
| ETLSEVA” | |
| SEQ ID NO 7 | |
| ORIGIN | |
| 1 | ctcaaaaggg gccggatttc cttctcctgg aggcagatgt tgcctctctc tctcgctcgg |
| 61 | attggttcag tgcactctag aaacactgct gtggtggaga aactggaccc caggtctgga |
| 121 | gcgaattcca gcctgcaggg ctgataagcg aggcattagt gagattgaga gagactttac |
| 181 | cccgccgtgg tggttggagg gcgcgcagta gagcagcagc acaggcgcgg gtcccgggag |
| 241 | gccggctctg ctcgcgccga gatgtggaat ctccttcacg aaaccgactc ggctgtggcc |
| 301 | accgcgcgcc gcccgcgctg gctgtgcgct ggggcgctgg tgctggcggg tggcttcttt |
| 361 | ctcctcggct tcctcttcgg gtggtttata aaatcctcca atgaagctac taacattact |
| 421 | ccaaagcata atatgaaagc atttttggat gaattgaaag ctgagaacat caagaagttc |
| 481 | ttatataatt ttacacagat accacattta gcaggaacag aacaaaactt tcagcttgca |
| 541 | aagcaaattc aatcccagtg gaaagaattt ggcctggatt ctgttgagct agcacattat |
| 601 | gatgtcctgt tgtcctaccc aaataagact catcccaact acatctcaat aattaatgaa |
| 661 | gatggaaatg agattttcaa cacatcatta tttgaaccac ctcctccagg atatgaaaat |
| 721 | gtttcggata ttgtaccacc tttcagtgct ttctctcctc aaggaatgcc agagggcgat |
| 781 | ctagtgtatg ttaactatgc acgaactgaa gacttcttta aattggaacg ggacatgaaa |
| 841 | atcaattgct ctgggaaaat tgtaattgcc agatatggga aagttttcag aggaaataag |
| 901 | gttaaaaatg cccagctggc aggggccaaa ggagtcattc tctactccga ccctgctgac |
| 961 | tactttgctc ctggggtgaa gtcctatcca gatggttgga atcttcctgg aggtggtgtc |
| 1021 | cagcgtggaa atatcctaaa tctgaatggt gcaggagacc ctctcacacc aggttaccca |
| 1081 | gcaaatgaat atgcttatag gcgtggaatt gcagaggctg ttggtcttcc aagtattcct |
| 1141 | gttcatccaa ttggatacta tgatgcacag aagctcctag aaaaaatggg tggctcagca |
| 1201 | ccaccagata gcagctggag aggaagtctc aaagtgccct acaatgttgg acctggcttt |
| 1261 | actggaaact tttctacaca aaaagtcaag atgcacatcc actctaccaa tgaagtgaca |
| 1321 | agaatttaca atgtgatagg tactctcaga ggagcagtgg aaccagacag atatgtcatt |
| 1381 | ctgggaggtc accgggactc atgggtgttt ggtggtattg accctcagag tggagcagct |
| 1441 | gttgttcatg aaattgtgag gagctttgga acactgaaaa aggaagggtg gagacctaga |
| 1501 | agaacaattt tgtttgcaag ctgggatgca gaagaatttg gtcttcttgg ttctactgag |
| 1561 | tgggcagagg agaattcaag actccttcaa gagcgtggcg tggcttatat taatgctgac |
| 1621 | tcatctatag aaggaaacta cactctgaga gttgattgta caccgctgat gtacagcttg |
| 1681 | gtacacaacc taacaaaaga gctgaaaagc cctgatgaag gctttgaagg caaatctctt |
| 1741 | tatgaaagtt ggactaaaaa aagtccttcc ccagagttca gtggcatgcc caggataagc |
| 1801 | aaattgggat ctggaaatga ttttgaggtg ttcttccaac gacttggaat tgcttcaggc |
| 1861 | agagcacggt atactaaaaa ttgggaaaca aacaaattca gcggctatcc actgtatcac |
| 1921 | agtgtctatg aaacatatga gttggtggaa aagttttatg atccaatgtt taaatatcac |
| 1981 | ctcactgtgg cccaggttcg aggagggatg gtgtttgagc tagccaattc catagtgctc |
| 2041 | ccttttgatt gtcgagatta tgctgtagtt ttaagaaagt atgctgacaa aatctacagt |
| 2101 | atttctatga aacatccaca ggaaatgaag acatacagtg tatcatttga ttcacttttt |
| 2161 | tctgcagtaa agaattttac agaaattgct tccaagttca gtgagagact ccaggacttt |
| 2221 | gacaaaagca acccaatagt attaagaatg atgaatgatc aactcatgtt tctggaaaga |
| 2281 | gcatttattg atccattagg gttaccagac aggccttttt ataggcatgt catctatgct |
| 2341 | ccaagcagcc acaacaagta tgcaggggag tcattcccag gaatttatga tgctctgttt |
| 2401 | gatattgaaa gcaaagtgga cccttccaag gcctggggag aagtgaagag acagatttat |
| 2461 | gttgcagcct tcacagtgca ggcagctgca gagactttga gtgaagtagc ctaagaggat |
| 2521 | tctttagaga atccgtattg aatttgtgtg gtatgtcact cagaaagaat cgtaatgggt |
| 2581 | atattgataa attttaaaat tggtatattt gaaataaagt tgaatattat atataaaaaa |
| 2641 | aaaaaaaaaa aaa |
| Human melanocyte-specific (pmel 17) gene, exons 2-5, and complete | |
| cds. | |
| ACCESSION U20093 | |
| VERSION U20093.1 GI:1142634 | |
| SEQ ID NO 70 | |
| /translation = “MDLVLKRCLLHLAVIGALLAVGATKVPRNQDWLGVSRQLRTKAWNRQLYPEWT | |
| EAQRLDCWRGGQVSLKVSNDGPTLIGANASFSIALNFPGSQKVLPDGQVIWVNNTIINGSQVWGGQPVY | |
| PQETDDACIFPDGGPCPSGSWSQKRSFVYVWKTWGQYWQVLGGPVSGLSIGTGRANLGTHTMEVTVYHR | |
| RGSRSYVPLAHSSSAFTITDQVPFSVSVSQLRALDGGNKHFLRNQPLTFALQLHDPSGYLAEADLSYTW | |
| DFGDSSGTLISRAPVVTHTYLEPGPVTAQVVLQAAIPLTSCGSSPVPGTTDGHRPTAEAPNTTAGQVPT | |
| TEVVGTTPGQAPTAEPSGTTSVQVPTTEVISTAPVQMPTAESTGMTPEKVPVSEVMGTTLAEMSTPEAT | |
| GMTPAEVSIVVLSGTTAAQVTTTEWVETTARELPIPEPEGPDASSIMSTESITGSLGPLLDGTATLRLV | |
| KRQVPLDCVLYRYGSFSVTLDIVQGIESAEILQAVPSGEGDAFELTVSCQGGLPKEACMEISSPGCQPP | |
| AQRLCQPVLPSPACQLVLHQILKGGSGTYCLNVSLADTNSLAVVSTQLIMPGQEAGLGQVPLIVGILLV | |
| LMAVVLASLIYRRRLMKQDFSVPQLPHSSSHWLRLPRIFCSCPIGENSPLLSGQQV” | |
| SEQ ID NO 80 | |
| ORIGIN | |
| 1 | gtgctaaaaa gatgccttct tcatttggct gtgataggtg ctttgtggct gtgggggcta |
| 61 | caaaagtacc cagaaaccag gactggcttg gtgtctcaag gcaactcaga accaaagcct |
| 121 | ggaacaggca gctgtatcca gagtggacag aagcccagag acttgactgc tggagaggtg |
| 181 | gtcaagtgtc cctcaaggtc agtaatgatg ggcctacact gattggtgca aatgcctcct |
| 241 | tctctattgc cttgaacttc cctggaagcc aaaaggtatt gccagatggg caggttatct |
| 301 | gggtcaacaa taccatcatc aatgggagcc aggtgtgggg aggacagcca gtgtatcccc |
| 361 | aggaaactga cgatgcctgc atcttccctg atggtggacc ttgcccatct ggctcttggt |
| 421 | ctcagaagag aagctttgtt tatgtctgga agacctgggg tgagggactc ccttctcagc |
| 481 | ctatcatcca cacttgtgtt tacttctttc tacctgatca cctttctttt ggccgcccct |
| 541 | tccaccttaa cttctgtgat tttctctaat cttcattttc ctcttagatc ttttctcttt |
| 601 | cttagcacct agcccccttc aagctctatc ataattcttt ctggcaactc ttggcctcaa |
| 661 | ttgtagtcct accccatgga atgcctcatt aggacccctt ccctgtcccc ccatatcaca |
| 721 | gccttccaaa caccctcaga agtaatcata cttcctgacc tcccatctcc agtgccgttt |
| 781 | cgaagcctgt ccctcagtcc cctttgacca gtaatctctt cttccttgct tttcattcca |
| 841 | aaaatgcttc aggccaatac tggcaagttc tagggggccc agtgtctggg ctgagcattg |
| 901 | ggacaggcag ggcaatgctg ggcacacaca ccatggaagt gactgtctac catcgccggg |
| 961 | gatcccggag ctatgtgcct cttgctcatt ccagctcagc cttcaccatt actggtaagg |
| 1021 | gttcaggaag ggcaaggcca gttgtagggc aaagagaagg cagggaggct tggatggact |
| 1081 | gcaaaggaga aaggtgaaat gctgtgcaaa cttaaagtag aagggccagg aagacctagg |
| 1141 | cagagaaatg tgaggcttag tgccagtgaa gggccagcca gtcagcttgg agttggaggg |
| 1201 | tgtggctgtg aaaggagaag ctgtggctca ggcctggttc tcaccttttc tggctccaat |
| 1261 | cccagaccag gtgcctttct ccgtgagcgt gtcccagttg cgggccttgg atggagggaa |
| 1321 | caagcacttc ctgagaaatc agcctctgac ctttgccctc cagctccatg accccagtgg |
| 1381 | ctatctggct gaagctgacc tctcctacac ctgggacttt ggagacagta gtggaaccct |
| 1441 | gatctctcgg gcacctgtgg tcactcatac ttacctggag cctggcccag tcactgccca |
| 1501 | ggtggtcctg caggctgcca ttcctctcac ctcctgtggc tcctccccag ttccaggcac |
| 1561 | cacagatggg cacaggccaa ctgcagaggc ccctaacacc acagctggcc aagtgcctac |
| 1621 | tacagaagtt gtgggtacta cacctggtca ggcgccaact gcagagccct ctggaaccac |
| 1681 | atctgtgcag gtgccaacca ctgaagtcat aagcactgca cctgtgcaga tgccaactgc |
| 1741 | agagagcaca ggtatgacac ctgagaaggt gccagtttca gaggtcatgg gtaccacact |
| 1801 | ggcagagatg tcaactccag aggctacagg tatgacacct gcagaggtat caattgtggt |
| 1861 | gctttctgga accacagctg cacaggtaac aactacagag tgggtggaga ccacagctag |
| 1921 | agagctacct atccctgagc ctgaaggtcc agatgccagc tcaatcatgt ctacggaaag |
| 1981 | tattacaggt tccctgggcc ccctgctgga tggtacagcc accttaaggc tggtgaagag |
| 2041 | acaagtcccc ctggattgtg ttctgtatcg atatggttcc ttttccgtca ccctggacat |
| 2101 | tgtccagggt attgaaagtg ccgagatcct gcaggctgtg ccgtccggtg agggggatgc |
| 2161 | atttgagctg actgtgtcct gccaaggcgg gctgcccaag gaagcctgca tggagatctc |
| 2221 | atcgccaggg tgccagcccc ctgcccagcg gctgtgccag cctgtgctac ccagcccagc |
| 2281 | ctgccagctg gttctgcacc agatactgaa gggtggctcg gggacatact gcctcaatgt |
| 2341 | gtctctggct gataccaaca gcctggcagt ggtcagcacc cagcttatca tgcctggtag |
| 2401 | gtccttggac agagactaag tgaggaggga agtggataga ggggacagct ggcaagcagc |
| 2461 | agacatgagt gaagcagtgc ctgggattct tctcacaggt caagaagcag gccttgggca |
| 2521 | ggttccgctg atcgtgggca tcttgctggt gttgatggct gtggtccttg catctctgat |
| 2581 | atataggcgc agacttatga agcaagactt ctccgtaccc cagttgccac atagcagcag |
| 2641 | tcactggctg cgtctacccc gcatcttctg ctcttgtccc attggtgaga atagccccct |
| 2701 | cctcagtggg cagcaggtct gagtactctc atatgatgct gtgattttcc tggagttgac |
| 2761 | agaaacacct atatttcccc cagtcttccc tgggagacta ctattaactg aaataaa |
| // | |
| Homo sapiens kallikrein 3, (prostate specific antigen) (KLK3), mRNA. | |
| ACCESSION NM_001648 | |
| VERSION NM_001648.1 GI:4502172 | |
| SEQ ID NO 78 | |
| /translation = “MWVPVVFLTLSVTWIGAAPLILSRIVGGWECEKHSQPWQVLVASRGRAVCGGV | |
| LVHPQWVLTAAHCIRNKSVILLGRHSLFHPEDTGQVFQVSHSFPHPLYDMSLLKNRFLRPGDDSSHDLM | |
| LLRLSEPAELTDAVKVMDLPTQEPALGTTCYASGWGSIEPEEFLTPKKLQCVDLHVISNDVCAQVHPQK | |
| VTKFMLCAGRWTGGKSTCSGDSGGPLVCNGVLQGITSWGSEPCALPERPSLYTKVVHYRKWIKDTIVAN | |
| P” | |
| SEQ ID NO 86 | |
| ORIGIN | |
| 1 | agccccaagc ttaccacctg caccoggaga gctgtgtgtc accatgtggg tcccggttgt |
| 61 | cttcctcacc ctgtccgtga cgtggattgg tgctgcaccc ctcatcctgt ctcggattgt |
| 121 | gggaggctgg gagtgcgaga agcattccca accctggcag gtgcttgtgg cctctcgtgg |
| 181 | cagggcagtc tgcggcggtg ttctggtgca cccccagtgg gtcctcacag ctgcccactg |
| 241 | catcaggaac aaaagcgtga tcttgctggg tcggcacagc ctgtttcatc ctgaagacac |
| 301 | aggccaggta tttcaggtca gccacagctt cccacacccg ctctacgata tgagcctcct |
| 361 | gaagaatcga ttcctcaggc caggtgatga ctccagccac gacctcatgc tgctccgcct |
| 421 | gtcagagcct gccgagctca cggatgctgt gaaggtcatg gacctgccca cccaggagcc |
| 481 | agcactgggg accacctgct acgcctcagg ctggggcagc attgaaccag aggagttctt |
| 541 | gaccccaaag aaacttcagt gtgtggacct ccatgttatt tccaatgacg tgtgtgcgca |
| 601 | agttcaccct cagaaggtga ccaagttcat gctgtgtgct ggacgctgga cagggggcaa |
| 661 | aagcacctgc tcgggtgatt ctgggggccc acttgtctgt aatggtgtgc ttcaaggtat |
| 721 | cacgtcatgg ggcagtgaac catgtgccct gcccgaaagg ccttccctgt acaccaaggt |
| 781 | ggtgcattac cggaagtgga tcaaggacac catcgtggcc aacccctgag cacccctatc |
| 841 | aaccccctat tgtagtaaac ttggaacctt ggaaatgacc aggccaagac tcaagcctcc |
| 901 | ccagttctac tgacctttgt ccttaggtgt gaggtccagg gttgctagga aaagaaatca |
| 961 | gcagacacag gtgtagacca gagtgtttct taaatggtgt aattttgtcc tctctgtgtc |
| 1021 | ctggggaata ctggccatgc ctggagacat atcactcaat ttctctgagg acacagatag |
| 1081 | gatggggtgt ctgtgttatt tgtggggtac agagatgaaa gaggggtggg atccacactg |
| 1141 | agagagtgga gagtgacatg tgctggacac tgtccatgaa gcactgagca gaagctggag |
| 1201 | gcacaacgca ccagacactc acagcaagga tggagctgaa aacataaccc actctgtcct |
| 1261 | ggaggcactg ggaagcctag agaaggctgt gagccaagga gggagggtct tcctttggca |
| 1321 | tgggatgggg atgaagtaag gagagggact ggaccccctg gaagctgatt cactatgggg |
| 1381 | ggaggtgtat tgaagtcctc cagacaaccc tcagatttga tgatttccta gtagaactca |
| 1441 | cagaaataaa gagctgttat actgtg |
| // | |
| Human autoimmunogenic cancer/testis antigen NY-ESO-1 mRNA, | |
| complete cds. | |
| ACCESSION U87459 | |
| VERSIONU 87459.1 GI:1890098 | |
| SEQ ID NO 74 | |
| /translation = “MQAEGRGTGGSTGDADGPGGPGIPDGPGGNAGGPGEAGATGGRGPRGAGAARA | |
| SGPGGGAPRGPHGGAASGLNGCCRCGARGPESRLLEFYLAMPFATPMEAELARRSLAQDAPPLPVPGVL | |
| LKEFTVSGNILTIRLTAADHRQLQLSISSCLQQLSLLMWITQCFLPVFLAQPPSGQRR” | |
| SEQ ID NO 84 | |
| ORIGIN | |
| 1 | atcctcgtgg gccctgacct tctctctgag agccgggcag aggctccgga gccatgcagg |
| 61 | ccgaaggccg gggcacaggg ggttcgacgg gcgatgctga tggcccagga ggccctggca |
| 121 | ttcctgatgg cccagggggc aatgctggcg gcccaggaga ggcgggtgcc acgggcggca |
| 181 | gaggtccccg gggcgcaggg gcagcaaggg cctcggggcc gggaggaggc gccccgcggg |
| 241 | gtccgcatgg cggcgcggct tcagggctga atggatgctg cagatgcggg gccagggggc |
| 301 | cggagagccg cctgcttgag ttctacctcg ccatgccttt cgcgacaccc atggaagcag |
| 361 | agctggcccg caggagcctg gcccaggatg ccccaccgct tcccgtgcca ggggtgcttc |
| 421 | tgaaggagtt cactgtgtcc ggcaacatac tgactatccg actgactgct gcagaccacc |
| 481 | gccaactgca gctctccatc agctcctgtc tccagcagct ttccctgttg atgtggatca |
| 541 | cgcagtgctt tctgcccgtg tttttggctc agcctccctc agggcagagg cgctaagccc |
| 601 | agcctggcgc cccttcctag gtcatgcctc ctcccctagg gaatggtccc agcacgagtg |
| 661 | gccagttcat tgtgggggcc tgattgtttg tcgctggagg aggacggctt acatgtttgt |
| 721 | ttctgtagaa aataaaactg agctacgaaa aa |
| // | |
| LAGE-1a protein [Homo sapiens]. | |
| ACCESSION CAA11116 | |
| PID g3255959 | |
| VERSION CAA11116.1 GI:3255959 | |
| SEQ ID NO 75 | |
| ORIGIN | |
| 1 | mqaegrgtgg stgdadgpgg pgipdgpggn aggpgeagat ggrgprgaga arasgprgga |
| 61 | prgphggaas aqdgrcpcga rrpdsrllel hitmpfsspm eaelvrrils rdaaplprpg |
| 121 | avlkdftvsg nllfirltaa dhrqlqlsis sclqqlsllm witqcflpvf laqapsgqrr |
| 181 | |
| // | |
| LAGE-1b protein [Homo sapiens] | |
| ACCESSION@@CAA11117 | |
| PID@@@@@@@@g3255960 | |
| VERSION@@@@CAA11117.1@@GI:3255960 | |
| SEQ ID NO 76 | |
| ORIGIN | |
| 1 | mqaegrgtgg stgdadgpgg pgipdgpggn aggpgeagat ggrgprgaga arasgprgga |
| 61 | prgphggaas aqdgrcpcga rrpdsrllel hitmpfsspm eaelvrrils rdaaplprpg |
| 121 | avlkdfftvsg nllffmsvwdq dregagrmrv vgwglgsasp egqkardlrt pkhkvseqrp |
| 181 | gtpgppppeg aqgdgcrgva fnvmfsaphi |
| // | |
| Human antigen (MAGE-1) gene, complete cds. | |
| ACCESSION M77481 | |
| VERSION M77481.1 GI:416114 | |
| SEQ ID NO 71 | |
| /translation = “MSLEQRSLHCKPEEALEAQQEALGLVCVQAATSSSSPLVLGTLEEVPTAGSTD | |
| PPQSPQGASAFPTTINFTRQRQPSEGSSSREEEGPSTSCILESLFRAVITKKVADLVGFLLLKYRAREP | |
| VTKAEMLESVIKNYKHCFPEIFGKASESLQLVFGIDVKEADPTGHSYVLVTCLGLSYDGLLGDNQIMPK | |
| TGFLIIVLVMIAMEGGHAPEEEIWEELSVMEVYDGREHSAYGEPRKLLTQDLVQEKYLEYRQVPDSDPA | |
| RYEFLWGPRALAETSYVKVLEYVIKVSARVRFFFPSLREAALREEEEGV” | |
| SEQ ID NO 81 | |
| ORIGIN | |
| 1 | ggatccaggc cctgccagga aaaatataag ggccctgcgt gagaacagag ggggtcatcc |
| 61 | actgcatgag agtggggatg tcacagagtc cagcccaccc tcctggtagc actgagaagc |
| 121 | cagggctgtg cttgcggtct gcaccctgag ggcccgtgga ttcctcttcc tggagctcca |
| 181 | ggaaccaggc agtgaggcct tggtctgaga cagtatcctc aggtcacaga gcagaggatg |
| 241 | cacagggtgt gccagcagtg aatgtttgcc ctgaatgcac accaagggcc ccacctgcca |
| 301 | caggacacat aggactccac agagtctggc ctcacctccc tactgtcagt cctgtagaat |
| 361 | cgacctctgc tggccggctg taccctgagt accctctcac ttcctccttc aggttttcag |
| 421 | gggacaggcc aacccagagg acaggattcc ctggaggcca cagaggagca ccaaggagaa |
| 481 | gatctgtaag taggcctttg ttagagtctc caaggttcag ttctcagctg aggcctctca |
| 541 | cacactccct ctctccccag gcctgtgggt cttcattgcc cagctcctgc ccacactcct |
| 601 | gcctgctgcc ctgacgagag tcatcatgtc tcttgagcag aggagtctgc actgcaagcc |
| 661 | tgaggaagcc cttgaggccc aacaagaggc cctgggcctg gtgtgtgtgc aggctgccac |
| 721 | ctcctcctcc tctcctctgg tcctgggcac cctggaggag gtgcccactg ctgggtcaac |
| 781 | agatcctccc cagagtcctc agggagcctc cgcctttccc actaccatca acttcactcg |
| 841 | acagaggcaa cccagtgagg gttccagcag ccgtgaagag gaggggccaa gcacctcttg |
| 901 | tatcctggag tccttgttcc gagcagtaat cactaagaag gtggctgatt tggttggttt |
| 961 | tctgctcctc aaatatcgag ccagggagcc agtcacaaag gcagaaatgc tggagagtgt |
| 1021 | catcaaaaat tacaagcact gttttcctga gatcttcggc aaagcctctg agtccttgca |
| 1081 | gctggtcttt ggcattgacg tgaaggaagc agaccccacc ggccactcct atgtccttgt |
| 1141 | cacctgccta ggtctctcct atgatggcct gctgggtgat aatcagatca tgcccaagac |
| 1201 | aggcttcctg ataattgtcc tggtcatgat tgcaatggag ggcggccatg ctcctgagga |
| 1261 | ggaaatctgg gaggagctga gtgtgatgga ggtgtatgat gggagggagc acagtgccta |
| 1321 | tggggagccc aggaagctgc tcacccaaga tttggtgcag gaaaagtacc tggagtaccg |
| 1381 | gcaggtgccg gacagtgatc ccgcacgcta tgagttcctg tggggtccaa gggccctcgc |
| 1441 | tgaaaccagc tatgtgaaag tccttgagta tgtgatcaag gtcagtgcaa gagttcgctt |
| 1501 | tttcttccca tccctgcgtg aagcagcttt gagagaggag gaagagggag tctgagcatg |
| 1561 | agttgcagcc aaggccagtg ggagggggac tgggccagtg caccttccag ggccgcgtcc |
| 1621 | agcagcttcc cctgcctcgt gtgacatgag gcccattctt cactctgaag agagcggtca |
| 1681 | gtgttctcag tagtaggttt ctgttctatt gggtgacttg gagatttatc tttgttctct |
| 1741 | tttggaattg ttcaaatgtt tttttttaag ggatggttga atgaacttca gcatccaagt |
| 1801 | ttatgaatga cagcagtcac acagttctgt gtatatagtt taagggtaag agtcttgtgt |
| 1861 | tttattcaga ttgggaaatc cattctattt tgtgaattgg gataataaca gcagtggaat |
| 1921 | aagtacttag aaatgtgaaa aatgagcagt aaaatagatg agataaagaa ctaaagaaat |
| 1981 | taagagatag tcaattcttg ccttatacct cagtctattc tgtaaaattt ttaaagatat |
| 2041 | atgcatacct ggatttcctt ggcttctttg agaatgtaag agaaattaaa tctgaataaa |
| 2101 | gaattcttcc tgttcactgg ctcttttctt ctccatgcac tgagcatctg ctttttggaa |
| 2161 | ggccctgggt tagtagtgga gatgctaagg taagccagac tcatacccac ccatagggtc |
| 2221 | gtagagtcta ggagctgcag tcacgtaatc gaggtggcaa gatgtcctct aaagatgtag |
| 2281 | ggaaaagtga gagaggggtg agggtgtggg gctccgggtg agagtggtgg agtgtcaatg |
| 2341 | ccctgagctg gggcattttg ggctttggga aactgcagtt ccttctgggg gagctgattg |
| 2401 | taatgatctt gggtggatcc |
| // | |
| Human MAGE-2 gene exons 1-4, complete cds. | |
| ACCESSION L18920 | |
| VERSION L18920.1 GI:436180 | |
| SEQ ID NO 72 | |
| /translation = ”MPLEQRSQHCKPEEGLEARGEALGLVGAQAPATEEQQTASSSSTLVEVTLGEV | |
| PAADSPSPPHSPQGASSFSTTINYTLWRQSDEGSSNQEEEGPPNFPDLESEFQAAISRKMVELVHFLLL | |
| KYRAREPVTKAEMLESVLRNCQDFFPVIFSKASEYLQLVFGIEVVEVVPISHLYILVTCLGLSYDGLLG | |
| DNQVMPKTGLLIIVLAIIAIEGDCAPEEKIWEELSMLEVFEGREDSVFAHPRKLLMQDLVQENYLEYRQ | |
| VPGSDPACYEFLWGPRALIETSYVKVLHHTLKIGGEPHISYPPLHERALREGEE” | |
| SEQ ID NO 82 | |
| ORIGIN | |
| 1 | attccttcat caaacagcca ggagtgagga agaggaccct cctgagtgag gactgaggat |
| 61 | ccaccctcac cacatagtgg gaccacagaa tccagctcag cccctcttgt cagccctggt |
| 121 | acacactggc aatgatctca ccccgagcac acccctcccc ccaatgccac ttcgggccga |
| 181 | ctcagagtca gagacttggt ctgaggggag cagacacaat cggcagagga tggcggtcca |
| 241 | ggctcagtct ggcatccaag tcaggacctt gagggatgac caaaggcccc tcccaccccc |
| 301 | aactcccccg accccaccag gatotacago ctcaggatcc ccgtcccaat ccctacccct |
| 361 | acaccaacac catcttcatg cttaccccca cccccccatc cagatcccca tccgggcaga |
| 421 | atccggttcc acccttgccg tgaacccagg gaagtcacgg gcccggatgt gacgccactg |
| 481 | acttgcacat tggaggtcag aggacagcga gattctcgcc ctgagcaacg gcctgacgtc |
| 541 | ggcggaggga agcaggcgca ggctccgtga ggaggcaagg taagacgccg agggaggact |
| 601 | gaggcgggcc tcaccccaga cagagggccc ccaataatcc agcgctgcct ctgctgccgg |
| 661 | gcctggacca ccctgcaggg gaagacttct caggctcagt cgccaccacc tcaccccgcc |
| 721 | accccccgcc gctttaaccg cagggaactc tggcgtaaga gctttgtgtg accagggcag |
| 781 | ggctggttag aagtgctcag ggcccagact cagccaggaa tcaaggtcag gaccccaaga |
| 841 | ggggactgag ggcaacccac cccctaccct cactaccaat cccatccccc aacaccaacc |
| 901 | ccacccccat ccctcaaaca ccaaccccac ccccaaaccc cattcccatc tcctccccca |
| 961 | ccaccatcct ggcagaatcc ggctttgccc ctgcaatcaa cccacggaag ctccgggaat |
| 1021 | ggcggccaag cacgcggatc ctgacgttca catgtacggc taagggaggg aaggggttgg |
| 1081 | gtctcgtgag tatggccttt gggatgcaga ggaagggccc aggcctcctg gaagacagtg |
| 1141 | gagtccttag gggacccagc atgccaggac agggggccca ctgtacccct gtctcaaact |
| 1201 | gagccacctt ttcattcagc cgagggaatc ctagggatgc agacccactt cagcaggggg |
| 1261 | ttggggccca gcctgcgagg agtcaagggg aggaagaaga gggaggactg aggggacctt |
| 1321 | ggagtccaga tcagtggcaa ccttgggctg ggggatcctg ggcacagtgg ccgaatgtgc |
| 1381 | cccgtgctca ttgcaccttc agggtgacag agagttgagg gctgtggtct gagggctggg |
| 1441 | acttcaggtc agcagaggga ggaatcccag gatctgccgg acccaaggtg tgcccccttc |
| 1501 | atgaggactg gggatacccc cggcccagaa agaagggatg ccacagagtc tggaagtccc |
| 1561 | ttgttcttag ctctggggga acctgatcag ggatggccct aagtgacaat ctcatttgta |
| 1621 | ccacaggcag gaggttgggg aaccctcagg gagataaggt gttggtgtaa agaggagctg |
| 1681 | tctgctcatt tcagggggtt gggggttgag aaagggcagt ccctggcagg agtaaagatg |
| 1741 | agtaacccac aggaggccat cataacgttc accctagaac caaaggggtc agccctggac |
| 1801 | aacgcacgtg ggggtaacag gatgtggccc ctcctcactt gtctttccag atctcaggga |
| 1861 | gttgatgacc ttgttttcag aaggtgactc aggtcaacac aggggcccca tctggtcgac |
| 1921 | agatgcagtg gttctaggat ctgccaagca tccaggtgga gagcctgagg taggattgag |
| 1981 | ggtacccctg ggccagaatg cagcaagggg gccccataga aatctgccct gcccctgcgg |
| 2041 | ttacttcaga gaccctgggc agggctgtca gctgaagtcc ctccattatc ctgggatctt |
| 2101 | tgatgtcagg gaaggggagg ccttggtctg aaggggctgg agtcaggtca gtagagggag |
| 2161 | ggtctcaggc cctgccagga gtggacgtga ggaccaagcg gactcgtcac ccaggacacc |
| 2221 | tggactccaa tgaatttgga catctctcgt tgtccttcgc gggaggacct ggtcacgtat |
| 2281 | ggccagatgt gggtcccctc atatccttct gtaccatatc agggatgtga gttcttgaca |
| 2341 | tgagagattc tcaagccagc aaaagggtgg gattaggccc tacaaggaga aaggtgaggg |
| 2401 | ccctgagtga gcacagaggg gaccctccac ccaagtagag tggggacctc acggagtctg |
| 2461 | gccaaccctg ctgagacttc tgggaatccg tggctgtgct tgcagtctgc acactgaagg |
| 2521 | cccgtgcatt cctctcccag gaatcaggag ctccaggaac caggcagtga ggccttggtc |
| 2581 | tgagtcagtg tcctcaggtc acagagcaga ggggacgcag acagtgccaa cactgaaggt |
| 2641 | ttgcctggaa tgcacaccaa gggccccacc cgcccagaac aaatgggact ccagagggcc |
| 2701 | tggcctcacc ctccctattc tcagtcctgc agcctgagca tgtgctggcc ggctgtaccc |
| 2761 | tgaggtgccc tcccacttcc tccttcaggt tctgaggggg acaggctgac aagtaggacc |
| 2821 | cgaggcactg gaggagcatt gaaggagaag atctgtaagt aagcctttgt cagagcctcc |
| 2881 | aaggttcagt tcagttctca cctaaggcct cacacacgct ccttctctcc ccaggcctgt |
| 2941 | gggtcttcat tgcccagctc ctgcccgcac tcctgcctgc tgccctgacc agagtcatca |
| 3001 | tgcctcttga gcagaggagt cagcactgca agcctgaaga aggccttgag gcccgaggag |
| 3061 | aggccctggg cctggtgggt gcgcaggctc ctgctactga ggagcagcag accgcttctt |
| 3121 | cctcttctac tctagtggaa gttaccctgg gggaggtgcc tgctgccgac tcaccgagtc |
| 3181 | ctccccacag tcctcaggga gcctccagct tctcgactac catcaactac actctttgga |
| 3241 | gacaatccga tgagggctcc agcaaccaag aagaggaggg gccaagaatg tttcccgacc |
| 3301 | tggagtccga gttccaagca gcaatcagta ggaagatggt tgagttggtt cattttctgc |
| 3361 | tcctcaagta tcgagccagg gagccggtca caaaggcaga aatgctggag agtgtcctca |
| 3421 | gaaattgcca ggacttcttt cccgtgatct tcagcaaagc ctccgagtac ttgcagctgg |
| 3481 | tctttggcat cgaggtggtg gaagtggtcc ccatcagcca cttgtacatc cttgtcacct |
| 3541 | gcctgggcct ctcctacgat ggcctgctgg gcgacaatca ggtcatgccc aagacaggcc |
| 3601 | tcctgataat cgtcctggcc ataatcgcaa tagagggcga ctgtgcccct gaggagaaaa |
| 3661 | tctgggagga gctgagtatg ttggaggtgt ttgaggggag ggaggacagt gtcttcgcac |
| 3721 | atcccaggaa gctgctcatg caagatctgg tgcaggaaaa ctacctggag taccggcagg |
| 3781 | tgcccggcag tgatcctgca tgctacgagt tcctgtgggg tccaagggcc ctcattgaaa |
| 3841 | ccagctatgt gaaagtcctg caccatacac taaagatcgg tggagaacct cacatttcct |
| 3901 | acccacccct gcatgaacgg gctttgagag agggagaaga gtgagtctca gcacatgttg |
| 3961 | cagccagggc cagtgggagg gggtctgggc cagtgcacct tccagggccc catccattag |
| 4021 | cttccactgc ctcgtgtgat atgaggccca ttcctgcctc tttgaagaga gcagtcagca |
| 4081 | ttcttagcag tgagtttctg ttctgttgga tgactttgag atttatcttt ctttcctgtt |
| 4141 | ggaattgttc aaatgttcct tttaacaaat ggttggatga acttcagcat ccaagtttat |
| 4201 | gaatgacagt agtcacacat agtgctgttt atatagttta ggggtaagag tcctgttttt |
| 4261 | tattcagatt gggaaatcca ttccattttg tgagttgtca cataataaca gcagtggaat |
| 4321 | atgtatttgc ctatattgtg aacgaattag cagtaaaata catgatacaa ggaactcaaa |
| 4381 | agatagttaa ttcttgcctt atacctcagt ctattatgta aaattaaaaa tatgtgtatg |
| 4441 | tttttgcttc tttgagaatg caaaagaaat taaatctgaa taaattcttc ctgttcactg |
| 4501 | gctcatttct ttaccattca ctcagcatct gctctgtgga aggccctggt agtagtggg |
| // | |
| Human MAGE-3 antigen (MAGE-3) gene, complete cds. | |
| ACCESSION U03735 | |
| VERSION U03735.1 GI:468825 | |
| SEQ ID NO 73 | |
| /translation = “MPLEQRSQHCKPEEGLEARGEALGLVGAQAPATEEQEAASSSSTLVEVTLGEV | |
| PAAESPDPPQSPQGASSLPTTMNYPLWSQSYEDSSNQEEEGPSTFPDLESEFQAALSRKVAELVHFLLL | |
| KYRAREPVTKAEMLGSVVGNWQYFFPVIFSKASSSLQLVFGIELMEVDPIGHLYIFATCLGLSYDGLLG | |
| DNQIMPKAGLLIIVLAIIAREGDCAPEEKIWEELSVLEVFEGREDSILGDPKKLLTQHFVQENYLEYRQ | |
| VPGSDPACYEFLWGPRALVETSYVKVLHHMVKISGGPHISYPPLHEWVLREGEE” | |
| SEQ ID NO 83 | |
| ORIGIN | |
| 1 | acgcaggcag tgatgtcacc cagaccacac cccttccccc aatgccactt cagggggtac |
| 61 | tcagagtcag agacttggtc tgaggggagc agaagcaatc tgcagaggat ggcggtccag |
| 121 | gctcagccag gcatcaactt caggaccctg agggatgacc gaaggccccg cccacccacc |
| 181 | cccaactccc ccgaccccac caggatctac agcctcagga cccccgtccc aatccttacc |
| 241 | ccttgcccca tcaccatctt catgcttacc tccaccccca tccgatcccc atccaggcag |
| 301 | aatccagttc cacccctgcc cggaacccag ggtagtaccg ttgccaggat gtgacgccac |
| 361 | tgacttgcgc attggaggtc agaagaccgc gagattctcg ccctgagcaa cgagcgacgg |
| 421 | cctgacgtcg gcggagggaa gccggcccag gctcggtgag gaggcaaggt aagacgctga |
| 481 | gggaggactg aggcgggcct cacctcagac agagggcctc aaataatcca gtgctgcctc |
| 541 | tgctgccggg cctgggccac cccgcagggg aagacttcca ggctgggtcg ccactacctc |
| 601 | accccgccga cccccgccgc tttagccacg gggaactctg gggacagagc ttaatgtggc |
| 661 | cagggcaggg ctggttagaa gaggtcaggg cccacgctgt ggcaggaatc aaggtcagga |
| 721 | ccccgagagg gaactgaggg cagcctaacc accaccctca ccaccattcc cgtcccccaa |
| 781 | cacccaaccc cacccccatc ccccattccc atccccaccc ccacccctat cctggcagaa |
| 841 | tccgggcttt gcccctggta tcaagtcacg gaagctccgg gaatggcggc caggcacgtg |
| 901 | agtcctgagg ttcacatcta cggctaaggg agggaagggg ttcggtatcg cgagtatggc |
| 961 | cgttgggagg cagcgaaagg gcccaggcct cctggaagac agtggagtcc tgaggggacc |
| 1021 | cagcatgcca ggacaggggg cccactgtac ccctgtctca aaccgaggca ccttttcatt |
| 1081 | cggctacggg aatcctaggg atgcagaccc acttcagcag ggggttgggg cccagccctg |
| 1141 | cgaggagtca tggggaggaa gaagagggag gactgagggg accttggagt ccagatcagt |
| 1201 | ggcaaccttg ggctggggga tgctgggcac agtggccaaa tgtgctctgt gctcattgcg |
| 1261 | ccttcagggt gaccagagag ttgagggctg tggtctgaag agtgggactt caggtcagca |
| 1321 | gagggaggaa tcccaggatc tgcagggccc aaggtgtacc cccaaggggc ccctatgtgg |
| 1381 | tggacagatg cagtggtcct aggatctgcc aagcatccag gtgaagagac tgagggagga |
| 1441 | ttgagggtac ccctgggaca gaatgcggac tgggggcccc ataaaaatct gccctgctcc |
| 1501 | tgctgttacc tcagagagcc tgggcagggc tgtcagctga ggtccctcca ttatcctagg |
| 1561 | atcactgatg tcagggaagg ggaagccttg gtctgagggg gctgcactca gggcagtaga |
| 1621 | gggaggctct cagaccctac taggagtgga ggtgaggacc aagcagtctc ctcacccagg |
| 1681 | gtacatggac ttcaataaat ttggacatct ctcgttgtcc tttccgggag gacctgggaa |
| 1741 | tgtatggcca gatgtgggtc ccctcatgtt tttctgtacc atatcaggta tgtgagttct |
| 1801 | tgacatgaga gattctcagg ccagcagaag ggagggatta ggccctataa ggagaaaggt |
| 1861 | gagggccctg agtgagcaca gaggggatcc tccaccccag tagagtgggg acctcacaga |
| 1921 | gtctggccaa ccctcctgac agttctggga atccgtggct gcgtttgctg tctgcacatt |
| 1981 | gggggcccgt ggattcctct cccaggaatc aggagctcca ggaacaaggc agtgaggact |
| 2041 | tggtctgagg cagtgtcctc aggtcacaga gtagaggggg ctcagatagt gccaacggtg |
| 2101 | aaggtttgcc ttggattcaa accaagggcc ccacctgccc cagaacacat ggactccaga |
| 2161 | gcgcctggcc tcaccctcaa tactttcagt cctgcagcct cagcatgcgc tggccggatg |
| 2221 | taccctgagg tgccctctca cttcctcctt caggttctga ggggacaggc tgacctggag |
| 2281 | gaccagaggc ccccggagga gcactgaagg agaagatctg taagtaagcc tttgttagag |
| 2341 | cctccaaggt tccattcagt actcagctga ggtctctcac atgctccctc tctccccagg |
| 2401 | ccagtgggtc tccattgccc agctcctgcc cacactcccg cctgttgccc tgaccagagt |
| 2461 | catcatgcct cttgagcaga ggagtcagca ctgcaagcct gaagaaggcc ttgaggcccg |
| 2521 | aggagaggcc ctgggcctgg tgggtgcgca ggctcctgct actgaggagc aggaggctgc |
| 2581 | ctcctcctct tctactctag ttgaagtcac cctgggggag gtgcctgctg ccgagtcacc |
| 2641 | agatcctccc cagagtcctc agggagcctc cagcctcccc actaccatga actaccctct |
| 2701 | ctggagccaa tcctatgagg actccagcaa ccaagaagag gaggggccaa gcaccttccc |
| 2761 | tgacctggag tccgagttcc aagcagcact cagtaggaag gtggccgagt tggttcattt |
| 2821 | tctgctcctc aagtatcgag ccagggagcc ggtcacaaag gcagaaatgc tggggagtgt |
| 2881 | cgtcggaaat tggcagtatt tctttcctgt gatcttcagc aaagcttcca gttccttgca |
| 2941 | gctggtcttt ggcatcgagc tgatggaagt ggaccccatc ggccacttgt acatctttgc |
| 3001 | cacctgcctg ggcctctcct acgatggcct gctgggtgac aatcagatca tgcccaaggc |
| 3061 | aggcctcctg ataatcgtcc tggccataat cgcaagagag ggcgactgtg cccctgagga |
| 3121 | gaaaatctgg gaggagctga gtgtgttaga ggtgtttgag gggagggaag acagtatctt |
| 3181 | gggggatccc aagaagctgc tcacccaaca tttcgtgcag gaaaactacc tggagtaccg |
| 3241 | gcaggtcccc ggcagtgatc ctgcatgtta tgaattcctg tggggtccaa gggccctcgt |
| 3301 | tgaaaccagc tatgtgaaag tcctgcacca tatggtaaag atcagtggag gacctcacat |
| 3361 | ttcctaccca cccctgcatg agtgggtttt gagagagggg gaagagtgag tctgagcacg |
| 3421 | agttgcagcc agggccagtg ggagggggtc tgggccagtg caccttccgg ggccgcatcc |
| 3481 | cttagtttcc actgcctcct gtgacgtgag gcccattctt cactctttga agcgagcagt |
| 3541 | cagcattctt agtagtgggt ttctgttctg ttggatgact ttgagattat tctttgtttc |
| 3601 | ctgttggagt tgttcaaatg ttccttttaa cggatggttg aatgagcgtc agcatccagg |
| 3661 | tttatgaatg acagtagtca cacatagtgc tgtttatata gtttaggagt aagagtcttg |
| 3721 | ttttttactc aaattgggaa atccattcca ttttgtgaat tgtgacataa taatagcagt |
| 3781 | ggtaaaagta tttgcttaaa attgtgagcg aattagcaat aacatacatg agataactca |
| 3841 | agaaatcaaa agatagttga ttcttgcctt gtacctcaat ctattctgta aaattaaaca |
| 3901 | aatatgcaaa ccaggatttc cttgacttct ttgagaatgc aagcgaaatt aaatctgaat |
| 3961 | aaataattct tcctcttcac tggctcgttt cttttccgtt cactcagcat ctgctctgtg |
| 4021 | ggaggccctg ggttagtagt ggggatgcta aggtaagcca gactcacgcc tacccatagg |
| 4081 | gctgtagagc ctaggacctg cagtcatata attaaggtgg tgagaagtcc tgtaagatgt |
| 4141 | agaggaaatg taagagaggg gtgagggtgt ggcgctccgg gtgagagtag tggagtgtca |
| 4201 | gtgc |
| // | |
| Homo sapiens prostate stem cell antigen (PSCA) mRNA, complete cds. | |
| ACCESSION AF043498 | |
| VERSION AF043498.1 GI:2909843 | |
| SEQ ID NO 79 | |
| /translation = “MKAVLLALLMAGLALQPGTALLCYSCKAQVSNEDCLQVENCTQLGEQCWTARI | |
| RAVGLLTVISKGCSLNCVDDSQDYYVGKKNITCCDTDLCNASGAHALQPAAAILALLPALGLLLWGPGQ | |
| L” | |
| SEQ ID NO 87 | |
| ORIGIN | |
| 1 | agggagaggc agtgaccatg aaggctgtgc tgcttgccct gttgatggca ggcttggccc |
| 61 | tgcagccagg cactgccctg ctgtgctact cctgcaaagc ccaggtgagc aacgaggact |
| 121 | gcctgcaggt ggagaactgc acccagctgg gggagcagtg ctggaccgcg cgcatccgcg |
| 181 | cagttggcct cctgaccgtc atcagcaaag gctgcagctt gaactgcgtg gatgactcac |
| 241 | aggactacta cgtgggcaag aagaacatca cgtgctgtga caccgacttg tgcaacgcca |
| 301 | gcggggccca tgccctgcag ccggctgccg ccatccttgc gctgctccct gcactcggcc |
| 361 | tgctgctctg gggacccggc cagctatagg ctctgggggg ccccgctgca gcccacactg |
| 421 | ggtgtggtgc cccaggcctt tgtgccactc ctcacagaac ctggcccagt gggagcctgt |
| 481 | cctggttcct gaggcacatc ctaacgcaag tttgaccatg tatgtttgca ccccttttcc |
| 541 | ccnaaccctg accttcccat gggccttttc caggattccn accnggcaga tcagttttag |
| 601 | tganacanat ccgcntgcag atggcccctc caaccntttn tgttgntgtt tccatggccc |
| 661 | agcattttcc acccttaacc ctgtgttcag gcacttnttc ccccaggaag ccttccctgc |
| 721 | ccaccccatt tatgaattga gccaggtttg gtccgtggtg tcccccgcac ccagcagggg |
| 781 | acaggcaatc aggagggccc agtaaaggct gagatgaagt ggactgagta gaactggagg |
| 841 | acaagagttg acgtgagttc ctgggagttt ccagagatgg ggcctggagg cctggaggaa |
| 901 | ggggccaggc ctcacatttg tggggntccc gaatggcagc ctgagcacag cgtaggccct |
| 961 | taataaacac ctgttggata agccaaaaaa |
| // | |
| GLANDULAR KALLIKREIN 1 PRECURSOR (TISSUE KALLIKREIN) | |
| (KIDNEY/PANCREAS/SALIVARY GLAND KALLIKREIN). | |
| ACCESSION P06870 | |
| PID g125170 | |
| VERSION P06870 GI:125170 | |
| SEQ ID NO 105 | |
| ORIGIN | |
| 1 | mwflvlclal slggtgaapp iqsrivggwe ceqhsqpwqa alyhfstfqc ggilvhrqwv |
| 61 | ltaahcisdn yqlwlgrhnl fddentaqfv hvsesfphpg fnmsllenht rqadedyshd |
| 121 | lmllritepa dtitdavkvv elptqepevg stclasgwgs iepenfsfpd dlqcvdlkil |
| 181 | pndecekahv qkvtdfmlcv ghleggkdtc vgdsggplmc dgvlqgvtsw gyvpcgtpnk |
| 241 | psvavrvlsy vkwiedtiae ns |
| // | |
| ELASTASE 2A PRECURSOR. | |
| ACCESSION P08217 | |
| PID g119255 | |
| VERSION P08217 GI:119255 | |
| SEQ ID NO 106 | |
| ORIGIN | |
| 1 | mirtlllstl vagalscgdp typpyvtrvv ggeearpnsw pwqvslqyss ngkwyhtcgg |
| 61 | slianswvlt aahcisssrt yrvglgrhnl yvaesgslav svskivvhkd wnsnqiskgn |
| 121 | diallklanp vsltdkiqla clppagtilp nnypcyvtgw grlqtngavp dvlqqgrllv |
| 181 | vdyatcsssa wwgssvktsm icaggdgvis scngdsggpl ncqasdgrwq vhgivsfgsr |
| 241 | lgcnyyhkps vftrvsnyid winsviann |
| // | |
| pancreatic elastase IIB [Homo sapiens] | |
| ACCESSION NP_056933 | |
| PID g7705648 | |
| VERSION NP_056933.1 GI:7705648 | |
| SEQ ID NO 107 | |
| ORIGIN | |
| 1 | mirtlllstl vagalscgvs tyapdmsrml ggeearpnsw pwqvslqyss ngqwyhtcgg |
| 61 | slianswvlt aahcisssri yrvmlgqhnl yvaesgslav svskivvhkd wnsnqvskgn |
| 121 | diallklanp vsltdkiqla clppagtilp nnypcyvtgw grlqtngalp ddlkqgrllv |
| 181 | vdyatcsssg wwgstvktnm icaggdgvic tcngdsggpl ncqasdgrwe vhgigsltsv |
| 241 | lgcnyyykps iftrvsnynd winsviann |
| // | |
| PRAME Homo sapiens preferentially expressed antigen in melanoma | |
| (PRAME), mRNA. | |
| ACCESSION NM_006115 | |
| VERSION NM_006115.1 GI:5174640 | |
| SEQ ID NO 77 | |
| /translation = “MERRRLWGSIQSRYISMSVWTSPRRLVELAGQSLLKDEALAIAALELLPRELF | |
| PPLFMAAFDGRHSQTLKANVQAWPFTCLPLGVLMKGQHLHLETFKAVLDGLDVLLAQEVRPRRWKLQVL | |
| DLRKNSHQDFWTVWSGNRASLYSFPEPEAAQPMTKKRKVDGLSTEAEQPFIPVEVLVDLFLKEGACDEL | |
| FSYLIEKVKRKKNVLRLCCKKLKIFAMPMQDIKMILKMVQLDSTEDLEVTCTWKLPTLAKFSPYLGQMI | |
| NLRRLLLSHIHASSYISPEKEEQYIAQFTSQFLSLQCLQALYVDSLFFLRGRLDQLLRHVMNPLETLSI | |
| TNCRLSEGDVMHLSQSPSVSQLSVLSLSGVMLTDVSPEPLQALLERASATLQDLVFDECGITDDQLLAL | |
| LPSLSHCSQLTTLSFYGNSISISALQSLLQHLIGLSNLTHVLYPVPLESYEDIHGTLHLERLAYLHARL | |
| RELLCELGRPSMVWLSANPCPHCGDRTFYDPEPILCPCFMPN” | |
| SEQ ID NO 85 | |
| ORIGIN | |
| 1 | gcttcagggt acagctcccc cgcagccaga agccgggcct gcagcccctc agcaccgctc |
| 61 | cgggacaccc cacccgcttc ccaggcgtga cctgtcaaca gcaacttcgc ggtgtggtga |
| 121 | actctctgag gaaaaaccat tttgattatt actctcagac gtgcgtggca acaagtgact |
| 181 | gagacctaga aatccaagcg ttggaggtcc tgaggccagc ctaagtcgct tcaaaatgga |
| 241 | acgaaggcgt ttgtggqgtt ccattcagag ccgatacatc agcatgagtg tgtggacaag |
| 301 | cccacggaga cttgtggagc tggcagggca gagcctgctg aaggatgagg ccctggccat |
| 361 | tgccgccctg gagttgctgc ccagggagct cttcccgcca ctcttcatgg cagcctttga |
| 421 | cgggagacac agccagaccc tgaaggcaat ggtgcaggcc tggcccttca cctgcctccc |
| 481 | tctgggagtg ctgatgaagg gacaacatct tcacctggag accttcaaag ctgtgcttga |
| 541 | tggacttgat gtgctccttg cccaggaggt tcgccccagg aggtggaaac ttcaagtgct |
| 601 | ggatttacgg aagaactctc atcaggactt ctggactgta tggtctggaa acagggccag |
| 661 | tctgtactca tttccagagc cagaagcagc tcagcccatg acaaagaagc gaaaagtaga |
| 721 | tggtttgagc acagaggcag agcagccctt cattccagta gaggtgctcg tagacctgtt |
| 781 | cctcaaggaa ggtgcctgtg atgaattgtt ctcctacctc attgagaaag tgaagcgaaa |
| 841 | gaaaaatgta ctacgcctgt gctgtaagaa gctgaagatt tttgcaatgc ccatgcagga |
| 901 | tatcaagatg atcctgaaaa tggtgcagct ggactctatt gaagatttgg aagtgacttg |
| 961 | tacctggaag ctacccacct tggcgaaatt ttctccttac ctgggccaga tgattaatct |
| 1021 | gcgtagactc ctcctctccc acatccatgc atcttcctac atttccccgg agaaggaaga |
| 1081 | gcagtatatc gcccagttca cctctcagtt cctcagtctg cagtgcctgc aggctctcta |
| 1141 | tgtggactct ttatttttcc ttagaggccg cctggatcag ttgctcaggc acgtgatgaa |
| 1201 | ccccttggaa accctctcaa taactaactg ccggctttcg gaaggggatg tgatgcatct |
| 1261 | gtcccagagt cccagcgtca gtcagctaag tgtcctgagt ctaagtgggg tcatgctgac |
| 1321 | cgatgtaagt cccgagcccc tccaagctct gctggagaga gcctctgcca ccctccagga |
| 1381 | cctggtcttt gatgagtgtg ggatcacgga tgatcagctc cttgccctcc tgccttccct |
| 1441 | gagccactgc tcccagctta caaccttaag cttctacggg aattccatct ccatatctgc |
| 1501 | cttgcagagt ctcctgcagc acctcatcgg gctgagcaat ctgacccacg tgctgtatcc |
| 1561 | tgtccccctg gagagttatg aggacatcca tggtaccctc cacctggaga ggcttgccta |
| 1621 | tctgcatgcc aggctcaggg aqttgctgtg tgagttgggg cggcccagca tggtctggct |
| 1681 | tagtgccaac ccctgtcctc actgtgggga cagaaccttc tatgacccgg agcccatcct |
| 1741 | gtgcccctgt ttcatgccta actagctggg tgcacatatc aaatgcttca ttctgcatac |
| 1801 | ttggacacta aagccaggat gtgcatgcat cttgaagcaa caaagcagcc acagtttcag |
| 1861 | acaaatgttc agtgtgagtg aggaaaacat gttcagtgag gaaaaaacat tcagacaaat |
| 1921 | gttcagtgag gaaaaaaagg ggaagttggg gataggcaga tgttgacttg aggagttaat |
| 1981 | gtgatctttg gggagataca tcttatagag ttagaaatag aatctgaatt tctaaaggga |
| 2041 | gattctggct tgggaagtac atgtaggaqt taatccctgt gtagactgtt gtaaagaaac |
| 2101 | tgttgaaaat aaagagaagc aatgtgaagc aaaaaaaaaa aaaaaaaa |
| // | |
| CEA Homo sapiens carcinoembryonic antigen-related cell adhesion | |
| molecule 5 (CEACAM5), mRNA. | |
| ACCESSION NM_004363 | |
| VERSION NM_004363.1 GI:11386170 | |
| SEQ ID NO 88 | |
| /translation = “MESPSAPPHRWCIPWQRLLLTASLLTFWNPPTTAKLTIESTPFNVAEGKEVLL | |
| LVHNLPQHLFGYSWYKGERVDGNRQIIGYVIGTQQATPGPAYSGREIIYPNASLLIQNIIQNDTGFYTL | |
| HVIKSDLVNEEATGQFRVYPELPKPSISSNNSKPVEDKDAVAFTCEPETQDATYLWWVNNQSLPVSPRL | |
| QLSNGNRTLTLFNVTRNDTASYKCETQNPVSARRSDSVILNVLYGPDAPTISPLNTSYRSGENLNLSCH | |
| AASNPPAQYSWFVNGTFQQSTQELFIPNITVNNSGSYTCQAHNSDTGLNRTTVTTITVYAEPPKPFITS | |
| NNSNPVEDEDAVALTCEPEIQNTTYLWWVNNQSLPVSPRLQLSNDNRTLTLLSVTRNDVGPYECGIQNE | |
| LSVDHSDPVILNVLYGPDDPTISPSYTYYRPGVNLSLSCHAASNPPAQYSWLIDGNIQQHTQELFISNI | |
| TEKNSGLYTCQANNSASGHSRTTVKTITVSAELPKPSISSNNSKPVEDKDAVAFTCEPEAQNTTYLWWV | |
| NGQSLPVSPRLQLSNGNRTLTLFNVTRNDARAYVCGIQNSVSANRSDPVTLDVLYGPDTPIISPPDSSY | |
| LSGANLNLSCHSASNPSPQYSWRINGIPQQHTQVLFIAKITPNNNGTYACFVSNLATGRNNSIVKSITV | |
| SASGTSPGLSAGATVGIMIGVLVGVALI” | |
| SEQ ID NO 89 | |
| ORIGIN | |
| 1 | ctcagggcag agggaggaag gacagcagac cagacagtca cagcagcctt gacaaaacgt |
| 61 | tcctggaact caagctcttc tccacagagg aggacagagc agacagcaga gaccatggag |
| 121 | tctccctcgg cccctcccca cagatggtgc atcccctggc agaggctcct gctcacagcc |
| 181 | tcacttctaa ccttctggaa cccgcccacc actgccaagc tcactattga atccacgccg |
| 241 | ttcaatgtcg cagaggggaa ggaggtgctt ctacttgtcc acaatctgcc ccagcatctt |
| 301 | tttggctaca gctggtacaa aggtgaaaga gtggatggca accgtcaaat tataggatat |
| 361 | gtaataggaa ctcaacaagc taccccaggg cccgcataca gtggtcgaga gataatatac |
| 421 | cccaatgcat ccctgctgat ccagaacatc atccagaatg acacaggatt ctacacccta |
| 481 | cacgtcataa agtcagatct tgtgaatgaa gaagcaactg gccagttccg ggtatacccg |
| 541 | gagctgccca agccctccat ctccagcaac aactccaaac ccgtggagga caaggatgct |
| 601 | gtggccttca cctgtgaacc tgagactcag gacgcaacct acctgtggtg ggtaaacaat |
| 661 | cagagcctcc cggtcagtcc caggctgcag ctgtccaatg gcaacaggac cctcactcta |
| 721 | ttcaatgtca caagaaatga cacagcaagc tacaaatgtg aaacccagaa cccagtgagt |
| 781 | gccaggcgca gtgattcagt catcctgaat gtcctctatg gcccggatgc ccccaccatt |
| 841 | tcccctctaa acacatctta cagatcaggg gaaaatctga acctctcctg ccacgcagcc |
| 901 | tctaacccac ctgcacagta ctcttggttt gtcaatggga ctttccagca atccacccaa |
| 961 | gagctcttta tccccaacat cactgtgaat aatagtggat cctatacgtg ccaagcccat |
| 1021 | aactcagaca ctggcctcaa taggaccaca gtcacgacga tcacagtcta tgcagagcca |
| 1081 | cccaaaccct tcatcaccag caacaactcc aaccccgtgg aggatgagga tgctgtagcc |
| 1141 | ttaacctgtg aacctgagat tcagaacaca acctacctgt ggtgggtaaa taatcagagc |
| 1201 | ctcccggtca gtcccaggct gcagctgtcc aatgacaaca ggaccctcac tctactcagt |
| 1261 | gtcacaagga atgatgtagg accctatgag tgtggaatcc agaacgaatt aagtgttgac |
| 1321 | cacagcgacc cagtcatcct gaatgtcctc tatggcccag acgaccccac catttccccc |
| 1381 | tcatacacct attaccgtcc aggggtgaac ctcagcctct cctgccatgc agcctctaac |
| 1441 | ccacctgcac agtattcttg gctgattgat gggaacatcc agcaacacac acaagagctc |
| 1501 | tttatctcca acatcactga gaagaacagc ggactctata cctgccaggc caataactca |
| 1561 | gccagtggcc acagcaggac tacagtcaag acaatcacag tctctgcgga gctgcccaag |
| 1621 | ccctccatct ccagcaacaa ctccaaaccc gtggaggaca aggatgctgt ggccttcacc |
| 1681 | tgtgaacctg aggctcagaa cacaacctac ctgtggtggg taaatggtca gagcctccca |
| 1741 | gtcagtccca ggctgcagct gtccaatggc aacaggaccc tcactctatt caatgtcaca |
| 1801 | agaaatgacg caagagccta tgtatgtgga atccagaact cagtgagtgc aaaccgcagt |
| 1861 | gacccagtca ccctggatgt cctctatggg ccggacaccc ccatcatttc ccccccagac |
| 1921 | tcgtcttacc tttcgggagc gaadctcaac ctctcctgcc actcggcctc taacccatcc |
| 1981 | ccgcagtatt cttggcgtat caatgggata ccgcagcaac acacacaagt tctctttatc |
| 2041 | gccaaaatca cgccaaataa taacgggacc tatgcctgtt ttgtctctaa cttggctact |
| 2101 | ggccgcaata attccatagt caagagcatc acagtctctg catctggaac ttctcctggt |
| 2161 | ctctcagctg gggccactgt cggcatcatg attggagtgc tggttggggt tgctctgata |
| 2221 | tagcagccct ggtgtagttt cttcatttca ggaagactga cagttgtttt gcttcttcct |
| 2281 | taaagcattt gcaacagcta cagtctaaaa ttgcttcttt accaaggata tttacagaaa |
| 2341 | agactctgac cagagatcga gaccatccta gccaacatcg tgaaacccca tctctactaa |
| 2401 | aaatacaaaa atgagctggg cttggtggcg cgcacctgta gtcccagtta ctcgggaggc |
| 2461 | tgaggcagga gaatcgcttg aacccgggag gtggagattg cagtgagccc agatcgcacc |
| 2521 | actgcactcc agtctggcaa cagagcaaga ctccatctca aaaagaaaag aaaagaagac |
| 2581 | tctgacctgt actcttgaat acaagtttct gataccactg cactgtctga gaatttccaa |
| 2641 | aactttaatg aactaactga cagcttcatg aaactgtcca ccaagatcaa gcagagaaaa |
| 2701 | taattaattt catgggacta aatgaactaa tgaggattgc tgattcttta aatgtcttgt |
| 2761 | ttcccagatt tcaggaaact ttttttcttt taagctatcc actcttacag caatttgata |
| 2821 | aaatatactt ttgtgaacaa aaattgagac atttacattt tctccctatg tggtcgctcc |
| 2881 | agacttggga aactattcat gaatatttat attgtatggt aatatagtta ttgcacaagt |
| 2941 | tcaataaaaa tctgctcttt gtataacaga aaaa |
| // | |
| H r2/Neu Human tyrosine kinase-type receptor (HER2) mRNA, complete | |
| cds. | |
| ACCESSION M11730 | |
| VERSION M11730.1 GI:183986 | |
| SEQ ID NO 90 | |
| /translation = “MELAALCRWGLLLALLPPGAASTQVCTGTDMKLRLPASPETHLDMLRHLYQGC | |
| QVVQGNLELTYLPTNASLSFLQDIQEVQGYVLIAHNQVRQVPLQRLRIVRGTQLFEDNYALAVLDNGDP | |
| LNNTTPVTGASPGGLRELQLRSLTEILKGGVLIQRNPQLCYQDTILWKDIFHKNNQLALTLIDTNRSRA | |
| CHPCSPMCKGSRCWGESSEDCQSLTRTVCAGGCARCKGPLPTDCCHEQCAAGCTGPKHSDCLACLHFNH | |
| SGICELHCPALVTYNTDTFESMPNPEGRYTFGASCVTACPYNYLSTDVGSCTLVCPLHNQEVTAEDGTQ | |
| RCEKCSKPCARVCYGLGMEHLREVRAVTSANIQEFAGCKKIFGSLAFLPESFDGDPASNTAPLQPEQLQ | |
| VFETLEEITGYLYISAWPDSLPDLSVFQNLQVIRGRILHNGAYSLTLQGLGISWLGLRSLRELGSGLAL | |
| IHHNTRLCFVHTVPWDQLFRNPHQALLHTANRPEDECVGEGLACHQLCARGHCWGPGPTQCVNCSQFLR | |
| GQECVEECRVLQGLPREYVNARHCLPCHPECQPQNGSVTCFGPEADQCVACAHYKDPPFCVARCPSGVK | |
| PDLSYMPIWKFPDEEGACQPCPINCTHSCVDLDDKGCPAEQRASPLTSIVSAVVGILLVVVLGVVFGIL | |
| IKRRQQKIRKYTMRRLLQETELVEPLTPSGANPNQAQMRILKETELRKVKVLGSGAFGTVYKGIWIPDG | |
| ENVKIPVAIKVLRENTSPKANKEILDEAYVMAGVGSPYVSRLLGICLTSTVQLVTQLMPYGCLLDHVRE | |
| NRGRLGSQDLLNWCMQIAKGMSYLEDVRLVHRDLAARNVLVKSPNHVKITDFGLARLLDIDETEYHADG | |
| GKVPIKWMALESILRRRFTHQSDVWSYGVTVWELMTFGAKPYDGIPAREIPDLLEKGERLPQPPICTID | |
| VYMIMVKCWMIDSECRPRFRELVSEFSRMARDPQRFVVIQNEDLGPASPLDSTFYRSLLEDDDMGDLVD | |
| AEEYLVPQQGFFCPDPAPGAGGMVHHRHRSSSTRSGGGDLTLGLEPSEEEAPRSPLAPSEGAGSDVFDG | |
| DLGMGAAKGLQSLPTHDPSPLQRYSEDPTVPLPSETDGYVAPLTCSPQPEYVNQPDVRPQPPSPREGPL | |
| PAARPAGATLERAKTLSPGKNGVVKDVFAFGGAVENPEYLTPQGGAAPQPHPPPAFSPAFDNLYYWDQD | |
| PPERGAPPSTFKGTPTAENPEYLGLDVPV” | |
| SEQ ID NO 91 | |
| ORIGIN Chromosome 17q21-q22. | |
| 1 | aattctcgag ctcgtcgacc ggtcgacgag ctcgagggtc gacgagctcg agggcgcgcg |
| 61 | cccggccccc acccctcgca gcaccccgcg ccccgcgccc tcccagccgg gtccagccgg |
| 121 | agccatgggg ccggagccgc agtgagcacc atggagctgg cggccttgtg ccgctggggg |
| 181 | ctcctcctcg ccctcttgcc ccccggagcc gcgagcaccc aagtgtgcac cggcacagac |
| 241 | atgaagctgc ggctccctgc cagtcccgag acccacctgg acatgctccg ccacctctac |
| 301 | cagggctgcc aggtggtgca gggaaacctg gaactcacct acctgcccac caatgccagc |
| 361 | ctgtccttcc tgcaggatat ccaggaggtg cagggctacg tgctcatcgc tcacaaccaa |
| 421 | gtgaggcagg tcccactgca gaggctgcgg attgtgcgag gcacccagct ctttgaggac |
| 481 | aactatgccc tggccgtgct agacaatgga gacccgctga acaataccac ccctgtcaca |
| 541 | ggggcctccc caggaggoct gcgggagctg cagcttcgaa gcctcacaga gatcttgaaa |
| 601 | ggaggggtct tgatccagcg gaacccccag ctctgctacc aggacacgat tttgtggaag |
| 661 | gacatcttcc acaagaacaa ccagctggct ctcacactga tagacaccaa ccgctctcgg |
| 721 | gcctgccacc cctgttctcc gatgtgtaag ggctcccgct gctggggaga gagttctgag |
| 781 | gattgtcaga gcctgacgcg cactgtctgt gccggtggct gtgcccgctg caaggggcca |
| 841 | ctgcccactg actgctgcca tgagcagtgt gctgccggct gcacgggccc caagcactct |
| 901 | gactgcctgg cctgcctcca cttcaaccac agtggcatct gtgagctgca ctgcccagcc |
| 961 | ctggtcacct acaacacaga cacgtttgag tccatgccca atcccgaggg ccggtataca |
| 1021 | ttcggcgcca gctgtgtgac tgcctgtccc tacaactacc tttctacgga cgtgggatcc |
| 1081 | tgcaccctcg tctgccccct gcacaaccaa gaggtgacag cagaggatgg aacacagcgg |
| 1141 | tgtgagaagt gcagcaagcc ctgtgcccga gtgtgctatg gtctgggcat ggagcacttg |
| 1201 | cgagaggtga gggcagttac cagtgccaat atccaggagt ttgctggctg caagaagatc |
| 1261 | tttgggagcc tggcatttct gccggagagc tttgatgggg acccagcctc caacactgcc |
| 1321 | ccgctccagc cagagcagct ccaagtgttt gagactctgg aagagatcac aggttaccta |
| 1381 | tacatctcag catggccgga cagcctgcct gacctcagcg tcttccagaa cctgcaagta |
| 1441 | atccggggac gaattctgca caatggcgcc tactcgctga ccctgcaagg gctgggcatc |
| 1501 | agctggctgg ggctgcgctc actgagggaa ctgggcagtg gactggccct catccaccat |
| 1561 | aacacccacc tctgcttcgt gcacacggtg ccctgggacc agctctttcg gaacccgcac |
| 1621 | caagctctgc tccacactgc caaccggcca gaggacgagt gtgtgggcga gggcctggcc |
| 1681 | tgccaccagc tgtgcgcccg agggcactgc tggggtccag ggcccaccca gtgtgtcaac |
| 1741 | tgcagccagt tccttcgggg ccaggagtgc gtggaggaat gccgagtact gcaggggctc |
| 1801 | cccagggagt atgtgaatgc caggcactgt ttgccgtgcc accctgagtg tcagccccag |
| 1861 | aatggctcag tgacctgttt tggaccggag gctgaccagt gtgtggcctg tgcccactat |
| 1921 | aaggaccctc ccttctgcgt ggcccgctgc cccagcggtg tgaaacctga cctctcctac |
| 1981 | atgcccatct ggaagtttcc agatgaggag ggcgcatgcc agccttgccc catcaactgc |
| 2041 | acccactcct gtgtggacct ggatgacaag ggctgccccg ccgagcagag agccagccct |
| 2101 | ctgacgtcca tcgtctctgc ggtggttggc attctgctgg tcgtggtctt gggggtggtc |
| 2161 | tttgggatcc tcatcaagcg acggcagcag aagatccgga agtacacgat gcggagactg |
| 2221 | ctgcaggaaa cggagctggt ggagccgctg acacctagcg gagcgatgcc caaccaggcg |
| 2281 | cagatgcgga tcctgaaaga gacggagctg aggaaggtga aggtgcttgg atctggcgct |
| 2341 | tttggcacag tctacaaggg catctggatc cctgatgggg agaatgtgaa aattccagtg |
| 2401 | gccatcaaag tgttgaggga aaacacatcc cccaaagcca acaaagaaat cttagacgaa |
| 2461 | gcatacgtga tggctggtgt gggctcccca tatgtctccc gccttctggg catctgcctg |
| 2521 | acatccacgg tgcagctggt gacacagctt atgccctatg gctgcctctt agaccatgtc |
| 2581 | cgggaaaacc gcggacgcct gggctcccag gacctgctga actggtgtat ggagattgcc |
| 2641 | aaggggatga gctacctgga ggatgtgcgg ctcgtacaca gggacttggc cgctcggaac |
| 2701 | gtgctggtca agagtcccaa ccatgtcaaa attacagact tcgggctggc tcggctgctg |
| 2761 | gacattgacg agacagagta ccatgcagat gggggcaagg tgcccatcaa gtggatggcg |
| 2821 | ctggagtcca ttctccgccg gcggttcacc caccagagtg atgtgtggag ttatggtgtg |
| 2881 | actgtgtggg agctgatgac ttttggggcc aaaccttacg atgggatccc agcccgggag |
| 2941 | atccctgacc tgctggaaaa gggggagcgg ctgccccagc cccccatctg caccattgat |
| 3001 | gtctacatga tcatggtcaa atgttggatg attgactctg aatgtcggcc aagattccgg |
| 3061 | gagttggtgt ctgaattctc ccgcatggcc agggaccccc agcgctttgt ggtcatccag |
| 3121 | aatgaggact tgggcccagc cagtcccttg gacagcacct tctaccgctc actgctggag |
| 3181 | gacgatgaca tgggggacct ggtggatgct gaggagtatc tggtacccca gcagggcttc |
| 3241 | ttctgtccag accctgcccc gggcgctggg ggcatggtcc accacaggca ccgcagctca |
| 3301 | tctaccagga gtggcggtgg ggacctgaca ctagggctgg agccctctga agaggaggcc |
| 3361 | cccaggtctc cactggcacc ctccgaaggg gctggctccg atgtatttga tggtgacctg |
| 3421 | ggaatggggg cagccaaggg gctgcaaagc ctccccacac atgaccccag ccctctacag |
| 3481 | cggtacagtg aggaccocac agtacccctg ccctctgaga ctgatggcta cgttgccccc |
| 3541 | ctgacctgca gcccccagcc tgaatatgtg aaccagccag atgttcggcc ccagccccct |
| 3601 | tcgccccgag agggccctct gcctgctgcc cgacctgctg gtgccactct ggaaagggcc |
| 3661 | aagactctct ccccagggaa gaatggggtc gtcaaagacg tttttgcctt tgggggtgcc |
| 3721 | gtggagaacc ccgagtactt gacaccccag ggaggagctg cccctcagcc ccaccctcct |
| 3781 | cctgccttca gcccagcctt cgacaacctc tattactggg accaggaccc accagagcgg |
| 3841 | ggggctccac ccagcacctt caaagggaca cctacggcag agaacccaga gtacctgggt |
| 3901 | ctggacgtgc cagtgtgaac cagaaggcca agtccgcaga agccctgatg tgtcctcagg |
| 3961 | gagcagggaa ggcctgactt ctgctggcat caagaggtgg gagggccctc cgaccacttc |
| 4021 | caggggaacc tgccatgcca ggaacctgtc ctaaggaacc ttccttcctg cttgagttcc |
| 4081 | cagatggctg gaaggggtcc agcctcgttg gaagaggaac agcactgggg agtctttgtg |
| 4141 | gattctgagg ccctgcccaa tgagactcta gggtccagtg gatgccacag cccagcttgg |
| 4201 | ccctttcctt ccagatcctg ggtactgaaa gccttaggga agctggcctg agaggggaag |
| 4261 | cggccctaag ggagtgtcta agaacaaaag cgacccattc agagactgtc cctgaaacct |
| 4321 | agtactgccc cccatgagga aggaacagca atggtgtcag tatccaggct ttgtacagag |
| 4381 | tgcttttctg tttagttttt actttttttg ttttgttttt ttaaagacga aataaagacc |
| 4441 | caggggagaa tgggtgttgt atggggaggc aagtgtgggg ggtccttctc cacacccact |
| 4501 | ttgtccattt gcaaatatat tttggaaaac |
| // | |
| H. sapiens mRNA for SCP1 protein. | |
| ACCESSION X95654 | |
| VERSION X95654.1 GI:1212982 | |
| SEQ ID NO 92 | |
| /translation = “MEKQKPFALFVPPRSSSSQVSAVKPQTLGGDSTFFKSFNKCTEDDLEFPFAKT | |
| NLSKNGENIDSDPALQKVNFLPVLEQVGNSDCHYQEGLKDSDLENSEGLSRVFSKLYKEAEKIKKWKVS | |
| TEAELRQKESKLQENRKIIEAQRKAIQELQFGNEKVSLKLEEGIQENKDLIKENNATRHLCNLLKETCA | |
| RSAEKTKKYEYEREETRQVYMDLNNNIEKMITAHGELRVQAENSRLEMHFKLKEDYEKIQHLEQEYKKE | |
| INDKEKQVSLLLIQITEKENKMKDLTFLLEESRDKVNQLEEKTKLQSENLKQSIEKQHHLTKELEDIKV | |
| SLQRSVSTQKALEEDLQIATKTICQLTEEKETQMEESNKARAAHSFVVTEFETTVCSLEELLRTEQQRL | |
| EKNEDQLKILTMELQKKSSELEEMTKLTNNKEVELEELKKVLGEKETLLYENKQFEKIAEELKGTEQEL | |
| IGLLQAREKEVRDLEIQLTAITTSEQYYSKEVKDLKTELENEKLKNTELTSHCNKLSLENKELTQETSD | |
| MTLELKNQQEDINNNKKQEERMLKQIENLQETETQLRNELEYVREELKQKRDEVKCKLDKSEENCNNLR | |
| KQVENKNKYIEELQQENKALKKKGTAESKQLNVYEIKVNKLELELESAKQKFGEITDTYQKEIEDKKIS | |
| EENLLEEVEKAKVIADEAVKLQKEIDKRCQHKIAEMVALMEKHKHQYDKIIEERDSELGLYKSKEQEQS | |
| SLRASLEIELSNLKAELLSVKKQLEIEREEKEKLKREAKENTATLKEKKDKKTQTFLLETPEIYWKLDS | |
| KAVPSQTVSRNFTSVDHGISKDKRDYLWTSAKNTLSTPLPKAYTVKTPTKPKLQQRENLNIPIEESKKK | |
| RKMAFEFDINSDSSETTDLLSMVSEEETLKTLYRNNNPPASHLCVKTPKKAPSSLTTPGPTLKFGAIRK | |
| MREDRWAVIAKMDRKKKLKEAEKLFV” | |
| SEQ ID NO 93 | |
| ORIGIN | |
| 1 | gccctcatag accgtttgtt gtagttcgcg tgggaacagc aacccacggt ttcccgatag |
| 61 | ttcttcaaag atatttacaa ccgtaacaga gaaaatggaa aagcaaaagc cctttgcatt |
| 121 | gttcgtacca ccgagatcaa gcagcagtca ggtgtctgcg gtgaaacctc agaccctggg |
| 181 | aggcgattcc actttcttca agagtttcaa caaatgtact gaagatgatt tggagtttcc |
| 241 | atttgcaaag actaatctct ccaaaaatgg ggaaaacatt gattcagatc ctgctttaca |
| 301 | aaaagttaat ttcttgcccg tgcttgagca ggttggtaat tctgactgtc actatcagga |
| 361 | aggactaaaa gactctgatt tggagaattc agagggattg agcagagtgt tttcaaaact |
| 421 | gtataaggag gctgaaaaga taaaaaaatg gaaagtaagt acagaagctg aactgagaca |
| 481 | gaaagaaagt aagttgcaag aaaacagaaa gataattgaa gcacagcgaa aagccattca |
| 541 | ggaactgcaa tttggaaatg aaaaagtaag tttgaaatta gaagaaggaa tacaagaaaa |
| 601 | taaagattta ataaaagaga ataatgccac aaggcattta tgtaatctac tcaaagaaac |
| 661 | ctgtgctaga tctgcagaaa agacaaagaa atatgaatat gaacgggaag aaaccaggca |
| 721 | agtttatatg gatctaaata ataacattga gaaaatgata acagctcatg gggaacttcg |
| 781 | tgtgcaagct gagaattcca gactggaaat gcattttaag ttaaaggaag attatgaaaa |
| 841 | aatccaacac cttgaacaag aatacaagaa ggaaataaat gacaaggaaa agcaggtatc |
| 901 | actactattg atccaaatca ctgagaaaga aaataaaatg aaagatttaa catttctgct |
| 961 | agaggaatcc agagataaag ttaatcaatt agaggaaaag acaaaattac agagtgaaaa |
| 1021 | cttaaaacaa tcaattgaga aacagcatca tttgactaaa gaactagaag atattaaagt |
| 1081 | gtcattacaa agaagtgtga gtactcaaaa ggctttagag gaagatttac agatagcaac |
| 1141 | aaaaacaatt tgtcagctaa ctgaagaaaa agaaactcaa atggaagaat ctaataaagc |
| 1201 | tagagctgct cattcgtttg tggttactga atttgaaact actgtctgca gcttggaaga |
| 1261 | attattgaga acagaacagc aaagattgga aaaaaatgaa gatcaattga aaatacttac |
| 1321 | catggagctt caaaagaaat caagtgagct ggaagagatg actaagctta caaataacaa |
| 1381 | agaagtagaa cttgaagaat tgaaaaaagt cttgggagaa aaggaaacac ttttatatga |
| 1441 | aaataaacaa tttgagaaga ttgctgaaga attaaaagga acagaacaag aactaattgg |
| 1501 | tcttctccaa gccagagaga aagaagtac.a tgatttggaa atacagttaa ctgccattac |
| 1561 | cacaagtgaa cagtattatt caaaagaggt taaagatcta aaaactgagc ttgaaaacga |
| 1621 | gaagcttaag aatactgaat taacttcaca ctgcaacaag ctttcactag aaaacaaaga |
| 1681 | gctcacacag gaaacaagtg atatgaccct agaactcaag aatcagcaag aagatattaa |
| 1741 | taataacaaa aagcaagaag aaaggatgtt gaaacaaata gaaaatcttc aagaaacaga |
| 1801 | aacccaatta agaaatgaac tagaatatgt gagagaagag ctaaaacaga aaagagatga |
| 1861 | agttaaatgt aaattggaca agagtgaaga aaattgtaac aatttaagga aacaagttga |
| 1921 | aaataaaaac aagtatattg aagaacttca gcaggagaat aaggccttga aaaaaaaagg |
| 1981 | tacagcagaa agcaagcaac tgaatgttta tgagataaag gtcaataaat tagagttaga |
| 2041 | actagaaagt gccaaacaga aatttggaga aatcacagac acctatcaga aagaaattga |
| 2101 | ggacaaaaag atatcagaag aaaatctttt ggaagaggtt gagaaagcaa aagtaatagc |
| 2161 | tgatgaagca gtaaaattac agaaagaaat tgataagcga tgtcaacata aaatagctga |
| 2221 | aatggtagca cttatggaaa aacataagca ccaatatgat aagatcattg aagaaagaga |
| 2281 | ctcagaatta ggactttata agagcaaaga acaagaacag tcatcactga gagcatcttt |
| 2341 | ggagattgaa ctatccaatc tcaaagctga acttttgtct gttaagaagc aacttgaaat |
| 2401 | agaaagagaa gagaaggaaa aactcaaaag agaggcaaaa gaaaacacag ctactcttaa |
| 2461 | agaaaaaaaa gacaagaaaa cacaaacatt tttattggaa acacctgaaa tttattggaa |
| 2521 | attggattct aaagcagttc cttcacaaac tgtatctcga aatttcacat cagttgatca |
| 2581 | tggcatatcc aaagataaaa gagactatct gtggacatct gccaaaaata ctttatctac |
| 2641 | accattgcca aaggcatata cagtgaagac accaacaaaa ccaaaactac agcaaagaga |
| 2701 | aaacttgaat atacccattg aagaaagtaa aaaaaagaga aaaatggcct ttgaatttga |
| 2761 | tattaattca gatagttcag aaactactga tcttttgagc atggtttcag aagaagagac |
| 2821 | attgaaaaca ctgtatagga acaataatcc accagcttct catctttgtg tcaaaacacc |
| 2881 | aaaaaaggcc ccttcatctc taacaacccc tggacctaca ctgaagtttg gagctataag |
| 2941 | aaaaatgcgg gaggaccgtt gggctgtaat tgctaaaatg gatagaaaaa aaaaactaaa |
| 3001 | agaagctgaa aagttatttg tttaatttca gagaatcagt gtagttaagg agcctaataa |
| 3061 | cgtgaaactt atagttaata ttttgttctt atttgccaga gccacatttt atctggaagt |
| 3121 | tgagacttaa aaaatacttg catgaatgat ttgtgtttct ttatattttt agcctaaatg |
| 3181 | ttaactacat attgtctgga aacctgtcat tgtattcaga taattagatg attatatatt |
| 3241 | gttgttactt tttcttgtat tcatgaaaac tgtttttact aagttttcaa atttgtaaag |
| 3301 | ttagcctttg aatgctagga atgcattatt gagggtcatt ctttattctt tactattaaa |
| 3361 | atattttgga tgcaaaaaaa aaaaaaaaaa aaa |
| // | |
| Homo sapiens synovial sarcoma, X breakpoint 4 (SSX4), mRNA. | |
| ACCESSION NM_005636 | |
| VERSION NM_005636.1 GI:5032122 | |
| SEQ ID NO 94 | |
| /translation = “MNGDDAFARRPRDDAQISEKLRKAFDDIAKYFSKKEWEKMKSSEKIVYVYMKL | |
| NYEVMTKLGFKVTLPPFMRSKRAADFHGNDFGNDRNHRNQVERPQMTFGSLQRIFPKIMPKKPAEEENG | |
| LKEVPEASGPQNDGKQLCPPGNPSTLEKINKTSGPKRGKHAWTHRLRERKQLVVYEEISDPEEDDE” | |
| SEQ ID NO 95 | |
| ORIGIN | |
| 1 | atgaacggag acgacgcctt tgcaaggaga cccagggatg atgctcaaat atcagagaag |
| 61 | ttacgaaagg ccttcgatga tattgccaaa tacttctcta agaaagagtg ggaaaagatg |
| 121 | aaatcctcgg agaaaatcgt ctatgtgtat atgaagctaa actatgaggt catgactaaa |
| 181 | ctaggtttca aggtcaccct cccacctttc atgcgtagta aacgggctgc agacttccac |
| 241 | gggaatgatt ttggtaacga tcgaaaccac aggaatcagg ttgaacgtcc tcagatgact |
| 301 | ttcggcagcc tccagagaat cttcccgaag atcatgccca agaagccagc agaggaagaa |
| 361 | aatggtttga aggaagtgcc agaggcatct ggcccacaaa atgatgggaa acagctgtgc |
| 421 | cccccgggaa atccaagtac cttggagaag attaacaaga catctggacc caaaaggggg |
| 481 | aaacatgcct ggacccacag actgcgtgag agaaagcagc tggtggttta tgaagagatc |
| 541 | agcgaccctg aggaagatga cgagtaactc ccctcg |
| U19142. Human GAGE-1 prot . . . [gi:914898] | |
| LOCUS H5U19142 646 bp mRNA linear | |
| DEFINITION Human GAGE-1 protein mRNA, complete cds. | |
| ACCESSION U19142 | |
| VERSION U19142.1 GI:914898 | |
| SEQ ID No. 96 | |
| /translation = “MSWRGRSTYRPRPRRYVEPPEMIGPMRPEQFSDEVEPATPEEGEPATQRQDPA | |
| AAQEGEDEGASAGQGPKPEADSQEQGHPQTGCECEDGPDGQEMDPPNPEEVKTPEEEMRSHYVAQTGIL | |
| WLLMNNCFLNLSPRKP” | |
| SEQ ID NO. 97 | |
| 1 | ctgccgtccg gactcttttt cctctactga gattcatctg tgtgaaatat gagttggcga |
| 61 | ggaagatcga cctatcggcc tagaccaaga cgctacgtag agcctcctga aatgattggg |
| 121 | cctatgcggc ccgagcagtt cagtgatgaa gtggaaccag caacacctga agaaggggaa |
| ] | |
| 181 | ccagcaactc aacgtcagga tcctgcagct gctcaggagg gagaggatga gggagcatct |
| 241 | gcaggtcaag ggccgaagcc tgaagctgat agccaggaac agggtcaccc acagactggg |
| 301 | tgtgagtgtg aagatggtcc tgatgggcag gagatggacc cgccaaatcc agaggaggtg |
| 361 | aaaacgcctg aagaagagat gaggtctcac tatgttgccc agactgggat tctctggctt |
| 421 | ttaatgaaca attgcttctt aaatctttcc ccacggaaac cttgagtgac tgaaatatca |
| 481 | aatggcgaga gaccgtttag ttcctatcat ctgtggcatg tgaagggcaa tcacagtgtt |
| 541 | aaaagaagac atgctgaaat gttgcaggct gctcctatgt tggaaaattc ttcattgaag |
| 601 | ttctcccaat aaagctttac agccttctgc aaagaaaaaa aaaaaa |
| // | |
| NM_001168. Homo sapiens bacu . . . [gi:4502144] | |
| LOCUS BIRC5 1619 bp mRNA linear | |
| DEFINITION Homo sapiens baculoviral IAP repeat-containing 5 | |
| (survivin) (BIRC5), mRNA. | |
| ACCESSION NM_001168 | |
| VERSION NM_001168.1 GI:4502144 | |
| SEQ ID NO. 98 | |
| /translation = “MGAPTLPPAWQPFLKDHRISTFKNWPFLEGCACTPERMAEAGFIHCPTENEPD | |
| LAQCFFCFKELEGWEPDDDPIEEHKKHSSGCAFLSVKKQFEELTLGEFLKLDRERAKNKIAKETNNKKK | |
| EFEETAKKVRPAIEQLAAMD” | |
| SEQ ID NO. 99 | |
| 1 | ccgccagatt tgaatcgcgg gacccgttgg cagaggtggc ggcggcggca tgggtgcccc |
| 61 | gacgttgccc cctgcctggc agccctttct caaggaccac cgcatctcta cattcaagaa |
| 121 | ctggcccttc ttggagggct gcgcctgcac cccggagcgg atggccgagg ctggcttcat |
| 181 | ccactgcccc actgagaacg agccagactt ggcccagtgt ttcttctgct tcaaggagct |
| 241 | ggaaggctgg gagccagatg acgaccccat agaggaacat aaaaagcatt cgtccggttg |
| 301 | cgctttcctt tctgtcaaga agcagtttga agaattaacc cttggtgaat ttttgaaact |
| 361 | ggacagagaa agagccaaga acaaaattgc aaaggaaacc aacaataaga agaaagaatt |
| 421 | tgaggaaact gcgaagaaag tgcgccgtgc catcgagcag ctggctgcca tggattgagg |
| 481 | cctctggccg gagctgcctg gtcccagagt ggctgcacca cttccagggt ttattccctg |
| 541 | gtgccaccag ccttcctgtg ggccccttag caatgtctta ggaaaggaga tcaacatttt |
| 601 | caaattagat gtttcaactg tgctcctgtt ttgtcttgaa agtggcacca gaggtgcttc |
| 661 | tgcctgtgca gcgggtgctg ctggtaacag tggctgcttc tctctctctc tctctttttt |
| 721 | gggggctcat ttttgctgtt ttgattcccg ggcttaccag gtgagaagtg agggaggaag |
| 781 | aaggcagtgt cccttttgct agagctgaca gctttgttcg cgtgggcaga gccttccaca |
| 841 | gtgaatgtgt ctggacctca tgttgttgag gctgtcacag tcctgagtgt ggacttggca |
| 901 | ggtgcctgtt gaatctgagc tgcaggttcc ttatctgtca cacctgtgcc tcctcagagg |
| 961 | acagtttttt tgttgttgtg tttttttgtt tttttttttt ggtagatgca tgacttgtgt |
| 1021 | gtgatgagag aatggagaca gagtccctgg ctcctctact gtttaacaac atggctttct |
| 1081 | tattttgttt gaattgttaa ttcacagaat agcacaaact acaattaaaa ctaagcacaa |
| 1141 | agccattcta agtcattggg gaaacggggt gaacttcagg tggatgagga gacagaatag |
| 1201 | agtgatagga agcgtctggc agatactcct tttgccactg ctgtgtgatt agacaggccc |
| 1261 | agtgagccgc ggggcacatg ctggccgctc ctccctcaga aaaaggcagt ggcctaaatc |
| 1321 | ctttttaaat gacttggctc gatgctgtgg gggactggct gggctgctgc aggccgtgtg |
| 1381 | tctgtcagcc caaccttcac atctgtcacg ttctccacac gggggagaga cgcagtccgc |
| 1441 | ccaggtcccc gctttctttg gaggcagcag ctcccgcagg gctgaagtct ggcgtaagat |
| 1501 | gatggatttg attcgccctc ctccctgtca tagagctgca gggtggattg ttacagcttc |
| 1561 | gctggaaacc tctggaggtc atctcggctg ttcctgagaa ataaaaagcc tgtcatttc |
| // | |
| U06452. Human melanoma an . . . [gi:476131] | |
| LOCUS H5U06452 1524 bp mRNA linear | |
| DEFINITION Human melanoma antigen recognized by T-cells (MART-1) | |
| mRNA. | |
| ACCESSION U06452 | |
| VERSION U06452.1 GI:476131 | |
| SEQ ID NO. 100 | |
| /translation = “MPREDAHFIYGYPKKGHGHSYTTAEEAAGIGILTVILGVLLLIGCWYCRRRNG | |
| YRALMDKSLHVGTQCALTRRCPQEGFDHRDSKVSLQEKNCEPVVPNAPPAYEKLSAEQSPPPYSP” | |
| SEQ ID NO. 101 | |
| 1 | agcagacaga ggactctcat taaggaaggt gtcctgtgcc ctgaccctac aagatgccaa |
| 61 | gagaagatgc tcacttcatc tatggttacc ccaagaaggg gcacggccac tcttacacca |
| 121 | cggctgaaga ggccgctggg atcggcatcc tgacagtgat cctgggagtc ttactgctca |
| 181 | tcggctgttg gtattgtaga agacgaaatg gatacagagc cttgatggat aaaagtcttc |
| 241 | atgttggcac tcaatgtgcc ttaacaagaa gatgcccaca agaagggttt gatcatcggg |
| 301 | acagcaaagt gtctcttcaa gagaaaaact gtgaacctgt ggttcccaat gctccacctg |
| 361 | cttatgagaa actctctgca gaacagtcac caccacctta ttcaccttaa gagccagcga |
| 421 | gacacctgag acatgctgaa attatttctc tcacactttt gcttgaattt aatacagaca |
| 481 | tctaatgttc tcctttggaa tggtgtagga aaaatgcaag ccatctctaa taataagtca |
| 541 | gtgttaaaat tttagtaggt ccgctagcag tactaatcat gtgaggaaat gatgagaaat |
| 601 | attaaattgg gaaaactcca tcaataaatg ttgcaatgca tgatactatc tgtgccagag |
| 661 | gtaatgttag taaatccatg gtgttatttt ctgagagaca gaattcaagt gggtattctg |
| 721 | gggccatcca atttctcttt acttgaaatt tggctaataa caaactagtc aggttttcga |
| 781 | accttgaccg acatgaactg tacacagaat tgttccagta ctatggagtg ctcacaaagg |
| 841 | atacttttac aggttaagac aaagggttga ctggcctatt tatctgatca agaacatgtc |
| 901 | agcaatgtct ctttgtgctc taaaattcta ttatactaca ataatatatt gtaaagatcc |
| 961 | tatagctctt tttttttgag atggagtttc gcttttgttg cccaggctgg agtgcaatgg |
| 1021 | cgcgatcttg gctcaccata acctccgcct cccaggttca agcaattctc ctgccttagc |
| 1081 | ctcctgagta gctgggatta caggcgtgcg ccactatgcc tgactaattt tgtagtttta |
| 1141 | gtagagacgg ggtttctcca tgttggtcag gctggtctca aactcctgac ctcaggtgat |
| 1201 | ctgcccgcct cagcctccca aagtgctgga attacaggcg tgagccacca cgcctggctg |
| 1261 | gatcctatat cttaggtaag acatataacg cagtctaatt acatttcact tcaaggctca |
| 1321 | atgctattct aactaatgac aagtattttc tactaaacca gaaattggta gaaggattta |
| 1381 | aataagtaaa agctactatg tactgcctta gtgctgatgc ctgtgtactg ccttaaatgt |
| 1441 | acctatggca atttagctct cttgggttcc caaatccctc tcacaagaat gtgcagaaga |
| 1501 | aatcataaag gatcagagat tctg |
| // | |

U19180. Human B melanoma . . . [qi:726039]
LOCUS HSU19180 1004 bp mRNA linear
DEFINITION Human B melanoma antigen (BAGE) mRNA, complete cds.
ACCESSION U19180
VERSION U19180.1 GI:726039
SEQ IS NO. 102
/translation = “MAARAVFLALSAQLLQARLMKEESPVVSWRLEPEDGTALCFIF”
SEQ ID NO. 103

| | |
|---|---|
| 1 | cgccaattta gggtctccgg tatctcccgc tgagctgctc tgttcccggc ttagaggacc |
| 61 | aggagaaggg ggagctggag gctggagcct gtaacaccgt ggctcgtctc actctggatg |
| 121 | gtggtggcaa cagagatggc agcgcagctg gagtgttagg agggcggcct gagcggtagg |
| 181 | agtggggctg gagcagtaag atggcggcca gagcggtttt tctggcattg tctgcccagc |
| 241 | tgctccaagc caggctgatg aaggaggagt cccctgtggt gagctggagg ttggagcctg |
| 301 | aagacggcac agctctgtgc ttcatcttct gaggttgtgg cagccacggt gatggagacg |
| 361 | gcagctcaac aggagcaata ggaggagatg gagtttcact gtgtcagcca ggatggtctc |
| 421 | gatctcctga cctcgtgatc cgcccgcctt ggccttccaa agtgccgaga ttacagcgat |
| 481 | gtgcattttg taagcacttt ggagccacta tcaaatgctg tgaagagaaa tgtacccaga |
| 541 | tgtatcatta tccttgtgct gcaggagccg gctcctttca ggatttcagt cacatcttcc |
| 601 | tgctttgtcc agaacacatt gaccaagctc ctgaaagatg taagtttact acgcatagac |
| 661 | ttttaaactt caaccaatgt atttactgaa aataacaaat gttgtaaatt ccctgagtgt |
| 721 | tattctactt gtattaaaag gtaataatac ataatcatta aaatctgagg gatcattgcc |
| 781 | agagattgtt ggggagggaa atgttatcaa cggtttcatt gaaattaaat ccaaaaagtt |
| 841 | atttcctcag aaaaatcaaa taaagtttgc atgtttttta ttcttaaaac attttaaaaa |
| 901 | ccactgtaga atgatgtaaa tagggactgt gcagtatttc tgacatatac tataaaatta |
| 961 | ttaaaaagtc aatcagtatt caacatcttt tacactaaaa agcc |
| // | |

The entire contents of all patents and publications discussed herein are incorporated by reference in their entirety to the same extent as if each individual publication was specifically and individually indicated to be incorporated by reference in its entirety Furthermore, the teachings and embodiments disclosed in any of the publications, including patents, patent publications and non-patent publications, disclosed herein are contemplated as supporting principals and embodiments related to and useful in connection with the present invention.

The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions indicates the exclusion of equivalents of the features shown and described or portions thereof. It is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of the embodiments of this invention.

What is claimed is:

1. A polypeptide, comprising a component selected from the group consisting of:
(i) a polypeptide epitope having the sequence as disclosed in TABLE 1B;
(ii) an epitope cluster comprising the polypeptide of (i);
(iii) a polypeptide having substantial similarity to (i) or (ii);
(iv) a polypeptide having functional similarity to any of (i) through (iii); and
(v) a nucleic acid encoding the polypeptide of any of (i) through (iv).

2. The polypeptide of claim 1, wherein the polypeptide is immunologically active.

3. The polypeptide of claim 1, wherein the polypeptide is less than about 30 amino acids in length.

4. The polypeptide of claim 1, wherein the polypeptide is 8 to 10 amino acids in length.

5. The polypeptide of claim 1, wherein the substantial or functional similarity comprises addition of at least one amino acid.

6. The polypeptide of claim 5, wherein the at least one additional amino acid is at an N-terminus of the polypeptide.

7. The polypeptide of claim 1, wherein the substantial or functional similarity comprises a substitution of at least one amino acid.

8. The polypeptide of claim 1, the polypeptide having affinity to an HLA-A2 molecule.

9. The polypeptide of claim 8, wherein the affinity is determined by an assay of binding.

10. The polypeptide of claim 8, wherein the affinity is determined by an assay of restriction of epitope recognition.

11. The polypeptide of claim 8, wherein the affinity is determined by a prediction algorithm.

12. The polypeptide of claim 1, the polypeptide having affinity to an HLA-B7 or HLA-B51 molecule.

13. The polypeptide of claim 1, wherein the polypeptide is a housekeeping epitope.

14. The polypeptide of claim 1, wherein the polypeptide corresponds to an epitope displayed on a tumor cell.

15. The polypeptide of claim 1, wherein the polypeptide corresponds to an epitope displayed on a neovasculature cell.

16. The polypeptide of claim 1, wherein the polypeptide is an immune epitope.

17. The polypeptide of claim 1, wherein the polypeptide is encoded by a nucleic acid.

18. A composition comprising the polypeptide of claim 1 and a pharmaceutically acceptable adjuvant, carrier, diluent, or excipient.

19. The composition of claim 18, where the adjuvant is a polynucleotide.

20. The composition of claim 19 wherein the polynucleotide comprises a CpG dinucleotide.

21. The composition of claim 18, wherein the adjuvant is encoded by a polynucleotide.

22. The composition of claim 18 wherein the adjuvant is a cytokine.

23. The composition of claim 23 wherein the cytokine is GM-CSF.

24. The composition of claim 18 further comprising a professional antigen-presenting cell (pAPC).

25. The composition of claim 18, further comprising a second epitope.

26. The composition of claim 25, wherein the second epitope is a polypeptide.

27. The composition of claim 25, wherein the second epitope is a nucleic acid.

28. The composition of claim 25, wherein the second epitope is a housekeeping epitope.

29. The composition of claim 25, wherein the second epitope is an immune epitope.

30. A recombinant construct comprising the nucleic acid of claim 1.

31. The construct of claim 30, further comprising a plasmid, a viral vector, a bacterial vector, or an artificial chromosome.

32. The construct of claim 30, further comprising a sequence encoding at least one feature selected from the group consisting of a second epitope, an IRES, an ISS, an NIS, and ubiquitin.

33. A composition comprising at least one component selected from the group consisting of the epitope of claim 1; a composition comprising the polypeptide or nucleic acid of claim 1; a composition comprising an isolated T cell expressing a T cell receptor specific for an MHC-peptide complex, the complex comprising the polypeptide of claim 1; a recombinant construct comprising the nucleic acid of claim 1; an isolated T cell expressing a T cell receptor specific for an MHC-peptide complex, the complex comprising the polypeptide of claim 1; a host cell expressing a recombinant construct comprising a nucleic acid encoding a T cell receptor binding domain specific for an MHC-peptide complex and a composition comprising the same, and a host cell expressing a recombinant construct comprising the nucleic acid of claim 1 and a composition comprising the same; with a pharmaceutically acceptable adjuvant, carrier, diluent, or excipient.

34. A method of treating an animal, comprising:
administering to an animal the composition of claim 33.

35. The method of claim 34, wherein the administering step comprises a mode of delivery selected from the group consisting of transdermal, intranodal, perinodal, oral, intravenous, intradermal, intramuscular, intraperitoneal, mucosal, aerosol inhalation, and instillation.

36. The method of claim 34, further comprising a step of assaying to determine a characteristic indicative of a state of a target cell or target cells.

37. The method of claim 36, comprising a first assaying step and a second assaying step, wherein the first assaying step precedes the administering step, and wherein the second assaying step follows the administering step.

38. The method of claim 37, further comprising a step of comparing the characteristic determined in the first assaying step with the characteristic determined in the second assaying step to obtain a result.

39. The method of claim 38, wherein the result is selected from the group consisting of: evidence of an immune response, a diminution in number of target cells, a loss of mass or size of a tumor comprising target cells, a decrease in number or concentration of an intracellular parasite infecting target cells.

40. A method of making a vaccine, comprising:
combining at least one component selected from the group consisting of the polypeptide of claim 1; a composition comprising the polypeptide or nucleic acid of claim 1; a composition comprising an isolated T cell expressing a T cell receptor specific for an MHC-peptide complex, the complex comprising the polypeptide of claim 1; a composition comprising a host cell expressing a recombinant construct, the construct comprising the nucleic acid of claim 1, or the construct encoding a protein molecule comprising the binding domain of a T cell receptor specific for an MHC-peptide complex; a recombinant construct comprising the nucleic acid of claim 1; an isolated T cell expressing a T cell receptor specific for an MHC-peptide complex, the complex comprising the polypeptide of claim 1; and a host cell expressing a recombinant construct, the construct comprising the nucleic acid of claim 1, or the construct encoding a protein molecule comprising the binding domain of a T cell receptor specific for an MHC-peptide complex; with a pharmaceutically acceptable adjuvant, carrier, diluent, or excipient.


SEQUENCE LISTING (610 sequences)

SEQ ID NO: 1 - protein, 10 aa, Homo sapiens
FLPWHRLFLL

SEQ ID NO: 2 - protein, 529 aa, Homo sapiens [sequence omitted]

SEQ ID NO: 3 - protein, 188 aa, Homo sapiens
MNGDDAFARRPTVGAQIPEKIQKAFDDIAKYFSKEEWEKMKASEKIFYVYMKRKYEAMTKLGFKATLPPFMCNKRAEDFQGNDLDNDPNRGNQVERPQMTFGRLQGISPKIMPKKPAEEGNDSEEVPEASGPQNDGKELCPPGKPTTSEKIHERSGPKRGEHAWTHRLRERKQLVIYEEISDPEEDDE

SEQ ID NO: 4 - protein, 750 aa, Homo sapiens
MWNLLHETDSAVATARRPRWLCAGALVLAGGFFLLGFLFGWFIKSSNEATNITPKHNMKAFLDELKAENIKKFLYNFTQIPHLAGTEQNFQLAKQIQSQWKEFGLDSVELAHYDVLLSYPNKTHPNYISIINEDGNEIFNTSLFEPPPPGYENVSDIVPPFSAFSPQGMPEGDLVYVNYARTEDFFKLERDMKINCSGKIVIARYGKVFRGNKVKNAQLAGAKGVILYSDPADYFAPGVKSYPDGWNLPGGGVQRGNILNLNGAGDPLTPGYPANEYAYRRGIAEAVGLPSIPVHPIGYYDAQKLLEKMGGSAPPDSSWRGSLKVPYNVGPGFTGNFSTQKVKMHIHSTNEVTRIYNVIGTLRGAVEPDRYVILGGHRDSWVFGGIDPQSGAAVVHEIVRSFGTLKKEGWRPRRTILFASWDAEEFGLLGSTEWAEENSRLLQERGVAYINADSSIEGNYTLRVDCTPLMYSLVHNLTKELKSPDEGFEGKSLYESWTKKSPSPEFSGMPRISKLGSGNDFEVFFQRLGIASGRARYTKNWETNKFSGYPLYHSVYETYELVEKFYDPMFKYHLTVAQVRGGMVFELANSIVLPFDCRDYAVVLRKYADKIYSISMKHPQEMKTYSVSFDSLFSAVKNFTEIASKFSERLQDFDKSNPIVLRMMNDQLMFLERAFIDPLGLPDRPFYRHVIYAPSSHNKYAGESFPGIYDALFDIESKVDPSKAWGEVKRQIYVAAFTVQAAAETLSEVA

SEQ ID NO: 5 - DNA, 1964 bp, Homo sapiens
atcactgtagtagtagctggaaagagaaatctgtgactccaattagccagttcctgcagaccttgtgaggactagaggaagaatgctcctggctgttttgtactgcctgctgtggagtttccagacctccgctggccatttccctagagcctgtgtctcctctaagaacctgatggagaaggaatgctgtccaccgtggagcggggacaggagtccctgtggccagctttcaggcagaggttcctgtcagaatatccttctgtccaatgcaccacttgggcctcaatttcccttcacaggggtggatgaccgggagtcgtggccttccgtcttttataataggacctgccagtgctctggcaacttcatgggattcaactgtggaaactgcaagtttggcttttggggaccaaactgcacagagagacgactcttggtgagaagaaacatcttcgatttgagtgccccagagaaggacaaattttttgcctacctcactttagcaaagcataccatcagctcagactatgtcatccccatagggacctatggccaaatgaaaaatggatcaacacccatgtttaacgacatcaatatttatgacctctttgtctggatgcattattatgtgtcaatggatgcactgcttgggggatctgaaatctggagagacattgattttgcccatgaagcaccagcttttctgccttggcatagactcttcttgttgcggtgggaacaagaaatccagaagctgacaggagatgaaaacttcactattccatattgggactggcgggatgcagaaaagtgtgacatttgcacagatgagtacatgggaggtcagcaccccacaaatcctaacttactcagcccagcatcattcttctcctcttggcagattgtctgtagccgattggaggagtacaacagccatcagtctttatgcaatggaacgcccgagggacctttacggcgtaatcctggaaaccatgacaaatccagaaccccaaggctcccctcttcagctgatgtagaattttgcctgagtttgacccaatatgaatctggttccatggataaagctgccaatttcagctttagaaatacactggaaggatttgctagtccacttactgggatagcggatgcctctcaaagcagcatgcacaatgccttgcacatctatatgaatggaacaatgtcccaggtacagggatctgccaacgatcctatcttccttcttcaccatgcatttgttgacagtatttttgagcagtggctccgaaggcaccgtcctcttcaagaagtttatccagaagccaatgcacccattggacataaccgggaatcctacatggttccttttataccactgtacagaaatggtgatttctttatttcatccaaagatctgggctatgactatagctatctacaagattcagacccagactcttttcaagactacattaagtcctatttggaacaagcgagtcggatctggtcatggctccttggggcggcgatggtaggggccgtcctcactgccctgctggcagggcttgtgagcttgctgtgtcgtcacaagagaaagcagcttcctgaagaaaagcagccactcctcatggagaaagaggattaccacagcttgtatcagagccatttataaaaggcttaggcaatagagtagggccaaaaagcctgacctcactctaactcaaagtaatgtccaggttcccagagaatatctgctggtatttttctgtaaagaccatttgcaaaattgtaacctaatacaaagtgtagccttcttccaactcaggtagaacacacctgtctttgtcttgctgttttcactcagcccttttaacattttcccctaagcccatatgtctaaggaaaggatgctatttggtaatgaggaactgttatttgtatgtgaattaaagtgctcttatttt

SEQ ID NO: 6 - DNA, 766 bp, Homo sapiens
ctctctttcgattcttccatactcagagtacgcacggtctgattttctctttggattcttccaaaatcagagtcagactgctcccggtgccatgaacggagacgacgcctttgcaaggagacccacggttggtgctcaaataccagagaagatccaaaaggccttcgatgatattgccaaatacttctctaaggaagagtgggaaaagatgaaagcctcggagaaaatcttctatgtgtatatgaagagaaagtatgaggctatgactaaactaggtttcaaggccaccctcccacctttcatgtgtaataaacgggccgaagacttccaggggaatgatttggataatgaccctaaccgtgggaatcaggttgaacgtcctcagatgactttcggcaggctccagggaatctccccgaagatcatgcccaagaagccagcagaggaaggaaatgattcggaggaagtgccagaagcatctggcccacaaaatgatgggaaagagctgtgccccccgggaaaaccaactacctctgagaagattcacgagagatctggacccaaaaggggggaacatgcctggacccacagactgcgtgagagaaaacagctggtgatttatgaagagatcagcgaccctgaggaagatgacgagtaactcccctcagggatacgacacatgcccatgatgagaagcagaacgtggtgacctttcacgaacatgggcatggctgcggacccctcgtcatcaggtgcatagcaagtg

SEQ ID NO: 7 - DNA, 2653 bp, Homo sapiens
ctcaaaaggggccggatttccttctcctggaggcagatgttgcctctctctctcgctcggattggttcagtgcactctagaaacactgctgtggtggagaaactggaccccaggtctggagcgaattccagcctgcagggctgataagcgaggcattagtgagattgagagagactttaccccgccgtggtggttggagggcgcgcagtagagcagcagcacaggcgcgggtcccgggaggccggctctgctcgcgccgagatgtggaatctccttcacgaaaccgactcggctgtggccaccgcgcgccgcccgcgctggctgtgcgctggggcgctggtgctggcgggtggcttctttctcctcggcttcctcttcgggtggtttataaaatcctccaatgaagctactaacattactccaaagcataatatgaaagcatttttggatgaattgaaagctgagaacatcaagaagttcttatataattttacacagataccacatttagcaggaacagaacaaaactttcagcttgcaaagcaaattcaatcccagtggaaagaatttggcctggattctgttgagctagcacattatgatgtcctgttgtcctacccaaataagactcatcccaactacatctcaataattaatgaagatggaaatgagattttcaacacatcattatttgaaccacctcctccaggatatgaaaatgtttcggatattgtaccacctttcagtgctttctctcctcaaggaatgccagagggcgatctagtgtatgttaactatgcacgaactgaagacttctttaaattggaacgggacatgaaaatcaattgctctgggaaaattgtaattgccagatatgggaaagttttcagaggaaataaggttaaaaatgcccagctggcaggggccaaaggagtcattctctactccgaccctgctgactactttgctcctggggtgaagtcctatccagatggttggaatcttcctggaggtggtgtccagcgtggaaatatcctaaatctgaatggtgcaggagaccctctcacaccaggttacccagcaaatgaatatgcttataggcgtggaattgcagaggctgttggtcttccaagtattcctgttcatccaattggatactatgatgcacagaagctcctagaaaaaatgggtggctcagcaccaccagatagcagctggagaggaagtctcaaagtgccctacaatgttggacctggctttactggaaacttttctacacaaaaagtcaagatgcacatccactctaccaatgaagtgacaagaatttacaatgtgataggtactctcagaggagcagtggaaccagacagatatgtcattctgggaggtcaccgggactcatgggtgtttggtggtattgaccctcagagtggagcagctgttgttcatgaaattgtgaggagctttggaacactgaaaaaggaagggtggagacctagaagaacaattttgtttgcaagctgggatgcagaagaatttggtcttcttggttctactgagtgggcagaggagaattcaagactccttcaagagcgtggcgtggcttatattaatgctgactcatctatagaaggaaactacactctgagagttgattgtacaccgctgatgtacagcttggtacacaacctaacaaaagagctgaaaagccctgatgaaggctttgaaggcaaatctctttatgaaagttggactaaaaaaagtccttccccagagttcagtggcatgcccaggataagcaaattgggatctggaaatgattttgaggtgttcttccaacgacttggaattgcttcaggcagagcacggtatactaaaaattgggaaacaaacaaattcagcggctatccactgtatcacagtgtctatgaaacatatgagttggtggaaaagttttatgatccaatgtttaaatatcacctcactgtggcccaggttcgaggagggatggtgtttgagctagccaattccatagtgctcccttttgattgtcgagattatgctgtagttttaagaaagtatgctgacaaaatctacagtatttctatgaaacatccacaggaaatgaagacatacagtgtatcatttgattcacttttttctgcagtaaagaattttacagaaattgcttccaagttcagtgagagactccaggactttgacaaaagcaacccaatagtattaagaatgatgaatgatcaactcatgtttctggaaagagcatttattgatccattagggttaccagacaggcctttttataggcatgtcatctatgctccaagcagccacaacaagtatgcaggggagtcattcccaggaatttatgatgctctgtttgatattgaaagcaaagtggacccttccaaggcctggggagaagtgaagagacagatttatgttgcagccttcacagtgcaggcagctgcagagactttgagtgaagtagcctaagaggattctttagagaatccgtattgaatttgtgtggtatgtcactcagaaagaatcgtaatgggtatattgataaattttaaaattggtatatttgaaataaagttgaatattatatataaaaaaaaaaaaaaaaaaa

SEQ ID NO: 8 - protein, 9 aa, Homo sapiens
FLPWHRLFL

SEQ ID NO: 9 - protein, 9 aa, Homo sapiens
LPWHRLFLL

SEQ ID NO: 10 - protein, 38 aa, Homo sapiens
YFSKEEWEKMKASEKIFYVYMKRKYEAMTKLGFKATLP

SEQ ID NO: 11 - protein, 9 aa, Homo sapiens
FSKEEWEKM

SEQ ID NO: 12 - protein, 9 aa, Homo sapiens
KMKASEKIF

SEQ ID NO: 13 - protein, 9 aa, Homo sapiens
MKASEKIFY

SEQ ID NO: 14 - protein, 10 aa, Homo sapiens
KMKASEKIFY

SEQ ID NO: 15 - protein, 9 aa, Homo sapiens
KASEKIFYV

SEQ ID NO: 16 - protein, 10 aa, Homo sapiens
MKASEKIFYV

SEQ ID NO: 17 - protein, 10 aa, Homo sapiens
KASEKIFYVY

SEQ ID NO: 18 - protein, 9 aa, Homo sapiens
ASEKIFYVY

SEQ ID NO: 19 - protein, 9 aa, Homo sapiens
RKYEAMTKL

SEQ ID NO: 20 - protein, 10 aa, Homo sapiens
KRKYEAMTKL

SEQ ID NO: 21 - protein, 10 aa, Homo sapiens
KYEAMTKLGF

SEQ ID NO: 22 - protein, 9 aa, Homo sapiens
YEAMTKLGF

SEQ ID NO: 23 - protein, 8 aa, Homo sapiens
EAMTKLGF

SEQ ID NO: 24 - protein, 10 aa, Homo sapiens
FLPSDYFPSV

SEQ ID NO: 25 - protein, 9 aa, Homo sapiens
AEMGKYSFY

SEQ ID NO: 26 - protein, 9 aa, Homo sapiens
KYSEKISYV

SEQ ID NO: 27 - protein, 9 aa, Homo sapiens
KVSEKIVYV

SEQ ID NO: 28 - protein, 9 aa, Homo sapiens
KSSEKIVYV

SEQ ID NO: 29 - protein, 9 aa, Homo sapiens
KASEKIIYV

SEQ ID NO: 30 - protein, 30 aa, Homo sapiens
AFSPQGMPEGDLVYVNYARTEDFFKLERDM

SEQ ID NO: 31 - protein, 23 aa, Homo sapiens
GMPEGDLVYVNYARTEDFFKLER

SEQ ID NO: 32 - protein, 9 aa, Homo sapiens
MPEGDLVYV

SEQ ID NO: 33 - protein, 10 aa, Homo sapiens
GMPEGDLVYV

SEQ ID NO: 34 - protein, 9 aa, Homo sapiens
GMPEGDLVY

SEQ ID NO: 35 - protein, 10 aa, Homo sapiens
QGMPEGDLVY

SEQ ID NO: 36 - protein, 8 aa, Homo sapiens
MPEGDLVY

SEQ ID NO: 37 - protein, 9 aa, Homo sapiens
EGDLVYVNY

SEQ ID NO: 38 - protein, 10 aa, Homo sapiens
PEGDLVYVNY

SEQ ID NO: 39 - protein, 10 aa, Homo sapiens
LVYVNYARTE

SEQ ID NO: 40 - protein, 9 aa, Homo sapiens
VNYARTEDF

SEQ ID NO: 41 - protein, 10 aa, Homo sapiens
YVNYARTEDF

SEQ ID NO: 42 - protein, 9 aa, Homo sapiens
NYARTEDFF

SEQ ID NO: 43 - protein, 8 aa, Homo sapiens
YARTEDFF

SEQ ID NO: 44 - protein, 9 aa, Homo sapiens
RTEDFFKLE

SEQ ID NO: 45 - protein, 30 aa, Homo sapiens
RGIAEAVGLPSIPVHPIGYYDAQKLLEKMG

SEQ ID NO: 46 - protein, 25 aa, Homo sapiens
IAEAVGLPSIPVHPIGYYDAQKLLE

SEQ ID NO: 47 - protein, 9 aa, Homo sapiens
LPSIPVHPI

SEQ ID NO: 48 - protein, 10 aa, Homo sapiens
GLPSIPVHPI

SEQ ID NO: 49 - protein, 9 aa, Homo sapiens
IGYYDAQKL

SEQ ID NO: 50 - protein, 10 aa, Homo sapiens
PIGYYDAQKL

SEQ ID NO: 51 - protein, 9 aa, Homo sapiens
SIPVHPIGY

SEQ ID NO: 52 - protein, 10 aa, Homo sapiens
PSIPVHPIGY

SEQ ID NO: 53 - protein, 8 aa, Homo sapiens
IPVHPIGY

SEQ ID NO: 54 - protein, 9 aa, Homo sapiens
YYDAQKLLE

SEQ ID NO: 55 - protein, 27 aa, Homo sapiens
SSIEGNYTLRVDCTPLMYSLVHLTKEL

SEQ ID NO: 56 - protein, 9 aa, Homo sapiens
IEGNYTLRV

SEQ ID NO: 57 - protein, 10 aa, Homo sapiens
SIEGNYTLRV

SEQ ID NO: 58 - protein, 8 aa, Homo sapiens
EGNYTLRV

SEQ ID NO: 59 - protein, 9 aa, Homo sapiens
TLRVDCTPL

SEQ ID NO: 60 - protein, 10 aa, Homo sapiens
YTLRVDCTPL

SEQ ID NO: 61 - protein, 9 aa, Homo sapiens
LRVDCTPLM

SEQ ID NO: 62 - protein, 9 aa, Homo sapiens
RVDCTPLMY

SEQ ID NO: 63 - protein, 10 aa, Homo sapiens
LRVDCTPLMY

SEQ ID NO: 64 - protein, 35 aa, Homo sapiens
FDKSNPIVLRMMNDQLMFLERAFIDPLGLPDRPFY

SEQ ID NO: 65 - protein, 22 aa, Homo sapiens
VLRMMNDQLMFLERAFIDPLGL

SEQ ID NO: 66 - protein, 9 aa, Homo sapiens
MMNDQLMFL

SEQ ID NO: 67 - protein, 10 aa, Homo sapiens
RMMNDQLMFL

SEQ ID NO: 68 - protein, 9 aa, Homo sapiens
RMMNDQLMF

SEQ ID NO: 69 - protein, 17 aa, Homo sapiens
MLLAVLYCLLWSFQTSA

SEQ ID NO: 70 - protein, 661 aa, Homo sapiens [sequence omitted]

SEQ ID NO: 71 - protein, 309 aa, Homo sapiens
MSLEQRSLHCKPEEALEAQQEALGLVCVQAATSSSSPLVLGTLEEVPTAGSTDPPQSPQGASAFPTTINFTRQRQPSEGSSSREEEGPSTSCILESLFRAVITKKVADLVGFLLLKYRAREPVTKAEMLESVIKNYKHCFPEIFGKASESLQLVFGIDVKEADPTGHSYVLVTCLGLSYDGLLGDNQIMPKTGFLIIVLVMIAMEGGHAPEEEIWEELSVMEVYDGREHSAYGEPRKLLTQDLVQEKYLEYRQVPDSDPARYEFLWGPRALAETSYVKVLEYVIKVSARVRFFFPSLREAALREEEEGV

SEQ ID NO: 72 - protein, 314 aa, Homo sapiens
MPLEQRSQHCKPEEGLEARGEALGLVGAQAPATEEQQTASSSSTLVEVTLGEVPAADSPSPPHSPQGASSFSTTINYTLWRQSDEGSSNQEEEGPRMFPDLESEFQAAISRKMVELVHFLLLKYRAREPVTKAEMLESVLRNCQDFFPVIFSKASEYLQLVFGIEVVEVVPISHLYILVTCLGLSYDGLLGDNQVMPKTGLLIIVLAIIAIEGDCAPEEKIWEELSMLEVFEGREDSVFAHPRKLLMQDLVQENYLEYRQVPGSDPACYEFLWGPRALIETSYVKVLHHTLKIGGEPHISYPPLHERALREGEE

SEQ ID NO: 73 - protein, 314 aa, Homo sapiens
MPLEQRSQHCKPEEGLEARGEALGLVGAQAPATEEQEAASSSSTLVEVTLGEVPAAESPDPPQSPQGASSLPTTMNYPLWSQSYEDSSNQEEEGPSTFPDLESEFQAALSRKVAELVHFLLLKYRAREPVTKAEMLGSVVGNWQYFFPVIFSKASSSLQLVFGIELMEVDPIGHLYIFATCLGLSYDGLLGDNQIMPKAGLLIIVLAIIAREGDCAPEEKIWEELSVLEVFEGREDSILGDPKKLLTQHFVQENYLEYRQVPGSDPACYEFLWGPRALVETSYVKVLHHMVKISGGPHISYPPLHEWVLREGEE

SEQ ID NO: 74 - protein, 180 aa, Homo sapiens
MQAEGRGTGGSTGDADGPGGPGIPDGPGGNAGGPGEAGATGGRGPRGAGAARASGPGGGAPRGPHGGAASGLNGCCRCGARGPESRLLEFYLAMPFATPMEAELARRSLAQDAPPLPVPGVLLKEFTVSGNILTIRLTAADHRQLQLSISSCLQQLSLLMWITQCFLPVFLAQPPSGQRR

SEQ ID NO: 75 - protein, 180 aa, Homo sapiens
MQAEGRGTGGSTGDADGPGGPGIPDGPGGNAGGPGEAGATGGRGPRGAGAARASGPRGGAPRGPHGGAASAQDGRCPCGARRPDSRLLELHITMPFSSPMEAELVRRILSRDAAPLPRPGAVLKDFTVSGNLLFIRLTAADHRQLQLSISSCLQQLSLLMWITQCFLPVFLAQAPSGQRR

SEQ ID NO: 76 - protein, 210 aa, Homo sapiens [sequence omitted]

SEQ ID NO: 77 - protein, 509 aa, Homo sapiens
MERRRLWGSIQSRYISMSVWTSPRRLVELAGQSLLKDEALAIAALELLPRELFPPLFMAAFDGRHSQTLKAMVQAWPFTCLPLGVLMKGQHLHLETFKAVLDGLDVLLAQEVRPRRWKLQVLDLRKNSHQDFWTVWSGNRASLYSFPEPEAAQPMTKKRKVDGLSTEAEQPFIPVEVLVDLFLKEGACDELFSYLIEKVKRKKNVLRLCCKKLKIFAMPMQDIKMILKMVQLDSIEDLEVTCTWKLPTLAKFSPYLGQMINLRRLLLSHIHASSYISPEKEEQYIAQFTSQFLSLQCLQALYVDSLFFLRGRLDQLLRHVMNPLETLSITNCRLSEGDVMHLSQSPSVSQLSVLSLSGVMLTDVSPEPLQALLERASATLQDLVFDECGITDDQLLALLPSLSHCSQLTTLSFYGNSISISALQSLLQHLIGLSNLTHVLYPVPLESYEDIHGTLHLERLAYLHARLRELLCELGRPSMVWLSANPCPHCGDRTFYDPEPILCPCFMPN

SEQ ID NO: 78 - protein, 261 aa, Homo sapiens
MWVPVVFLTLSVTWIGAAPLILSRIVGGWECEKHSQPWQVLVASRGRAVCGGVLVHPQWVLTAAHCIRNKSVILLGRHSLFHPEDTGQVFQVSHSFPHPLYDMSLLKNRFLRPGDDSSHDLMLLRLSEPAELTDAVKVMDLPTQEPALGTTCYASGWGSIEPEEFLTPKKLQCVDLHVISNDVCAQVHPQKVTKFMLCAGRWTGGKSTCSGDSGGPLVCNGVLQGITSWGSEPCALPERPSLYTKVVHYRKWIKDTIVANP

SEQ ID NO: 79 - protein, 123 aa, Homo sapiens
MKAVLLALLMAGLALQPGTALLCYSCKAQVSNEDCLQVENCTQLGEQCWTARIRAVGLLTVISKGCSLNCVDDSQDYYVGKKNITCCDTDLCNASGAHALQPAAAILALLPALGLLLWGPGQL

SEQ ID NO: 80 - DNA, 2817 bp, Homo sapiens
gtgctaaaaagatgccttcttcatttggctgtgataggtgctttgtggctgtgggggctacaaaagtacccagaaaccaggactggcttggtgtctcaaggcaactcagaaccaaagcctggaacaggcagctgtatccagagtggacagaagcccagagacttgactgctggagaggtggtcaagtgtccctcaaggtcagtaatgatgggcctacactgattggtgcaaatgcctccttctctattgccttgaacttccctggaagccaaaaggtattgccagatgggcaggttatctgggtcaacaataccatcatcaatgggagccaggtgtggggaggacagccagtgtatccccaggaaactgacgatgcctgcatcttccctgatggtggaccttgcccatctggctcttggtctcagaagagaagctttgtttatgtctggaagacctggggtgagggactcccttctcagcctatcatccacacttgtgtttacttctttctacctgatcacctttcttttggccgccccttccaccttaacttctgtgattttctctaatcttcattttcctcttagatcttttctctttcttagcacctagcccccttcaagctctatcataattctttctggcaactcttggcctcaattgtagtcctaccccatggaatgcctcattaggaccccttccctgtccccccatatcacagccttccaaacaccctcagaagtaatcatacttcctgacctcccatctccagtgccgtttcgaagcctgtccctcagtcccctttgaccagtaatctcttcttccttgcttttcattccaaaaatgcttcaggccaatactggcaagttctagggggcccagtgtctgggctgagcattgggacaggcagggcaatgctgggcacacacaccatggaagtgactgtctaccatcgccggggatcccggagctatgtgcctcttgctcattccagctcagccttcaccattactggtaagggttcaggaagggcaaggccagttgtagggcaaagagaaggcagggaggcttggatggactgcaaaggagaaaggtgaaatgctgtgcaaacttaaagtagaagggccaggaagacctaggcagagaaatgtgaggcttagtgccagtgaagggccagccagtcagcttggagttggagggtgtggctgtgaaaggagaagctgtggctcaggcctggttctcaccttttctggctccaatcccagaccaggtgcctttctccgtgagcgtgtcccagttgcgggccttggatggagggaacaagcacttcctgagaaatcagcctctgacctttgccctccagctccatgaccccagtggctatctggctgaagctgacctctcctacacctgggactttggagacagtagtggaaccctgatctctcgggcacctgtggtcactcatacttacctggagcctggcccagtcactgcccaggtggtcctgcaggctgccattcctctcacctcctgtggctcctccccagttccaggcaccacagatgggcacaggccaactgcagaggcccctaacaccacagctggccaagtgcctactacagaagttgtgggtactacacctggtcaggcgccaactgcagagccctctggaaccacatctgtgcaggtgccaaccactgaagtcataagcactgcacctgtgcagatgccaactgcagagagcacaggtatgacacctgagaaggtgccagtttcagaggtcatgggtaccacactggcagagatgtcaactccagaggctacaggtatgacacctgcagaggtatcaattgtggtgctttctggaaccacagctgcacaggtaacaactacagagtgggtggagaccacagctagagagctacctatccctgagcctgaaggtccagatgccagctcaatcatgtctacggaaagtattacaggttccctgggccccctgctggatggtacagccaccttaaggctggtgaagagacaagtccccctggattgtgttctgtatcgatatggttccttttccgtcaccctggacattgtccagggtattgaaagtgccgagatcctgcaggctgtgccgtccggtgagggggatgcatttgagctgactgtgtcctgccaaggcgggctgcccaaggaagcctgcatggagatctcatcgccagggtgccagccccctgcccagcggctgtgccagcctgtgctacccagcccagcctgccagctggttctgcaccagatactgaagggtggctcggggacatactgcctcaatgtgtctctggctgataccaacagcctggcagtggtcagcacccagcttatcatgcctggtaggtccttggacagagactaagtgaggagggaagtggatagaggggacagctggcaagcagcagacatgagtgaagcagtgcctgggattcttctcacaggtcaagaagcaggccttgggcaggttccgctgatcgtgggcatcttgctggtgttgatggctgtggtccttgcatctctgatatataggcgcagacttatgaagcaagacttctccgtaccccagttgccacatagcagcagtcactggctgcgtctaccccgcatcttctgctcttgtcccattggtgagaatagccccctcctcagtgggcagcaggtctgagtactctcatatgatgctgtgattttcctggagttgacagaaacacctatatttcccccagtcttccctgggagactactattaactgaaataaa

SEQ ID NO: 81 - DNA, 2420 bp, Homo sapiens
ggatccaggccctgccaggaaaaatataagggccctgcgtgagaacagagggggtcatccactgcatgagagtggggatgtcacagagtccagcccaccctcctggtagcactgagaagccagggctgtgcttgcggtctgcaccctgagggcccgtggattcctcttcctggagctccaggaaccaggcagtgaggccttggtctgagacagtatcctcaggtcacagagcagaggatgcacagggtgtgccagcagtgaatgtttgccctgaatgcacaccaagggccccacctgccacaggacacataggactccacagagtctggcctcacctccctactgtcagtcctgtagaatcgacctctgctggccggctgtaccctgagtaccctctcacttcctccttcaggttttcaggggacaggccaacccagaggacaggattccctggaggccacagaggagcaccaaggagaagatctgtaagtaggcctttgttagagtctccaaggttcagttctcagctgaggcctctcacacactccctctctccccaggcctgtgggtcttcattgcccagctcctgcccacactcctgcctgctgccctgacgagagtcatcatgtctcttgagcagaggagtctgcactgcaagcctgaggaagcccttgaggcccaacaagaggccctgggcctggtgtgtgtgcaggctgccacctcctcctcctctcctctggtcctgggcaccctggaggaggtgcccactgctgggtcaacagatcctccccagagtcctcagggagcctccgcctttcccactaccatcaacttcactcgacagaggcaacccagtgagggttccagcagccgtgaagaggaggggccaagcacctcttgtatcctggagtccttgttccgagcagtaatcactaagaaggtggctgatttggttggttttctgctcctcaaatatcgagccagggagccagtcacaaaggcagaaatgctggagagtgtcatcaaaaattacaagcactgttttcctgagatcttcggcaaagcctctgagtccttgcagctggtctttggcattgacgtgaaggaagcagaccccaccggccactcctatgtccttgtcacctgcctaggtctctcctatgatggcctgctgggtgataatcagatcatgcccaagacaggcttcctgataattgtcctggtcatgattgcaatggagggcggccatgctcctgaggaggaaatctgggaggagctgagtgtgatggaggtgtatgatgggagggagcacagtgcctatggggagcccaggaagctgctcacccaagatttggtgcaggaaaagtacctggagtaccggcaggtgccggacagtgatcccgcacgctatgagttcctgtggggtccaagggccctcgctgaaaccagctatgtgaaagtccttgagtatgtgatcaaggtcagtgcaagagttcgctttttcttcccatccctgcgtgaagcagctttgagagaggaggaagagggagtctgagcatgagttgcagccaaggccagtgggagggggactgggccagtgcaccttccagggccgcgtccagcagcttcccctgcctcgtgtgacatgaggcccattcttcactctgaagagagcggtcagtgttctcagtagtaggtttctgttctattgggtgacttggagatttatctttgttctcttttggaattgttcaaatgtttttttttaagggatggttgaatgaacttcagcatccaagtttatgaatgacagcagtcacacagttctgtgtatatagtttaagggtaagagtcttgtgttttattcagattgggaaatccattctattttgtgaattgggataataacagcagtggaataagtacttagaaatgtgaaaaatgagcagtaaaatagatgagataaagaactaaagaaattaagagatagtcaattcttgccttatacctcagtctattctgtaaaatttttaaagatatatgcatacctggatttccttggcttctttgagaatgtaagagaaattaaatctgaataaagaattcttcctgttcactggctcttttcttctccatgcactgagcatctgctttttggaaggccctgggttagtagtggagatgctaaggtaagccagactcatacccacccatagggtcgtagagtctaggagctgcagtcacgtaatcgaggtggcaagatgtcctctaaagatgtagggaaaagtgagagaggggtgagggtgtggggctccgggtgagagtggtggagtgtcaatgccctgagctggggcattttgggctttgggaaactgcagttccttctgggggagctgattgtaatgatcttgggtggatcc

SEQ ID NO: 82 - DNA, 4559 bp, Homo sapiens
attccttcatcaaacagccaggagtgaggaagaggaccctcctgagtgaggactgaggatccaccctcaccacatagtgggaccacagaatccagctcagcccctcttgtcagccctggtacacactggcaatgatctcaccccgagcacacccctccccccaatgccacttcgggccgactcagagtcagagacttggtctgaggggagcagacacaatcggcagaggatggcggtccaggctcagtctggcatccaagtcaggaccttgagggatgaccaaaggcccctcccacccccaactcccccgaccccaccaggatctacagcctcaggatccccgtcccaatccctacccctacaccaacaccatcttcatgcttacccccacccccccatccagatccccatccgggcagaatccggttccacccttgccgtgaacccagggaagtcacgggcccggatgtgacgccactgacttgcacattggaggtcagaggacagcgagattctcgccctgagcaacggcctgacgtcggcggagggaagcaggcgcaggctccgtgaggaggcaaggtaagacgccgagggaggactgaggcgggcctcaccccagacagagggcccccaataatccagcgctgcctctgctgccgggcctggaccaccctgcaggggaagacttctcaggctcagtcgccaccacctcaccccgccaccccccgccgctttaaccgcagggaactctggcgtaagagctttgtgtgaccagggcagggctggttagaagtgctcagggcccagactcagccaggaatcaaggtcaggaccccaagaggggactgagggcaacccaccccctaccctcactaccaatcccatcccccaacaccaaccccacccccatccctcaaacaccaaccccacccccaaaccccattcccatctcctcccccaccaccatcctggcagaatccggctttgcccctgcaatcaacccacggaagctccgggaatggcggccaagcacgcggatcctgacgttcacatgtacggctaagggagggaaggggttgggtctcgtgagtatggcctttgggatgcagaggaagggcccaggcctcctggaagacagtggagtccttaggggacccagcatgccaggacagggggcccactgtacccctgtctcaaactgagccaccttttcattcagccgagggaatcctagggatgcagacccacttcagcagggggttggggcccagcctgcgaggagtcaaggggaggaagaagagggaggactgaggggaccttggagtccagatcagtggcaaccttgggctgggggatcctgggcacagtggccgaatgtgccccgtgctcattgcaccttcagggtgacagagagttgagggctgtggtctgagggctgggacttcaggtcagcagagggaggaatcccaggatctgccggacccaaggtgtgcccccttcatgaggactggggatacccccggcccagaaagaagggatgccacagagtctggaagtcccttgttcttagctctgggggaacctgatcagggatggccctaagtgacaatctcatttgtaccacaggcaggaggttggggaaccctcagggagataaggtgttggtgtaaagaggagctgtctgctcatttcagggggttgggggttgagaaagggcagtccctggcaggagtaaagatgagtaacccacaggaggccatcataacgttcaccctagaaccaaaggggtcagccctggacaacgcacgtgggggtaacaggatgtggcccctcctcacttgtctttccagatctcagggagttgatgaccttgttttcagaaggtgactcaggtcaacacaggggccccatctggtcgacagatgcagtggttctaggatctgccaagcatccaggtggagagcctgaggtaggattgagggtacccctgggccagaatgcagcaagggggccccatagaaatctgccctgcccctgcggttacttcagagaccctgggcagggctgtcagctgaagtccctccattatcctgggatctttgatgtcagggaaggggaggccttggtctgaaggggctggagtcaggtcagtagagggagggtctcaggccctgccaggagtggacgtgaggaccaagcggactcgtcacccaggacacctggactccaatgaatttggacatctctcgttgtccttcgcgggaggacctggtcacgtatggccagatgtgggtcccctcatatccttctgtaccatatcagggatgtgagttcttgacatgagagattctcaagccagcaaaagggtgggattaggccctacaaggagaaaggtgagggccctgagtgagcacagaggggaccctccacccaagtagagtggggacctcacggagtctggccaaccctgctgagacttctgggaatccgtggctgtgcttgcagtctgcacactgaaggcccgtgcattcctctcccaggaatcaggagctccaggaaccaggcagtgaggccttggtctgagtcagtgtcctcaggtcacagagcagaggggacgcagacagtgccaacactgaaggtttgcctggaatgcacaccaagggccccacccgcccagaacaaatgggactccagagggcctggcctcaccctccctattctcagtcctgcagcctgagcatgtgctggccggctgtaccctgaggtgccctcccacttcctccttcaggttctgagggggacaggctgacaagtaggacccgaggcactggaggagcattgaaggagaagatctgtaagtaagcctttgtcagagcctccaaggttcagttcagttctcacctaaggcctcacacacgctccttctctccccaggcctgtgggtcttcattgcccagctcctgcccgcactcctgcctgctgccctgaccagagtcatcatgcctcttgagcagaggagtcagcactgcaagcctgaagaaggccttgaggcccgaggagaggccctgggcctggtgggtgcgcaggctcctgctactgaggagcagcagaccgcttcttcctcttctactctagtggaagttaccctgggggaggtgcctgctgccgactcaccgagtcctccccacagtcctcagggagcctccagcttctcgactaccatcaactacactctttggagacaatccgatgagggctccagcaaccaagaagaggaggggccaagaatgtttcccgacctggagtccgagttccaagcagcaatcagtaggaagatggttgagttggttcattttctgctcctcaagtatcgagccagggagccggtcacaaaggcagaaatgctggagagtgtcctcagaaattgccaggacttctttcccgtgatcttcagcaaagcctccgagtacttgcagctggtctttggcatcgaggtggtggaagtggtccccatcagccacttgtacatccttgtcacctgcctgggcctctcctacgatggcctgctgggcgacaatcaggtcatgcccaagacaggcctcctgataatcgtcctggccataatcgcaatagagggcgactgtgcccctgaggagaaaatctgggaggagctgagtatgttggaggtgtttgaggggagggaggacagtgtcttcgcacatcccaggaagctgctcatgcaagatctggtgcaggaaaactacctggagtaccggcaggtgcccggcagtgatcctgcatgctacgagttcctgtggggtccaagggccctcattgaaaccagctatgtgaaagtcctgcaccatacactaaagatcggtggagaacctcacatttcctacccacccctgcatgaacgggctttgagagagggagaagagtgagtctcagcacatgttgcagccagggccagtgggagggggtctgggccagtgcaccttccagggccccatccattagcttccactgcctcgtgtgatatgaggcccattcctgcctctttgaagagagcagtcagcattcttagcagtgagtttctgttctgttggatgactttgagatttatctttctttcctgttggaattgttcaaatgttccttttaacaaatggttggatgaacttcagcatccaagtttatgaatgacagtagtcacacatagtgctgtttatatagtttaggggtaagagtcctgttttttattcagattgggaaatccattccattttgtgagttgtcacataataacagcagtggaatatgtatttgcctatattgtgaacgaattagcagtaaaatacatgatacaaggaactcaaaagatagttaattcttgccttatacctcagtctattatgtaaaattaaaaatatgtgtatgtttttgcttctttgagaatgcaaaagaaattaaatctgaataaattcttcctgttcactggctcatttctttaccattcactcagcatctgctctgtggaaggccctggtagtagtggg

SEQ ID NO: 83 - DNA, 4204 bp, Homo sapiens
acgcaggcagtgatgtcacccagaccacaccccttcccccaatgccacttcagggggtactcagagtcagagacttggtctgaggggagcagaagcaatctgcagaggatggcggtccaggctcagccaggcatcaacttcaggaccctgagggatgaccgaaggccccgcccacccacccccaactcccccgaccccaccaggatctacagcctcaggacccccgtcccaatccttaccccttgccccatcaccatcttcatgcttacctccacccccatccgatccccatccaggcagaatccagttccacccctgcccggaacccagggtagtaccgttgccaggatgtgacgccactgacttgcgcattggaggtcagaagaccgcgagattctcgccctgagcaacgagcgacggcctgacgtcggcggagggaagccggcccaggctcggtgaggaggcaaggtaagacgctgagggaggactgaggcgggcctcacctcagacagagggcctcaaataatccagtgctgcctctgctgccgggcctgggccaccccgcaggggaagacttccaggctgggtcgccactacctcaccccgccgacccccgccgctttagccacggggaactctggggacagagcttaatgtggccagggcagggctggttagaagaggtcagggcccacgctgtggcaggaatcaaggtcaggaccccgagagggaactgagggcagcctaaccaccaccctcaccaccattcccgtcccccaacacccaaccccacccccatcccccattcccatccccacccccacccctatcctggcagaatccgggctttgcccctggtatcaagtcacggaagctccgggaatggcggccaggcacgtgagtcctgaggttcacatctacggctaagggagggaaggggttcggtatcgcgagtatggccgttgggaggcagcgaaagggcccaggcctcctggaagacagtggagtcctgaggggacccagcatgccaggacagggggcccactgtacccctgtctcaaaccgaggcaccttttcattcggctacgggaatcctagggatgcagacccacttcagcagggggttggggcccagccctgcgaggagtcatggggaggaagaagagggaggactgaggggaccttggagtccagatcagtggcaaccttgggctgggggatgctgggcacagtggccaaatgtgctctgtgctcattgcgccttcagggtgaccagagagttgagggctgtggtctgaagagtgggacttcaggtcagcagagggaggaatcccaggatctgcagggcccaaggtgtacccccaaggggcccctatgtggtggacagatgcagtggtcctaggatctgccaagcatccaggtgaagagactgagggaggattgagggtacccctgggacagaatgcggactgggggccccataaaaatctgccctgctcctgctgttacctcagagagcctgggcagggctgtcagctgaggtccctccattatcctaggatcactgatgtcagggaaggggaagccttggtctgagggggctgcactcagggcagtagagggaggctctcagaccctactaggagtggaggtgaggaccaagcagtctcctcacccagggtacatggacttcaataaatttggacatctctcgttgtcctttccgggaggacctgggaatgtatggccagatgtgggtcccctcatgtttttctgtaccatatcaggtatgtgagttcttgacatgagagattctcaggccagcagaagggagggattaggccctataaggagaaaggtgagggccctgagtgagcacagaggggatcctccaccccagtagagtggggacctcacagagtctggccaaccctcctgacagttctgggaatccgtggctgcgtttgctgtctgcacattgggggcccgtggattcctctcccaggaatcaggagctccaggaacaaggcagtgaggacttggtctgaggcagtgtcctcaggtcacagagtagagggggctcagatagtgccaacggtgaaggtttgccttggattcaaaccaagggccccacctgccccagaacacatggactccagagcgcctggcctcaccctcaatactttcagtcctgcagcctcagcatgcgctggccggatgtaccctgaggtgccctctcacttcctccttcaggttctgaggggacaggctgacctggaggaccagaggcccccggaggagcactgaaggagaagatctgtaagtaagcctttgttagagcctccaaggttccattcagtactcagctgaggtctctcacatgctccctctctccccaggccagtgggtctccattgcccagctcctgcccacactcccgcctgttgccctgaccagagtcatcatgcctcttgagcagaggagtcagcactgcaagcctgaagaaggccttgaggcccgaggagaggccctgggcctggtgggtgcgcaggctcctgctactgaggagcaggaggctgcctcctcctcttctactctagttgaagtcaccctgggggaggtgcctgctgccgagtcaccagatcctccccagagtcctcagggagcctccagcctccccactaccatgaactaccctctctggagccaatcctatgaggactccagcaaccaagaagaggaggggccaagcaccttccctgacctggagtccgagttccaagcagcactcagtaggaaggtggccgagttggttcattttctgctcctcaagtatcgagccagggagccggtcacaaaggcagaaatgctggggagtgtcgtcggaaattggcagtatttctttcctgtgatcttcagcaaagcttccagttccttgcagctggtctttggcatcgagctgatggaagtggaccccatcggccacttgtacatctttgccacctgcctgggcctctcctacgatggcctgctgggtgacaatcagatcatgcccaaggcaggcctcctgataatcgtcctggccataatcgcaagagagggcgactgtgcccctgaggagaaaatctgggaggagctgagtgtgttagaggtgtttgaggggagggaagacagtatcttgggggatcccaagaagctgctcacccaacatttcgtgcaggaaaactacctggagtaccggcaggtccccggcagtgatcctgcatgttatgaattcctgtggggtccaagggccctcgttgaaaccagctatgtgaaagtcctgcaccatatggtaaagatcagtggaggacctcacatttcctacccacccctgcatgagtgggttttgagagagggggaagagtgagtctgagcacgagttgcagccagggccagtgggagggggtctgggccagtgcaccttccggggccgcatcccttagtttccactgcctcctgtgacgtgaggcccattcttcactctttgaagcgagcagtcagcattcttagtagtgggtttctgttctgttggatgactttgagattattctttgtttcctgttggagttgttcaaatgttccttttaacggatggttgaatgagcgtcagcatccaggtttatgaatgacagtagtcacacatagtgctgtttatatagtttaggagtaagagtcttgttttttactcaaattgggaaatccattccattttgtgaattgtgacataataatagcagtggtaaaagtatttgcttaaaattgtgagcgaattagcaataacatacatgagataactcaagaaatcaaaagatagttgattcttgccttgtacctcaatctattctgtaaaattaaacaaatatgcaaaccaggatttccttgacttctttgagaatgcaagcgaaattaaatctgaataaataattcttcctcttcactggctcgtttcttttccgttcactcagcatctgctctgtgggaggccctgggttagtagtggggatgctaaggtaagccagactcacgcctacccatagggctgtagagcctaggacctgcagtcatataattaaggtggtgagaagtcctgtaagatgtagaggaaatgtaagagaggggtgagggtgtggcgctccgggtgagagtagtggagtgtcagtgc

SEQ ID NO: 84 - DNA, 752 bp, Homo sapiens
atcctcgtgggccctgaccttctctctgagagccgggcagaggctccggagccatgcaggccgaaggccggggcacagggggttcgacgggcgatgctgatggcccaggaggccctggcattcctgatggcccagggggcaatgctggcggcccaggagaggcgggtgccacgggcggcagaggtccccggggcgcaggggcagcaagggcctcggggccgggaggaggcgccccgcggggtccgcatggcggcgcggcttcagggctgaatggatgctgcagatgcggggccagggggccggagagccgcctgcttgagttctacctcgccatgcctttcgcgacacccatggaagcagagctggcccgcaggagcctggcccaggatgccccaccgcttcccgtgccaggggtgcttctgaaggagttcactgtgtccggcaacatactgactatccgactgactgctgcagaccaccgccaactgcagctctccatcagctcctgtctccagcagctttccctgttgatgtggatcacgcagtgctttctgcccgtgtttttggctcagcctccctcagggcagaggcgctaagcccagcctggcgccccttcctaggtcatgcctcctcccctagggaatggtcccagcacgagtggccagttcattgtgggggcctgattgtttgtcgctggaggaggacggcttacatgtttgtttctgtagaaaataaaactgagctacgaaaaa

SEQ ID NO: 85 - DNA, 2148 bp, Homo sapiens
  misc_feature (1)...(2): n = A,T,C or G
gcttcagggtacagctcccccgcagccagaagccgggcctgcagcccctcagcaccgctccgggacaccccacccgcttcccaggcgtgacctgtcaacagcaacttcgcggtgtggtgaactctctgaggaaaaaccattttgattattactctcagacgtgcgtggcaacaagtgactgagacctagaaatccaagcgttggaggtcctgaggccagcctaagtcgcttcaaaatggaacgaaggcgtttgtggggttccattcagagccgatacatcagcatgagtgtgtggacaagcccacggagacttgtggagctggcagggcagagcctgctgaaggatgaggccctggccattgccgccctggagttgctgcccagggagctcttcccgccactcttcatggcagcctttgacgggagacacagccagaccctgaaggcaatggtgcaggcctggcccttcacctgcctccctctgggagtgctgatgaagggacaacatcttcacctggagaccttcaaagctgtgcttgatggacttgatgtgctccttgcccaggaggttcgccccaggaggtggaaacttcaagtgctggatttacggaagaactctcatcaggacttctggactgtatggtctggaaacagggccagtctgtactcatttccagagccagaagcagctcagcccatgacaaagaagcgaaaagtagatggtttgagcacagaggcagagcagcccttcattccagtagaggtgctcgtagacctgttcctcaaggaaggtgcctgtgatgaattgttctcctacctcattgagaaagtgaagcgaaagaaaaatgtactacgcctgtgctgtaagaagctgaagatttttgcaatgcccatgcaggatatcaagatgatcctgaaaatggtgcagctggactctattgaagatttggaagtgacttgtacctggaagctacccaccttggcgaaattttctccttacctgggccagatgattaatctgcgtagactcctcctctcccacatccatgcatcttcctacatttccccggagaaggaagagcagtatatcgcccagttcacctctcagttcctcagtctgcagtgcctgcaggctctctatgtggactctttatttttccttagaggccgcctggatcagttgctcaggcacgtgatgaaccccttggaaaccctctcaataactaactgccggctttcggaaggggatgtgatgcatctgtcccagagtcccagcgtcagtcagctaagtgtcctgagtctaagtggggtcatgctgaccgatgtaagtcccgagcccctccaagctctgctggagagagcctctgccaccctccaggacctggtctttgatgagtgtgggatcacggatgatcagctccttgccctcctgccttccctgagccactgctcccagcttacaaccttaagcttctacgggaattccatctccatatctgccttgcagagtctcctgcagcacctcatcgggctgagcaatctgacccacgtgctgtatcctgtccccctggagagttatgaggacatccatggtaccctccacctggagaggcttgcctatctgcatgccaggctcagggagttgctgtgtgagttggggcggcccagcatggtctggcttagtgccaacccctgtcctcactgtggggacagaaccttctatgacccggagcccatcctgtgcccctgtttcatgcctaactagctgggtgcacatatcaaatgcttcattctgcatacttggacactaaagccaggatgtgcatgcatcttgaagcaacaaagcagccacagtttcagacaaatgttcagtgtgagtgaggaaaacatgttcagtgaggaaaaaacattcagacaaatgttcagtgaggaaaaaaaggggaagttggggataggcagatgttgacttgaggagttaatgtgatctttggggagatacatcttatagagttagaaatagaatctgaatttctaaagggagattctggcttgggaagtacatgtaggagttaatccctgtgtagactgttgtaaagaaactgttgaaaataaagagaagcaatgtgaagcaaaaaaaaaaaaaaaaaa

SEQ ID NO: 86 - DNA, 1466 bp, Homo sapiens
agccccaagcttaccacctgcacccggagagctgtgtgtcaccatgtgggtcccggttgtcttcctcaccctgtccgtgacgtggattggtgctgcacccctcatcctgtctcggattgtgggaggctgggagtgcgagaagcattcccaaccctggcaggtgcttgtggcctctcgtggcagggcagtctgcggcggtgttctggtgcacccccagtgggtcctcacagctgcccactgcatcaggaacaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagccacgacctcatgctgctccgcctgtcagagcctgccgagctcacggatgctgtgaaggtcatggacctgcccacccaggagccagcactggggaccacctgctacgcctcaggctggggcagcattgaaccagaggagttcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgggtgattctgggggcccacttgtctgtaatggtgtgcttcaaggtatcacgtcatggggcagtgaaccatgtgccctgcccgaaaggccttccctgtacaccaaggtggtgcattaccggaagtggatcaaggacaccatcgtggccaacccctgagcacccctatcaaccccctattgtagtaaacttggaaccttggaaatgaccaggccaagactcaagcctccccagttctactgacctttgtccttaggtgtgaggtccagggttgctaggaaaagaaatcagcagacacaggtgtagaccagagtgtttcttaaatggtgtaattttgtcctctctgtgtcctggggaatactggccatgcctggagacatatcactcaatttctctgaggacacagataggatggggtgtctgtgttatttgtggggtacagagatgaaagaggggtgggatccacactgagagagtggagagtgacatgtgctggacactgtccatgaagcactgagcagaagctggaggcacaacgcaccagacactcacagcaaggatggagctgaaaacataacccactctgtcctggaggcactgggaagcctagagaaggctgtgagccaaggagggagggtcttcctttggcatgggatggggatgaagtaaggagagggactggaccccctggaagctgattcactatggggggaggtgtattgaagtcctccagacaaccctcagatttgatgatttcctagtagaactcacagaaataaagagctgttatactgtg

SEQ ID NO: 87 - DNA, 990 bp, Homo sapiens
  misc_feature (1)...(990): n = A,T,C or G
agggagaggcagtgaccatgaaggctgtgctgcttgccctgttgatggcaggcttggccctgcagccaggcactgccctgctgtgctactcctgcaaagcccaggtgagcaacgaggactgcctgcaggtggagaactgcacccagctgggggagcagtgctggaccgcgcgcatccgcgcagttggcctcctgaccgtcatcagcaaaggctgcagcttgaactgcgtggatgactcacaggactactacgtgggcaagaagaacatcacgtgctgtgacaccgacttgtgcaacgccagcggggcccatgccctgcagccggctgccgccatccttgcgctgctccctgcactcggcctgctgctctggggacccggccagctataggctctggggggccccgctgcagcccacactgggtgtggtgccccaggcctttgtgccactcctcacagaacctggcccagtgggagcctgtcctggttcctgaggcacatcctaacgcaagtttgaccatgtatgtttgcaccccttttccccnaaccctgaccttcccatgggccttttccaggattccnaccnggcagatcagttttagtganacanatccgcntgcagatggcccctccaaccntttntgttgntgtttccatggcccagcattttccacccttaaccctgtgttcaggcacttnttcccccaggaagccttccctgcccaccccatttatgaattgagccaggtttggtccgtggtgtcccccgcacccagcaggggacaggcaatcaggagggcccagtaaaggctgagatgaagtggactgagtagaactggaggacaagagttgacgtgagttcctgggagtttccagagatggggcctggaggcctggaggaaggggccaggcctcacatttgtggggntcccgaatggcagcctgagcacagcgtaggcccttaataaacacctgttggataagccaaaaaa

SEQ ID NO: 88 - protein, 702 aa, Homo sapiens
MESPSAPPHRWCIPWQRLLLTASLLTFWNPPTTAKLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELPKPSISSNNSKPVEDKDAVAFTCEPETQDATYLWWVNNQSLPVSPRLQLSNGNRTLTLFNVTRNDTASYKCETQNPVSARRSDSVILNVLYGPDAPTISPLNTSYRSGENLNLSCHAASNPPAQYSWFVNGTFQQSTQELFIPNITVNNSGSYTCQAHNSDTGLNRTTVTTITVYAEPPKPFITSNNSNPVEDEDAVALTCEPEIQNTTYLWWVNNQSLPVSPRLQLSNDNRTLTLLSVTRNDVGPYECGIQNELSVDHSDPVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASGTSPGLSAGATVGIMIGVLVGVALI

SEQ ID NO: 89 - DNA, 2974 bp, Homo sapiens
ctcagggcagagggaggaaggacagcagaccagacagtcacagcagccttgacaaaacgttcctggaactcaagctcttctccacagaggaggacagagcagacagcagagaccatggagtctccctcggcccctccccacagatggtgcatcccctggcagaggctcctgctcacagcctcacttctaaccttctggaacccgcccaccactgccaagctcactattgaatccacgccgttcaatgtcgcagaggggaaggaggtgcttctacttgtccacaatctgccccagcatctttttggctacagctggtacaaaggtgaaagagtggatggcaaccgtcaaattataggatatgtaataggaactcaacaagctaccccagggcccgcatacagtggtcgagagataatataccccaatgcatccctgctgatccagaacatcatccagaatgacacaggattctacaccctacacgtcataaagtcagatcttgtgaatgaagaagcaactggccagttccgggtatacccggagctgcccaagccctccatctccagcaacaactccaaacccgtggaggacaaggatgctgtggccttcacctgtgaacctgagactcaggacgcaacctacctgtggtgggtaaacaatcagagcctcccggtcagtcccaggctgcagctgtccaatggcaacaggaccctcactctattcaatgtcacaagaaatgacacagcaagctacaaatgtgaaacccagaacccagtgagtgccaggcgcagtgattcagtcatcctgaatgtcctctatggcccggatgcccccaccatttcccctctaaacacatcttacagatcaggggaaaatctgaacctctcctgccacgcagcctctaacccacctgcacagtactcttggtttgtcaatgggactttccagcaatccacccaagagctctttatccccaacatcactgtgaataatagtggatcctatacgtgccaagcccataactcagacactggcctcaataggaccacagtcacgacgatcacagtctatgcagagccacccaaacccttcatcaccagcaacaactccaaccccgtggaggatgaggatgctgtagccttaacctgtgaacctgagattcagaacacaacctacctgtggtgggtaaataatcagagcctcccggtcagtcccaggctgcagctgtccaatgacaacaggaccctcactctactcagtgtcacaaggaatgatgtaggaccctatgagtgtggaatccagaacgaattaagtgttgaccacagcgacccagtcatcctgaatgtcctctatggcccagacgaccccaccatttccccctcatacacctattaccgtccaggggtgaacctcagcctctcctgccatgcagcctctaacccacctgcacagtattcttggctgattgatgggaacatccagcaacacacacaagagctctttatctccaacatcactgagaagaacagcggactctatacctgccaggccaataactcagccagtggccacagcaggactacagtcaagacaatcacagtctctgcggagctgcccaagccctccatctccagcaacaactccaaacccgtggaggacaaggatgctgtggccttcacctgtgaacctgaggctcagaacacaacctacctgtggtgggtaaatggtcagagcctcccagtcagtcccaggctgcagctgtccaatggcaacaggaccctcactctattcaatgtcacaagaaatgacgcaagagcctatgtatgtggaatccagaactcagtgagtgcaaaccgcagtgacccagtcaccctggatgtcctctatgggccggacacccccatcatttcccccccagactcgtcttacctttcgggagcgaacctcaacctctcctgccactcggcctctaacccatccccgcagtattcttggcgtatcaatgggataccgcagcaacacacacaagttctctttatcgccaaaatcacgccaaataataacgggacctatgcctgttttgtctctaacttggctactggccgcaataattccatagtcaagagcatcacagtctctgcatctggaacttctcctggtctctcagctggggccactgtcggcatcatgattggagtgctggttggggttgctctgatatagcagccctggtgtagtttcttcatttcaggaagactgacagttgttttgcttcttccttaaagcatttgcaacagctacagtctaaaattgcttctttaccaaggatatttacagaaaagactctgaccagagatcgagaccatcctagccaacatcgtgaaaccccatctctactaaaaatacaaaaatgagctgggcttggtggcgcgcacctgtagtcccagttactcgggaggctgaggcaggagaatcgcttgaacccgggaggtggagattgcagtgagcccagatcgcaccactgcactccagtctggcaacagagcaagactccatctcaaaaagaaaagaaaagaagactctgacctgtactcttgaatacaagtttctgataccactgcactgtctgagaatttccaaaactttaatgaactaactgacagcttcatgaaactgtccaccaagatcaagcagagaaaataattaatttcatgggactaaatgaactaatgaggattgctgattctttaaatgtcttgtttcccagatttcaggaaactttttttcttttaagctatccactcttacagcaatttgataaaatatacttttgtgaacaaaaattgagacatttacattttctccctatgtggtcgctccagacttgggaaactattcatgaatatttatattgtatggtaatatagttattgcacaagttcaataaaaatctgctctttgtataacagaaaaa

SEQ ID NO: 90 - protein, 1255 aa, Homo sapiens
MELAALCRWGLLLALLPPGAASTQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRASPLTSIVSAVVGILLVVVLGVVFGILIKRRQQKIRKYTMRRLLQETELVEPLTPSGAMPNQAQMRILKETELRKVKVLGSGAFGTVYKGIWIPDGENVKIPVAIKVLRENTSPKANKEILDEAYVMAGVGSPYVSRLLGICLTSTVQLVTQLMPYGCLLDHVRENRGRLGSQDLLNWCMQIAKGMSYLEDVRLVHRDLAARNVLVKSPNHVKITDFGLARLLDIDETEYHADGGKVPIKWMALESILRRRFTHQSDVWSYGVTVWELMTFGAKPYDGIPAREIPDLLEKGERLPQPPICTIDVYMIMVKCWMIDSECRPRFRELVSEFSRMARDPQRFVVIQNEDLGPASPLDSTFYRSLLEDDDMGDLVDAEEYLVPQQGFFCPDPAPGAGGMVHHRHRSSSTRSGGGDLTLGLEPSEEEAPRSPLAPSEGAGSDVFDGDLGMGAAKGLQSLPTHDPSPLQRYSEDPTVPLPSETDGYVAPLTCSPQPEYVNQPDVRPQPPSPREGPLPAARPAGATLERAKTLSPGKNGVVKDVFAFGGAVENPEYLTPQGGAAPQPHPPPAFSPAFDNLYYWDQDPPERGAPPSTFKGTPTAENPEYLGLDVPV

SEQ ID NO: 91 - DNA, 4530 bp, Homo sapiens
aattctcgagctcgtcgaccggtcgacgagctcgagggtcgacgagctcgagggcgcgcgcccggcccccacccctcgcagcaccccgcgccccgcgccctcccagccgggtccagccggagccatggggccggagccgcagtgagcaccatggagctggcggccttgtgccgctgggggctcctcctcgccctcttgccccccggagccgcgagcacccaagtgtgcaccggcacagacatgaagctgcggctccctgccagtcccgagacccacctggacatgctccgccacctctaccagggctgccaggtggtgcagggaaacctggaactcacctacctgcccaccaatgccagcctgtccttcctgcaggatatccaggaggtgcagggctacgtgctcatcgctcacaaccaagtgaggcaggtcccactgcagaggctgcggattgtgcgaggcacccagctctttgaggacaactatgccctggccgtgctagacaatggagacccgctgaacaataccacccctgtcacaggggcctccccaggaggcctgcgggagctgcagcttcgaagcctcacagagatcttgaaaggaggggtcttgatccagcggaacccccagctctgctaccaggacacgattttgtggaaggacatcttccacaagaacaaccagctggctctcacactgatagacaccaaccgctctcgggcctgccacccctgttctccgatgtgtaagggctcccgctgctggggagagagttctgaggattgtcagagcctgacgcgcactgtctgtgccggtggctgtgcccgctgcaaggggccactgcccactgactgctgccatgagcagtgtgctgccggctgcacgggccccaagcactctgactgcctggcctgcctccacttcaaccacagtggcatctgtgagctgcactgcccagccctggtcacctacaacacagacacgtttgagtccatgcccaatcccgagggccggtatacattcggcgccagctgtgtgactgcctgtccctacaactacctttctacggacgtgggatcctgcaccctcgtctgccccctgcacaaccaagaggtgacagcagaggatggaacacagcggtgtgagaagtgcagcaagccctgtgcccgagtgtgctatggtctgggcatggagcacttgcgagaggtgagggcagttaccagtgccaatatccaggagtttgctggctgcaagaagatctttgggagcctggcatttctgccggagagctttgatggggacccagcctccaacactgccccgctccagccagagcagctccaagtgtttgagactctggaagagatcacaggttacctatacatctcagcatggccggacagcctgcctgacctcagcgtcttccagaacctgcaagtaatccggggacgaattctgcacaatggcgcctactcgctgaccctgcaagggctgggcatcagctggctggggctgcgctcactgagggaactgggcagtggactggccctcatccaccataacacccacctctgcttcgtgcacacggtgccctgggaccagctctttcggaacccgcaccaagctctgctccacactgccaaccggccagaggacgagtgtgtgggcgagggcctggcctgccaccagctgtgcgcccgagggcactgctggggtccagggcccacccagtgtgtcaactgcagccagttccttcggggccaggagtgcgtggaggaatgccgagtactgcaggggctccccagggagtatgtgaatgccaggcactgtttgccgtgccaccctgagtgtcagccccagaatggctcagtgacctgttttggaccggaggctgaccagtgtgtggcctgtgcccactataaggaccctcccttctgcgtggcccgctgccccagcggtgtgaaacctgacctctcctacatgcccatctggaagtttccagatgaggagggcgcatgccagccttgccccatcaactgcacccactcctgtgtggacctggatgacaagggctgccccgccgagcagagagccagccctctgacgtccatcgtctctgcggtggttggcattctgctggtcgtggtcttgggggtggtctttgggatcctcatcaagcgacggcagcagaagatccggaagtacacgatgcggagactgctgcaggaaacggagctggtggagccgctgacacctagcggagcgatgcccaaccaggcgcagatgcggatcctgaaagagacggagctgaggaaggtgaaggtgcttggatctggcgcttttggcacagtctacaagggcatctggatccctgatggggagaatgtgaaaattccagtggccatcaaagtgttgagggaaaacacatcccccaaagccaacaaagaaatcttagacgaagcatacgtgatggctggtgtgggctccccatatgtctcccgccttctgggcatctgcctgacatccacggtgcagctggtgacacagcttatgccctatggctgcctcttagaccatgtccgggaaaaccgcggacgcctgggctcccaggacctgctgaactggtgtatgcagattgccaaggggatgagctacctggaggatgtgcggctcgtacacagggacttggccgctcggaacgtgctggtcaagagtcccaaccatgtcaaaattacagacttcgggctggctcggctgctggacattgacgagacagagtaccatgcagatgggggcaaggtgcccatcaagtggatggcgctggagtccattctccgccggcggttcacccaccagagtgatgtgtggagttatggtgtgactgtgtgggagctgatgacttttggggccaaaccttacgatgggatcccagcccgggagatccctgacctgctggaaaagggggagcggctgccccagccccccatctgcaccattgatgtctacatgatcatggtcaaatgttggatgattgactctgaatgtcggccaagattccgggagttggtgtctgaattctcccgcatggccagggacccccagcgctttgtggtcatccagaatgaggacttgggcccagccagtcccttggacagcaccttctaccgctcactgctggaggacgatgacatgggggacctggtggatgctgaggagtatctggtaccccagcagggcttcttctgtccagaccctgccccgggcgctgggggcatggtccaccacaggcaccgcagctcatctaccaggagtggcggtggggacctgacactagggctggagccctctgaagaggaggcccccaggtctccactggcaccctccgaaggggctggctccgatgtatttgatggtgacctgggaatgggggcagccaaggggctgcaaagcctccccacacatgaccccagccctctacagcggtacagtgaggaccccacagtacccctgccctctgagactgatggctacgttgcccccctgacctgcagcccccagcctgaatatgtgaaccagccagatgttcggccccagcccccttcgccccgagagggccctctgcctgctgcccgacctgctggtgccactctggaaagggccaagactctctccccagggaagaatggggtcgtcaaagacgtttttgcctttgggggtgccgtggagaaccccgagtacttgacaccccagggaggagctgcccctcagccccaccctcctcctgccttcagcccagccttcgacaacctctattactgggaccaggacccaccagagcggggggctccacccagcaccttcaaagggacacctacggcagagaacccagagtacctgggtctggacgtgccagtgtgaaccagaaggccaagtccgcagaagccctgatgtgtcctcagggagcagggaaggcctgacttctgctggcatcaagaggtgggagggccctccgaccacttccaggggaacctgccatgccaggaacctgtcctaaggaaccttccttcctgcttgagttcccagatggctggaaggggtccagcctcgttggaagaggaacagcactggggagtctttgtggattctgaggccctgcccaatgagactctagggtccagtggatgccacagcccagcttggccctttccttccagatcctgggtactgaaagccttagggaagctggcctgagaggggaagcggccctaagggagtgtctaagaacaaaagcgacccattcagagactgtccctgaaacctagtactgccccccatgaggaaggaacagcaatggtgtcagtatccaggctttgtacagagtgcttttctgtttagtttttactttttttgttttgtttttttaaagacgaaataaagacccaggggagaatgggtgttgtatggggaggcaagtgtggggggtccttctccacacccactttgtccatttgcaaatatattttggaaaac

SEQ ID NO: 92 - protein, 976 aa, Homo sapiens [sequence omitted]

SEQ ID NO: 93 - DNA, 3393 bp, Homo sapiens
gccctcatagaccgtttgttgtagttcgcgtgggaacagcaacccacggtttcccgatagttcttcaaagatatttacaaccgtaacagagaaaatggaaaagcaaaagccctttgcattgttcgtaccaccgagatcaagcagcagtcaggtgtctgcggtgaaacctcagaccctgggaggcgattccactttcttcaagagtttcaacaaatgtactgaagatgatttggagtttccatttgcaaagactaatctctccaaaaatggggaaaacattgattcagatcctgctttacaaaaagttaatttcttgcccgtgcttgagcaggttggtaattctgactgtcactatcaggaaggactaaaagactctgatttggagaattcagagggattgagcagagtgttttcaaaactgtataaggaggctgaaaagataaaaaaatggaaagtaagtacagaagctgaactgagacagaaagaaagtaagttgcaagaaaacagaaagataattgaagcacagcgaaaagccattcaggaactgcaatttggaaatgaaaaagtaagtttgaaattagaagaaggaatacaagaaaataaagatttaataaaagagaataatgccacaaggcatttatgtaatctactcaaagaaacctgtgctagatctgcagaaaagacaaagaaatatgaatatgaacgggaagaaaccaggcaagtttatatggatctaaataataacattgagaaaatgataacagctcatggggaacttcgtgtgcaagctgagaattccagactggaaatgcattttaagttaaaggaagattatgaaaaaatccaacaccttgaacaagaatacaagaaggaaataaatgacaaggaaaagcaggtatcactactattgatccaaatcactgagaaagaaaataaaatgaaagatttaacatttctgctagaggaatccagagataaagttaatcaattagaggaaaagacaaaattacagagtgaaaacttaaaacaatcaattgagaaacagcatcatttgactaaagaactagaagatattaaagtgtcattacaaagaagtgtgagtactcaaaaggctttagaggaagatttacagatagcaacaaaaacaatttgtcagctaactgaagaaaaagaaactcaaatggaagaatctaataaagctagagctgctcattcgtttgtggttactgaatttgaaactactgtctgcagcttggaagaattattgagaacagaacagcaaagattggaaaaaaatgaagatcaattgaaaatacttaccatggagcttcaaaagaaatcaagtgagctggaagagatgactaagcttacaaataacaaagaagtagaacttgaagaattgaaaaaagtcttgggagaaaaggaaacacttttatatgaaaataaacaatttgagaagattgctgaagaattaaaaggaacagaacaagaactaattggtcttctccaagccagagagaaagaagtacatgatttggaaatacagttaactgccattaccacaagtgaacagtattattcaaaagaggttaaagatctaaaaactgagcttgaaaacgagaagcttaagaatactgaattaacttcacactgcaacaagctttcactagaaaacaaagagctcacacaggaaacaagtgatatgaccctagaactcaagaatcagcaagaagatattaataataacaaaaagcaagaagaaaggatgttgaaacaaatagaaaatcttcaagaaacagaaacccaattaagaaatgaactagaatatgtgagagaagagctaaaacagaaaagagatgaagttaaatgtaaattggacaagagtgaagaaaattgtaacaatttaaggaaacaagttgaaaataaaaacaagtatattgaagaacttcagcaggagaataaggccttgaaaaaaaaaggtacagcagaaagcaagcaactgaatgtttatgagataaaggtcaataaattagagttagaactagaaagtgccaaacagaaatttggagaaatcacagacacctatcagaaagaaattgaggacaaaaagatatcagaagaaaatcttttggaagaggttgagaaagcaaaagtaatagctgatgaagcagtaaaattacagaaagaaattgataagcgatgtcaacataaaatagctgaaatggtagcacttatggaaaaacataagcaccaatatgataagatcattgaagaaagagactcagaattaggactttataagagcaaagaacaagaacagtcatcactgagagcatctttggagattgaactatccaatctcaaagctgaacttttgtctgttaagaagcaacttgaaatagaaagagaagagaaggaaaaactcaaaagagaggcaaaagaaaacacagctactcttaaagaaaaaaaagacaagaaaacacaaacatttttattggaaacacctgaaatttattggaaattggattctaaagcagttccttcacaaactgtatctcgaaatttcacatcagttgatcatggcatatccaaagataaaagagactatctgtggacatctgccaaaaatactttatctacaccattgccaaaggcatatacagtgaagacaccaacaaaaccaaaactacagcaaagagaaaacttgaatatacccattgaagaaagtaaaaaaaagagaaaaatggcctttgaatttgatattaattcagatagttcagaaactactgatcttttgagcatggtttcagaagaagagacattgaaaacactgtataggaacaataatccaccagcttctcatctttgtgtcaaaacaccaaaaaaggccccttcatctctaacaacccctggacctacactgaagtttggagctataagaaaaatgcgggaggaccgttgggctgtaattgctaaaatggatagaaaaaaaaaactaaaagaagctgaaaagttatttgtttaatttcagagaatcagtgtagttaaggagcctaataacgtgaaacttatagttaatattttgttcttatttgccagagccacattttatctggaagttgagacttaaaaaatacttgcatgaatgatttgtgtttctttatatttttagcctaaatgttaactacatattgtctggaaacctgtcattgtattcagataattagatgattatatattgttgttactttttcttgtattcatgaaaactgtttttactaagttttcaaatttgtaaagttagcctttgaatgctaggaatgcattattgagggtcattctttattctttactattaaaatattttggatgcaaaaaaaaaaaaaaaaaaaa

SEQ ID NO: 94 - protein, 188 aa, Homo sapiens
MNGDDAFARRPRDDAQISEKLRKAFDDIAKYFSKKEWEKMKSSEKIVYVYMKLNYEVMTKLGFKVTLPPFMRSKRAADFHGNDFGNDRNHRNQVERPQMTFGSLQRIFPKIMPKKPAEEENGLKEVPEASGPQNDGKQLCPPGNPSTLEKINKTSGPKRGKHAWTHRLRERKQLVVYEEISDPEEDDE

SEQ ID NO: 95 - DNA, 576 bp, Homo sapiens
atgaacggagacgacgcctttgcaaggagacccagggatgatgctcaaatatcagagaagttacgaaaggccttcgatgatattgccaaatacttctctaagaaagagtgggaaaagatgaaatcctcggagaaaatcgtctatgtgtatatgaagctaaactatgaggtcatgactaaactaggtttcaaggtcaccctcccacctttcatgcgtagtaaacgggctgcagacttccacgggaatgattttggtaacgatcgaaaccacaggaatcaggttgaacgtcctcagatgactttcggcagcctccagagaatcttcccgaagatcatgcccaagaagccagcagaggaagaaaatggtttgaaggaagtgccagaggcatctggcccacaaaatgatgggaaacagctgtgccccccgggaaatccaagtaccttggagaagattaacaagacatctggacccaaaagggggaaacatgcctggacccacagactgcgtgagagaaagcagctggtggtttatgaagagatcagcgaccctgaggaagatgacgagtaactcccctcg

SEQ ID NO: 96 - protein, 94 aa, Homo sapiens
PATQRQDPAAAQEGEDEGASAGQGPKPEADSQEQGHPQTGCECEDGPDGQEMDPPNPEEVKTPEEEMRSHYVAQTGILWLLMNNCFLNLSPRKP

SEQ ID NO: 97 - DNA, 646 bp, Homo sapiens
ctgccgtccggactctttttcctctactgagattcatctgtgtgaaatatgagttggcgaggaagatcgacctatcggcctagaccaagacgctacgtagagcctcctgaaatgattgggcctatgcggcccgagcagttcagtgatgaagtggaaccagcaacacctgaagaaggggaaccagcaactcaacgtcaggatcctgcagctgctcaggagggagaggatgagggagcatctgcaggtcaagggccgaagcctgaagctgatagccaggaacagggtcacccacagactgggtgtgagtgtgaagatggtcctgatgggcaggagatggacccgccaaatccagaggaggtgaaaacgcctgaagaagagatgaggtctcactatgttgcccagactgggattctctggcttttaatgaacaattgcttcttaaatctttccccacggaaaccttgagtgactgaaatatcaaatggcgagagaccgtttagttcctatcatctgtggcatgtgaagggcaatcacagtgttaaaagaagacatgctgaaatgttgcaggctgctcctatgttggaaaattcttcattgaagttctcccaataaagctttacagccttctgcaaagaaaaaaaaaaaa

SEQ ID NO: 98 - protein, 98 aa, Homo sapiens
HCPTENEPDLAQCFFCFKELEGWEPDDDPIEEHKKHSSGCAFLSVKKQFEELTLGEFLKLDRERAKNKIAKETNNKKKEFEETAKKVRRAIEQLAAMD

SEQ ID NO: 99 - DNA, 1619 bp, Homo sapiens
ccgccagatttgaatcgcgggacccgttggcagaggtggcggcggcggcatgggtgccccgacgttgccccctgcctggcagccctttctcaaggaccaccgcatctctacattcaagaactggcccttcttggagggctgcgcctgcaccccggagcggatggccgaggctggcttcatccactgccccactgagaacgagccagacttggcccagtgtttcttctgcttcaaggagctggaaggctgggagccagatgacgaccccatagaggaacataaaaagcattcgtccggttgcgctttcctttctgtcaagaagcagtttgaagaattaacccttggtgaatttttgaaactggacagagaaagagccaagaacaaaattgcaaaggaaaccaacaataagaagaaagaatttgaggaaactgcgaagaaagtgcgccgtgccatcgagcagctggctgccatggattgaggcctctggccggagctgcctggtcccagagtggctgcaccacttccagggtttattccctggtgccaccagccttcctgtgggccccttagcaatgtcttaggaaaggagatcaacattttcaaattagatgtttcaactgtgctcctgttttgtcttgaaagtggcaccagaggtgcttctgcctgtgcagcgggtgctgctggtaacagtggctgcttctctctctctctctcttttttgggggctcatttttgctgttttgattcccgggcttaccaggtgagaagtgagggaggaagaaggcagtgtcccttttgctagagctgacagctttgttcgcgtgggcagagccttccacagtgaatgtgtctggacctcatgttgttgaggctgtcacagtcctgagtgtggacttggcaggtgcctgttgaatctgagctgcaggttccttatctgtcacacctgtgcctcctcagaggacagtttttttgttgttgtgtttttttgttttttttttttggtagatgcatgacttgtgtgtgatgagagaatggagacagagtccctggctcctctactgtttaacaacatggctttcttattttgtttgaattgttaattcacagaatagcacaaactacaattaaaactaagcacaaagccattctaagtcattggggaaacggggtgaacttcaggtggatgaggagacagaatagagtgataggaagcgtctggcagatactccttttgccactgctgtgtgattagacaggcccagtgagccgcggggcacatgctggccgctcctccctcagaaaaaggcagtggcctaaatcctttttaaatgacttggctcgatgctgtgggggactggctgggctgctgcaggccgtgtgtctgtcagcccaaccttcacatctgtcacgttctccacacgggggagagacgcagtccgcccaggtccccgctttctttggaggcagcagctcccgcagggctgaagtctggcgtaagatgatggatttgattcgccctcctccctgtcatagagctgcagggtggattgttacagcttcgctggaaacctctggaggtcatctcggctgttcctgagaaataaaaagcctgtcatttc

SEQ ID NO: 100 - protein, 74 aa, Homo sapiens
CWYCRRRNGYRALMDKSLHVGTQCALTRRCPQEGFDHRDSKVSLQEKNCEPVVPNAPPAYEKLSAEQSPPPYSP

SEQ ID NO: 101 - DNA, 1524 bp, Homo sapiens
agcagacagaggactctcattaaggaaggtgtcctgtgccctgaccctacaagatgccaagagaagatgctcacttcatctatggttaccccaagaaggggcacggccactcttacaccacggctgaagaggccgctgggatcggcatcctgacagtgatcctgggagtcttactgctcatcggctgttggtattgtagaagacgaaatggatacagagccttgatggataaaagtcttcatgttggcactcaatgtgccttaacaagaagatgcccacaagaagggtttgatcatcgggacagcaaagtgtctcttcaagagaaaaactgtgaacctgtggttcccaatgctccacctgcttatgagaaactctctgcagaacagtcaccaccaccttattcaccttaagagccagcgagacacctgagacatgctgaaattatttctctcacacttttgcttgaatttaatacagacatctaatgttctcctttggaatggtgtaggaaaaatgcaagccatctctaataataagtcagtgttaaaattttagtaggtccgctagcagtactaatcatgtgaggaaatgatgagaaatattaaattgggaaaactccatcaataaatgttgcaatgcatgatactatctgtgccagaggtaatgttagtaaatccatggtgttattttctgagagacagaattcaagtgggtattctggggccatccaatttctctttacttgaaatttggctaataacaaactagtcaggttttcgaaccttgaccgacatgaactgtacacagaattgttccagtactatggagtgctcacaaaggatacttttacaggttaagacaaagggttgactggcctatttatctgatcaagaacatgtcagcaatgtctctttgtgctctaaaattctattatactacaataatatattgtaaagatcctatagctctttttttttgagatggagtttcgcttttgttgcccaggctggagtgcaatggcgcgatcttggctcaccataacctccgcctcccaggttcaagcaattctcctgccttagcctcctgagtagctgggattacaggcgtgcgccactatgcctgactaattttgtagttttagtagagacggggtttctccatgttggtcaggctggtctcaaactcctgacctcaggtgatctgcccgcctcagcctcccaaagtgctggaattacaggcgtgagccaccacgcctggctggatcctatatcttaggtaagacatataacgcagtctaattacatttcacttcaaggctcaatgctattctaactaatgacaagtattttctactaaaccagaaattggtagaaggatttaaataagtaaaagctactatgtactgccttagtgctgatgcctgtgtactgccttaaatgtacctatggcaatttagctctcttgggttcccaaatccctctcacaagaatgtgcagaagaaatcataaaggatcagagattctg

SEQ ID NO: 102 - protein, 43 aa, Homo sapiens
MAARAVFLALSAQLLQARLMKEESPVVSWRLEPEDGTALCFIF

SEQ ID NO: 103 - DNA, 1004 bp, Homo sapiens
cgccaatttagggtctccggtatctcccgctgagctgctctgttcccggcttagaggaccaggagaagggggagctggaggctggagcctgtaacaccgtggctcgtctcactctggatggtggtggcaacagagatggcagcgcagctggagtgttaggagggcggcctgagcggtaggagtggggctggagcagtaagatggcggccagagcggtttttctggcattgtctgcccagctgctccaagccaggctgatgaaggaggagtcccctgtggtgagctggaggttggagcctgaagacggcacagctctgtgcttcatcttctgaggttgtggcagccacggtgatggagacggcagctcaacaggagcaataggaggagatggagtttcactgtgtcagccaggatggtctcgatctcctgacctcgtgatccgcccgccttggccttccaaagtgccgagattacagcgatgtgcattttgtaagcactttggagccactatcaaatgctgtgaagagaaatgtacccagatgtatcattatccttgtgctgcaggagccggctcctttcaggatttcagtcacatcttcctgctttgtccagaacacattgaccaagctcctgaaagatgtaagtttactacgcatagacttttaaacttcaaccaatgtatttactgaaaataacaaatgttgtaaattccctgagtgttattctacttgtattaaaaggtaataatacataatcattaaaatctgagggatcattgccagagattgttggggagggaaatgttatcaacggtttcattgaaattaaatccaaaaagttatttcctcagaaaaatcaaataaagtttgcatgttttttattcttaaaacattttaaaaaccactgtagaatgatgtaaatagggactgtgcagtatttctgacatatactataaaattattaaaaagtcaatcagtattcaacatcttttacactaaaaagcc

SEQ ID NO: 104 - protein, 9 aa, Homo sapiens
WVLTAAHCI

SEQ ID NO: 105 - protein, 263 aa, Homo sapiens
PMWFLVLCLALSLGGTGAAPPIQSRIVGGWECEQHSQPWQAALYHFSTFQCGGILVHRQWVLTAAHCISDNYQLWLGRHNLFDDENTAQFVHVSESFPHPGFNMSLLENHTRQADEDYSHDLMLLRLTEPADTITDAVKVVELPTQEPEVGSTCLASGWGSIEPENFSFPDDLQCVDLKILPNDECEKAHVQKVTDFMLCVGHLEGGKDTCVGDSGGPLMCDGVLQGVTSWGYVPCGTPNKPSVAVRVLSYVKWIEDTIAENS

SEQ ID NO: 106 - protein, 270 aa, Homo sapiens
PMIRTLLLSTLVAGALSCGDPTYPPYVTRVVGGEEARPNSWPWQVSLQYSSNGKWYHTCGGSLIANSWVLTAAHCISSSRTYRVGLGRHNLYVAESGSLAVSVSKIVVHKDWNSNQISKGNDIALLKLANPVSLTDKIQLACLPPAGTILPNNYPCYVTGWGRLQTNGAVPDVLQQGRLLVVDYATCSSSAWWGSSVKTSMICAGGDGVISSCNGDSGGPLNCQASDGRWQVHGIVSFGSRLGCNYYHKPSVFTRVSNYIDWINSVIANN

SEQ ID NO: 107 - protein, 270 aa, Homo sapiens
PMIRTLLLSTLVAGALSCGVSTYAPDMSRMLGGEEARPNSWPWQVSLQYSSNGQWYHTCGGSLIANSWVLTAAHCISSSRIYRVMLGQHNLYVAESGSLAVSVSKIVVHKDWNSNQVSKGNDIALLKLANPVSLTDKIQLACLPPAGTILPNNYPCYVTGWGRLQTNGALPDDLKQGRLLVVDYATCSSSGWWGSTVKTNMICAGGDGVICTCNGDSGGPLNCQASDGRWEVHGIGSLTSVLGCNYYYKPSIFTRVSNYNDWINSVIANN

SEQ ID NO: 108 - protein, 9 aa, Homo sapiens
NIYDLFVWM

SEQ ID NO: 109 - protein, 10 aa, Homo sapiens
YDLFVWMHYY

SEQ ID NO: 110 - protein, 9 aa, Homo sapiens
DLFVWMHYY

SEQ ID NO: 111 - protein, 9 aa, Homo sapiens
DALLGGSEI

SEQ ID NO: 112 - protein, 10 aa, Homo sapiens
GSEIWRDIDF

SEQ ID NO: 113 - protein, 9 aa, Homo sapiens
SEIWRDIDF

SEQ ID NO: 114 - protein, 9 aa, Homo sapiens
EIWRDIDFA

SEQ ID NO: 115 - protein, 10 aa, Homo sapiens
LQEVYPEANA

SEQ ID NO: 116 - protein, 10 aa, Homosapiens
EVYPEANAPI

SEQ ID NO: 117 - protein, 9 aa, Homosapiens
VYPEANAPI

SEQ ID NO: 118 - protein, 8 aa, Homosapiens
YPEANAPI

SEQ ID NO: 119 - protein, 10 aa, Homosapiens
YPEANAPIGH

SEQ ID NO: 120 - protein, 10 aa, Homosapiens
APIGHNRESY

SEQ ID NO: 121 - protein, 9 aa, Homosapiens
PIGHNRESY

SEQ ID NO: 122 - protein, 10 aa, Homosapiens
PIGHNRESYM

SEQ ID NO: 123 - protein, 10 aa, Homosapiens
APIGHNRESY

SEQ ID NO: 124 - protein, 9 aa, Homosapiens
PIGHNRESY

SEQ ID NO: 125 - protein, 8 aa, Homosapiens
ESYMVPFI

SEQ ID NO: 126 - protein, 10 aa, Homosapiens
ESYMVPFIPL

SEQ ID NO: 127 - protein, 9 aa, Homosapiens
SYMVPFIPL

SEQ ID NO: 128 - protein, 10 aa, Homosapiens
SYMVPFIPLY

SEQ ID NO: 129 - protein, 9 aa, Homosapiens
YMVPFIPLY

SEQ ID NO: 130 - protein, 9 aa, Homosapiens
MVPFIPLYR

SEQ ID NO: 131 - protein, 10 aa, Homosapiens
MVPFIPLYRN

SEQ ID NO: 132 - protein, 8 aa, Homosapiens
VPFIPLYR

SEQ ID NO: 133 - protein, 8 aa, Homosapiens
IPLYRNGD

SEQ ID NO: 134 - protein, 10 aa, Homosapiens
IPLYRNGDFF

SEQ ID NO: 135 - protein, 9 aa, Homosapiens
PLYRNGDFF

SEQ ID NO: 136 - protein, 10 aa, Homosapiens
PLYRNGDFFI

SEQ ID NO: 137 - protein, 10 aa, Homosapiens
RNGDFFISSK

SEQ ID NO: 138 - protein, 9 aa, Homosapiens
NGDFFISSK

SEQ ID NO: 139 - protein, 9 aa, Homosapiens
YIKSYLEQA

SEQ ID NO: 140 - protein, 9 aa, Homosapiens
SYLEQASRI

SEQ ID NO: 141 - protein, 10 aa, Homosapiens
EQASRIWSWL

SEQ ID NO: 142 - protein, 9 aa, Homosapiens
QASRIWSWL

SEQ ID NO: 143 - protein, 8 aa, Homosapiens
ASRIWSWL

SEQ ID NO: 144 - protein, 9 aa, Homosapiens
ASRIWSWLL

SEQ ID NO: 145 - protein, 9 aa, Homosapiens
RIWSWLLGA

SEQ ID NO: 146 - protein, 9 aa, Homosapiens
GPAYSGREI

SEQ ID NO: 147 - protein, 10 aa, Homosapiens
GPAYSGREII

SEQ ID NO: 148 - protein, 8 aa, Homosapiens
PAYSGREI

SEQ ID NO: 149 - protein, 9 aa, Homosapiens
PAYSGREII

SEQ ID NO: 150 - protein, 10 aa, Homosapiens
PAYSGREIIY

SEQ ID NO: 151 - protein, 9 aa, Homosapiens
AYSGREIIY

SEQ ID NO: 152 - protein, 9 aa, Homosapiens
GREIIYPNA

SEQ ID NO: 153 - protein, 10 aa, Homosapiens
REIIYPNASL

SEQ ID NO: 154 - protein, 9 aa, Homosapiens
EIIYPNASL

SEQ ID NO: 155 - protein, 10 aa, Homosapiens
EIIYPNASLL

SEQ ID NO: 156 - protein, 8 aa, Homosapiens
IIYPNASL

SEQ ID NO: 157 - protein, 9 aa, Homosapiens
IIYPNASLL

SEQ ID NO: 158 - protein, 10 aa, Homosapiens
IIYPNASLLI

SEQ ID NO: 159 - protein, 8 aa, Homosapiens
YPNASLLI

SEQ ID NO: 160 - protein, 10 aa, Homosapiens
LLIQNIIQND

SEQ ID NO: 161 - protein, 10 aa, Homosapiens
EEATGQFRVY

SEQ ID NO: 162 - protein, 9 aa, Homosapiens
EATGQFRVY

SEQ ID NO: 163 - protein, 9 aa, Homosapiens
YPELPKPSI

SEQ ID NO: 164 - protein, 8 aa, Homosapiens
PELPKPSI

SEQ ID NO: 165 - protein, 9 aa, Homosapiens
RSDSVILNV

SEQ ID NO: 166 - protein, 10 aa, Homosapiens
RSDSVILNVL

SEQ ID NO: 167 - protein, 9 aa, Homosapiens
SDSVILNVL

SEQ ID NO: 168 - protein, 10 aa, Homosapiens
SDSVILNVLY

SEQ ID NO: 169 - protein, 9 aa, Homosapiens
DSVILNVLY

SEQ ID NO: 170 - protein, 10 aa, Homosapiens
VLYGPDAPTI

SEQ ID NO: 171 - protein, 9 aa, Homosapiens
LYGPDAPTI

SEQ ID NO: 172 - protein, 8 aa, Homosapiens
YGPDAPTI

SEQ ID NO: 173 - protein, 10 aa, Homosapiens
GPDAPTISPL

SEQ ID NO: 174 - protein, 9 aa, Homosapiens
PDAPTISPL

SEQ ID NO: 175 - protein, 8 aa, Homosapiens
DAPTISPL

SEQ ID NO: 176 - protein, 9 aa, Homosapiens
APTISPLNT

SEQ ID NO: 177 - protein, 10 aa, Homosapiens
PTISPLNTSY

SEQ ID NO: 178 - protein, 9 aa, Homosapiens
TISPLNTSY

SEQ ID NO: 179 - protein, 10 aa, Homosapiens
PTISPLNTSY

SEQ ID NO: 180 - protein, 9 aa, Homosapiens
TISPLNTSY

SEQ ID NO: 181 - protein, 10 aa, Homosapiens
NTSYRSGENL

SEQ ID NO: 182 - protein, 9 aa, Homosapiens
TSYRSGENL

SEQ ID NO: 183 - protein, 8 aa, Homosapiens
SYRSGENL

SEQ ID NO: 184 - protein, 10 aa, Homosapiens
SYRSGENLNL

SEQ ID NO: 185 - protein, 9 aa, Homosapiens
YRSGENLNL

SEQ ID NO: 186 - protein, 9 aa, Homosapiens
SGENLNLSC

SEQ ID NO: 187 - protein, 10 aa, Homosapiens
ENLNLSCHAA

SEQ ID NO: 188 - protein, 9 aa, Homosapiens
NLNLSCHAA

SEQ ID NO: 189 - protein, 10 aa, Homosapiens
HAASNPPAQY

SEQ ID NO: 190 - protein, 9 aa, Homosapiens
AASNPPAQY

SEQ ID NO: 191 - protein, 10 aa, Homosapiens
NPPAQYSWFV

SEQ ID NO: 192 - protein, 9 aa, Homosapiens
PPAQYSWFV

SEQ ID NO: 193 - protein, 8 aa, Homosapiens
PAQYSWFV

SEQ ID NO: 194 - protein, 9 aa, Homosapiens
FVNGTFQQS

SEQ ID NO: 195 - protein, 10 aa, Homosapiens
RTTVTTITVY

SEQ ID NO: 196 - protein, 9 aa, Homosapiens
TTVTTITVY

SEQ ID NO: 197 - protein, 9 aa, Homosapiens
YAEPPKPFI

SEQ ID NO: 198 - protein, 10 aa, Homosapiens
YAEPPKPFIT

SEQ ID NO: 199 - protein, 8 aa, Homosapiens
AEPPKPFI

SEQ ID NO: 200 - protein, 8 aa, Homosapiens
EPPKPFIT

SEQ ID NO: 201 - protein, 9 aa, Homosapiens
EPPKPFITS

SEQ ID NO: 202 - protein, 8 aa, Homosapiens
PPKPFITS

SEQ ID NO: 203 - protein, 10 aa, Homosapiens
SVTRNDVGPY

SEQ ID NO: 204 - protein, 9 aa, Homosapiens
VTRNDVGPY

SEQ ID NO: 205 - protein, 9 aa, Homosapiens
GPYECGIQN

SEQ ID NO: 206 - protein, 9 aa, Homosapiens
YECGIQNEL

SEQ ID NO: 207 - protein, 9 aa, Homosapiens
GIQNELSVD

SEQ ID NO: 208 - protein, 9 aa, Homosapiens
HSDPVILNV

SEQ ID NO: 209 - protein, 10 aa, Homosapiens
HSDPVILNVL

SEQ ID NO: 210 - protein, 9 aa, Homosapiens
SDPVILNVL

SEQ ID NO: 211 - protein, 10 aa, Homosapiens
SDPVILNVLY

SEQ ID NO: 212 - protein, 8 aa, Homosapiens
DPVILNVL

SEQ ID NO: 213 - protein, 9 aa, Homosapiens
DPVILNVLY

SEQ ID NO: 214 - protein, 10 aa, Homosapiens
ILNVLYGPDD

SEQ ID NO: 215 - protein, 10 aa, Homosapiens
VLYGPDDPTI

SEQ ID NO: 216 - protein, 9 aa, Homosapiens
LYGPDDPTI

SEQ ID NO: 217 - protein, 8 aa, Homosapiens
YGPDDPTI

SEQ ID NO: 218 - protein, 9 aa, Homosapiens
DPTISPSYT

SEQ ID NO: 219 - protein, 10 aa, Homosapiens
PTISPSYTYY

SEQ ID NO: 220 - protein, 9 aa, Homosapiens
TISPSYTYY

SEQ ID NO: 221 - protein, 10 aa, Homosapiens
PTISPSYTYY

SEQ ID NO: 222 - protein, 9 aa, Homosapiens
TISPSYTYY

SEQ ID NO: 223 - protein, 10 aa, Homosapiens
TISPSYTYYR

SEQ ID NO: 224 - protein, 10 aa, Homosapiens
YTYYRPGVNL

SEQ ID NO: 225 - protein, 9 aa, Homosapiens
TYYRPGVNL

SEQ ID NO: 226 - protein, 8 aa, Homosapiens
YYRPGVNL

SEQ ID NO: 227 - protein, 10 aa, Homosapiens
YYRPGVNLSL

SEQ ID NO: 228 - protein, 9 aa, Homosapiens
YRPGVNLSL

SEQ ID NO: 229 - protein, 8 aa, Homosapiens
RPGVNLSL

SEQ ID NO: 230 - protein, 10 aa, Homosapiens
RPGVNLSLSC

SEQ ID NO: 231 - protein, 9 aa, Homosapiens
GVNLSLSCH

SEQ ID NO: 232 - protein, 10 aa, Homosapiens
VNLSLSCHAA

SEQ ID NO: 233 - protein, 9 aa, Homosapiens
NLSLSCHAA

SEQ ID NO: 234 - protein, 10 aa, Homosapiens
HAASNPPAQY

SEQ ID NO: 235 - protein, 9 aa, Homosapiens
AASNPPAQY

SEQ ID NO: 236 - protein, 10 aa, Homosapiens
NPPAQYSWLI

SEQ ID NO: 237 - protein, 9 aa, Homosapiens
PPAQYSWLI

SEQ ID NO: 238 - protein, 8 aa, Homosapiens
PAQYSWLI

SEQ ID NO: 239 - protein, 10 aa, Homosapiens
WLIDGNIQQH

SEQ ID NO: 240 - protein, 9 aa, Homosapiens
LIDGNIQQH

SEQ ID NO: 241 - protein, 10 aa, Homosapiens
LIDGNIQQHT

SEQ ID NO: 242 - protein, 10 aa, Homosapiens
RSDPVTLDVL

SEQ ID NO: 243 - protein, 9 aa, Homosapiens
SDPVTLDVL

SEQ ID NO: 244 - protein, 10 aa, Homosapiens
SDPVTLDVLY

SEQ ID NO: 245 - protein, 8 aa, Homosapiens
DPVTLDVL

SEQ ID NO: 246 - protein, 9 aa, Homosapiens
DPVTLDVLY

SEQ ID NO: 247 - protein, 10 aa, Homosapiens
DVLYGPDTPI

SEQ ID NO: 248 - protein, 9 aa, Homosapiens
VLYGPDTPI

SEQ ID NO: 249 - protein, 10 aa, Homosapiens
PIISPPDSSY

SEQ ID NO: 250 - protein, 9 aa, Homosapiens
IISPPDSSY

SEQ ID NO: 251 - protein, 10 aa, Homosapiens
IISPPDSSYL

SEQ ID NO: 252 - protein, 8 aa, Homosapiens
SPPDSSYL

SEQ ID NO: 253 - protein, 9 aa, Homosapiens
PPDSSYLSG

SEQ ID NO: 254 - protein, 10 aa, Homosapiens
PPDSSYLSGA

SEQ ID NO: 255 - protein, 10 aa, Homosapiens
DSSYLSGANL

SEQ ID NO: 256 - protein, 9 aa, Homosapiens
SSYLSGANL

SEQ ID NO: 257 - protein, 10 aa, Homosapiens
SYLSGANLNL

SEQ ID NO: 258 - protein, 9 aa, Homosapiens
YLSGANLNL

SEQ ID NO: 259 - protein, 9 aa, Homosapiens
NLNLSCHSA

SEQ ID NO: 260 - protein, 10 aa, Homosapiens
NPSPQYSWRI

SEQ ID NO: 261 - protein, 8 aa, Homosapiens
SPQYSWRI

SEQ ID NO: 262 - protein, 9 aa, Homosapiens
WRINGIPQQ

SEQ ID NO: 263 - protein, 9 aa, Homosapiens
RINGIPQQH

SEQ ID NO: 264 - protein, 10 aa, Homosapiens
RINGIPQQHT

SEQ ID NO: 265 - protein, 9 aa, Homosapiens
GIPQQHTQV

SEQ ID NO: 266 - protein, 8 aa, Homosapiens
IPQQHTQV

SEQ ID NO: 267 - protein, 10 aa, Homosapiens
KITPNNNGTY

SEQ ID NO: 268 - protein, 9 aa, Homosapiens
ITPNNNGTY

SEQ ID NO: 269 - protein, 10 aa, Homosapiens
PNNNGTYACF

SEQ ID NO: 270 - protein, 9 aa, Homosapiens
NNNGTYACF

SEQ ID NO: 271 - protein, 8 aa, Homosapiens
NGTYACFV

SEQ ID NO: 272 - protein, 10 aa, Homosapiens
ATGRNNSIVK

SEQ ID NO: 273 - protein, 9 aa, Homosapiens
TGRNNSIVK

SEQ ID NO: 274 - protein, 9 aa, Homosapiens
RNNSIVKSI

SEQ ID NO: 275 - protein, 9 aa, Homosapiens
NSIVKSITV

SEQ ID NO: 276 - protein, 10 aa, Homosapiens
STYRPRPRRY

SEQ ID NO: 277 - protein, 9 aa, Homosapiens
TYRPRPRRY

SEQ ID NO: 278 - protein, 9 aa, Homosapiens
RPRPRRYVE

SEQ ID NO: 279 - protein, 8 aa, Homosapiens
YVEPPEMI

SEQ ID NO: 280 - protein, 10 aa, Homosapiens
MIGPMRPEQF

SEQ ID NO: 281 - protein, 9 aa, Homosapiens
IGPMRPEQF

SEQ ID NO: 282 - protein, 8 aa, Homosapiens
GPMRPEQF

SEQ ID NO: 283 - protein, 10 aa, Homosapiens
KTPEEEMRSH

SEQ ID NO: 284 - protein, 10 aa, Homosapiens
TPEEEMRSHY

SEQ ID NO: 285 - protein, 9 aa, Homosapiens
PEEEMRSHY

SEQ ID NO: 286 - protein, 10 aa, Homosapiens
EMRSHYVAQT

SEQ ID NO: 287 - protein, 9 aa, Homosapiens
SHYVAQTGI

SEQ ID NO: 288 - protein, 10 aa, Homosapiens
YVAQTGILWL

SEQ ID NO: 289 - protein, 9 aa, Homosapiens
VAQTGILWL

SEQ ID NO: 290 - protein, 10 aa, Homosapiens
VAQTGILWLL

SEQ ID NO: 291 - protein, 9 aa, Homosapiens
AQTGILWLL

SEQ ID NO: 292 - protein, 9 aa, Homosapiens
QTGILWLLM

SEQ ID NO: 293 - protein, 10 aa, Homosapiens
QTGILWLLMN

SEQ ID NO: 294 - protein, 10 aa, Homosapiens
GILWLLMNNC

SEQ ID NO: 295 - protein, 9 aa, Homosapiens
ILWLLMNNC

SEQ ID NO: 296 - protein, 8 aa, Homosapiens
LLMNNCFL

SEQ ID NO: 297 - protein, 9 aa, Homosapiens
WLLMNNCFL

SEQ ID NO: 298 - protein, 9 aa, Homosapiens
LWLLMNNCF

SEQ ID NO: 299 - protein, 10 aa, Homosapiens
ILWLLMNNCF

SEQ ID NO: 300 - protein, 9 aa, Homosapiens
ILWLLMNNC

SEQ ID NO: 301 - protein, 10 aa, Homosapiens
GILWLLMNNC

SEQ ID NO: 302 - protein, 10 aa, Homosapiens
QTGILWLLMN

SEQ ID NO: 303 - protein, 9 aa, Homosapiens
QTGILWLLM

SEQ ID NO: 304 - protein, 9 aa, Homosapiens
AQTGILWLL

SEQ ID NO: 305 - protein, 10 aa, Homosapiens
VAQTGILWLL

SEQ ID NO: 306 - protein, 9 aa, Homosapiens
VAQTGILWL

SEQ ID NO: 307 - protein, 10 aa, Homosapiens
YVAQTGILWL

SEQ ID NO: 308 - protein, 9 aa, Homosapiens
SHYVAQTGI

SEQ ID NO: 309 - protein, 9 aa, Homosapiens
SAFPTTINF

SEQ ID NO: 310 - protein, 10 aa, Homosapiens
ASAFPTTINF

SEQ ID NO: 311 - protein, 9 aa, Homosapiens
GASAFPTTI

SEQ ID NO: 312 - protein, 10 aa, Homosapiens
SPQGASAFPT

SEQ ID NO: 313 - protein, 8 aa, Homosapiens
FGKASESL

SEQ ID NO: 314 - protein, 9 aa, Homosapiens
IFGKASESL

SEQ ID NO: 315 - protein, 10 aa, Homosapiens
EIFGKASESL

SEQ ID NO: 316 - protein, 8 aa, Homosapiens
EIFGKASE

SEQ ID NO: 317 - protein, 8 aa, Homosapiens
IKNYKHCF

SEQ ID NO: 318 - protein, 9 aa, Homosapiens
VIKNYKHCF

SEQ ID NO: 319 - protein, 10 aa, Homosapiens
SVIKNYKHCF

SEQ ID NO: 320 - protein, 8 aa, Homosapiens
VIKNYKHC

SEQ ID NO: 321 - protein, 9 aa, Homosapiens
SVIKNYKHC

SEQ ID NO: 322 - protein, 9 aa, Homosapiens
MLESVIKNY

SEQ ID NO: 323 - protein, 10 aa, Homosapiens
EMLESVIKNY

SEQ ID NO: 324 - protein, 9 aa, Homosapiens
AEMLESVIK

SEQ ID NO: 325 - protein, 10 aa, Homosapiens
GPRALIETSY

SEQ ID NO: 326 - protein, 9 aa, Homosapiens
PRALIETSY

SEQ ID NO: 327 - protein, 9 aa, Homosapiens
RALIETSYV

SEQ ID NO: 328 - protein, 10 aa, Homosapiens
ALIETSYVKV

SEQ ID NO: 329 - protein, 9 aa, Homosapiens
LIETSYVKV

SEQ ID NO: 330 - protein, 10 aa, Homosapiens
LIETSYVKVL

SEQ ID NO: 331 - protein, 9 aa, Homosapiens
IETSYVKVL

SEQ ID NO: 332 - protein, 10 aa, Homosapiens
ETSYVKVLHH

SEQ ID NO: 333 - protein, 10 aa, Homosapiens
SYVKVLHHTL

SEQ ID NO: 334 - protein, 9 aa, Homosapiens
YVKVLHHTL

SEQ ID NO: 335 - protein, 9 aa, Homosapiens
KVLHHTLKI

SEQ ID NO: 336 - protein, 9 aa, Homosapiens
PLHERALRE

SEQ ID NO: 337 - protein, 8 aa, Homosapiens
PPLHERAL

SEQ ID NO: 338 - protein, 9 aa, Homosapiens
YPPLHERAL

SEQ ID NO: 339 - protein, 10 aa, Homosapiens
SYPPLHERAL

SEQ ID NO: 340 - protein, 9 aa, Homosapiens
ISYPPLHER

SEQ ID NO: 341 - protein, 10 aa, Homosapiens
HISYPPLHER

SEQ ID NO: 342 - protein, 8 aa, Homosapiens
KIGGEPHI

SEQ ID NO: 343 - protein, 9 aa, Homosapiens
LKIGGEPHI

SEQ ID NO: 344 - protein, 10 aa, Homosapiens
TLKIGGEPHI

SEQ ID NO: 345 - protein, 9 aa, Homosapiens
PLHEWVLRE

SEQ ID NO: 346 - protein, 8 aa, Homosapiens
PPLHEWVL

SEQ ID NO: 347 - protein, 9 aa, Homosapiens
YPPLHEWVL

SEQ ID NO: 348 - protein, 8 aa, Homosapiens
YPPLHEWV

SEQ ID NO: 349 - protein, 9 aa, Homosapiens
SYPPLHEWV

SEQ ID NO: 350 - protein, 10 aa, Homosapiens
ISYPPLHEWV

SEQ ID NO: 351 - protein, 10 aa, Homosapiens
HISYPPLHEW

SEQ ID NO: 352 - protein, 9 aa, Homosapiens
ISGGPHISY

SEQ ID NO: 353 - protein, 10 aa, Homosapiens
KISGGPHISY

SEQ ID NO: 354 - protein, 10 aa, Homosapiens
CWYCRRRNGY

SEQ ID NO: 355 - protein, 9 aa, Homosapiens
WYCRRRNGY

SEQ ID NO: 356 - protein, 9 aa, Homosapiens
YCRRRNGYR

SEQ ID NO: 357 - protein, 9 aa, Homosapiens
RRRNGYRAL

SEQ ID NO: 358 - protein, 10 aa, Homosapiens
RNGYRALMDK

SEQ ID NO: 359 - protein, 9 aa, Homosapiens
NGYRALMDK

SEQ ID NO: 360 - protein, 9 aa, Homosapiens
RALMDKSLH

SEQ ID NO: 361 - protein, 8 aa, Homosapiens
ALMDKSLH

SEQ ID NO: 362 - protein, 10 aa, Homosapiens
RALMDKSLHV

SEQ ID NO: 363 - protein, 9 aa, Homosapiens
ALMDKSLHV

SEQ ID NO: 364 - protein, 10 aa, Homosapiens
YISPEKEEQY

SEQ ID NO: 365 - protein, 9 aa, Homosapiens
ISPEKEEQY

SEQ ID NO: 366 - protein, 9 aa, Homosapiens
SPEKEEQYI

SEQ ID NO: 367 - protein, 8 aa, Homosapiens
PEKEEQYI

SEQ ID NO: 368 - protein, 10 aa, Homosapiens
EKEEQYIAQF

SEQ ID NO: 369 - protein, 9 aa, Homosapiens
KEEQYIAQF

SEQ ID NO: 370 - protein, 10 aa, Homosapiens
QYIAQFTSQF

SEQ ID NO: 371 - protein, 9 aa, Homosapiens
YIAQFTSQF

SEQ ID NO: 372 - protein, 10 aa, Homosapiens
YIAQFTSQFL

SEQ ID NO: 373 - protein, 9 aa, Homosapiens
IAQFTSQFL

SEQ ID NO: 374 - protein, 10 aa, Homosapiens
AQFTSQFLSL

SEQ ID NO: 375 - protein, 9 aa, Homosapiens
QFTSQFLSL

SEQ ID NO: 376 - protein, 9 aa, Homosapiens
SQFLSLQCL

SEQ ID NO: 377 - protein, 10 aa, Homosapiens
VLYPVPLESY

SEQ ID NO: 378 - protein, 9 aa, Homosapiens
LYPVPLESY

SEQ ID NO: 379 - protein, 10 aa, Homosapiens
ESYEDIHGTL

SEQ ID NO: 380 - protein, 10 aa, Homosapiens
YEDIHGTLHL

SEQ ID NO: 381 - protein, 9 aa, Homosapiens
EDIHGTLHL

SEQ ID NO: 382 - protein, 10 aa, Homosapiens
IHGTLHLERL

SEQ ID NO: 383 - protein, 10 aa, Homosapiens
TLHLERLAYL

SEQ ID NO: 384 - protein, 9 aa, Homosapiens
LHLERLAYL

SEQ ID NO: 385 - protein, 8 aa, Homosapiens
HLERLAYL

SEQ ID NO: 386 - protein, 10 aa, Homosapiens
HLERLAYLHA

SEQ ID NO: 387 - protein, 10 aa, Homosapiens
ERLAYLHARL

SEQ ID NO: 388 - protein, 9 aa, Homosapiens
RLAYLHARL

SEQ ID NO: 389 - protein, 10 aa, Homosapiens
RLAYLHARLR

SEQ ID NO: 390 - protein, 8 aa, Homosapiens
LAYLHARL

SEQ ID NO: 391 - protein, 9 aa, Homosapiens
LAYLHARLR

SEQ ID NO: 392 - protein, 10 aa, Homosapiens
AYLHARLREL

SEQ ID NO: 393 - protein, 9 aa, Homosapiens
YLHARLREL

SEQ ID NO: 394 - protein, 10 aa, Homosapiens
YLHARLRELL

SEQ ID NO: 395 - protein, 9 aa, Homosapiens
LHARLRELL

SEQ ID NO: 396 - protein, 8 aa, Homosapiens
HARLRELL

SEQ ID NO: 397 - protein, 9 aa, Homosapiens
HARLRELLC

SEQ ID NO: 398 - protein, 10 aa, Homosapiens
ELLCELGRPS

SEQ ID NO: 399 - protein, 9 aa, Homosapiens
LLCELGRPS

SEQ ID NO: 400 - protein, 10 aa, Homosapiens
QEPALGTTCY

SEQ ID NO: 401 - protein, 9 aa, Homosapiens
EPALGTTCY

SEQ ID NO: 402 - protein, 10 aa, Homosapiens
PEEFLTPKKL

SEQ ID NO: 403 - protein, 9 aa, Homosapiens
EEFLTPKKL

SEQ ID NO: 404 - protein, 9 aa, Homosapiens
FLTPKKLQC

SEQ ID NO: 405 - protein, 10 aa, Homosapiens
FLTPKKLQCV

SEQ ID NO: 406 - protein, 9 aa, Homosapiens
LTPKKLQCV

SEQ ID NO: 407 - protein, 8 aa, Homosapiens
TPKKLQCV

SEQ ID NO: 408 - protein, 9 aa, Homosapiens
TPKKLQCVD

SEQ ID NO: 409 - protein, 10 aa, Homosapiens
KLQCVDLHVI

SEQ ID NO: 410 - protein, 9 aa, Homosapiens
LQCVDLHVI

SEQ ID NO: 411 - protein, 9 aa, Homosapiens
DSQDYYVGK

SEQ ID NO: 412 - protein, 9 aa, Homosapiens
SQDYYVGKK

SEQ ID NO: 413 - protein, 10 aa, Homosapiens
SQDYYVGKKN

SEQ ID NO: 414 - protein, 9 aa, Homosapiens
DYYVGKKNI

SEQ ID NO: 415 - protein, 8 aa, Homosapiens
YYVGKKNI

SEQ ID NO: 416 - protein, 9 aa, Homosapiens
YVGKKNITC

SEQ ID NO: 417 - protein, 10 aa, Homosapiens
YVGKKNITCC

SEQ ID NO: 418 - protein, 10 aa, Homosapiens
WVFGGIDPQS

SEQ ID NO: 419 - protein, 10 aa, Homosapiens
GIDPQSGAAV

SEQ ID NO: 420 - protein, 9 aa, Homosapiens
IDPQSGAAV

SEQ ID NO: 421 - protein, 8 aa, Homosapiens
DPQSGAAV

SEQ ID NO: 422 - protein, 9 aa, Homosapiens
DPQSGAAVV

SEQ ID NO: 423 - protein, 10 aa, Homosapiens
DPQSGAAVVH

SEQ ID NO: 424 - protein, 9 aa, Homosapiens
PQSGAAVVH

SEQ ID NO: 425 - protein, 10 aa, Homosapiens
QSGAAVVHEI

SEQ ID NO: 426 - protein, 9 aa, Homosapiens
SGAAVVHEI

SEQ ID NO: 427 - protein, 8 aa, Homosapiens
GAAVVHEI

SEQ ID NO: 428 - protein, 9 aa, Homosapiens
GAAVVHEIV

SEQ ID NO: 429 - protein, 8 aa, Homosapiens
AAVVHEIV

SEQ ID NO: 430 - protein, 9 aa, Homosapiens
CRDYAVVLR

SEQ ID NO: 431 - protein, 10 aa, Homosapiens
RDYAVVLRKY

SEQ ID NO: 432 - protein, 9 aa, Homosapiens
DYAVVLRKY

SEQ ID NO: 433 - protein, 8 aa, Homosapiens
YAVVLRKY

SEQ ID NO: 434 - protein, 10 aa, Homosapiens
VVLRKYADKI

SEQ ID NO: 435 - protein, 9 aa, Homosapiens
VLRKYADKI

SEQ ID NO: 436 - protein, 10 aa, Homosapiens
VLRKYADKIY

SEQ ID NO: 437 - protein, 8 aa, Homosapiens
LRKYADKI

SEQ ID NO: 438 - protein, 9 aa, Homosapiens
LRKYADKIY

SEQ ID NO: 439 - protein, 10 aa, Homosapiens
RKYADKIYSI

SEQ ID NO: 440 - protein, 9 aa, Homosapiens
KYADKIYSI

SEQ ID NO: 441 - protein, 8 aa, Homosapiens
YADKIYSI

SEQ ID NO: 442 - protein, 10 aa, Homosapiens
MKHPQEMKTY

SEQ ID NO: 443 - protein, 9 aa, Homosapiens
KHPQEMKTY

SEQ ID NO: 444 - protein, 10 aa, Homosapiens
HPQEMKTYSV

SEQ ID NO: 445 - protein, 10 aa, Homosapiens
IDSDPALQKV

SEQ ID NO: 446 - protein, 9 aa, Homosapiens
DSDPALQKV

SEQ ID NO: 447 - protein, 10 aa, Homosapiens
ALQKVNFLPV

SEQ ID NO: 448 - protein, 9 aa, Homosapiens
KVNFLPVLE

SEQ ID NO: 449 - protein, 10 aa, Homosapiens
VNFLPVLEQV

SEQ ID NO: 450 - protein, 9 aa, Homosapiens
NFLPVLEQV

SEQ ID NO: 451 - protein, 10 aa, Homosapiens
PVLEQVGNSD

SEQ ID NO: 452 - protein, 9 aa, Homosapiens
VLEQVGNSD

SEQ ID NO: 453 - protein, 9 aa, Homosapiens
YEREETRQV

SEQ ID NO: 454 - protein, 10 aa, Homosapiens
YEREETRQVY

SEQ ID NO: 455 - protein, 9 aa, Homosapiens
EREETRQVY

SEQ ID NO: 456 - protein, 10 aa, Homosapiens
EREETRQVYM

SEQ ID NO: 457 - protein, 9 aa, Homosapiens
REETRQVYM

SEQ ID NO: 458 - protein, 10 aa, Homosapiens
YMDLNSNIEK

SEQ ID NO: 459 - protein, 9 aa, Homosapiens
DLNSNIEKM

SEQ ID NO: 460 - protein, 10 aa, Homosapiens
SNIEKMITAF

SEQ ID NO: 461 - protein, 9 aa, Homosapiens
NIEKMITAF

SEQ ID NO: 462 - protein, 8 aa, Homosapiens
IEKMITAF

SEQ ID NO: 463 - protein, 10 aa, Homosapiens
RLENYEDQLI

SEQ ID NO: 464 - protein, 9 aa, Homosapiens
LENYEDQLI

SEQ ID NO: 465 - protein, 10 aa, Homosapiens
LENYEDQLII

SEQ ID NO: 466 - protein, 9 aa, Homosapiens
ENYEDQLII

SEQ ID NO: 467 - protein, 10 aa, Homosapiens
ENYEDQLIIL

SEQ ID NO: 468 - protein, 9 aa, Homosapiens
NYEDQLIIL

SEQ ID NO: 469 - protein, 10 aa, Homosapiens
NYEDQLIILT

SEQ ID NO: 470 - protein, 9 aa, Homosapiens
YEDQLIILT

SEQ ID NO: 471 - protein, 10 aa, Homosapiens
YEDQLIILTM

SEQ ID NO: 472 - protein, 9 aa, Homosapiens
EDQLIILTM

SEQ ID NO: 473 - protein, 10 aa, Homosapiens
IILTMELQKT

SEQ ID NO: 474 - protein, 9 aa, Homosapiens
ILTMELQKT

SEQ ID NO: 475 - protein, 9 aa, Homosapiens
KLTNNKEVE

SEQ ID NO: 476 - protein, 10 aa, Homosapiens
KLTNNKEVEL

SEQ ID NO: 477 - protein, 9 aa, Homosapiens
LTNNKEVEL

SEQ ID NO: 478 - protein, 10 aa, Homosapiens
KEVELEELKK

SEQ ID NO: 479 - protein, 9 aa, Homosapiens
EVELEELKK

SEQ ID NO: 480 - protein, 10 aa, Homosapiens
EVELEELKKV

SEQ ID NO: 481 - protein, 9 aa, Homosapiens
VELEELKKV

SEQ ID NO: 482 - protein, 10 aa, Homosapiens
ETSDMTLELK

SEQ ID NO: 483 - protein, 9 aa, Homosapiens
TSDMTLELK

SEQ ID NO: 484 - protein, 9 aa, Homosapiens
NKKQEERML

SEQ ID NO: 485 - protein, 10 aa, Homosapiens
ERMLTQIENL

SEQ ID NO: 486 - protein, 9 aa, Homosapiens
RMLTQIENL

SEQ ID NO: 487 - protein, 8 aa, Homosapiens
MLTQIENL

SEQ ID NO: 488 - protein, 10 aa, Homosapiens
MLTQIENLQE

SEQ ID NO: 489 - protein, 10 aa, Homosapiens
ENLQETETQL

SEQ ID NO: 490 - protein, 9 aa, Homosapiens
NLQETETQL

SEQ ID NO: 491 - protein, 10 aa, Homosapiens
NLQETETQLR

SEQ ID NO: 492 - protein, 10 aa, Homosapiens
TQLRNELEYV

SEQ ID NO: 493 - protein, 9 aa, Homosapiens
QLRNELEYV

SEQ ID NO: 494 - protein, 10 aa, Homosapiens
NELEYVREEL

SEQ ID NO: 495 - protein, 9 aa, Homosapiens
ELEYVREEL

SEQ ID NO: 496 - protein, 8 aa, Homosapiens
LEYVREEL

SEQ ID NO: 497 - protein, 10 aa, Homosapiens
EYVREELKQK

SEQ ID NO: 498 - protein, 9 aa, Homosapiens
YVREELKQK

SEQ ID NO: 499 - protein, 10 aa, Homosapiens
LLEEVEKAKV

SEQ ID NO: 500 - protein, 9 aa, Homosapiens
LEEVEKAKV

SEQ ID NO: 501 - protein, 10 aa, Homosapiens
LEEVEKAKVI

SEQ ID NO: 502 - protein, 9 aa, Homosapiens
EEVEKAKVI

SEQ ID NO: 503 - protein, 10 aa, Homosapiens
KAKVIADEAV

SEQ ID NO: 504 - protein, 10 aa, Homosapiens
KVIADEAVKL

SEQ ID NO: 505 - protein, 9 aa, Homosapiens
VIADEAVKL

SEQ ID NO: 506 - protein, 8 aa, Homosapiens
IADEAVKL

SEQ ID NO: 507 - protein, 9 aa, Homosapiens
KEIDKRCQH

SEQ ID NO: 508 - protein, 10 aa, Homosapiens
KEIDKRCQHK

SEQ ID NO: 509 - protein, 9 aa, Homosapiens
EIDKRCQHK

SEQ ID NO: 510 - protein, 10 aa, Homosapiens
EIDKRCQHKI

SEQ ID NO: 511 - protein, 9 aa, Homosapiens
IDKRCQHKI

SEQ ID NO: 512 - protein, 8 aa, Homosapiens
DKRCQHKI

SEQ ID NO: 513 - protein, 9 aa, Homosapiens
KRCQHKIAE

SEQ ID NO: 514 - protein, 10 aa, Homosapiens
KRCQHKIAEM

SEQ ID NO: 515 - protein, 9 aa, Homosapiens
RCQHKIAEM

SEQ ID NO: 516 - protein, 10 aa, Homosapiens
QHKIAEMVAL

SEQ ID NO: 517 - protein, 9 aa, Homosapiens
HKIAEMVAL

SEQ ID NO: 518 - protein, 8 aa, Homosapiens
KIAEMVAL

SEQ ID NO: 519 - protein, 10 aa, Homosapiens
QEQSSLRASL

SEQ ID NO: 520 - protein, 9 aa, Homosapiens
EQSSLRASL

SEQ ID NO: 521 - protein, 8 aa, Homosapiens
QSSLRASL

SEQ ID NO: 522 - protein, 10 aa, Homosapiens
SLRASLEIEL

SEQ ID NO: 523 - protein, 9 aa, Homosapiens
LRASLEIEL

SEQ ID NO: 524 - protein, 8 aa, Homosapiens
RASLEIEL

SEQ ID NO: 525 - protein, 10 aa, Homosapiens
ASLEIELSNL

SEQ ID NO: 526 - protein, 9 aa, Homosapiens
SLEIELSNL

SEQ ID NO: 527 - protein, 10 aa, Homosapiens
SLEIELSNLK

SEQ ID NO: 528 - protein, 9 aa, Homosapiens
LEIELSNLK

SEQ ID NO: 529 - protein, 9 aa, Homosapiens
EIELSNLKA

SEQ ID NO: 530 - protein, 10 aa, Homosapiens
ELSNLKAELL

SEQ ID NO: 531 - protein, 9 aa, Homosapiens
LSNLKAELL

SEQ ID NO: 532 - protein, 10 aa, Homosapiens
SNLKAELLSV

SEQ ID NO: 533 - protein, 9 aa, Homosapiens
NLKAELLSV

SEQ ID NO: 534 - protein, 10 aa, Homosapiens
NLKAELLSVK

SEQ ID NO: 535 - protein, 9 aa, Homosapiens
LKAELLSVK

SEQ ID NO: 536 - protein, 10 aa, Homosapiens
LKAELLSVKK

SEQ ID NO: 537 - protein, 9 aa, Homosapiens
KAELLSVKK

SEQ ID NO: 538 - protein, 9 aa, Homosapiens
AELLSVKKQ

SEQ ID NO: 539 - protein, 10 aa, Homosapiens
EKKDKKTQTF

SEQ ID NO: 540 - protein, 9 aa, Homosapiens
KKDKKTQTF

SEQ ID NO: 541 - protein, 8 aa, Homosapiens
KDKKTQTF

SEQ ID NO: 542 - protein, 10 aa, Homosapiens
LLETPDIYWK

SEQ ID NO: 543 - protein, 9 aa, Homosapiens
LETPDIYWK

SEQ ID NO: 544 - protein, 10 aa, Homosapiens
LETPDIYWKL

SEQ ID NO: 545 - protein, 9 aa, Homosapiens
ETPDIYWKL

SEQ ID NO: 546 - protein, 8 aa, Homosapiens
TPDIYWKL

SEQ ID NO: 547 - protein, 9 aa, Homosapiens
SKAVPSQTV

SEQ ID NO: 548 - protein, 8 aa, Homosapiens
KAVPSQTV

SEQ ID NO: 549 - protein, 10 aa, Homosapiens
VPSQTVSRNF

SEQ ID NO: 550 - protein, 10 aa, Homosapiens
QTVSRNFTSV

SEQ ID NO: 551 - protein, 9 aa, Homosapiens
TVSRNFTSV

SEQ ID NO: 552 - protein, 10 aa, Homosapiens
TVSRNFTSVD

SEQ ID NO: 553 - protein, 10 aa, Homosapiens
SVDHGISKDK

SEQ ID NO: 554 - protein, 10 aa, Homosapiens
SKDKRDYLWT

SEQ ID NO: 555 - protein, 9 aa, Homosapiens
KRDYLWTSA

SEQ ID NO: 556 - protein, 10 aa, Homosapiens
KRDYLWTSAK

SEQ ID NO: 557 - protein, 9 aa, Homosapiens
RDYLWTSAK

SEQ ID NO: 558 - protein, 9 aa, Homosapiens
YLWTSAKNT

SEQ ID NO: 559 - protein, 10 aa, Homosapiens
YLWTSAKNTL

SEQ ID NO: 560 - protein, 8 aa, Homosapiens
WTSAKNTL

SEQ ID NO: 561 - protein, 10 aa, Homosapiens
KNTLSTPLPK

SEQ ID NO: 562 - protein, 9 aa, Homosapiens
NTLSTPLPK

SEQ ID NO: 563 - protein, 9 aa, Homosapiens
KRDYLWTSA

SEQ ID NO: 564 - protein, 10 aa, Homosapiens
KRDYLWTSAK

SEQ ID NO: 565 - protein, 9 aa, Homosapiens
RDYLWTSAK

SEQ ID NO: 566 - protein, 9 aa, Homosapiens
YLWTSAKNT

SEQ ID NO: 567 - protein, 8 aa, Homosapiens
SAKNTLST

SEQ ID NO: 568 - protein, 10 aa, Homosapiens
KNTLSTPLPK

SEQ ID NO: 569 - protein, 9 aa, Homosapiens
NTLSTPLPK

SEQ ID NO: 570 - protein, 10 aa, Homosapiens
TLSTPLPKAY

SEQ ID NO: 571 - protein, 9 aa, Homosapiens
LSTPLPKAY

SEQ ID NO: 572 - protein, 8 aa, Homosapiens
DAFARRPT

SEQ ID NO: 573 - protein, 9 aa, Homosapiens
FARRPTVGA

SEQ ID NO: 574 - protein, 10 aa, Homosapiens
ARRPTVGAQI

SEQ ID NO: 575 - protein, 9 aa, Homosapiens
RRPTVGAQI

SEQ ID NO: 576 - protein, 8 aa, Homosapiens
RPTVGAQI

SEQ ID NO: 577 - protein, 9 aa, Homosapiens
VGAQIPEKI

SEQ ID NO: 578 - protein, 8 aa, Homosapiens
GAQIPEKI

SEQ ID NO: 579 - protein, 10 aa, Homosapiens
AQIPEKIQKA

SEQ ID NO: 580 - protein, 9 aa, Homosapiens
QIPEKIQKA

SEQ ID NO: 581 - protein, 10 aa, Homosapiens
QIPEKIQKAF

SEQ ID NO: 582 - protein, 8 aa, Homosapiens
IPEKIQKA

SEQ ID NO: 583 - protein, 9 aa, Homosapiens
IPEKIQKAF

SEQ ID NO: 584 - protein, 8 aa, Homosapiens
PEKIQKAF

SEQ ID NO: 585 - protein, 9 aa, Homosapiens
ETNNKKKEF

SEQ ID NO: 586 - protein, 8 aa, Homosapiens
TNNKKKEF

SEQ ID NO: 587 - protein, 10 aa, Homosapiens
KEFEETAKKV

SEQ ID NO: 588 - protein, 9 aa, Homosapiens
EFEETAKKV

SEQ ID NO: 589 - protein, 8 aa, Homosapiens
TAKKVRRA

SEQ ID NO: 590 - protein, 9 aa, Homosapiens
ETAKKVRRA

SEQ ID NO: 591 - protein, 9 aa, Homosapiens
AKKVRRAIE

SEQ ID NO: 592 - protein, 10 aa, Homosapiens
KKVRRAIEQL

SEQ ID NO: 593 - protein, 9 aa, Homosapiens
KVRRAIEQL

SEQ ID NO: 594 - protein, 10 aa, Homosapiens
KVRRAIEQLA

SEQ ID NO: 595 - protein, 8 aa, Homosapiens
VRRAIEQL

SEQ ID NO: 596 - protein, 8 aa, Homosapiens
SPVVSWRL

SEQ ID NO: 597 - protein, 9 aa, Homosapiens
KEESPVVSW

SEQ ID NO: 598 - protein, 9 aa, Homosapiens
LMKEESPVV

SEQ ID NO: 599 - protein, 10 aa, Homosapiens
RLMKEESPVV

SEQ ID NO: 600 - protein, 9 aa, Homosapiens
RLMKEESPV

SEQ ID NO: 601 - protein, 9 aa, Homosapiens
LLQARLMKE

SEQ ID NO: 602 - protein, 10 aa, Homosapiens
QLLQARLMKE

SEQ ID NO: 603 - protein, 16 aa, Homosapiens
FLKDHRISTFKNWPFL

SEQ ID NO: 604 - protein, 33 aa, Homosapiens
KHSSGCAFLSVKKQFEELTLGEFLKLDRERAKN

SEQ ID NO: 605 - protein, 12 aa, Homosapiens
KVRRAIEQLAAM

SEQ ID NO: 606 - protein, 18 aa, Homosapiens
VAQTGILWLLMNNCFLNL

SEQ ID NO: 607 - protein, 11 aa, Homosapiens
FLALSAQLLQA

SEQ ID NO: 608 - protein, 10 aa, Homosapiens
RLMKEESPVV

SEQ ID NO: 609 - protein, 26 aa, Homosapiens
AARAVFLALSAQLLQARLMKEESPVV

SEQ ID NO: 610 - protein, 10 aa, Homosapiens
RLEPEDGTAL